(19) United States
(12) Patent Application Publication
(10) Pub. No.: US 2023/0397495 A1
(43) Pub. Date: Dec. 7, 2023

(54) ORGANIC ELECTROLUMINESCENT DEVICE

(71) Applicant: Merck Patent GmbH, Darmstadt (DE)

(72) Inventors: Amir Hossain PARHAM, Darmstadt (DE); Christian EHRENREICH, Darmstadt (DE)

(21) Appl. No.: 18/032,358

(22) PCT Filed: Oct. 25, 2021
(86) PCT No.: PCT/EP2021/079450

§ 371 Date: Apr. 18, 2023

(30) Foreign Application Priority Data
Oct. 27, 2020 (EP) 20204008.5

(51) Int. Cl. H10K 85/60; C09K 11/02; C09K 11/06; H10K 85/30

(52) U.S. Cl. CPC H10K 85/6574; C09K 11/02; C09K 11/06; H10K 85/654; H10K 85/6572; C07B 2200/05; H10K 85/6576; H10K 85/342; H10K 85/346; H10K 85/348; H10K 85/344; H10K 85/622

(57) ABSTRACT

The present invention relates to an organic electroluminescent device comprising a light-emitting layer comprising an electron-transporting host material and a hole-transporting host material, and to a formulation comprising a mixture of the host materials and to a mixture comprising the host materials. The electron-transporting host material corresponds to a compound of the formula (1) comprising diazadibenzofuran or diazadibenzothiophene units. The hole-transporting host material corresponds to a compound of the formula (2) from the class of the biscarbazoles or the derivatives thereof.

### SUBJECT-MATTER OF THE INVENTION

The present invention relates to an organic electroluminescent device comprising a light-emitting layer comprising an electron-transporting host material and a hole-transporting host material, and to a formulation comprising a mixture of the host materials and to a mixture comprising the host materials. The electron-transporting host material corresponds to a compound of the formula (1) comprising diazadibenzofuran or diazadibenzothiophene units. The hole-transporting host material corresponds to a compound of the formula (2) from the class of the biscarbazoles or the derivatives thereof.

### BACKGROUND OF THE INVENTION

The structure of organic electroluminescent devices (e.g. OLEDs—organic light-emitting diodes or OLECs—organic light-emitting electrochemical cells) in which organic semiconductors are used as functional materials has long been known. Emitting materials used here, aside from fluorescent emitters, are increasingly organometallic complexes which exhibit phosphorescence rather than fluorescence. In general terms, however, there is still a need for improvement in OLEDs, especially also in OLEDs which exhibit triplet emission (phosphorescence), for example with regard to efficiency, operating voltage and lifetime.

The properties of organic electroluminescent devices are not only determined by the emitters used. Also of particular significance here are especially the other materials used, such as host and matrix materials, hole blocker materials, electron transport materials, hole transport materials and electron or exciton blocker materials, and among these especially the host or matrix materials. Improvements to these materials can lead to distinct improvements to electroluminescent devices.

Host materials for use in organic electronic devices are well known to the person skilled in the art. The term “matrix material” is also frequently used in the prior art when what is meant is a host material for phosphorescent emitters. This use of the term is also applicable to the present invention. In the meantime, a multitude of host materials has been developed both for fluorescent and for phosphorescent electronic devices.

A further means of improving the performance data of electronic devices, especially of organic electroluminescent devices, is to use combinations of two or more materials, especially host materials or matrix materials.

U.S. Pat. No. 6,392,250 B1 discloses the use of a mixture consisting of an electron transport material, a hole transport material and a fluorescent emitter in the emission layer of an OLED. With the aid of this mixture, it was possible to improve the lifetime of the OLED compared to the prior art.

U.S. Pat. No. 6,803,720 B1 discloses the use of a mixture comprising a phosphorescent emitter and a hole transport material and an electron transport material in the emission layer of an OLED. Both the hole transport material and the electron transport material are small organic molecules.

WO2015169412 describes specific azadibenzofuran compounds or azadibenzothiophene compounds as host material for organic electroluminescent devices.

WO2015105315 and WO2015105316 disclose heterocycles comprising two nitrogen atoms and the use thereof in organic electroluminescent devices as a host material, optionally in combination with a further host material.

US2016013421 discloses benzothienopyrimidine compounds and the use thereof in an organic electroluminescent device as a host material.

US2016072078 describes electron-transporting host materials comprising carbazole units.

US2017200903 describes diazadibenzofuran compounds and diazadibenzothiophene compounds and the use thereof in an organic electroluminescent device, in particular as an electron transport material.

KR20160046077 and KR20160046078 describe an organic light-emitting device comprising a light-emitting layer comprising specific emitters in combination with various host materials.

KR20170113320 disclose specifically substituted dibenzofuran compounds and the use thereof as host material together with a biscarbazole in an organic electroluminescent device.

WO17109637 describes benzothienopyrimidine compounds and benzofuropyrimidine compounds and the use thereof in an organic electroluminescent device as a host material, wherein the benzothienopyrimidine compounds and benzofuropyrimidine compounds each bear two substituents comprising a furan, thiophene or pyrrole unit.

WO17186760 discloses diazacarbazole compounds and the use thereof in an organic electroluminescent device as a host material, electron transport material and hole blocker material.

WO18060218 discloses diazadibenzofuran compounds and diazadibenzothiophene compounds and the use thereof in an organic electroluminescent device, wherein the benzothienopyrimidine compounds and benzofuropyrimidine compounds each bear at least one substituent comprising an N-bonded carbazole unit.

KR20180010165 describes diazadibenzofuran compounds and diazadibenzothiophene compounds and the use thereof in an organic electroluminescent device.

WO18060307 describes diazadibenzofuran compounds and diazadibenzothiophene compounds and the use thereof in an organic electroluminescent device.

WO18234932, WO19059577, WO19229583 and WO19229584 describe diazadibenzofuran and diazadibenzothiophene derivatives which may be used as host materials in an electroluminescent device.

US2020161564 describes diazadibenzofuran compounds and diazadibenzothiophene compounds and the use thereof in an organic electroluminescent device.

US20200010476 disclose heterocyclic compounds, and describe the use thereof in organic electroluminescent devices.

WO20067657 describes a composition of materials and the use thereof in optoelectronic devices.

However, there is still need for improvement in the case of use of these materials or in the case of use of mixtures of the materials, especially in relation to efficiency, operating voltage and/or lifetime of the organic electroluminescent device.

The problem addressed by the present invention is therefore that of providing a combination of host materials which are suitable for use in an organic electroluminescent device, especially in a fluorescent or phosphorescent OLED, and lead to good device properties, especially with regard to an improved lifetime, and that of providing the corresponding electroluminescent device.

### SUMMARY OF THE INVENTION

It has now been found that this problem is solved, and the disadvantages from the prior art are eliminated, by the combination of at least one compound of the formula (1) as first host material and at least one hole-transporting compound of the formula (2) as second host material in a light-emitting layer of an organic electroluminescent device. The use of such a material combination for production of the light-emitting layer in an organic electroluminescent device leads to very good properties of these devices, especially with regard to lifetime, especially with equal or improved efficiency and/or operating voltage.

The advantages are especially also manifested in the presence of a light-emitting component in the emission layer, especially in the case of combination with emitters of the formula (3) or emitters of the formulae (I) to (VIII) at concentrations between 2% and 15% by weight or in combination with monoamines of formula (4) in the hole injection layer and/or hole transport layer.

The present invention therefore first provides an organic electroluminescent device comprising an anode, a cathode and at least one organic layer containing at least one light-emitting layer, wherein the at least one light-emitting layer contains at least one compound of the formula (1) as host material 1 and at least one compound of the formula (2) as host material 2

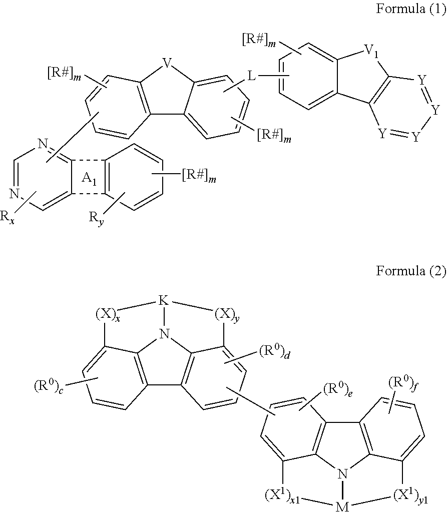

where the symbols and indices used are as follows:
ring A₁ in formula (1) conforms to the formula (1A)

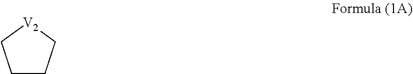

V is O or S;
V₁ is O, S, C(R¹)₂ or N-A;
V₂ is O or S;
Y is the same or different at each instance and is independently CH, CR or CA, or two adjacent Y groups together form a group of the formula (1B)

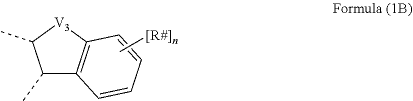

and the remaining Y groups are independently CH or CR, where V₃ is O, S, C(R¹)₂ or N-A and the dotted bonds show the linkage of this group to the remainder of the formula (1);
Rx is L₁-R*;
Ry is L₂-R*;
R* independently at each instance is an aromatic or heteroaromatic ring system which has 6 to 14 ring atoms and may be substituted by one or more R radicals;
L, L₁, L₂ are each independently a bond or a phenylene group which may each be substituted by one or more R radicals;
A is the same or different at each instance and is an aromatic or heteroaromatic ring system which has 6 to 30 ring atoms and may be substituted by one or more R radicals;
n is 0, 1, 2, 3 or 4;
m is 0, 1, 2 or 3;
R # is D or an aryl group which has 6 to 12 carbon atoms and may be substituted by one or more R radicals;
R¹ is the same or different at each instance and is an alkyl group having 1 to 20 carbon atoms, phenyl which may be substituted by one or more R radicals, or the two R¹ radicals together with the carbon atom to which they bind form a spiro compound;
R is the same or different at each instance and is selected from D, F, CN, a straight-chain alkyl group having 1 to 20 carbon atoms or a branched or cyclic alkyl group having 3 to 20 carbon atoms, where one or more nonadjacent CH₂ groups may be replaced by R²C═CR², O or S and where one or more hydrogen atoms may be replaced by D, F, or CN;
R² is the same or different at each instance and is selected from the group consisting of H, D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
K, M are each independently an unsubstituted or partly or fully deuterated or R*-monosubstituted aromatic ring system having 6 to 40 ring atoms when x and y are 0 and when x1 and y1 are 0, or
K, M each independently together with X or X¹ form a heteroaromatic ring system having 14 to 40 ring atoms when the value of x, x1, y and/or y1 is 1;
x, x1 at each instance are each independently 0 or 1;
y, y1 at each instance are each independently 0 or 1;
X and X¹ at each instance are each independently a bond or C(R⁺)₂;
R⁰ at each instance is independently an unsubstituted or partly or fully deuterated aromatic ring system having 6 to 18 carbon atoms;
R⁺ at each instance is independently a straight-chain or branched alkyl group having 1 to 4 carbon atoms and
c, d, e and f are independently 0 or 1.

The invention further provides a process for producing the organic electroluminescent devices and mixtures comprising at least one compound of the formula (1) and at least one compound of the formula (2), specific material combinations and formulations that contain such mixtures or material combinations. The corresponding preferred embodiments as described hereinafter likewise form part of the subject-matter of the present invention. The surprising and advantageous effects are achieved through specific selection of the compounds of the formula (1) and the compounds of the formula (2). The surprising and advantageous effects are achieved through specific selection of the compounds of the formula (1) and the compounds of the formula (2), preferably together with specific emitters in the light-emitting layer and with specific monoamines in the hole injection layer and/or hole transport layer.

### DETAILED DESCRIPTION OF THE INVENTION

The organic electroluminescent device of the invention is, for example, an organic light-emitting transistor (OLET), an organic field quench device (OFQD), an organic light-emitting electrochemical cell (OLEC, LEC, LEEC), an organic laser diode (O-laser) or an organic light-emitting diode (OLED). The organic electroluminescent device of the invention is especially an organic light-emitting diode or an organic light-emitting electrochemical cell. The device of the invention is more preferably an OLED.

The organic layer of the device of the invention that comprises the light-emitting layer comprising the material combination of at least one compound of the formula (1) and at least one compound of the formula (2), as described or described above or hereinafter, preferably comprises, in addition to this light-emitting layer (EML), a hole injection layer (HIL), a hole transport layer (HTL), an electron transport layer (ETL), an electron injection layer (EIL) and/or a hole blocker layer (HBL). It is also possible for the device of the invention to include multiple layers from this group selected from EML, HIL, HTL, ETL, EIL and HBL.

However, the device may also comprise inorganic materials or else layers formed entirely from inorganic materials.

It is preferable when the organic layer of the device of the invention comprises a hole injection layer and/or the hole transport layer wherein the hole-injecting material and hole-transporting material is a monoamine that does not contain a carbazole unit. A suitable selection of monoamine compounds and preferred monoamines is described hereinbelow.

It is preferable when the light-emitting layer comprising at least one compound of the formula (1) and at least one compound of the formula (2) is a phosphorescent layer which is characterized in that it comprises, in addition to the host material combination of compounds of the formula (1) and formula (2), as described above, at least one phosphorescent emitter. A suitable selection of emitters and preferred emitters is described hereinafter.

An aryl group in the context of this invention contains 6 to 40 aromatic ring atoms, preferably carbon atoms. A heteroaryl group in the context of this invention contains 5 to 40 aromatic ring atoms, where the ring atoms include carbon atoms and at least one heteroatom, with the proviso that the sum total of carbon atoms and heteroatoms adds up to at least 5. The heteroatoms are preferably selected from N, O and/or S. An aryl group or heteroaryl group is understood here to mean either a simple aromatic cycle, i.e. phenyl, derived from benzene, or a simple heteroaromatic cycle, for example derived from pyridine, pyrimidine or thiophene, or a fused aryl or heteroaryl group, for example derived from naphthalene, anthracene, phenanthrene, quinoline or isoquinoline. An aryl group having 6 to 18 carbon atoms is therefore preferably phenyl, naphthyl or phenanthryl, with no restriction in the attachment of the aryl group as substituent. The aryl or heteroaryl group in the context of this invention may bear one or more radicals R, where the substituent R is described below.

An aromatic ring system in the context of this invention contains 6 to 40 ring atoms. The aromatic ring system also includes aryl groups as described above.

A heteroaromatic ring system in the context of this invention contains 5 to 40 ring atoms and at least one heteroatom. A preferred heteroaromatic ring system has 10 to 40 ring atoms and at least one heteroatom. The heteroaromatic ring system also includes heteroaryl groups as described above. The heteroatoms in the heteroaromatic ring system are preferably selected from N, O and/or S.

An aromatic or heteroaromatic ring system in the context of this invention is understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic or heteroaromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, are likewise encompassed by the definition of the aromatic or heteroaromatic ring system.

An aromatic or heteroaromatic ring system which has 5-40 ring atoms and may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system having 6 to 18 carbon atoms as ring atoms is preferably selected from phenyl, 1,2-biphenyl, 1,3-biphenyl, 1,4-biphenyl, fluorenyl, naphthyl, phenanthryl and triphenylenyl, which may be substituted by one or more R radicals, where the substituent R is described hereinafter.

A preferred heteroaromatic ring system having 6 to 18 ring atoms is preferably selected from dibenzofuranyl and dibenzothiophenyl, which may be substituted by one or more R radicals, where the substituent R is described hereinafter.

The abbreviation A is the same or different at each instance and independently denotes an aromatic or heteroaromatic ring system which has 6 to 30 carbon atoms and may be substituted by one or more R radicals, where the R radical is defined as described above or hereinafter.

A cyclic alkyl group in the context of this invention is understood to mean a monocyclic, bicyclic or polycyclic group.

In the context of the present invention, a straight-chain, branched or cyclic C₁- to C₂₀-alkyl group is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals.

When the host materials of the light-emitting layer comprising at least one compound of the formula (1) as described above or described as preferred hereinafter and at least one compound of the formula (2) as described above or described hereinafter are used for a phosphorescent emitter, it is preferable when the triplet energy thereof is not significantly less than the triplet energy of the phosphorescent emitter. In respect of the triplet level, it is preferably the case that T₁(emitter)−T₁(matrix)≤0.2 eV, more preferably ≤0.15 eV, most preferably ≤0.1 eV. T₁(matrix) here is the triplet level of the matrix material in the emission layer, this condition being applicable to each of the two matrix materials, and T₁(emitter) is the triplet level of the phosphorescent emitter. If the emission layer contains more than two matrix materials, the abovementioned relationship is preferably also applicable to every further matrix material.

There follows a description of the host material 1 and its preferred embodiments that is/are present in the device of the invention. The preferred embodiments of the host material 1 of the formula (1) are also applicable to the mixture and/or formulation of the invention.

In compounds of the formula (1), the attachment of the diaza group is possible in any of positions 1, 2, 3 and 4 on the central heterocycle. Preference is given to the attachment of the diaza group in position 1 on the central heterocycle.

A preferred embodiment of the compounds of the formula (1) is therefore compounds of the formula (1a):

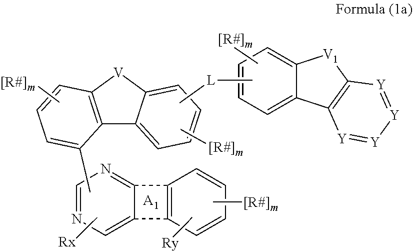

where the symbols Y, V, V₁, R #, m, Rx, Ry, L and ring A₁ used have a definition given above or specified as preferred hereinafter.

The invention further provides the organic electroluminescent device as described above, wherein the host material 1 conforms to the formula (1 a) as described above.

In compounds of the formula (1) or (1a), V₁ is O, S, C(R¹)₂ or N-A, where R¹ and A have a definition given above or specified as preferred hereinafter.

A preferred embodiment of the compounds of the formulae (1) and (1a) is therefore compounds in which V₁ is NA, which are described by the formula (1b),

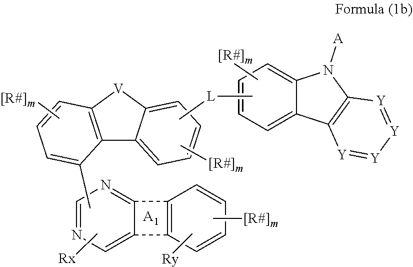

where the symbols Y, V, R #, m, Rx, Ry, L and ring A₁ used have a definition given above or specified as preferred hereinafter.

A preferred embodiment of the compounds of the formulae (1) and (1a) is therefore compounds in which V₁ is O, which are described by the formula (1c),

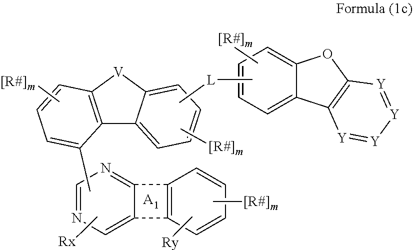

where the symbols Y, V, R #, m, Rx, Ry, L and ring A₁ used have a definition given above or specified as preferred hereinafter.

A preferred embodiment of the compounds of the formulae (1) and (1a) is therefore compounds in which V₁ is S, which are described by the formula (1d),

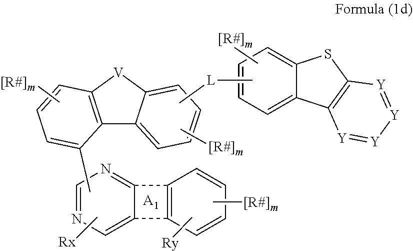

where the symbols Y, V, R #, m, Rx, Ry, L and ring A₁ used have a definition given above or specified as preferred hereinafter.

A preferred embodiment of the compounds of the formulae (1) and (1a) is therefore compounds in which V₁ is C(R¹)₂, which are described by the formula (1e),

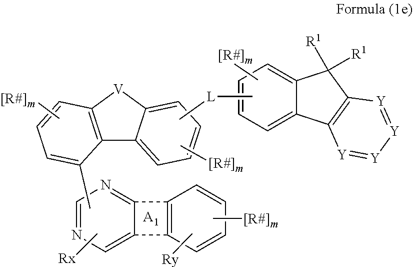

where the symbols Y, V, R #, m, Rx, Ry, L and ring A₁ used have a definition given above or specified as preferred hereinafter.

In compounds of the formulae (1), (1a) and (1b), Y at each instance is the same or different and is independently CH, CR or CA, or two adjacent Y groups together are a group of the formula (16), as described above. Preferably, V₃ is C(R¹)₂ or N-A in formula (16), where R¹ and A have a definition given above or one given with particular preference. More preferably, V₃ is C(R¹)₂ in formula (16), where R¹ has a definition given above or one given with particular preference. If V₃ is C(R¹)₂, R¹ is preferably the same and is an alkyl group having 1 to 10 carbon atoms or phenyl. If V₃ is C(R¹)₂, R¹ is more preferably the same and is a methyl group. If V₃ is N-A, A is preferably an aromatic ring system which has 6 to 18 ring atoms and may be substituted by one or more R radicals. If V₃ is N-A, A is more preferably phenyl which may be substituted by one or more R radicals. In formula (16), R # is preferably D or phenyl. If R # in the formula (1B) is D, n is preferably 1, 2, 3 or 4, more preferably 4. If R # in the formula (1B) is phenyl, n is preferably 1. In formula (16), n is most preferably 0.

In compounds of the formulae (1), (1a), (1b), (1c), (1d) and (1e), Y is the same or different at each instance and is preferably CH, CR or CA.

In compounds of the formulae (1), (1a), (1b), (1c), (1d) and (1e), Y is the same or different at each instance and is more preferably CH or CA.

If Y in compounds of the formulae (1), (1a), (1b), (1c), (1d) and (1e) is CA, A is preferably an aromatic or heteroaromatic ring system which has 6 to 24 ring atoms and may be substituted by one or more R radicals. If Y in compounds of the formulae (1), (1a), (1b), (1c), (1d) and (1e) is CA, A is more preferably carbazolyl, phenyl, 1,2-biphenyl, 1,4-biphenyl, triphenylenyl, phenylene-triphenylene, dibenzofuranyl or dibenzothiophenyl, which may be substituted by one or more R radicals. Preferably, in compounds of the formulae (1), (1a), (1b), (1c), (1d) and (1e), one Y is CA and the remaining Y are CH or CR, preferably CH. Preferably, Yin compounds of the formulae (1), (1a), (1b), (1c), (1d) and (1e) is the same and is CH or CR, preferably CH.

In compounds of the formulae (1), (1a) and (1e), R¹ is the same or different at each instance and is independently an alkyl group having 1 to 20 carbon atoms, phenyl which may be substituted by one or more R radicals, or the two R¹ groups together with the carbon atom to which they bind form a spiro compound. In compounds of the formulae (1), (1a) and (1e), R¹ is preferably the same and is an alkyl group having 1 to 10 carbon atoms, or the two R¹ groups together with the carbon atom to which they bind form a spiro compound. R¹ is more preferably the same and is a methyl group, or the two R¹ groups together with the carbon atom to which they bind form a spiro compound. R¹ is most preferably methyl.

A particularly preferred embodiment of the compounds of the formula (1) or (1a) is compounds of the formulae (1b) and (1c), as described above, where the symbols Y, V, R #, m, Rx, Ry, L and ring A₁ used have a definition given above or one specified as preferred hereinafter.

The invention therefore further provides an organic electroluminescent device as described above, wherein, in host material 1, V₁ is 0 or NA and A has a definition given above or one specified as preferred hereinafter.

In compounds of the formulae (1), (1a), (1b), (1c), (1d) and (1e) as described above or described as preferred, Lisa bond or is a phenylene group of the formulae L-1, L-2 or L-3,

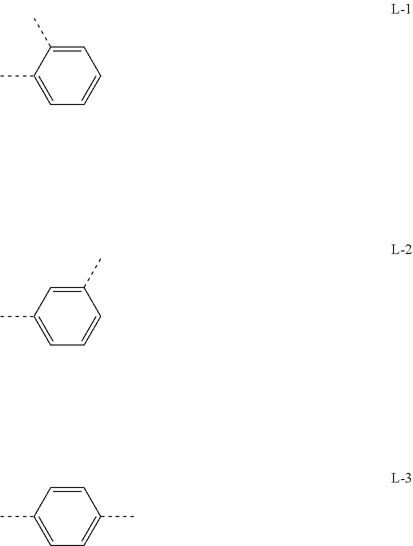

where the phenylene groups L-1, L-2 and L-3 may be substituted by one or more R radicals. The phenylene groups L-1, L-2 and L-3 are preferably unsubstituted. A preferred phenylene group is L-2. In compounds of the formulae (1), (1a), (1b), (1c), (1d) and (1e) as described above or described as preferred, L is preferably a bond.

The substituent R # in compounds of the formulae (1), (1a), (1b), (1c), (1d) and (1e), or compounds of the formulae (1), (1a), (1b), (1c), (1d) and (1e) that are described as preferred, at each instance is independently D or an aryl group which has 6 to 12 carbon atoms and may be substituted by one or more R radicals.

If R # is Din compounds of the formulae (1), (1a), (1b), (1c), (1d) and (1e), it is preferable that m at each instance is 3.

If R # is an aryl group which has 6 to 12 carbon atoms and may be substituted by one or more R radicals in compounds of the formulae (1), (1a), (1b), (1c), (1d) and (1e), mat each instance is independently preferably 0, 1 or 2, more preferably 0 or 1. The aryl group which has 6 to 12 carbon atoms and may be substituted by one or more R radicals is preferably phenyl which may be substituted by one or more R radicals, where R has a definition given above or given with preference. R # is preferably unsubstituted phenyl or fully or partly deuterated phenyl. R # is more preferably unsubstituted phenyl. In compounds of the formulae (1), (1a), (1b), (1c), (1d) and (1e), one m is preferably 1, where R # denotes an aryl group which has 6 to 12 carbon atoms and may be substituted by one or more R radicals, and all other indices m are 0. In compounds of the formulae (1), (1a), (1b), (1c), (1d) and (1e), m at each instance is preferably 0.

The substituent R in compounds of the formulae (1), (1a), (1b), (1c), (1d), (1e) or the group (1B), or compounds of the formulae (1), (1a), (1b), (1c), (1d), (1e) described as preferred or a preferred group (1B), at each instance is selected independently from D, F, CN, a straight-chain alkyl group having 1 to 20 carbon atoms or a branched or cyclic alkyl group having 3 to 20 carbon atoms, where one or more nonadjacent CH₂ groups may be replaced by R²C═CR², O or S and where one or more hydrogen atoms may be replaced by D, F, or CN. The substituent R at each instance is preferably selected independently from D, F, CN, a straight-chain alkyl group having 1 to 10 carbon atoms or a branched or cyclic alkyl group having 3 to 10 carbon atoms, wherein one or more hydrogen atoms of the alkyl group may be replaced by D, F, or CN.

The substituent R² is the same or different at each instance and is selected from the group consisting of H, D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN, and is preferably H or D.

The ring A₁ in the diaza substituent of the formula (1C) of the host material 1 of the formulae (1), (1a), (1b), (1c), (1d) and (1e), as described above or described as preferred,

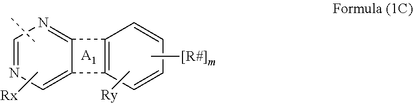

conforms to the formula (1A),

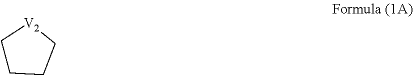

where V₂ is O or S and the dotted line in formula (1C) denotes the attachment to the remainder of the formula (1) or the remainder of the formula (1a) of the host material 1.

Preferred embodiments of the diaza substituent of the formula (1C) of the host material 1 of the formulae (1), (1a), (1b), (1c), (1d) and (1e), as described above or described as preferred, conform to the formulae (1C-1), (1C-2), (1C-3) and (1C-4)

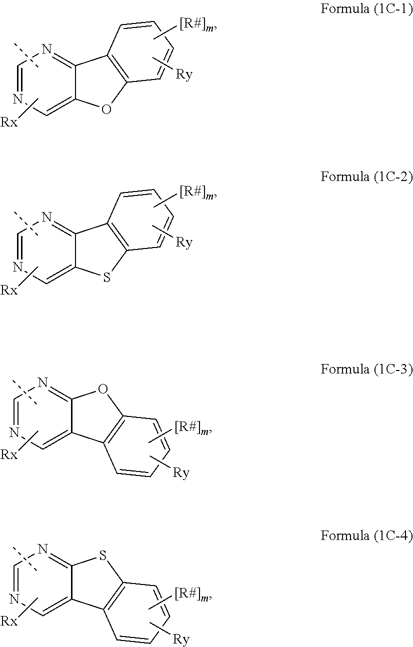

where the dotted line, Rx, Ry, R # and m have a definition given above or given hereinafter.

The attachment of the substituent Rx to the diaza substituent of the formulae (1C), (1C-1), (1C-2), (1C-3) or (1C-4) in the host material 1 of the formulae (1), (1a), (1b), (1c), (1d) and (1e), as described above or described as preferred, is not limited, but is possible at either position.

Preferred embodiments of the diaza substituent of the formula (1C-1) conform to the formulae (1C-5) and (1C-6)

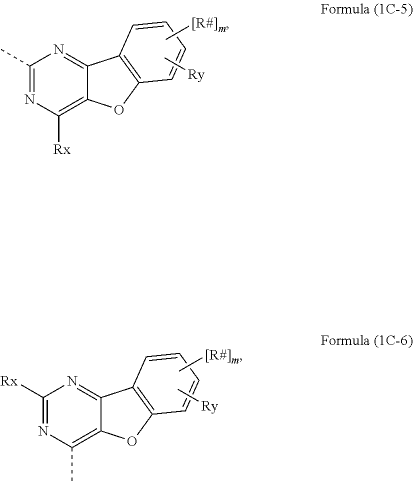

where the dotted line, Rx, Ry, R # and m have a definition given above or given hereinafter.

Preferred embodiments of the diaza substituent of the formula (1C-2) conform to the formulae (1C-7) and (1C-8)

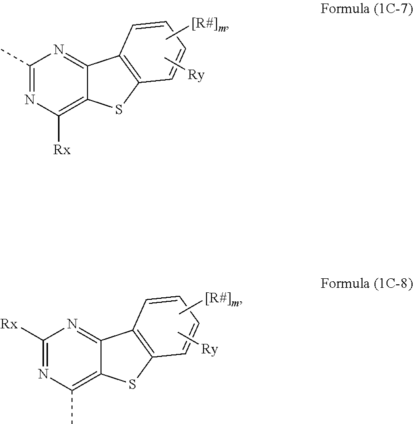

where the dotted line, Rx, Ry, R # and m have a definition given above or given hereinafter.

Preferred embodiments of the diaza substituent of the formula (1C-3) conform to the formulae (1C-9) and (1C-10)

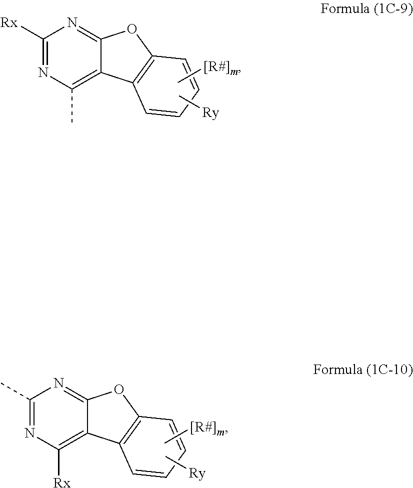

where the dotted line, Rx, Ry, R # and m have a definition given above or given hereinafter.

Preferred embodiments of the diaza substituent of the formula (1C-4) conform to the formulae (1C-11) and (1C-12)

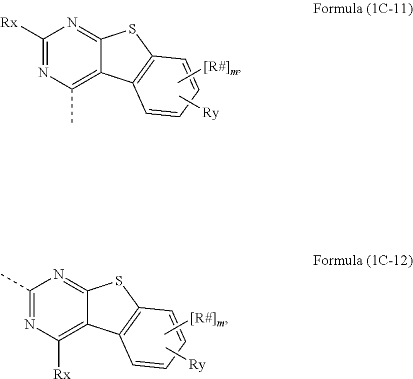

where the dotted line, Rx, Ry, R # and m have a definition given above or given hereinafter.

The invention further provides the organic electroluminescent device as described above, wherein the host material 1 conforms to one of the formulae (1), (1a), (1b), (1c), (1d) and (1e), as described above or described as preferred, and the diaza substituent of the formula (1C) is selected from one of the formulae (1C-1), (1C-2), (1C-3), (1C-4), (1C-5), (1C-6), (1C-7), (1C-8), (1C-9), (1C-10), (1C-11) and (1C-12).

In a preferred embodiment of the host material 1 of the formulae (1), (1a), (1b), (1c), (1d) and (1e), as described above or described as preferred, the diaza substituent of the formula (1C) is selected from one of the formulae (1C-5), (1C-7), (1C-9) and (1C-11).

In a preferred embodiment of the host material 1 of the formulae (1), (1a), (1b), (1c), (1d) and (1e), as described above or described as preferred, the diaza substituent of the formula (1C) is selected from one of the formulae (1C-5), (1C-6), (1C-9) and (1C-10); more preferably from the formula (1C-5) or (1C-9).

In a preferred embodiment of the host material 1 of the formulae (1), (1a), (1b), (1c), (1d) and (1e), as described above or described as preferred, the diaza substituent of the formula (1C) is selected from one of the formulae (1C-7), (1C-8), (1C-11) and (1C-12); more preferably from the formula (1C-7) or (1C-11).

The attachment of the substituent Ry to the diaza substituent of the formulae (1C), (1C-1), (1C-2), (1C-3) or (1C-4) in the host material 1 of the formulae (1), (1a), (1b), (1c), (1d) and (1e), as described above or described as preferred, is not limited, but is possible at any of the four positions.

Preferably, Ry is at these two positions, as depicted in the formulae (1C-13) and (1C-14)

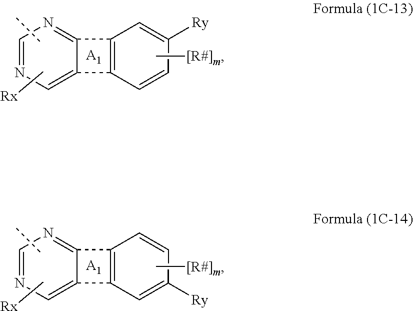

where the dotted line, ring A₁, Rx, Ry, R # and m have a definition given above or given hereinafter.

Particularly preferred embodiments of the diaza substituent of the formula (1C) of the host material 1 of the formulae (1), (1a), (1b), (1c), (1d) and (1e), as described above or described as preferred, conform to the formulae (1C-15) to (1C-30)

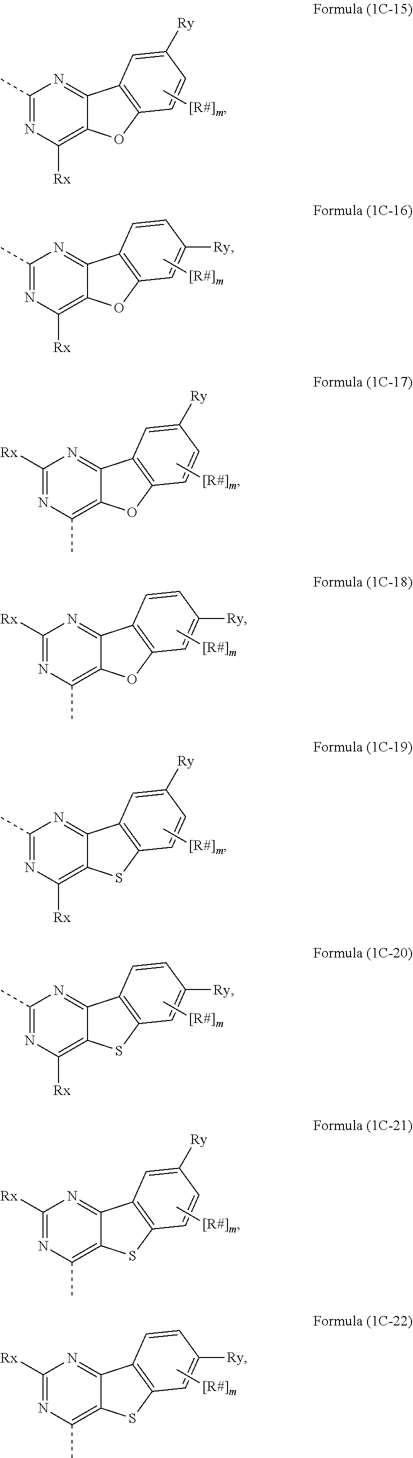

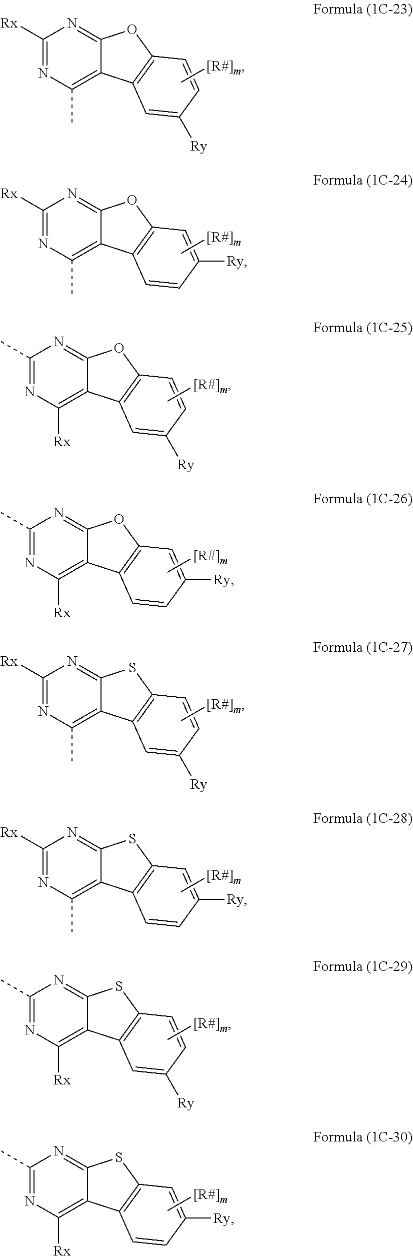

where the dotted line, Rx, Ry, R # and m have a definition given above or given hereinafter.

Particularly preferred embodiments of the diaza substituent of the formula (1C) of the host material 1 of the formulae (1), (1a), (1b), (1c), (1d) and (1e), as described above or described as preferred, conform to the formulae (1C-15), (1C-17), (1C-19), (1C-21), (1C-23), (1C-25), (1C-27) and (1C-29), as described above.

Very particularly preferred embodiments of the diaza substituent of the formula (1C) of the host material 1 of the formulae (1), (1a), (1b), (1c), (1d) and (1e), as described above or described as preferred, conform to the formulae (1C-15), (1C-16), (1C-19), (1C-20), (1C-23), (1C-24), (1C-27) and (1C-28), as described above. Very particularly preferred embodiments of the diaza substituent of the formula (1C) of the host material 1 of the formulae (1), (1a), (1b), (1c), (1d) and (1e), as described above or described as preferred, conform to the formulae (1C-15), (1C-16), (1C-23) and (1C-24), as described above. Very particularly preferred embodiments of the diaza substituent of the formula (1C) of the host material 1 of the formulae (1), (1a), (1b), (1c), (1d) and (1e), as described above or described as preferred, conform to the formulae (1C-19), (1C-20), (1C-27) and (1C-28), as described above.

In the host material of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, the substituent Rx is L₁-R* and the substituent Ry is L₂-R*, where R* at each instance is independently an aromatic or heteroaromatic ring system which has 6 to 14 ring atoms and may be substituted by one or more R radicals, where R has a definition given above and L₁ and L₂ have a definition given above and specified hereinafter.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, L₁ is a bond or is a phenylene group of the formula L-1, L-2 or L-3, where the phenylene groups L-1, L-2 and L-3 may be substituted by one or more R radicals. The phenylene groups L-1, L-2 and L-3 are preferably unsubstituted. Preferred phenyl groups for L₁ are the groups L-2 and L-3. In compounds of the formulae (1), (1a), (1b), (1c), (1d) and (1e) as described above or described as preferred, L₁ is preferably a bond.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, L₂ is a bond or is a phenylene group of the formulae L-1, L-2 or L-3, where the phenylene groups L-1, L-2 and L-3 may be substituted by one or more R radicals. The phenylene groups L-1, L-2 and L-3 are preferably unsubstituted. Preferred phenyl groups for L₁ are the groups L-2 and L-3. In compounds of the formulae (1), (1a), (1b), (1c), (1d) and (1e) as described above or described as preferred, L₂ is preferably a bond.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, R* independently at each instance is preferably phenyl, 1,2-biphenyl, 1,3-biphenyl, 1,4-biphenyl, fluorenyl, dibenzofuranyl or dibenzothiophenyl, which may be substituted by one or more R radicals. Preferably, R* is unsubstituted.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, R* in Ry is more preferably phenyl, fluorenyl, dibenzofuranyl or dibenzothiophenyl, which may be substituted by one or more R radicals. Preferably, R* in Ry is unsubstituted.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, R* in Ry is more preferably phenyl, 1,4-biphenyl, fluorenyl, dibenzofuranyl or dibenzothiophenyl, which may be substituted by one or more R radicals. Preferably, R* in Rx is unsubstituted.

In compounds of the formulae (1), (1a), (1b), (1c), (1d) or (1e) having a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, V is preferably O.

Accordingly, the invention further provides the organic electroluminescent device as described above, wherein, in the host material of the formulae (1), (1a), (1b), (1c), (1d) or (1e) with a diaza substituent of the formulae (1C), (1C-1), (1C-2), (1C-3), (1C-4), (1C-5), (1C-6), (1C-7), (1C-8), (1C-9), (1C-10), (1C-11), (1C-12), (1C-13), (1C-14), (1C-15), (1C-16), (1C-17), (1C-18), (1C-19), (1C-20), (1C-21), (1C-22), (1C-23), (1C-24), (1C-25), (1C-26), (1C-27), (1C-28), (1C-29) or (1C-30), as described above or described as preferred, V is O.

Examples of suitable host materials of the formulae (1) as described above or described as preferred that are selected in accordance with the invention, and are preferably used in combination with at least one compound of the formula (2) in the electroluminescent device of the invention, are the structures given below in table 1.

TABLE 1

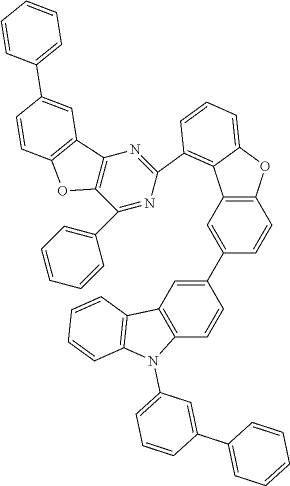 [SMILES: c1ccc(-c2cccc(-n3c4ccccc4c4cc(-c5ccc6oc7cccc(-c8nc(-c9ccccc9)c9oc%10ccc(-c%11ccccc%11)cc%10c9n8)c7c6c5)ccc43)c2)cc1]

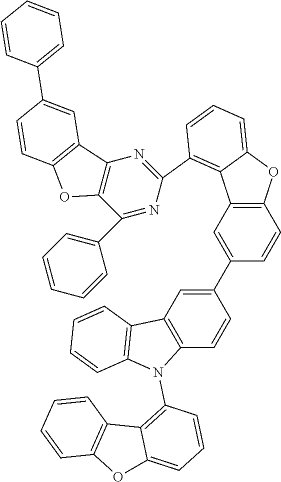 [SMILES: c1ccc(-c2ccc3oc4c(-c5ccccc5)nc(-c5cccc6oc7ccc(-c8ccc9c(c8)c8ccccc8n9-c8cccc9oc%10ccccc%10c89)cc7c56)nc4c3c2)cc1]

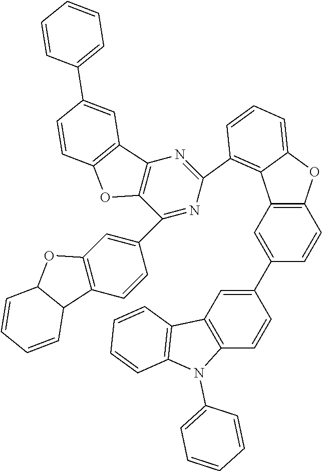 [SMILES: C1=CC2Oc3cc(-c4nc(-c5cccc6oc7ccc(-c8ccc9c(c8)c8ccccc8n9-c8ccccc8)cc7c56)nc5c4oc4ccc(-c6ccccc6)cc45)ccc3C2C=C1]

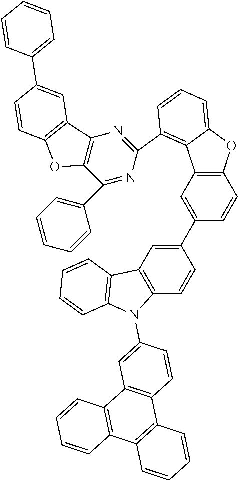 [SMILES: c1ccc(-c2ccc3oc4c(-c5ccccc5)nc(-c5cccc6oc7ccc(-c8ccc9c(c8)c8ccccc8n9-c8ccc9c%10ccccc%10c%10ccccc%10c9c8)cc7c56)nc4c3c2)cc1]

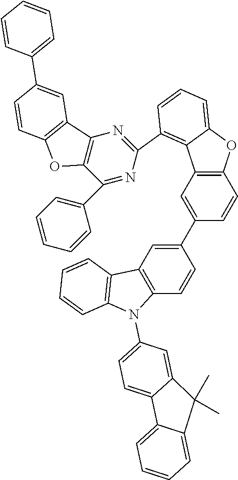 [SMILES: CC1(C)c2ccccc2-c2ccc(-n3c4ccccc4c4cc(-c5ccc6oc7cccc(-c8nc(-c9ccccc9)c9oc%10ccc(-c%11ccccc%11)cc%10c9n8)c7c6c5)ccc43)cc21]

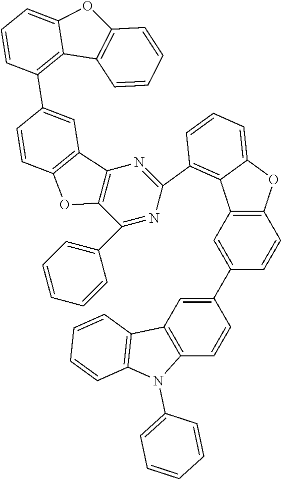 [SMILES: c1ccc(-c2nc(-c3cccc4oc5ccc(-c6ccc7c(c6)c6ccccc6n7-c6ccccc6)cc5c34)nc3c2oc2ccc(-c4cccc5oc6ccccc6c45)cc23)cc1]

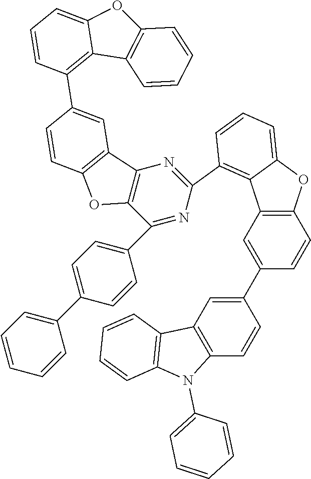 [SMILES: c1ccc(-c2ccc(-c3nc(-c4cccc5oc6ccc(-c7ccc8c(c7)c7ccccc7n8-c7ccccc7)cc6c45)nc4c3oc3ccc(-c5cccc6oc7ccccc7c56)cc34)cc2)cc1]

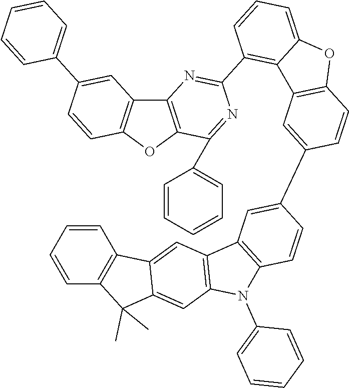 [SMILES: CC1(C)c2ccccc2-c2cc3c4cc(-c5ccc6oc7cccc(-c8nc(-c9ccccc9)c9oc%10ccc(-c%11ccccc%11)cc%10c9n8)c7c6c5)ccc4n(-c4ccccc4)c3cc21]

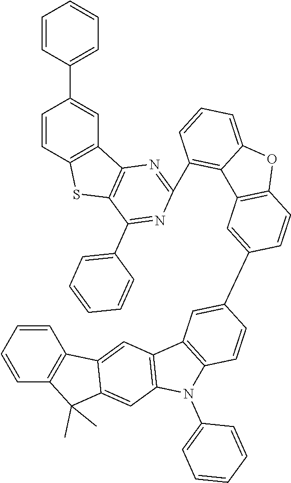 [SMILES: CC1(C)c2ccccc2-c2cc3c4cc(-c5ccc6oc7cccc(-c8nc(-c9ccccc9)c9sc%10ccc(-c%11ccccc%11)cc%10c9n8)c7c6c5)ccc4n(-c4ccccc4)c3cc21]

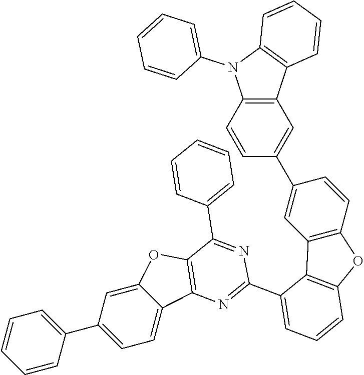 [SMILES: c1ccc(-c2ccc3c(c2)oc2c(-c4ccccc4)nc(-c4cccc5oc6ccc(-c7ccc8c(c7)c7ccccc7n8-c7ccccc7)cc6c45)nc23)cc1]

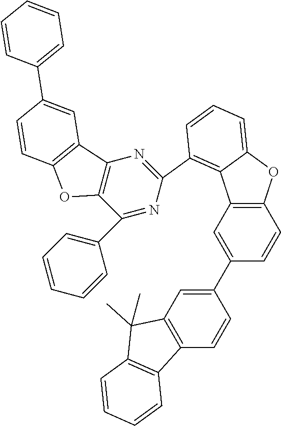 [SMILES: CC1(C)c2ccccc2-c2ccc(-c3ccc4oc5cccc(-c6nc(-c7ccccc7)c7oc8ccc(-c9ccccc9)cc8c7n6)c5c4c3)cc21]

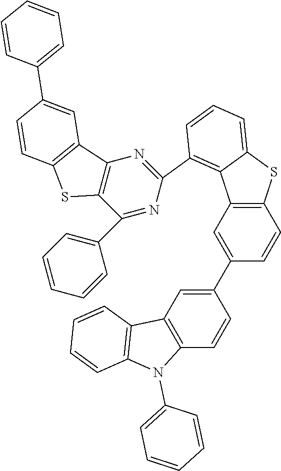 [SMILES: c1ccc(-c2ccc3sc4c(-c5ccccc5)nc(-c5cccc6sc7ccc(-c8ccc9c(c8)c8ccccc8n9-c8ccccc8)cc7c56)nc4c3c2)cc1]

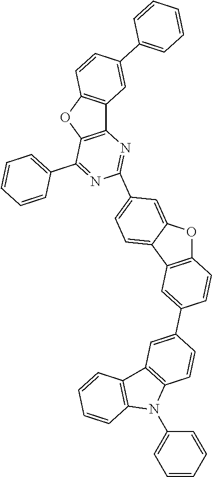 [SMILES: c1ccc(-c2ccc3oc4c(-c5ccccc5)nc(-c5ccc6c(c5)oc5ccc(-c7ccc8c(c7)c7ccccc7n8-c7ccccc7)cc56)nc4c3c2)cc1]

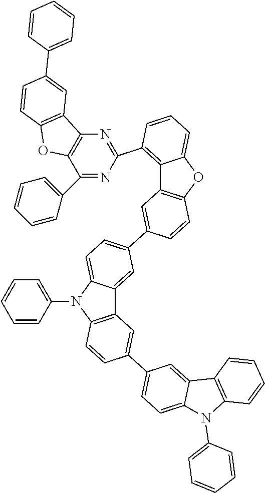 [SMILES: c1ccc(-c2ccc3oc4c(-c5ccccc5)nc(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)nc4c3c2)cc1]

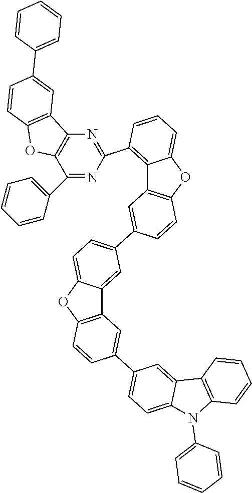 [SMILES: c1ccc(-c2ccc3oc4c(-c5ccccc5)nc(-c5cccc6oc7ccc(-c8ccc9oc%10ccc(-c%11ccc%12c(c%11)c%11ccccc%11n%12-c%11ccccc%11)cc%10c9c8)cc7c56)nc4c3c2)cc1]

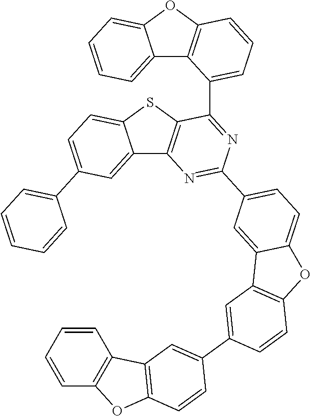 [SMILES: c1ccc(-c2ccc3sc4c(-c5cccc6oc7ccccc7c56)nc(-c5ccc6oc7ccc(-c8ccc9oc%10ccccc%10c9c8)cc7c6c5)nc4c3c2)cc1]

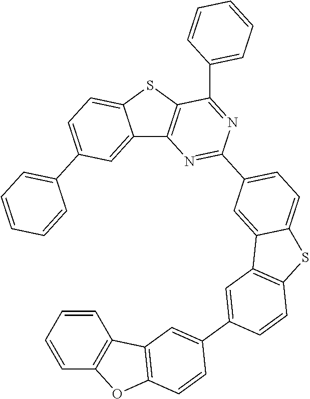 [SMILES: c1ccc(-c2ccc3sc4c(-c5ccccc5)nc(-c5ccc6sc7ccc(-c8ccc9oc%10ccccc%10c9c8)cc7c6c5)nc4c3c2)cc1]

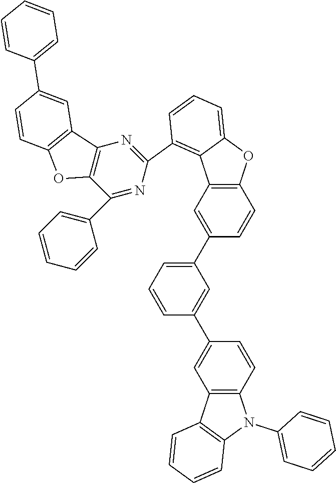 [SMILES: c1ccc(-c2ccc3oc4c(-c5ccccc5)nc(-c5cccc6oc7ccc(-c8cccc(-c9ccc%10c(c9)c9ccccc9n%10-c9ccccc9)c8)cc7c56)nc4c3c2)cc1]

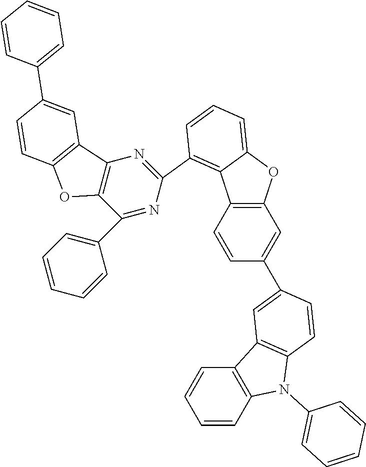 [SMILES: c1ccc(-c2ccc3oc4c(-c5ccccc5)nc(-c5cccc6oc7cc(-c8ccc9c(c8)c8ccccc8n9-c8ccccc8)ccc7c56)nc4c3c2)cc1]

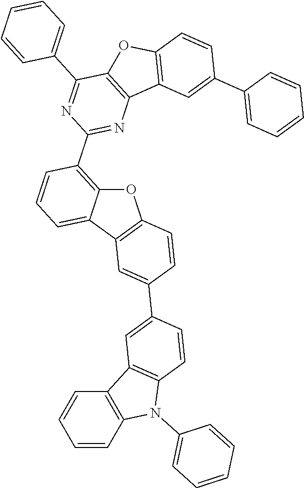 [SMILES: c1ccc(-c2ccc3oc4c(-c5ccccc5)nc(-c5cccc6c5oc5ccc(-c7ccc8c(c7)c7ccccc7n8-c7ccccc7)cc56)nc4c3c2)cc1]

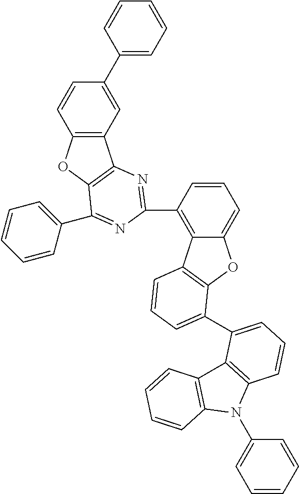 [SMILES: c1ccc(-c2ccc3oc4c(-c5ccccc5)nc(-c5cccc6oc7c(-c8cccc9c8c8ccccc8n9-c8ccccc8)cccc7c56)nc4c3c2)cc1]

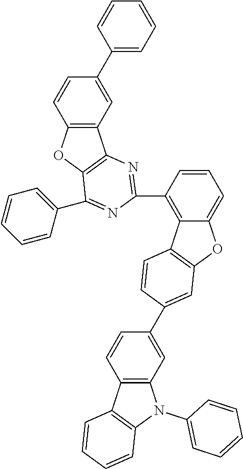 [SMILES: c1ccc(-c2ccc3oc4c(-c5ccccc5)nc(-c5cccc6oc7cc(-c8ccc9c%10ccccc%10n(-c%10ccccc%10)c9c8)ccc7c56)nc4c3c2)cc1]

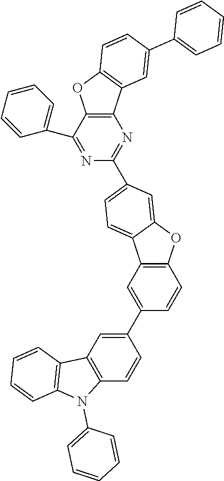 [SMILES: c1ccc(-c2ccc3oc4c(-c5ccccc5)nc(-c5ccc6c(c5)oc5ccc(-c7ccc8c(c7)c7ccccc7n8-c7ccccc7)cc56)nc4c3c2)cc1]

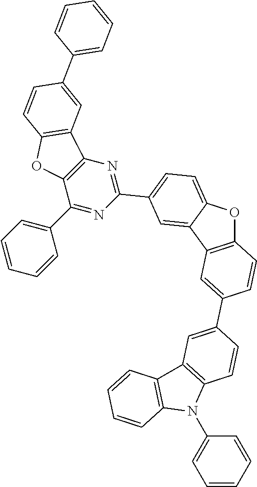 [SMILES: c1ccc(-c2ccc3oc4c(-c5ccccc5)nc(-c5ccc6oc7ccc(-c8ccc9c(c8)c8ccccc8n9-c8ccccc8)cc7c6c5)nc4c3c2)cc1]

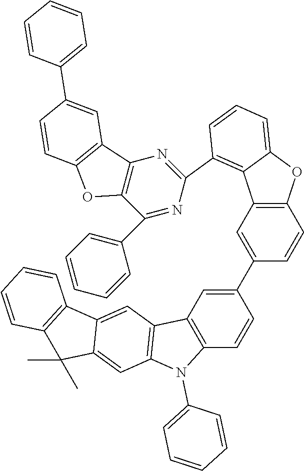 [SMILES: CC1(C)c2ccccc2-c2cc3c4cc(-c5ccc6oc7cccc(-c8nc(-c9ccccc9)c9oc%10ccc(-c%11ccccc%11)cc%10c9n8)c7c6c5)ccc4n(-c4ccccc4)c3cc21]

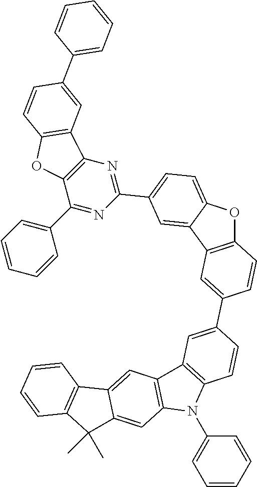 [SMILES: CC1(C)c2ccccc2-c2cc3c4cc(-c5ccc6oc7ccc(-c8nc(-c9ccccc9)c9oc%10ccc(-c%11ccccc%11)cc%10c9n8)cc7c6c5)ccc4n(-c4ccccc4)c3cc21]

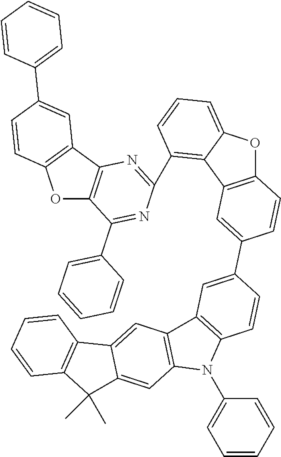 [SMILES: CC1(C)c2ccccc2-c2cc3c4cc(-c5ccc6oc7cccc(-c8nc(-c9ccccc9)c9oc%10ccc(-c%11ccccc%11)cc%10c9n8)c7c6c5)ccc4n(-c4ccccc4)c3cc21]

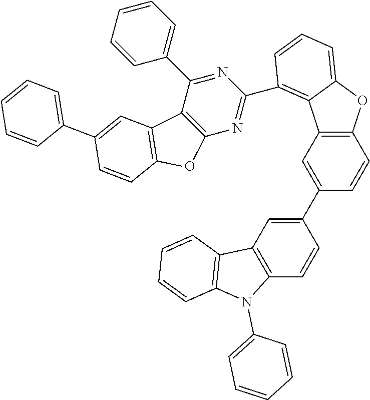 [SMILES: c1ccc(-c2ccc3oc4nc(-c5cccc6oc7ccc(-c8ccc9c(c8)c8ccccc8n9-c8ccccc8)cc7c56)nc(-c5ccccc5)c4c3c2)cc1]

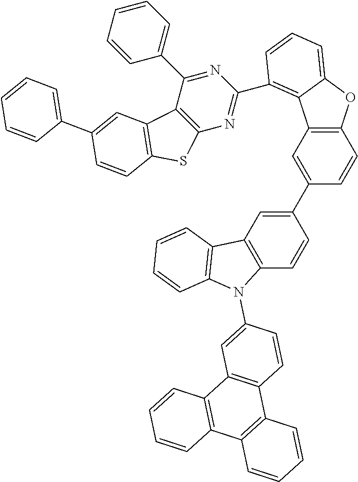 [SMILES: c1ccc(-c2ccc3sc4nc(-c5cccc6oc7ccc(-c8ccc9c(c8)c8ccccc8n9-c8ccc9c%10ccccc%10c%10ccccc%10c9c8)cc7c56)nc(-c5ccccc5)c4c3c2)cc1]

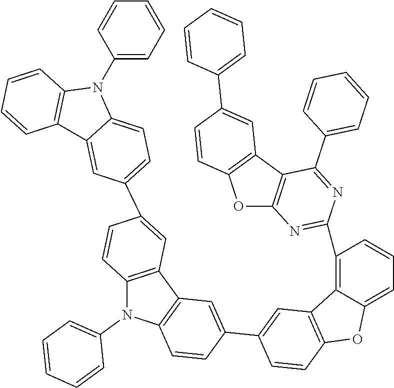 [SMILES: c1ccc(-c2ccc3oc4nc(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)nc(-c5ccccc5)c4c3c2)cc1]

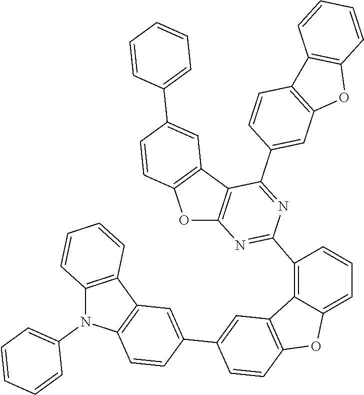 [SMILES: c1ccc(-c2ccc3oc4nc(-c5cccc6oc7ccc(-c8ccc9c(c8)c8ccccc8n9-c8ccccc8)cc7c56)nc(-c5ccc6c(c5)oc5ccccc56)c4c3c2)cc1]

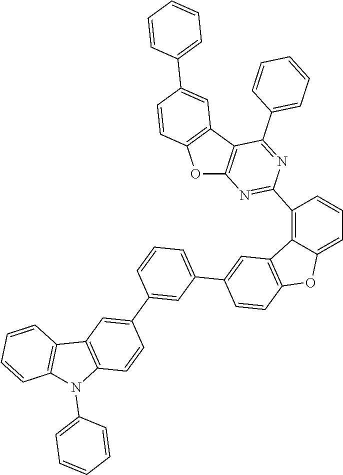 [SMILES: c1ccc(-c2ccc3oc4nc(-c5cccc6oc7ccc(-c8cccc(-c9ccc%10c(c9)c9ccccc9n%10-c9ccccc9)c8)cc7c56)nc(-c5ccccc5)c4c3c2)cc1]

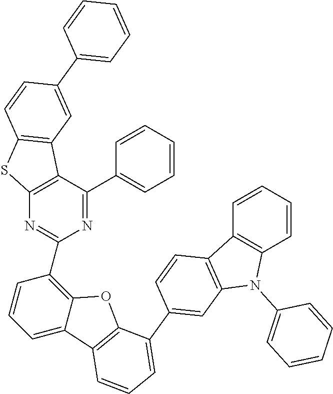 [SMILES: c1ccc(-c2ccc3sc4nc(-c5cccc6c5oc5c(-c7ccc8c9ccccc9n(-c9ccccc9)c8c7)cccc56)nc(-c5ccccc5)c4c3c2)cc1]

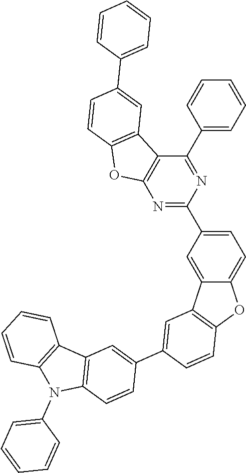 [SMILES: c1ccc(-c2ccc3oc4nc(-c5ccc6oc7ccc(-c8ccc9c(c8)c8ccccc8n9-c8ccccc8)cc7c6c5)nc(-c5ccccc5)c4c3c2)cc1]

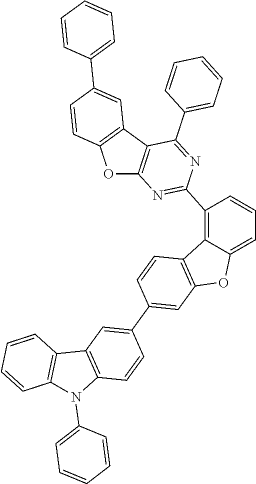 [SMILES: c1ccc(-c2ccc3oc4nc(-c5cccc6oc7cc(-c8ccc9c(c8)c8ccccc8n9-c8ccccc8)ccc7c56)nc(-c5ccccc5)c4c3c2)cc1]

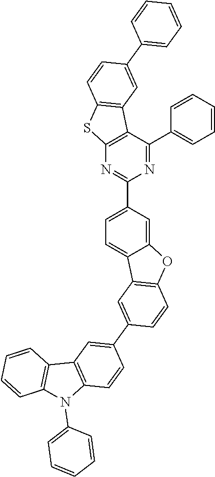 [SMILES: c1ccc(-c2ccc3sc4nc(-c5ccc6c(c5)oc5ccc(-c7ccc8c(c7)c7ccccc7n8-c7ccccc7)cc56)nc(-c5ccccc5)c4c3c2)cc1]

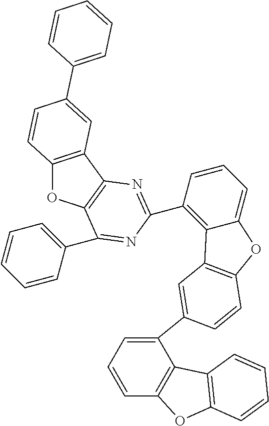 [SMILES: c1ccc(-c2ccc3oc4c(-c5ccccc5)nc(-c5cccc6oc7ccc(-c8cccc9oc%10ccccc%10c89)cc7c56)nc4c3c2)cc1]

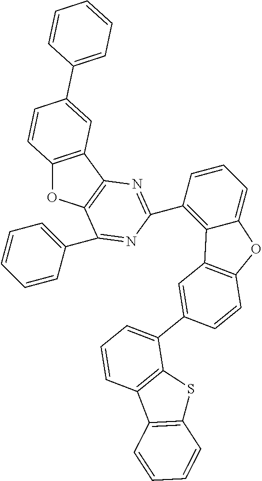 [SMILES: c1ccc(-c2ccc3oc4c(-c5ccccc5)nc(-c5cccc6oc7ccc(-c8cccc9c8sc8ccccc89)cc7c56)nc4c3c2)cc1]

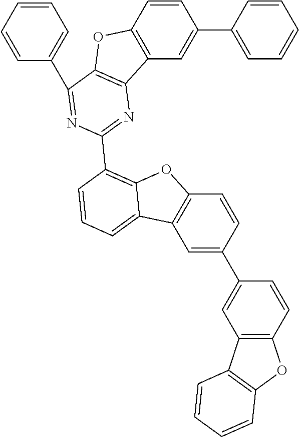 [SMILES: c1ccc(-c2ccc3oc4c(-c5ccccc5)nc(-c5cccc6c5oc5ccc(-c7ccc8oc9ccccc9c8c7)cc56)nc4c3c2)cc1]

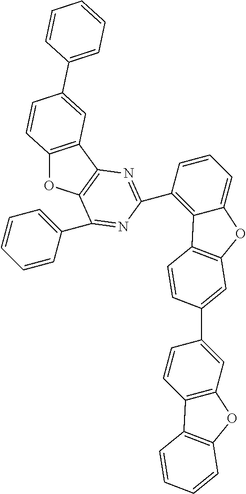 [SMILES: c1ccc(-c2ccc3oc4c(-c5ccccc5)nc(-c5cccc6oc7cc(-c8ccc9c(c8)oc8ccccc89)ccc7c56)nc4c3c2)cc1]

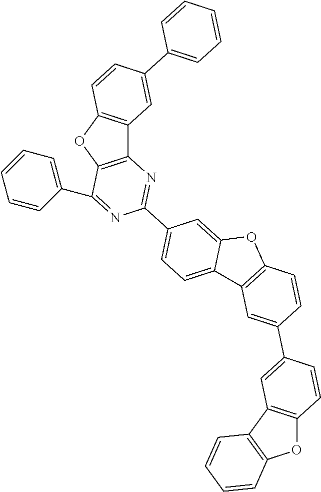 [SMILES: c1ccc(-c2ccc3oc4c(-c5ccccc5)nc(-c5ccc6c(c5)oc5ccc(-c7ccc8oc9ccccc9c8c7)cc56)nc4c3c2)cc1]

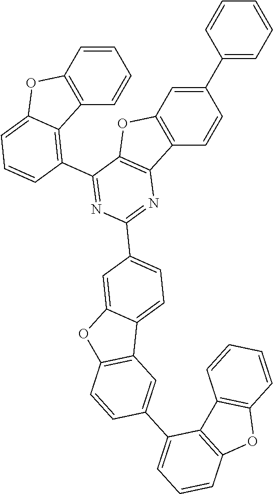 [SMILES: c1ccc(-c2ccc3c(c2)oc2c(-c4cccc5oc6ccccc6c45)nc(-c4ccc5c(c4)oc4ccc(-c6cccc7oc8ccccc8c67)cc45)nc23)cc1]

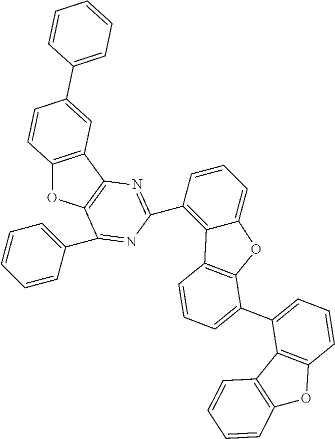 [SMILES: c1ccc(-c2ccc3oc4c(-c5ccccc5)nc(-c5cccc6oc7c(-c8cccc9oc%10ccccc%10c89)cccc7c56)nc4c3c2)cc1]

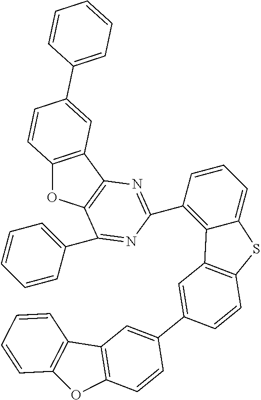 [SMILES: c1ccc(-c2ccc3oc4c(-c5ccccc5)nc(-c5cccc6sc7ccc(-c8ccc9oc%10ccccc%10c9c8)cc7c56)nc4c3c2)cc1]

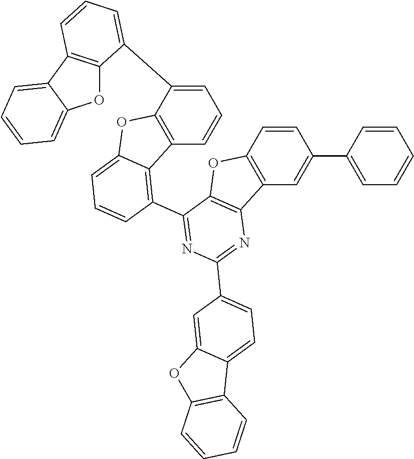 [SMILES: c1ccc(-c2ccc3oc4c(-c5cccc6oc7c(-c8cccc9c8oc8ccccc89)cccc7c56)nc(-c5ccc6c(c5)oc5ccccc56)nc4c3c2)cc1]

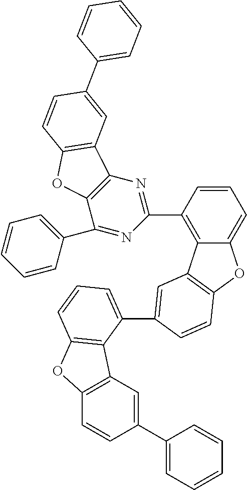 [SMILES: c1ccc(-c2ccc3oc4c(-c5ccccc5)nc(-c5cccc6oc7ccc(-c8cccc9oc%10ccc(-c%11ccccc%11)cc%10c89)cc7c56)nc4c3c2)cc1]

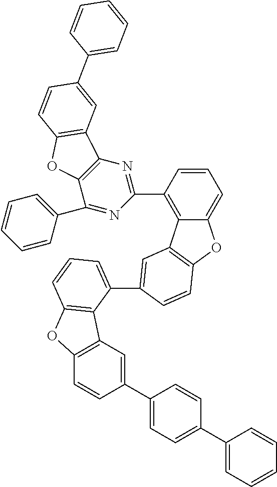 [SMILES: c1ccc(-c2ccc(-c3ccc4oc5cccc(-c6ccc7oc8cccc(-c9nc(-c%10ccccc%10)c%10oc%11ccc(-c%12ccccc%12)cc%11c%10n9)c8c7c6)c5c4c3)cc2)cc1]

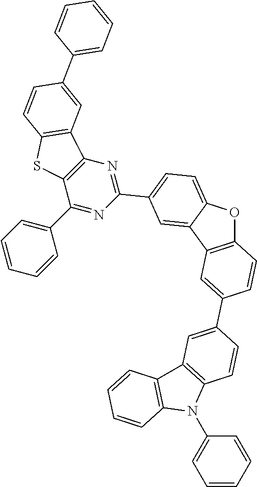 [SMILES: c1ccc(-c2ccc3sc4c(-c5ccccc5)nc(-c5ccc6oc7ccc(-c8ccc9c(c8)c8ccccc8n9-c8ccccc8)cc7c6c5)nc4c3c2)cc1]

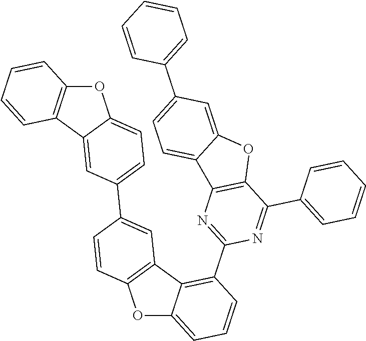 [SMILES: c1ccc(-c2ccc3c(c2)oc2c(-c4ccccc4)nc(-c4cccc5oc6ccc(-c7ccc8oc9ccccc9c8c7)cc6c45)nc23)cc1]

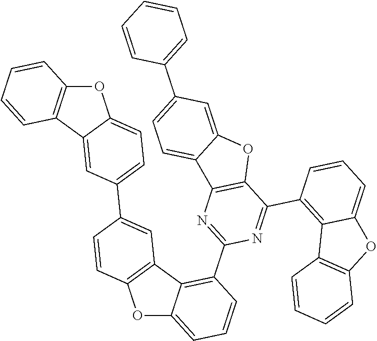 [SMILES: c1ccc(-c2ccc3c(c2)oc2c(-c4cccc5oc6ccccc6c45)nc(-c4cccc5oc6ccc(-c7ccc8oc9ccccc9c8c7)cc6c45)nc23)cc1]

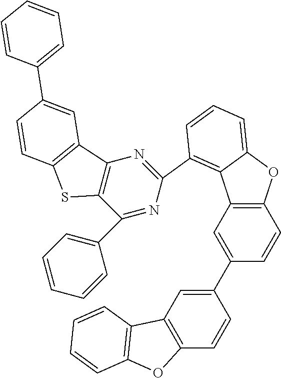 [SMILES: c1ccc(-c2ccc3sc4c(-c5ccccc5)nc(-c5cccc6oc7ccc(-c8ccc9oc%10ccccc%10c9c8)cc7c56)nc4c3c2)cc1]

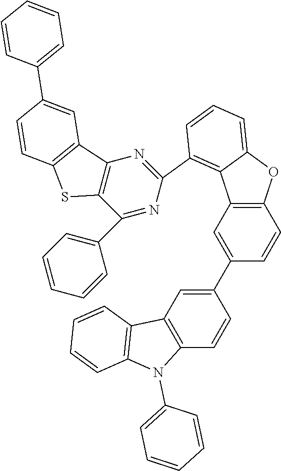 [SMILES: c1ccc(-c2ccc3sc4c(-c5ccccc5)nc(-c5cccc6oc7ccc(-c8ccc9c(c8)c8ccccc8n9-c8ccccc8)cc7c56)nc4c3c2)cc1]

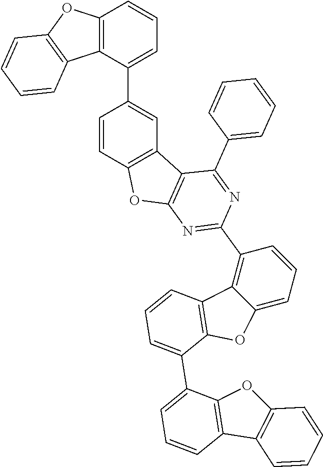 [SMILES: c1ccc(-c2nc(-c3cccc4oc5c(-c6cccc7c6oc6ccccc67)cccc5c34)nc3oc4ccc(-c5cccc6oc7ccccc7c56)cc4c23)cc1]

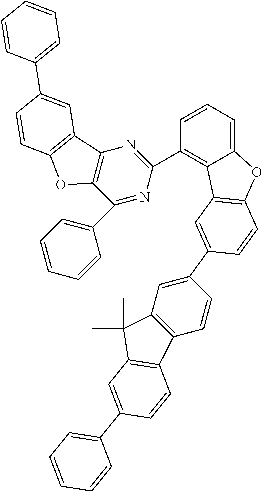 [SMILES: CC1(C)c2cc(-c3ccccc3)ccc2-c2ccc(-c3ccc4oc5cccc(-c6nc(-c7ccccc7)c7oc8ccc(-c9ccccc9)cc8c7n6)c5c4c3)cc21]

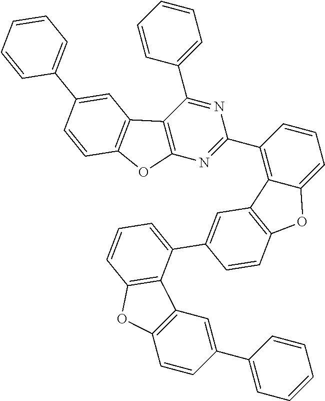 [SMILES: c1ccc(-c2ccc3oc4cccc(-c5ccc6oc7cccc(-c8nc(-c9ccccc9)c9c(n8)oc8ccc(-c%10ccccc%10)cc89)c7c6c5)c4c3c2)cc1]

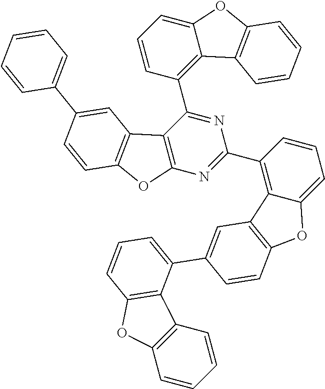 [SMILES: c1ccc(-c2ccc3oc4nc(-c5cccc6oc7ccc(-c8cccc9oc%10ccccc%10c89)cc7c56)nc(-c5cccc6oc7ccccc7c56)c4c3c2)cc1]

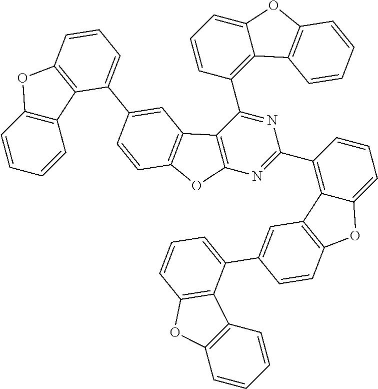 [SMILES: c1ccc2c(c1)oc1cccc(-c3ccc4oc5cccc(-c6nc(-c7cccc8oc9ccccc9c78)c7c(n6)oc6ccc(-c8cccc9oc%10ccccc%10c89)cc67)c5c4c3)c12]

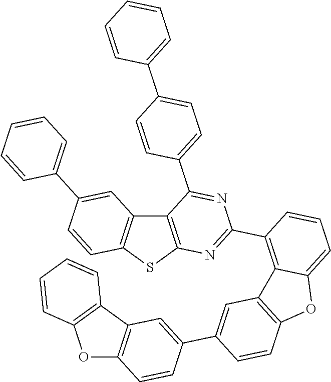 [SMILES: c1ccc(-c2ccc(-c3nc(-c4cccc5oc6ccc(-c7ccc8oc9ccccc9c8c7)cc6c45)nc4sc5ccc(-c6ccccc6)cc5c34)cc2)cc1]

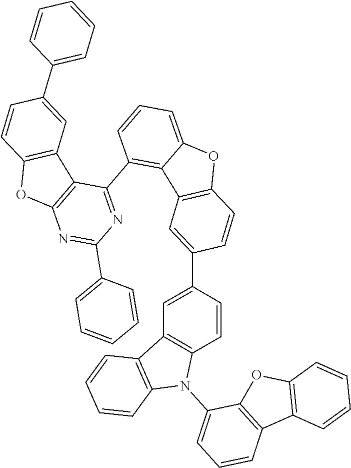 [SMILES: c1ccc(-c2ccc3oc4nc(-c5ccccc5)nc(-c5cccc6oc7ccc(-c8ccc9c(c8)c8ccccc8n9-c8cccc9c8oc8ccccc89)cc7c56)c4c3c2)cc1]

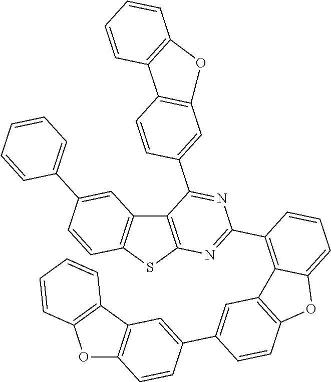 [SMILES: c1ccc(-c2ccc3sc4nc(-c5cccc6oc7ccc(-c8ccc9oc%10ccccc%10c9c8)cc7c56)nc(-c5ccc6c(c5)oc5ccccc56)c4c3c2)cc1]

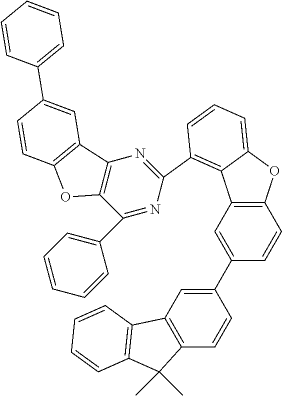

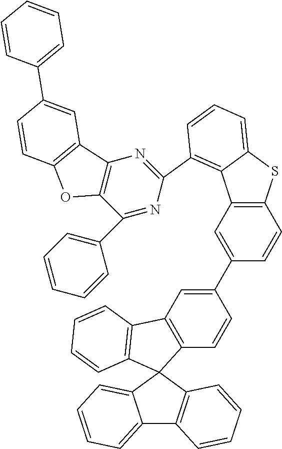

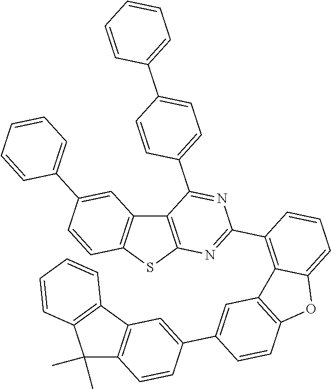 [SMILES: CC1(C)c2ccccc2-c2cc(-c3ccc4oc5cccc(-c6nc(-c7ccc(-c8ccccc8)cc7)c7c(n6)sc6ccc(-c8ccccc8)cc67)c5c4c3)ccc21]

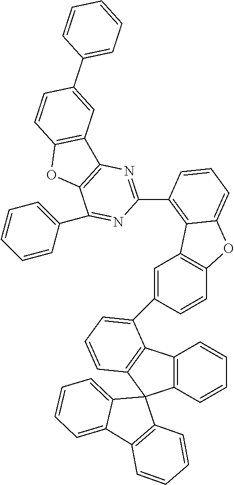 [SMILES: c1ccc(-c2ccc3oc4c(-c5ccccc5)nc(-c5cccc6oc7ccc(-c8cccc9c8-c8ccccc8C98c9ccccc9-c9ccccc98)cc7c56)nc4c3c2)cc1]

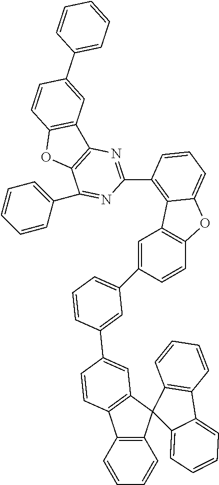 [SMILES: c1ccc(-c2ccc3oc4c(-c5ccccc5)nc(-c5cccc6oc7ccc(-c8cccc(-c9ccc%10c(c9)C9(c%11ccccc%11-c%11ccccc%119)c9ccccc9-%10)c8)cc7c56)nc4c3c2)cc1]

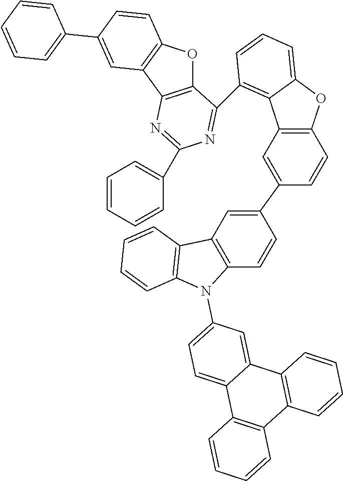 [SMILES: c1ccc(-c2ccc3oc4c(-c5cccc6oc7ccc(-c8ccc9c(c8)c8ccccc8n9-c8ccc9c%10ccccc%10c%10ccccc%10c9c8)cc7c56)nc(-c5ccccc5)nc4c3c2)cc1]

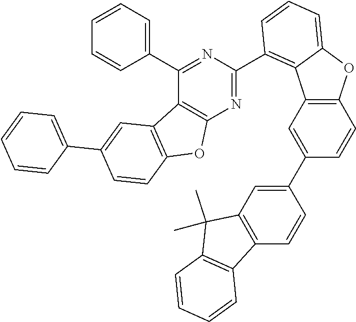 [SMILES: CC1(C)c2ccccc2-c2ccc(-c3ccc4oc5cccc(-c6nc(-c7ccccc7)c7c(n6)oc6ccc(-c8ccccc8)cc67)c5c4c3)cc21]

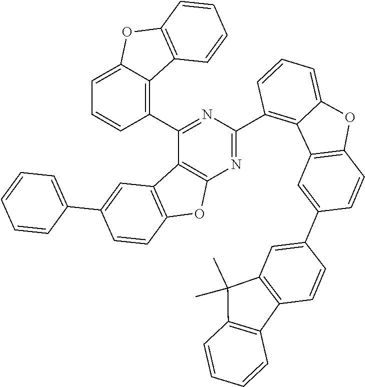 [SMILES: CC1(C)c2ccccc2-c2ccc(-c3ccc4oc5cccc(-c6nc(-c7cccc8oc9ccccc9c78)c7c(n6)oc6ccc(-c8ccccc8)cc67)c5c4c3)cc21]

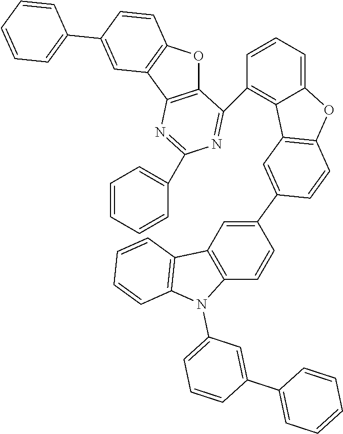 [SMILES: c1ccc(-c2cccc(-n3c4ccccc4c4cc(-c5ccc6oc7cccc(-c8nc(-c9ccccc9)nc9c8oc8ccc(-c%10ccccc%10)cc89)c7c6c5)ccc43)c2)cc1]

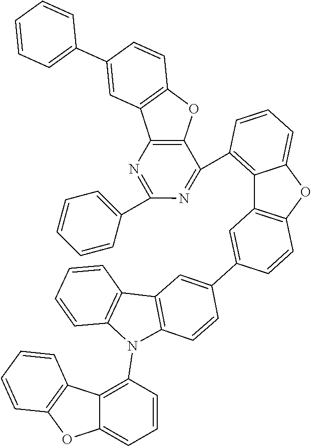 [SMILES: c1ccc(-c2ccc3oc4c(-c5cccc6oc7ccc(-c8ccc9c(c8)c8ccccc8n9-c8cccc9oc%10ccccc%10c89)cc7c56)nc(-c5ccccc5)nc4c3c2)cc1]

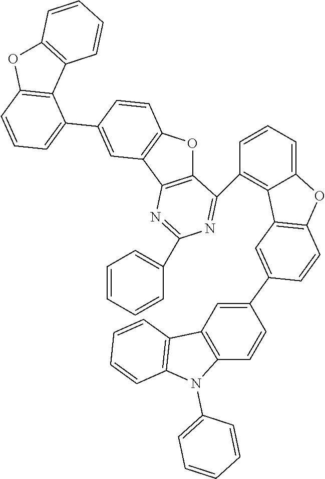 [SMILES: c1ccc(-c2nc(-c3cccc4oc5ccc(-c6ccc7c(c6)c6ccccc6n7-c6ccccc6)cc5c34)c3oc4ccc(-c5cccc6oc7ccccc7c56)cc4c3n2)cc1]

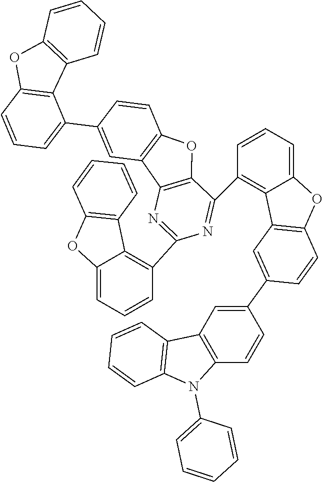 [SMILES: c1ccc(-n2c3ccccc3c3cc(-c4ccc5oc6cccc(-c7nc(-c8cccc9oc%10ccccc%10c89)nc8c7oc7ccc(-c9cccc%10oc%11ccccc%11c9%10)cc78)c6c5c4)ccc32)cc1]

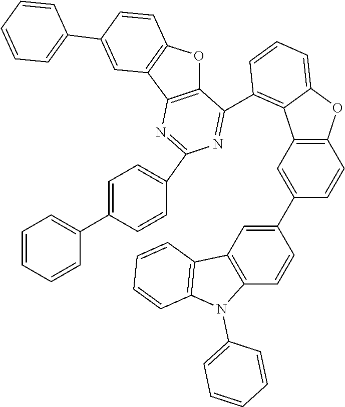 [SMILES: c1ccc(-c2ccc(-c3nc(-c4cccc5oc6ccc(-c7ccc8c(c7)c7ccccc7n8-c7ccccc7)cc6c45)c4oc5ccc(-c6ccccc6)cc5c4n3)cc2)cc1]

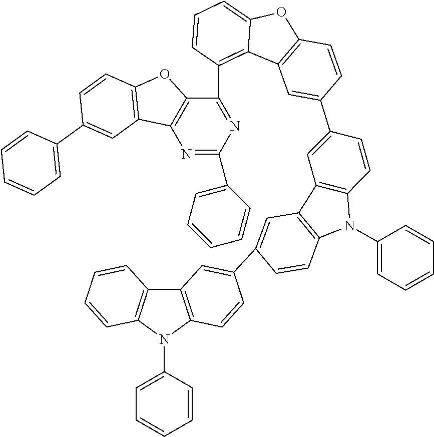 [SMILES: c1ccc(-c2ccc3oc4c(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)nc(-c5ccccc5)nc4c3c2)cc1]

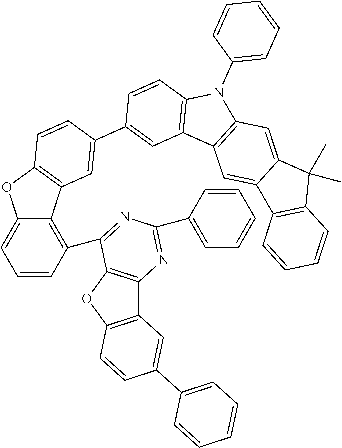 [SMILES: CC1(C)c2ccccc2-c2cc3c4cc(-c5ccc6oc7cccc(-c8nc(-c9ccccc9)nc9c8oc8ccc(-c%10ccccc%10)cc89)c7c6c5)ccc4n(-c4ccccc4)c3cc21]

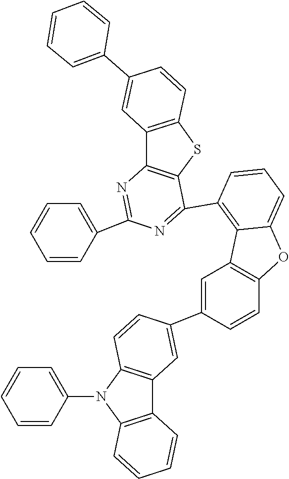 [SMILES: c1ccc(-c2ccc3sc4c(-c5cccc6oc7ccc(-c8ccc9c(c8)c8ccccc8n9-c8ccccc8)cc7c56)nc(-c5ccccc5)nc4c3c2)cc1]

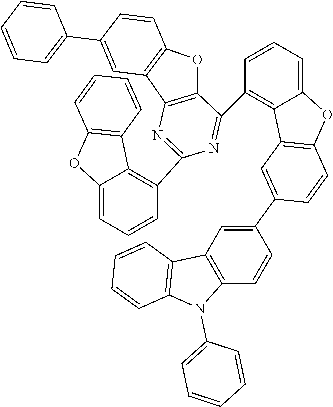 [SMILES: c1ccc(-c2ccc3oc4c(-c5cccc6oc7ccc(-c8ccc9c(c8)c8ccccc8n9-c8ccccc8)cc7c56)nc(-c5cccc6oc7ccccc7c56)nc4c3c2)cc1]

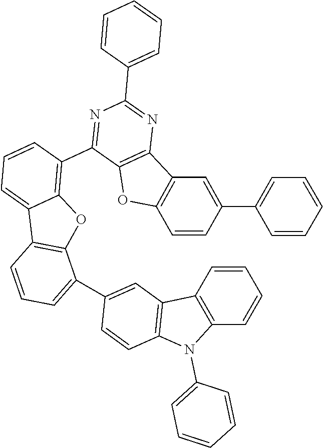 [SMILES: c1ccc(-c2ccc3oc4c(-c5cccc6c5oc5c(-c7ccc8c(c7)c7ccccc7n8-c7ccccc7)cccc56)nc(-c5ccccc5)nc4c3c2)cc1]

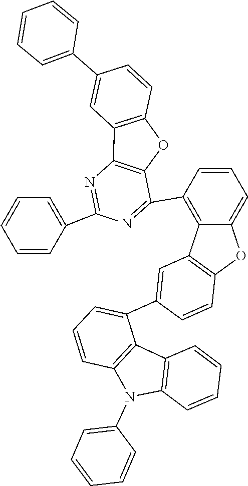 [SMILES: c1ccc(-c2ccc3oc4c(-c5cccc6oc7ccc(-c8cccc9c8c8ccccc8n9-c8ccccc8)cc7c56)nc(-c5ccccc5)nc4c3c2)cc1]

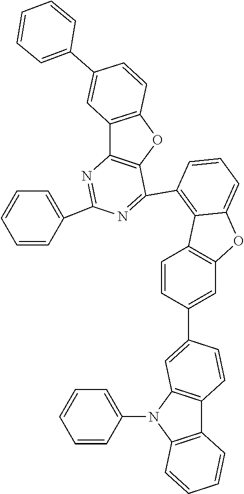 [SMILES: c1ccc(-c2ccc3oc4c(-c5cccc6oc7cc(-c8ccc9c%10ccccc%10n(-c%10ccccc%10)c9c8)ccc7c56)nc(-c5ccccc5)nc4c3c2)cc1]

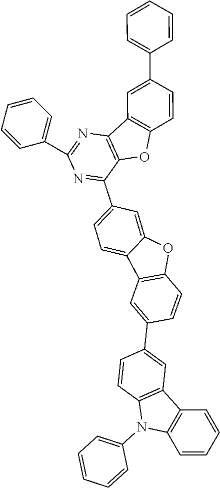 [SMILES: c1ccc(-c2ccc3oc4c(-c5ccc6c(c5)oc5ccc(-c7ccc8c(c7)c7ccccc7n8-c7ccccc7)cc56)nc(-c5ccccc5)nc4c3c2)cc1]

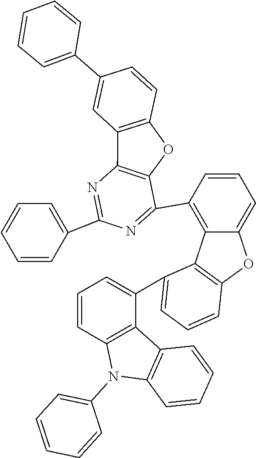 [SMILES: c1ccc(-c2ccc3oc4c(-c5cccc6oc7cccc(-c8cccc9c8c8ccccc8n9-c8ccccc8)c7c56)nc(-c5ccccc5)nc4c3c2)cc1]

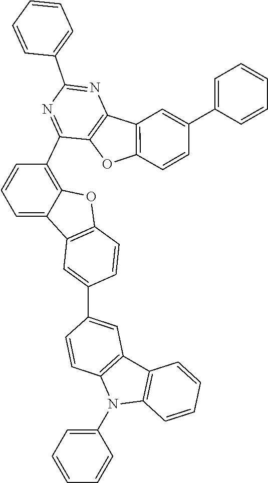 [SMILES: c1ccc(-c2ccc3oc4c(-c5cccc6c5oc5ccc(-c7ccc8c(c7)c7ccccc7n8-c7ccccc7)cc56)nc(-c5ccccc5)nc4c3c2)cc1]

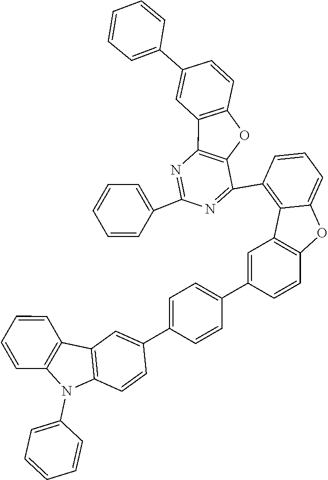 [SMILES: c1ccc(-c2ccc3oc4c(-c5cccc6oc7ccc(-c8ccc(-c9ccc%10c(c9)c9ccccc9n%10-c9ccccc9)cc8)cc7c56)nc(-c5ccccc5)nc4c3c2)cc1]

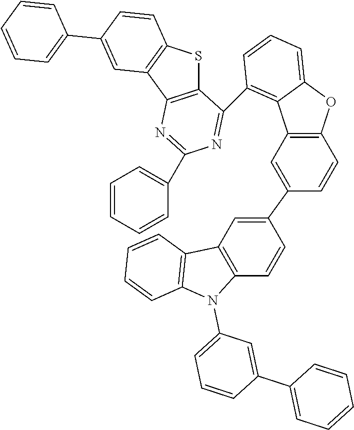 [SMILES: c1ccc(-c2cccc(-n3c4ccccc4c4cc(-c5ccc6oc7cccc(-c8nc(-c9ccccc9)nc9c8sc8ccc(-c%10ccccc%10)cc89)c7c6c5)ccc43)c2)cc1]

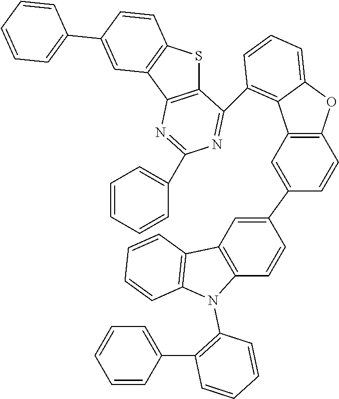 [SMILES: c1ccc(-c2ccc3sc4c(-c5cccc6oc7ccc(-c8ccc9c(c8)c8ccccc8n9-c8ccccc8-c8ccccc8)cc7c56)nc(-c5ccccc5)nc4c3c2)cc1]

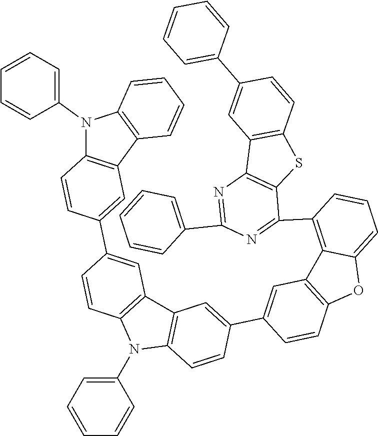 [SMILES: c1ccc(-c2ccc3sc4c(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)nc(-c5ccccc5)nc4c3c2)cc1]

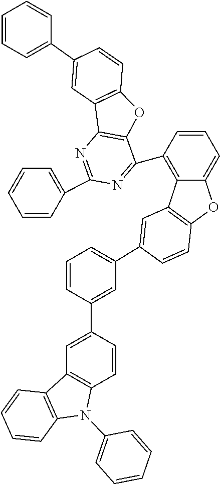 [SMILES: c1ccc(-c2ccc3oc4c(-c5cccc6oc7ccc(-c8cccc(-c9ccc%10c(c9)c9ccccc9n%10-c9ccccc9)c8)cc7c56)nc(-c5ccccc5)nc4c3c2)cc1]

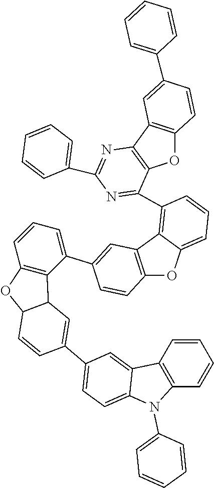 [SMILES: C1=CC2Oc3cccc(-c4ccc5oc6cccc(-c7nc(-c8ccccc8)nc8c7oc7ccc(-c9ccccc9)cc78)c6c5c4)c3C2C=C1c1ccc2c(c1)c1ccccc1n2-c1ccccc1]

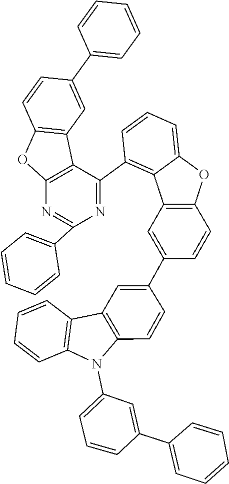 [SMILES: c1ccc(-c2cccc(-n3c4ccccc4c4cc(-c5ccc6oc7cccc(-c8nc(-c9ccccc9)nc9oc%10ccc(-c%11ccccc%11)cc%10c89)c7c6c5)ccc43)c2)cc1]

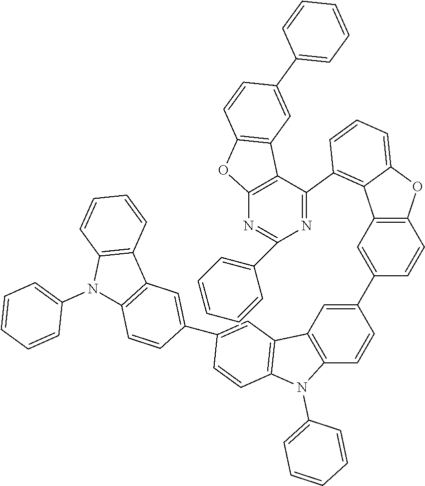 [SMILES: c1ccc(-c2ccc3oc4nc(-c5ccccc5)nc(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)c4c3c2)cc1]

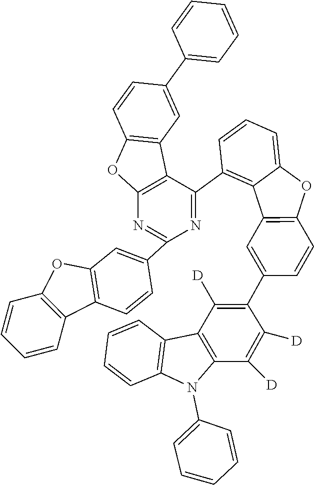 [SMILES: [2H]c1c(-c2ccc3oc4cccc(-c5nc(-c6ccc7c(c6)oc6ccccc67)nc6oc7ccc(-c8ccccc8)cc7c56)c4c3c2)c([2H])c2c3ccccc3n(-c3ccccc3)c2c1[2H]]

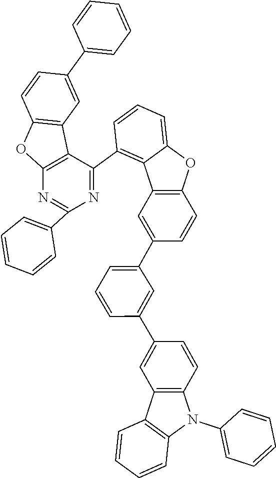 [SMILES: c1ccc(-c2ccc3oc4nc(-c5ccccc5)nc(-c5cccc6oc7ccc(-c8cccc(-c9ccc%10c(c9)c9ccccc9n%10-c9ccccc9)c8)cc7c56)c4c3c2)cc1]

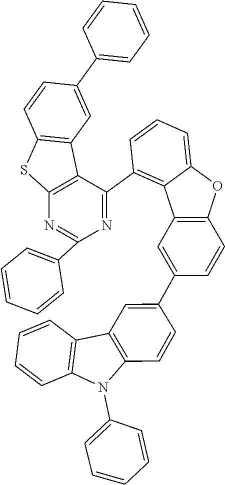 [SMILES: c1ccc(-c2ccc3sc4nc(-c5ccccc5)nc(-c5cccc6oc7ccc(-c8ccc9c(c8)c8ccccc8n9-c8ccccc8)cc7c56)c4c3c2)cc1]

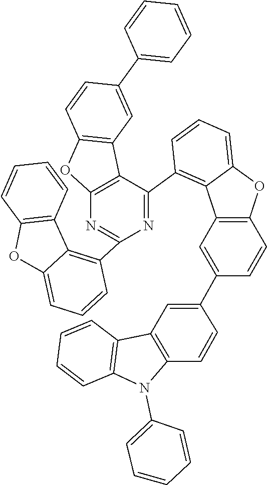 [SMILES: c1ccc(-c2ccc3oc4nc(-c5cccc6oc7ccccc7c56)nc(-c5cccc6oc7ccc(-c8ccc9c(c8)c8ccccc8n9-c8ccccc8)cc7c56)c4c3c2)cc1]

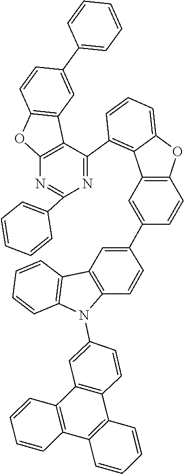 [SMILES: c1ccc(-c2ccc3oc4nc(-c5ccccc5)nc(-c5cccc6oc7ccc(-c8ccc9c(c8)c8ccccc8n9-c8ccc9c%10ccccc%10c%10ccccc%10c9c8)cc7c56)c4c3c2)cc1]

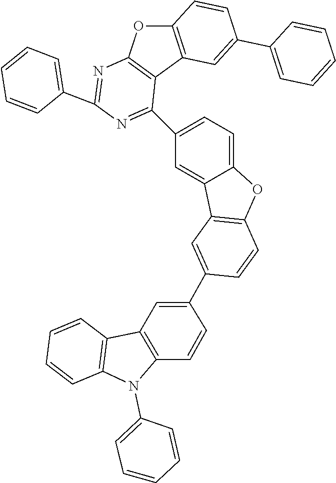 [SMILES: c1ccc(-c2ccc3oc4nc(-c5ccccc5)nc(-c5ccc6oc7ccc(-c8ccc9c(c8)c8ccccc8n9-c8ccccc8)cc7c6c5)c4c3c2)cc1]

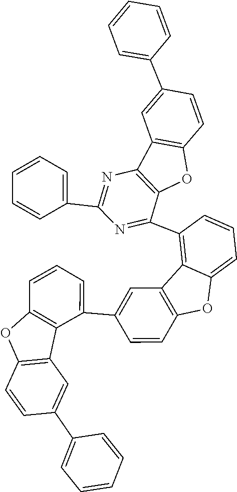 [SMILES: c1ccc(-c2ccc3oc4c(-c5cccc6oc7ccc(-c8cccc9oc%10ccc(-c%11ccccc%11)cc%10c89)cc7c56)nc(-c5ccccc5)nc4c3c2)cc1]

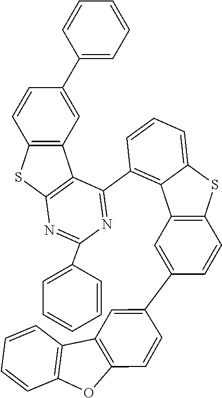 [SMILES: c1ccc(-c2ccc3sc4nc(-c5ccccc5)nc(-c5cccc6sc7ccc(-c8ccc9oc%10ccccc%10c9c8)cc7c56)c4c3c2)cc1]

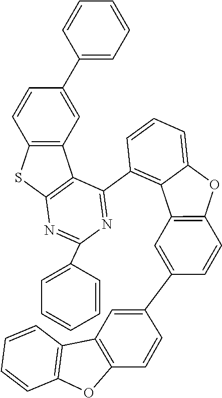 [SMILES: c1ccc(-c2ccc3sc4nc(-c5ccccc5)nc(-c5cccc6oc7ccc(-c8ccc9oc%10ccccc%10c9c8)cc7c56)c4c3c2)cc1]

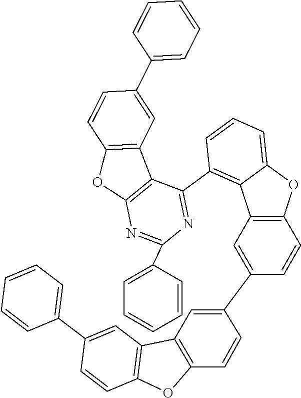 [SMILES: c1ccc(-c2ccc3oc4ccc(-c5ccc6oc7cccc(-c8nc(-c9ccccc9)nc9oc%10ccc(-c%11ccccc%11)cc%10c89)c7c6c5)cc4c3c2)cc1]

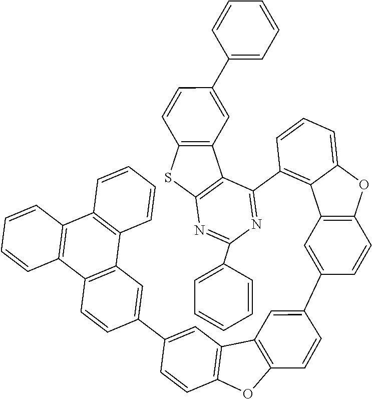 [SMILES: c1ccc(-c2ccc3sc4nc(-c5ccccc5)nc(-c5cccc6oc7ccc(-c8ccc9oc%10ccc(-c%11ccc%12c%13ccccc%13c%13ccccc%13c%12c%11)cc%10c9c8)cc7c56)c4c3c2)cc1]

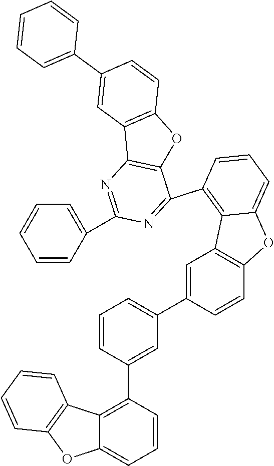 [SMILES: c1ccc(-c2ccc3oc4c(-c5cccc6oc7ccc(-c8cccc(-c9cccc%10oc%11ccccc%11c9%10)c8)cc7c56)nc(-c5ccccc5)nc4c3c2)cc1]

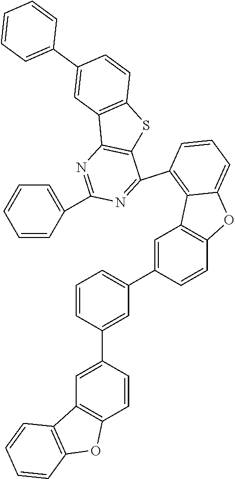 [SMILES: c1ccc(-c2ccc3sc4c(-c5cccc6oc7ccc(-c8cccc(-c9ccc%10oc%11ccccc%11c%10c9)c8)cc7c56)nc(-c5ccccc5)nc4c3c2)cc1]

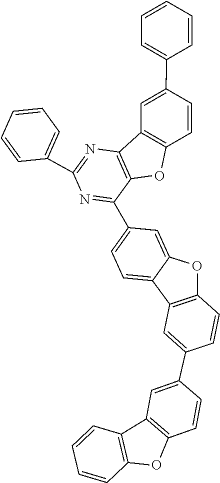 [SMILES: c1ccc(-c2ccc3oc4c(-c5ccc6c(c5)oc5ccc(-c7ccc8oc9ccccc9c8c7)cc56)nc(-c5ccccc5)nc4c3c2)cc1]

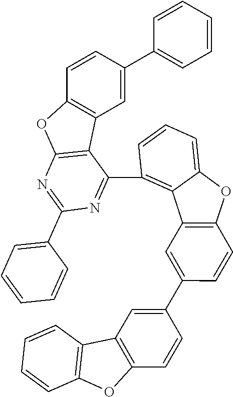 [SMILES: c1ccc(-c2ccc3oc4nc(-c5ccccc5)nc(-c5cccc6oc7ccc(-c8ccc9oc%10ccccc%10c9c8)cc7c56)c4c3c2)cc1]

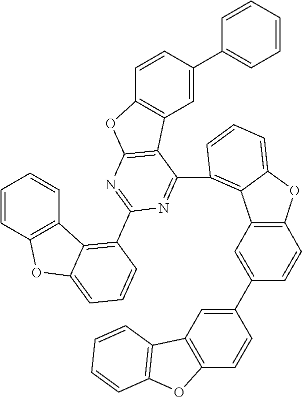 [SMILES: c1ccc(-c2ccc3oc4nc(-c5cccc6oc7ccccc7c56)nc(-c5cccc6oc7ccc(-c8ccc9oc%10ccccc%10c9c8)cc7c56)c4c3c2)cc1]

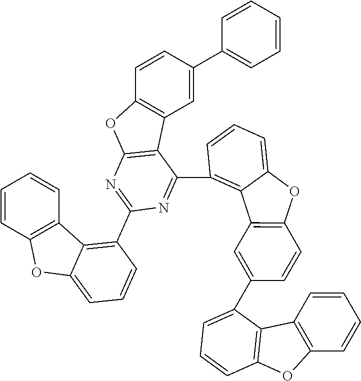 [SMILES: c1ccc(-c2ccc3oc4nc(-c5cccc6oc7ccccc7c56)nc(-c5cccc6oc7ccc(-c8cccc9oc%10ccccc%10c89)cc7c56)c4c3c2)cc1]

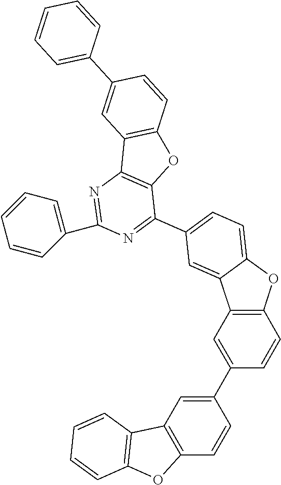 [SMILES: c1ccc(-c2ccc3oc4c(-c5ccc6oc7ccc(-c8ccc9oc%10ccccc%10c9c8)cc7c6c5)nc(-c5ccccc5)nc4c3c2)cc1]

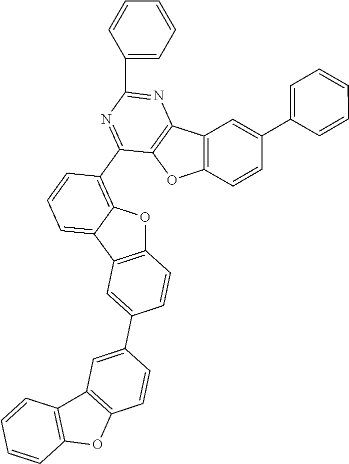 [SMILES: c1ccc(-c2ccc3oc4c(-c5cccc6c5oc5ccc(-c7ccc8oc9ccccc9c8c7)cc56)nc(-c5ccccc5)nc4c3c2)cc1]

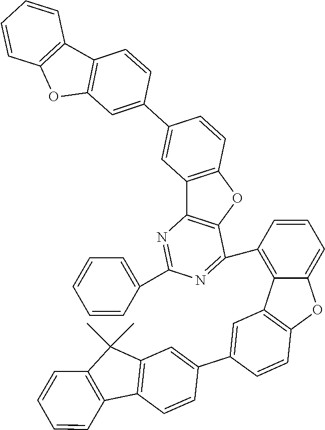 [SMILES: CC1(C)c2ccccc2-c2ccc(-c3ccc4oc5cccc(-c6nc(-c7ccccc7)nc7c6oc6ccc(-c8ccc9c(c8)oc8ccccc89)cc67)c5c4c3)cc21]

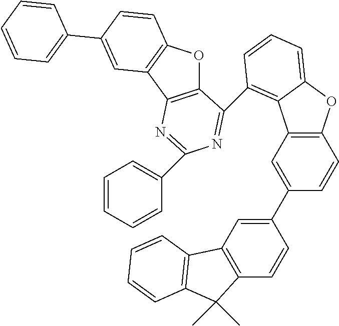 [SMILES: CC1(C)c2ccccc2-c2cc(-c3ccc4oc5cccc(-c6nc(-c7ccccc7)nc7c6oc6ccc(-c8ccccc8)cc67)c5c4c3)ccc21]

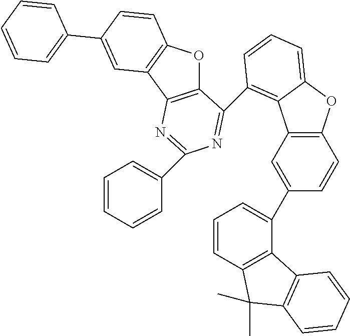 [SMILES: CC1(C)c2ccccc2-c2c(-c3ccc4oc5cccc(-c6nc(-c7ccccc7)nc7c6oc6ccc(-c8ccccc8)cc67)c5c4c3)cccc21]

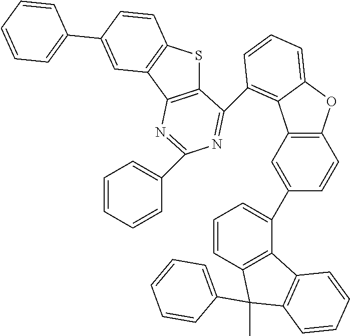 [SMILES: CC1(c2ccccc2)c2ccccc2-c2c(-c3ccc4oc5cccc(-c6nc(-c7ccccc7)nc7c6sc6ccc(-c8ccccc8)cc67)c5c4c3)cccc21]

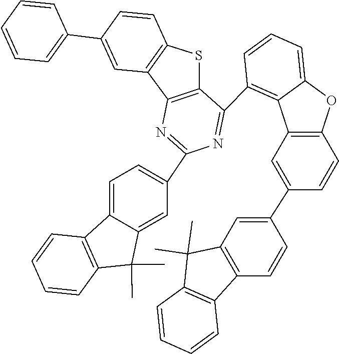 [SMILES: CC1(C)c2ccccc2-c2ccc(-c3ccc4oc5cccc(-c6nc(-c7ccc8c(c7)C(C)(C)c7ccccc7-8)nc7c6sc6ccc(-c8ccccc8)cc67)c5c4c3)cc21]

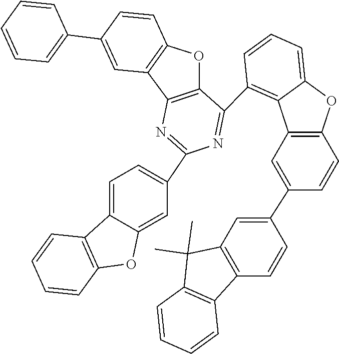 [SMILES: CC1(C)c2ccccc2-c2ccc(-c3ccc4oc5cccc(-c6nc(-c7ccc8c(c7)oc7ccccc78)nc7c6oc6ccc(-c8ccccc8)cc67)c5c4c3)cc21]

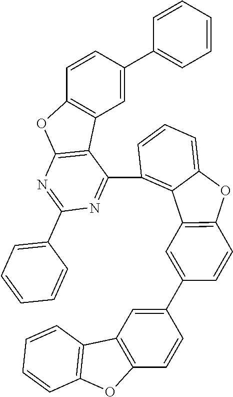 [SMILES: c1ccc(-c2ccc3oc4nc(-c5ccccc5)nc(-c5cccc6oc7ccc(-c8ccc9oc%10ccccc%10c9c8)cc7c56)c4c3c2)cc1]

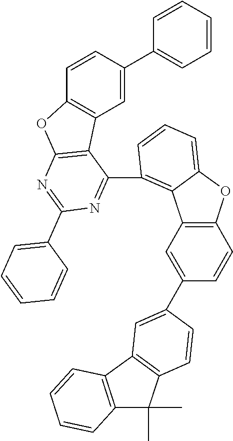 [SMILES: CC1(C)c2ccccc2-c2cc(-c3ccc4oc5cccc(-c6nc(-c7ccccc7)nc7oc8ccc(-c9ccccc9)cc8c67)c5c4c3)ccc21]

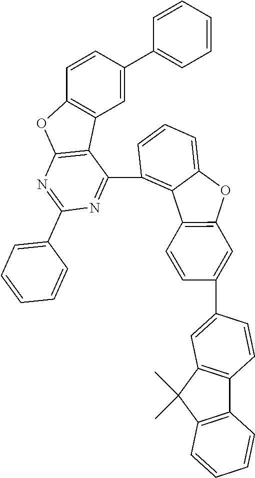 [SMILES: CC1(C)c2ccccc2-c2ccc(-c3ccc4c(c3)oc3cccc(-c5nc(-c6ccccc6)nc6oc7ccc(-c8ccccc8)cc7c56)c34)cc21]

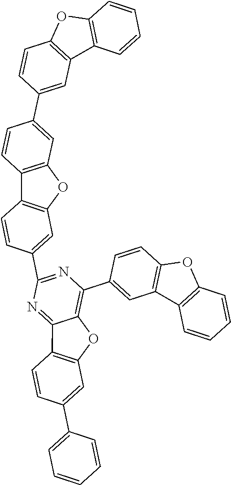 [SMILES: c1ccc(-c2ccc3c(c2)oc2c(-c4ccc5oc6ccccc6c5c4)nc(-c4ccc5c(c4)oc4cc(-c6ccc7oc8ccccc8c7c6)ccc45)nc23)cc1]

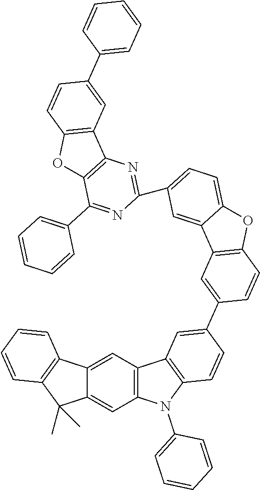 [SMILES: CC1(C)c2ccccc2-c2cc3c4cc(-c5ccc6oc7ccc(-c8nc(-c9ccccc9)c9oc%10ccc(-c%11ccccc%11)cc%10c9n8)cc7c6c5)ccc4n(-c4ccccc4)c3cc21]

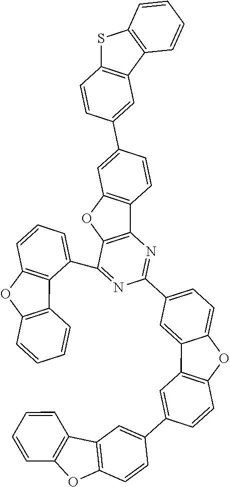 [SMILES: c1ccc2c(c1)oc1ccc(-c3ccc4oc5ccc(-c6nc(-c7cccc8oc9ccccc9c78)c7oc8cc(-c9ccc%10sc%11ccccc%11c%10c9)ccc8c7n6)cc5c4c3)cc12]

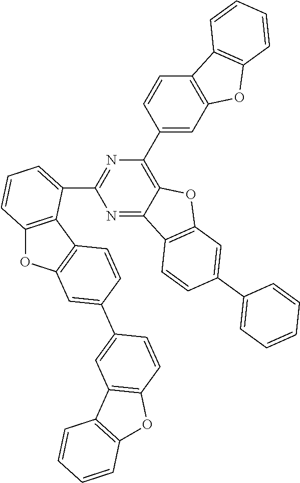 [SMILES: c1ccc(-c2ccc3c(c2)oc2c(-c4ccc5c(c4)oc4ccccc45)nc(-c4cccc5oc6cc(-c7ccc8oc9ccccc9c8c7)ccc6c45)nc23)cc1]

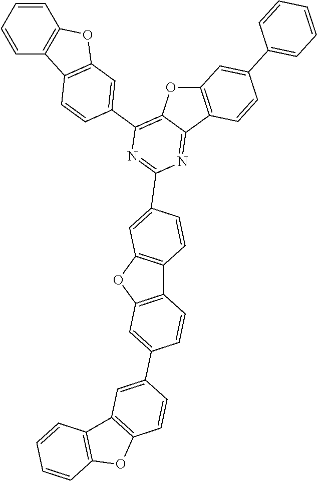 [SMILES: c1ccc(-c2ccc3c(c2)oc2c(-c4ccc5c(c4)oc4ccccc45)nc(-c4ccc5c(c4)oc4cc(-c6ccc7oc8ccccc8c7c6)ccc45)nc23)cc1]

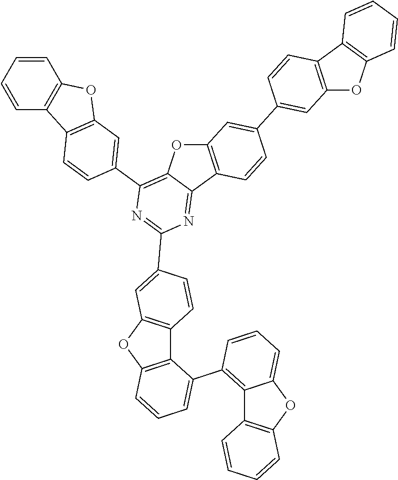 [SMILES: c1ccc2c(c1)oc1cc(-c3ccc4c(c3)oc3c(-c5ccc6c(c5)oc5ccccc56)nc(-c5ccc6c(c5)oc5cccc(-c7cccc8oc9ccccc9c78)c56)nc34)ccc12]

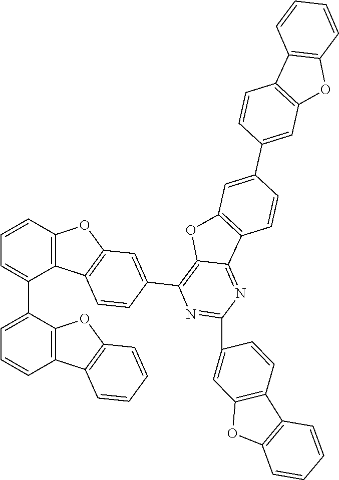 [SMILES: c1ccc2c(c1)oc1cc(-c3ccc4c(c3)oc3c(-c5ccc6c(c5)oc5cccc(-c7cccc8c7oc7ccccc78)c56)nc(-c5ccc6c(c5)oc5ccccc56)nc34)ccc12]

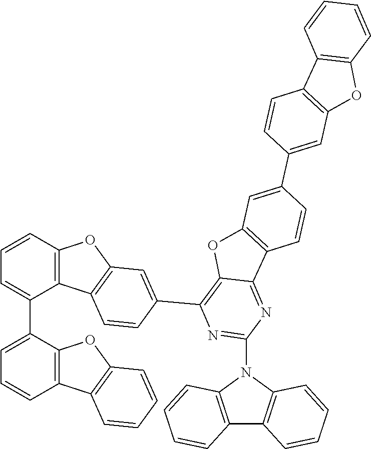 [SMILES: c1ccc2c(c1)oc1cc(-c3ccc4c(c3)oc3c(-c5ccc6c(c5)oc5cccc(-c7cccc8c7oc7ccccc78)c56)nc(-n5c6ccccc6c6ccccc65)nc34)ccc12]

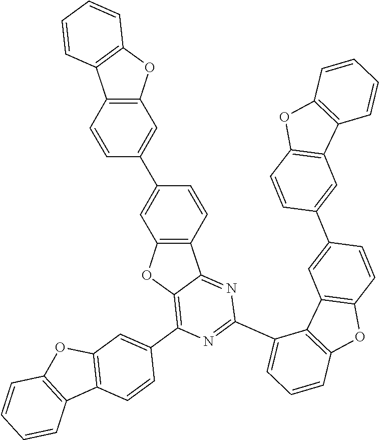 [SMILES: c1ccc2c(c1)oc1cc(-c3ccc4c(c3)oc3c(-c5ccc6c(c5)oc5ccccc56)nc(-c5cccc6oc7ccc(-c8ccc9oc%10ccccc%10c9c8)cc7c56)nc34)ccc12]

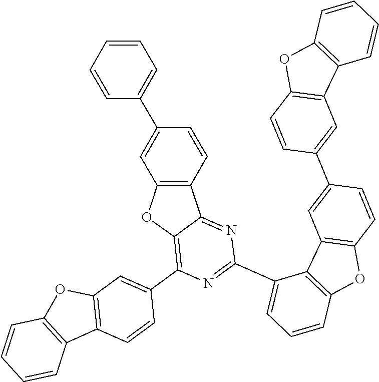 [SMILES: c1ccc(-c2ccc3c(c2)oc2c(-c4ccc5c(c4)oc4ccccc45)nc(-c4cccc5oc6ccc(-c7ccc8oc9ccccc9c8c7)cc6c45)nc23)cc1]

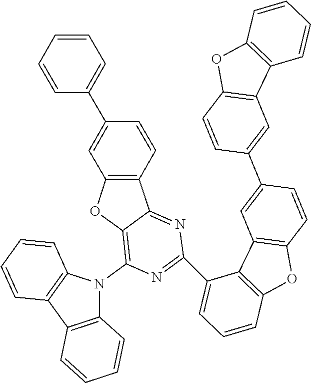 [SMILES: c1ccc(-c2ccc3c(c2)oc2c(-n4c5ccccc5c5ccccc54)nc(-c4cccc5oc6ccc(-c7ccc8oc9ccccc9c8c7)cc6c45)nc23)cc1]

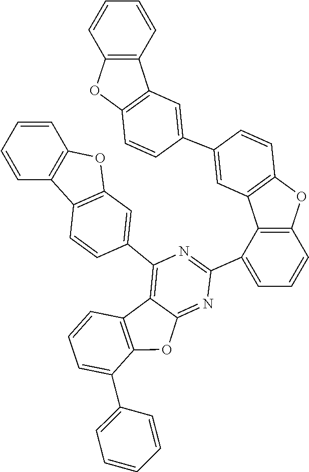 [SMILES: c1ccc(-c2cccc3c2oc2nc(-c4cccc5oc6ccc(-c7ccc8oc9ccccc9c8c7)cc6c45)nc(-c4ccc5c(c4)oc4ccccc45)c23)cc1]

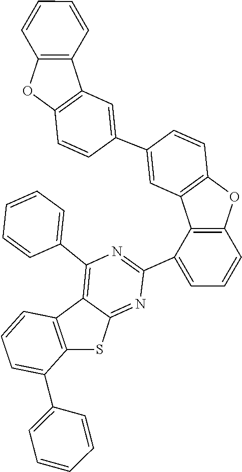 [SMILES: c1ccc(-c2cccc3c2sc2nc(-c4cccc5oc6ccc(-c7ccc8oc9ccccc9c8c7)cc6c45)nc(-c4ccccc4)c23)cc1]

Further examples of compounds of the formula (1) are described in the implementation section.

Particularly suitable compounds of the formula (1) that are preferably used in combination with at least one compound of the formula (2) in the electroluminescent device of the invention are the compounds E1 to E27:

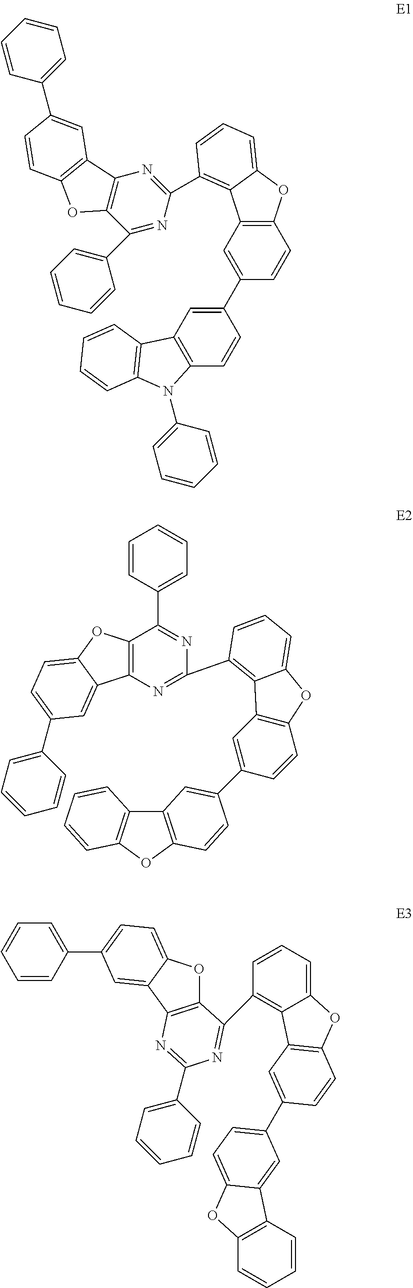

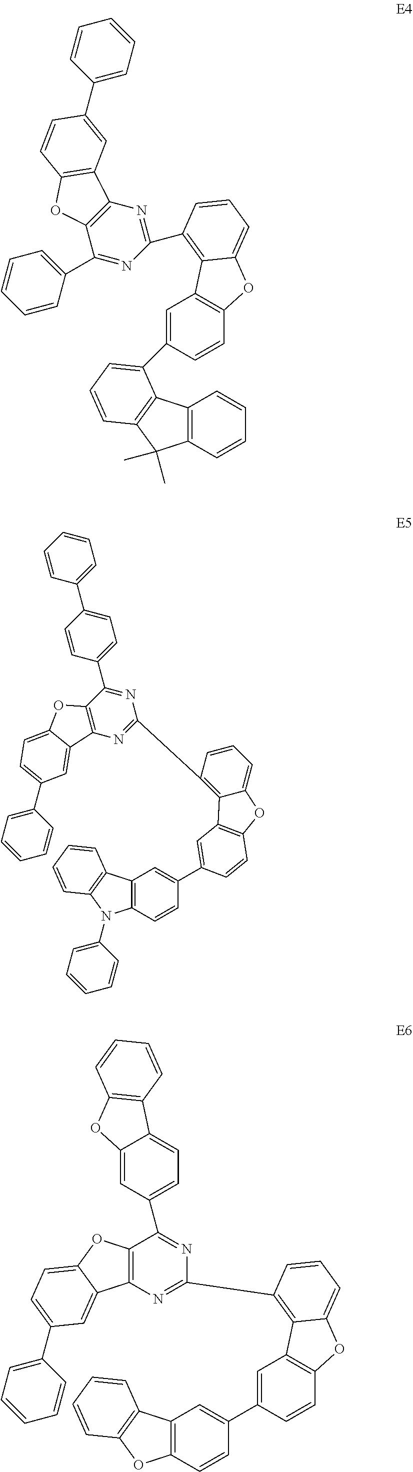

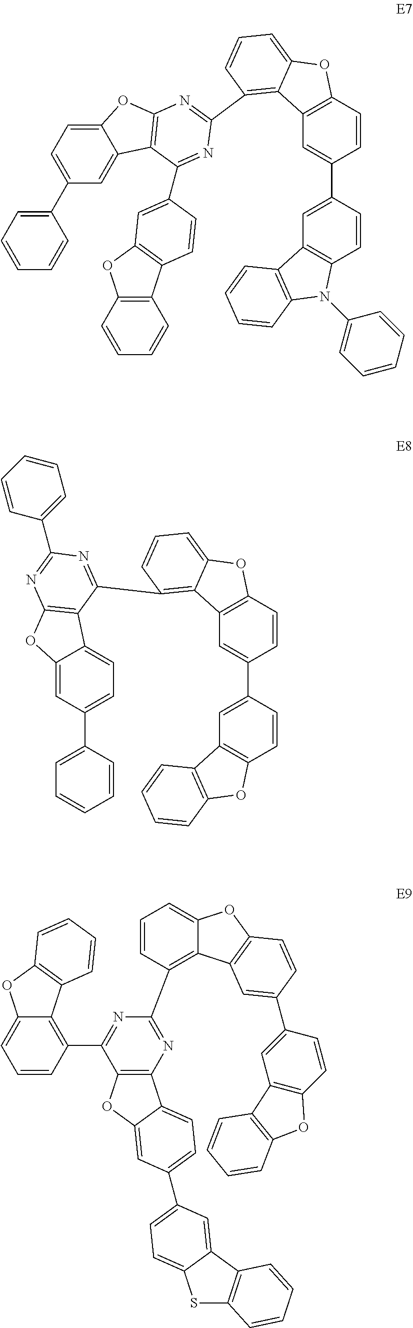 [SMILES: c1ccc(-c2ccc3oc4nc(-c5cccc6oc7ccc(-c8ccc9c(c8)c8ccccc8n9-c8ccccc8)cc7c56)nc(-c5ccc6c(c5)oc5ccccc56)c4c3c2)cc1 ; c1ccc(-c2ccc3c(c2)oc2nc(-c4ccccc4)nc(-c4cccc5oc6ccc(-c7ccc8oc9ccccc9c8c7)cc6c45)c23)cc1 ; c1ccc2c(c1)oc1ccc(-c3ccc4oc5cccc(-c6nc(-c7cccc8oc9ccccc9c78)c7oc8cc(-c9ccc%10sc%11ccccc%11c%10c9)ccc8c7n6)c5c4c3)cc12]

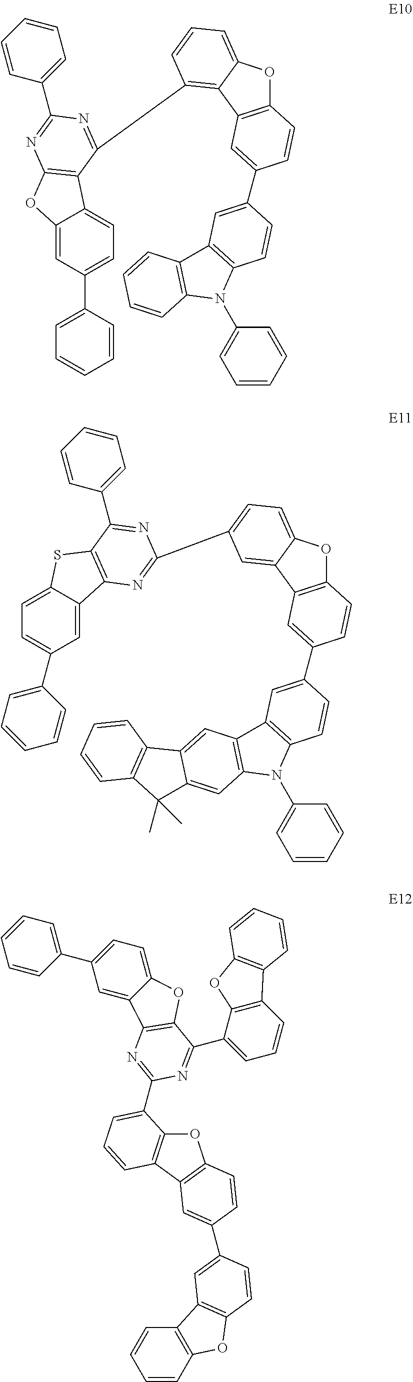 [SMILES: c1ccc(-c2ccc3c(c2)oc2nc(-c4ccccc4)nc(-c4cccc5oc6ccc(-c7ccc8c(c7)c7ccccc7n8-c7ccccc7)cc6c45)c23)cc1 ; CC1(C)c2ccccc2-c2cc3c4cc(-c5ccc6oc7ccc(-c8nc(-c9ccccc9)c9sc%10ccc(-c%11ccccc%11)cc%10c9n8)cc7c6c5)ccc4n(-c4ccccc4)c3cc21 ; c1ccc(-c2ccc3oc4c(-c5cccc6c5oc5ccccc56)nc(-c5cccc6c5oc5ccc(-c7ccc8oc9ccccc9c8c7)cc56)nc4c3c2)cc1]

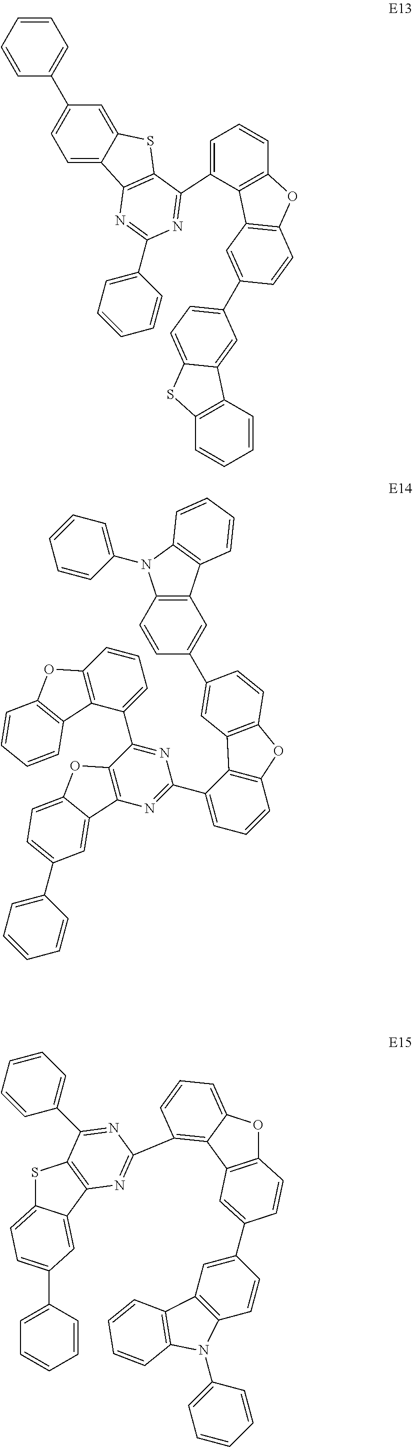

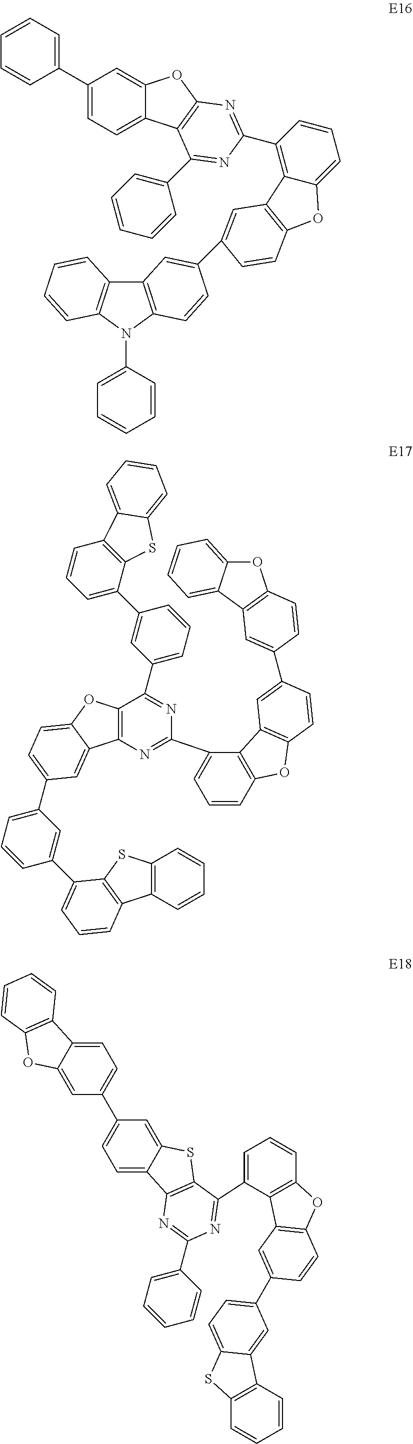

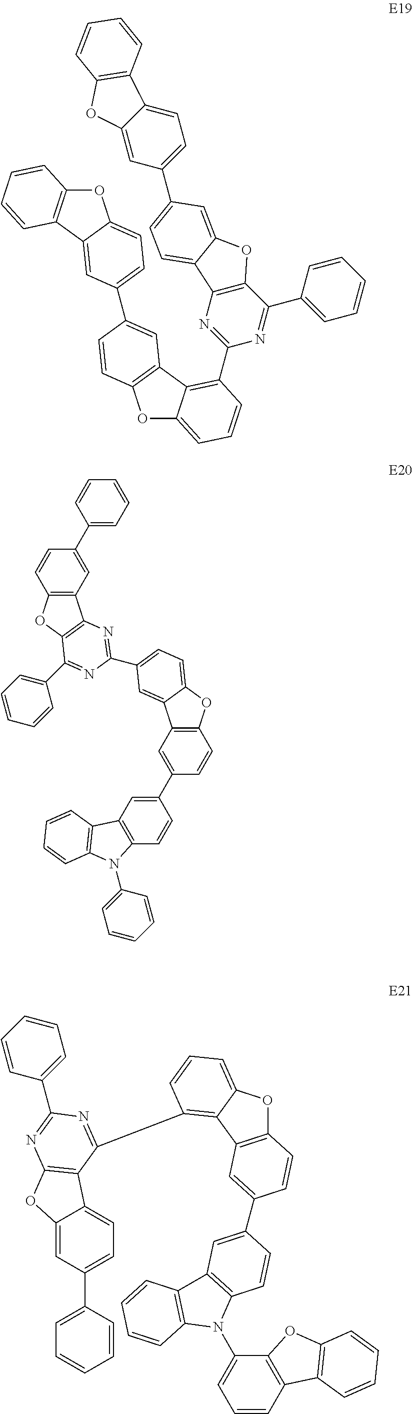 [SMILES: c1ccc(-c2nc(-c3cccc4oc5ccc(-c6ccc7oc8ccccc8c7c6)cc5c34)nc3c2oc2cc(-c4ccc5c(c4)oc4ccccc45)ccc23)cc1 ; c1ccc(-c2ccc3oc4c(-c5ccccc5)nc(-c5ccc6oc7ccc(-c8ccc9c(c8)c8ccccc8n9-c8ccccc8)cc7c6c5)nc4c3c2)cc1 ; c1ccc(-c2ccc3c(c2)oc2nc(-c4ccccc4)nc(-c4cccc5oc6ccc(-c7ccc8c(c7)c7ccccc7n8-c7cccc8c7oc7ccccc78)cc6c45)c23)cc1]

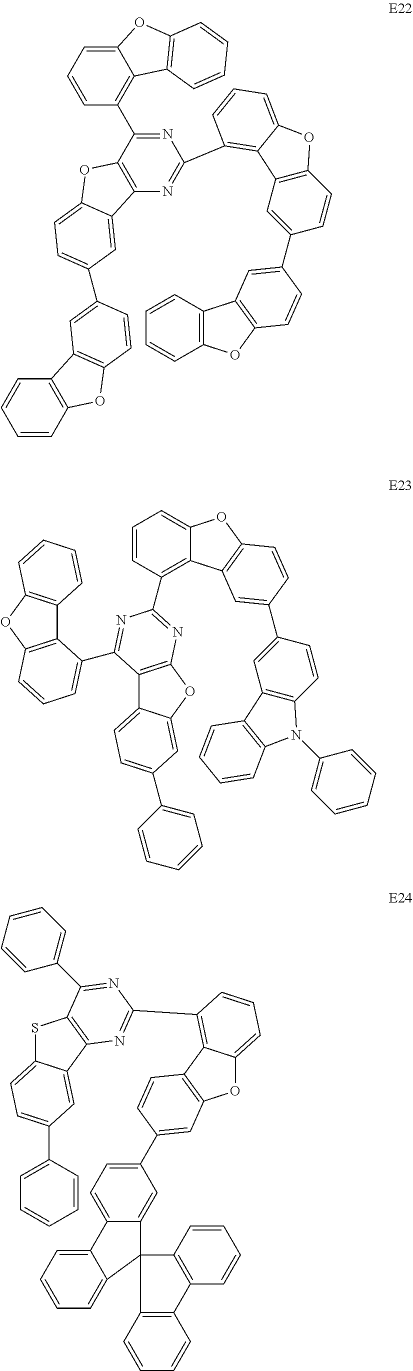 [SMILES: c1ccc2c(c1)oc1ccc(-c3ccc4oc5c(-c6cccc7oc8ccccc8c67)nc(-c6cccc7oc8ccc(-c9ccc%10oc%11ccccc%11c%10c9)cc8c67)nc5c4c3)cc12 ; c1ccc(-c2ccc3c(c2)oc2nc(-c4cccc5oc6ccc(-c7ccc8c(c7)c7ccccc7n8-c7ccccc7)cc6c45)nc(-c4cccc5oc6ccccc6c45)c23)cc1 ; c1ccc(-c2ccc3sc4c(-c5ccccc5)nc(-c5cccc6oc7cc(-c8ccc9c(c8)C8(c%10ccccc%10-c%10ccccc%108)c8ccccc8-9)ccc7c56)nc4c3c2)cc1]

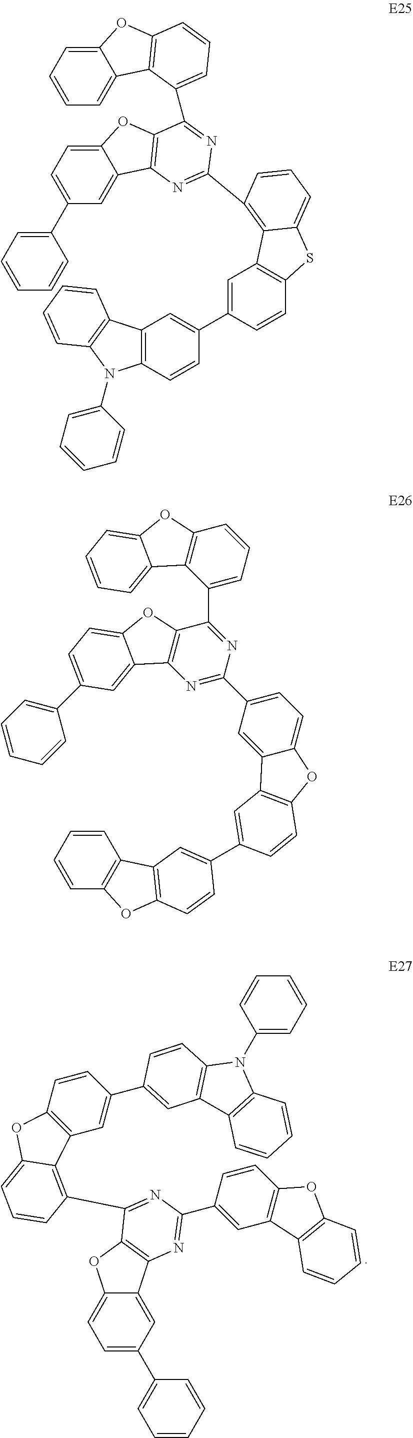 [SMILES: c1ccc(-c2ccc3oc4c(-c5cccc6oc7ccccc7c56)nc(-c5cccc6sc7ccc(-c8ccc9c(c8)c8ccccc8n9-c8ccccc8)cc7c56)nc4c3c2)cc1 ; c1ccc(-c2ccc3oc4c(-c5cccc6oc7ccccc7c56)nc(-c5ccc6oc7ccc(-c8ccc9oc%10ccccc%10c9c8)cc7c6c5)nc4c3c2)cc1 ; c1ccc(-c2ccc3oc4c(-c5cccc6oc7ccc(-c8ccc9c(c8)c8ccccc8n9-c8ccccc8)cc7c56)nc(-c5ccc6oc7ccccc7c6c5)nc4c3c2)cc1 ; "."]

The preparation of the compounds of the formula (1) or of the preferred compounds from table 1 and of the compounds E1 to E27 is known to those skilled in the art. The compounds may be prepared by synthesis steps known to the person skilled in the art, for example halogenation, preferably bromination, and a subsequent organometallic coupling reaction, for example Suzuki coupling, Heck coupling or Hartwig-Buchwald coupling.

Precursor compounds for compounds of the formula (1) can be prepared according to scheme 1 below, where the symbols and indices have one of the definitions given above and L denotes a bond, and m and R # have one of the definitions specified above or specified as preferred.

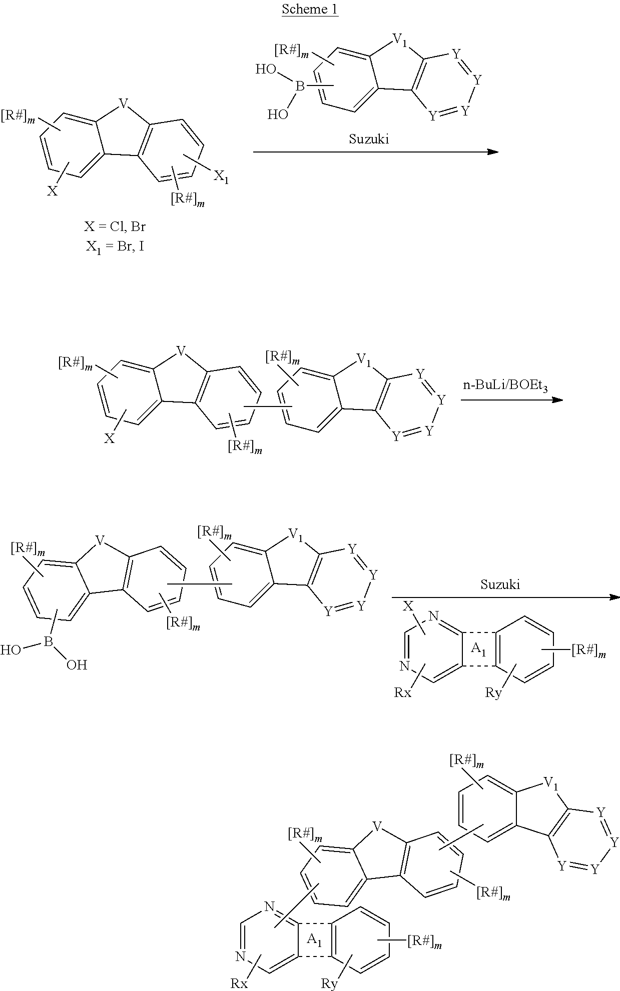

The adjustment of the preparation of the host material 1 containing a linker L is sufficiently well known to the person skilled in the art and is described in the implementation section. The conditions of the preparation as described in the implementation section are also applicable to the synthesis of the host material 1 according to scheme 1.

There follows a description of the host material 2 and its preferred embodiments that is/are present in the device of the invention. The preferred embodiments of the host material 1 of the formula (1) are also applicable to the mixture and/or formulation of the invention.

Host material 2 is at least one compound of the formula (2),

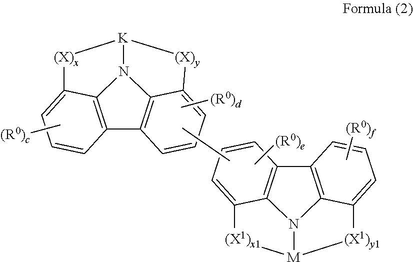

where the symbols and indices used are as follows:
K, M are each independently an unsubstituted or partly or fully deuterated or R*-monosubstituted aromatic ring system having 6 to 40 ring atoms when x and y are 0 and when x1 and y1 are 0, or
K, M each independently together with X or X¹ form a heteroaromatic ring system having 14 to 40 ring atoms when the value of x, x1, y and/or y1 is 1;
x, x1 at each instance are each independently 0 or 1;
y, y1 at each instance are each independently 0 or 1;
X and X¹ at each instance are each independently a bond or C(R⁺)₂;
R⁰ at each instance is independently an unsubstituted or partly or fully deuterated aromatic ring system having 6 to 18 carbon atoms;
R⁺ at each instance is independently a straight-chain or branched alkyl group having 1 to 4 carbon atoms and
c, d, e and f are independently 0 or 1.

In one embodiment of the invention, for the device of the invention, compounds of the formula (2) as described above are selected, and these are used in the light-emitting layer with compounds of the formula (1) as described above or described as preferred or with the compounds from table 1 or the compounds E1 to E27.

In a preferred embodiment of the device of the invention, compounds of the formula (2) in which x, y, x1 and y1 are 0 are used as host material 2. Compounds of the formula (2) in which x, x1, y and y1 at each instance are 0 may be represented by the following formula (2a):

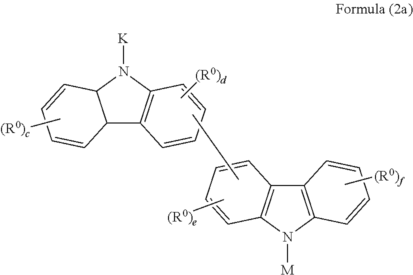

where R⁰, c, d, e and f are as defined above or hereinafter and
K and M are each independently an unsubstituted or partly or fully deuterated or R*-monosubstituted aromatic ring system having 6 to 40 ring atoms.

In preferred compounds of the formula (2a), the sum total of the indices c+d+e+f is preferably 0 or 1 and R⁰ is as defined as preferred above or hereinafter.

In compounds of the formula (2) or (2a), R⁰ at each instance is preferably independently an unsubstituted aromatic ring system having 6 to 18 carbon atoms. R⁰ at each instance is preferably independently phenyl, 1,3-biphenyl, 1,4-biphenyl, naphthyl or triphenylenyl. R⁰ at each instance is more preferably independently phenyl.

In compounds of the formula (2) or (2a) the indices c, d, e and f are more preferably 0.

In compounds of the formula (2) or (2a), K and M at each instance are preferably independently an unsubstituted or partly deuterated aromatic ring system having 6 to 40 ring atoms as described above. K and M in compounds of the formula (2) or (2a) at each instance are more preferably independently phenyl, deuterated phenyl, 1,3-biphenyl, 1,4-biphenyl, terphenyl, partly deuterated terphenyl, quaterphenyl, naphthyl, fluorenyl, 9,9-diphenylfluorenyl, bispirofluorenyl or triphenylenyl.

The invention accordingly further provides an organic electroluminescent device as described above or described as preferred, wherein the at least one compound of the formula (2) corresponds to a compound of the formula (2a) or to a preferred embodiment of the compound of the formula (2a).

In a preferred embodiment of the device of the invention, compounds of the formula (2) in which x1 and y1 are 0, x and y are 0 or 1 and the sum of x and y is 1 or 2 are used as host material 2. Compounds of the formula (2) in which x1 and y1 are 0, x and y are 0 or 1 and the sum of x and y is 1 or 2 may be represented by the following formula (2b),

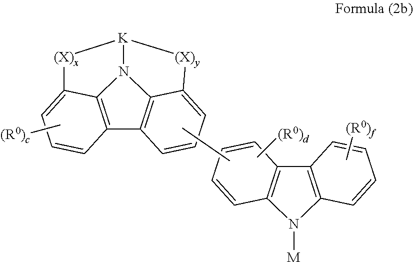

where X, x, y, R⁰, c, d, e and f are as defined above or hereinafter,
M is an unsubstituted or partly or fully deuterated or R*-monosubstituted aromatic ring system having 6 to 40 ring atoms and
K together with X forms a heteroaromatic ring system having 14 to 40 ring atoms when the value of x or y is 1 or both values x and y are 1.

In preferred compounds of the formula (2b), the sum total of the indices c+d+e+f is preferably 0, 1 or 2 and R⁰ is as defined as described above or described as preferred.

In compounds of the formula (2) or (2b), the indices c, d, e and f are more preferably 0 or 1. In compounds of the formula (2) or (2b), the indices c, d, e and f are even more preferably 0. In compounds of the formula (2) or (2b), the indices c, d, e and f are even more preferably 1. In compounds of the formula (2) or (2b), the indices c, d, e and f are even more preferably 2.

In compounds of the formula (2) or (2b), K preferably forms a heteroaromatic ring system when the sum of x+y is 1 or 2. In compounds of the formula (2) or (2b), X is preferably a direct bond or C(CH₃)₂.

Preferred compounds of the formula (2) or (2b) may be represented by the formulae (2b-1) to (2b-6),

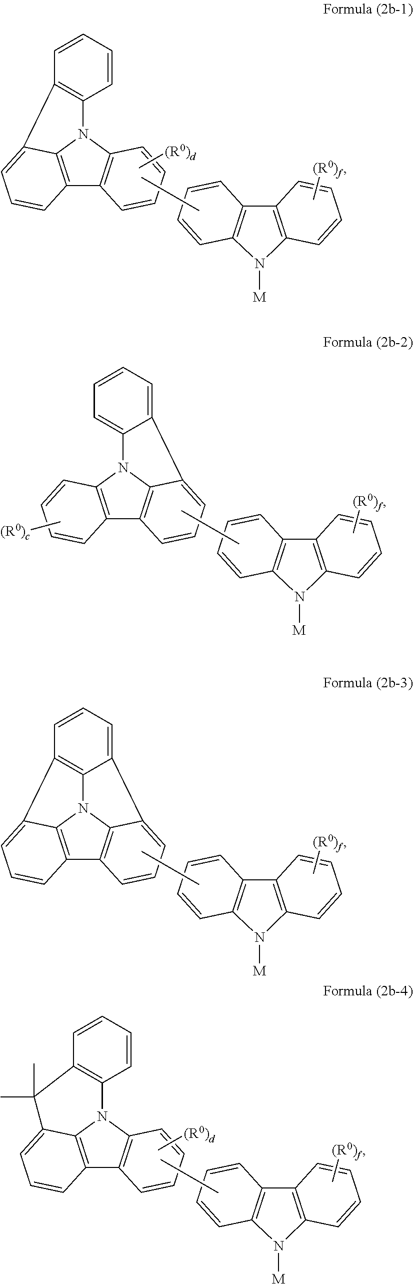

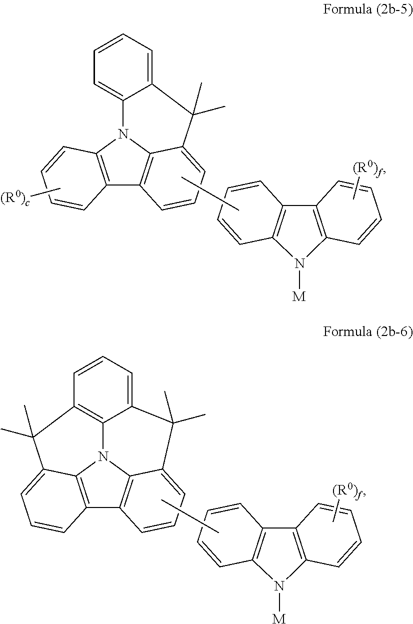

where M, R⁰, c, d, e and f are defined as described above or described as preferred.

In compounds of the formulae (2), (2b), (2b-1), (2b-2), (2b-3), (2b-4), (2b-5) or (2b-6), M is preferably an unsubstituted or partly deuterated aromatic ring system having 6 to 40 ring atoms as described above. M in compounds of the formulae (2), (2b), (2b-1), (2b-2), (2b-3), (2b-4), (2b-5) or (2b-6) is more preferably phenyl, deuterated phenyl, 1,3-biphenyl, 1,4-biphenyl, terphenyl, partially deuterated terphenyl, quaterphenyl, naphthyl, fluorenyl, 9,9-diphenylfluorenyl, bispirofluorenyl or triphenylenyl.

In compounds of the formulae (2b-1), (2b-2), (2b-3), (2b-4), (2b-5) or (2b-6), c, d, e and f are preferably 0 or 1.

The invention accordingly further provides an organic electroluminescent device as described above or described as preferred, wherein the at least one compound of the formula (2) corresponds to a compound of the formula (2b), (2b-1), (2b-2), (2b-3), (2b-4), (2b-5) or (2b-6) or to a preferred embodiment of these compounds.

In a preferred embodiment of the device of the invention, compounds of the formula (2) in which c and f are 0 or 1, d and e are 0 and x, x1, y and y1 independently at each instance represent 0 or 1, but the sum of x and y is at least 1 and the sum of x1 and y1 is at least 1, are used as host material 2. Such compounds of the formula (2) as described above may preferably be represented by the following formula (2c),

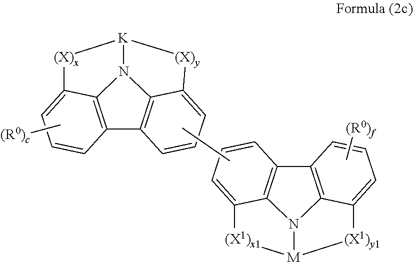

where X and X¹ are as defined above or hereinafter,
K and M each independently together with X or X 1 form a heteroaromatic ring system having 14 to 40 ring atoms,
x, x1, y and/or y1 are 0 or 1 and the sum of x and y is at least 1 and the sum of x1 and y1 is at least 1.

In preferred compounds of the formula (2c) the sum of x and y is 1 or 2 and the sum of x1 and y1 is 1. In particularly preferred compounds of the formula (2c) the sum of x and y is 1 and the sum of x1 and y1 is in each case 1.

In compounds of the formula (2) or (2c) K and M thus preferably form a heteroaromatic ring system. In compounds of the formula (2) or (2c) X and X¹ are preferably a direct bond or C(CH₃)₂.

Preferred compounds of the formula (2) or (2c) may be represented by the formulae (2c-1) to (2c-8),

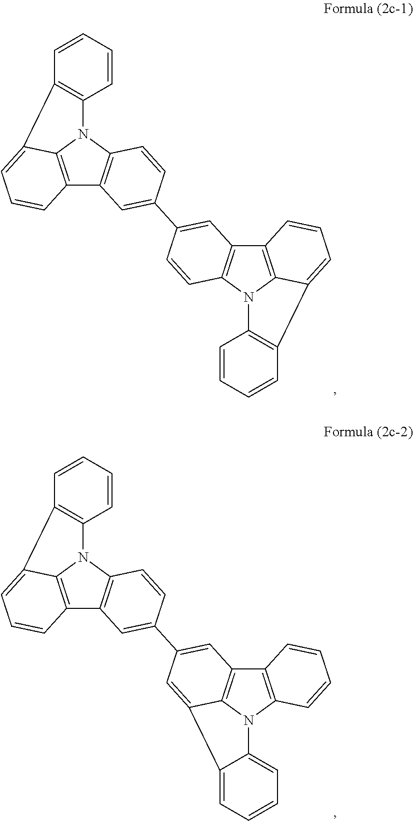 [SMILES: c1ccc2c(c1)c1cccc3c4cc(-c5ccc6c(c5)c5cccc7c8ccccc8n6c75)ccc4n2c13 ; c1ccc2c(c1)c1cccc3c4cc(-c5cc6c7ccccc7n7c8ccccc8c(c5)c67)ccc4n2c13]

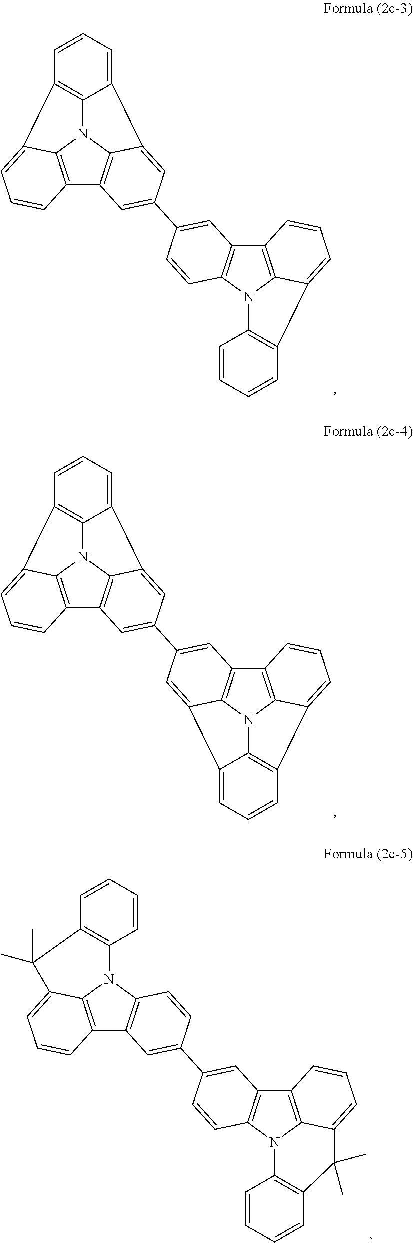

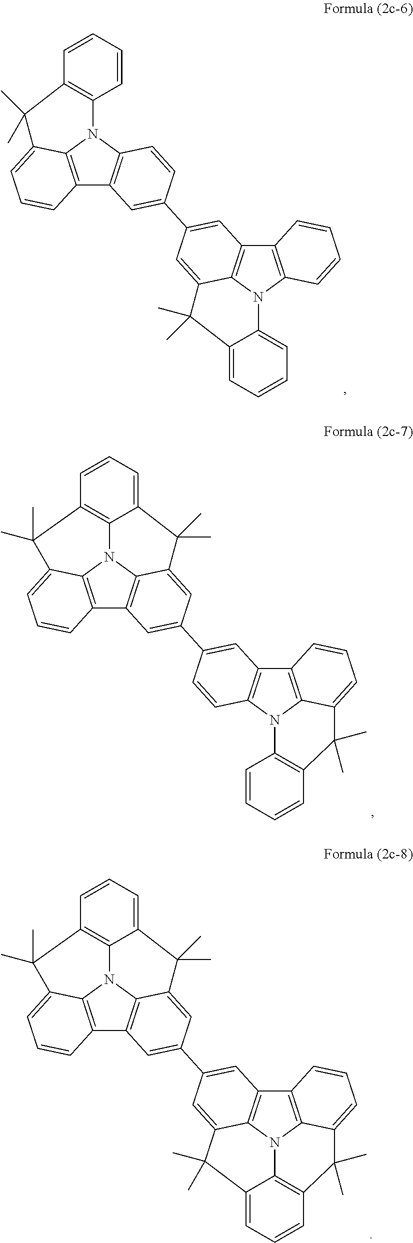

Preferred compounds of the formula (2c) are also the compounds H9, H11, H12, H13, H14, H15, H19 and H20 as described hereinafter.

The invention accordingly further provides an organic electroluminescent device as described above or described as preferred, wherein the at least one compound of the formula (2) corresponds to a compound of the formulae (2c), (2c-1), (2c-2), (2c-3), (2c-4), (2c-5), (2c-6), (2c-7) or (2c-8).

In a preferred embodiment of the compounds of the formulae (2), (2a), (2b), (2b-1), (2b-2), (2b-3), (2b-4), (2b-5) or (2b-6), the carbazole and the bridged carbazole are joined to one another, each in the 3 position.

In a preferred embodiment of the compounds of the formula (2c), the two bridged carbazoles are joined to one another, each in the 3 position.

Examples of suitable host materials of the formulae (2), (2a), (2b), (2b-1), (2b-2), (2b-3), (2b-4), (2b-5) and (2c) that are selected in accordance with the invention and are preferably used in combination with at least one compound of the formula (1) in the electroluminescent device of the invention are the structures given below in table 2.

TABLE 2

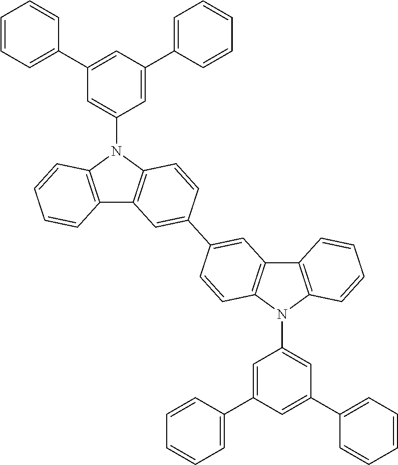 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

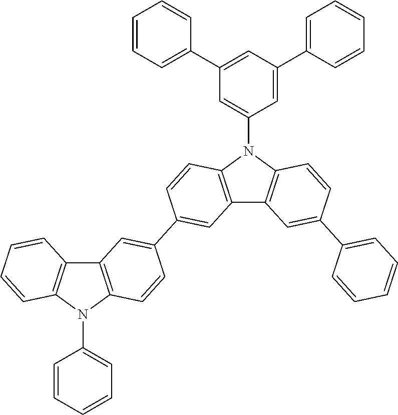 [SMILES: c1ccc(-c2cc(-c3ccccc3)cc(-n3c4ccc(-c5ccccc5)cc4c4cc(-c5ccc6c(c5)c5ccccc5n6-c5ccccc5)ccc43)c2)cc1]

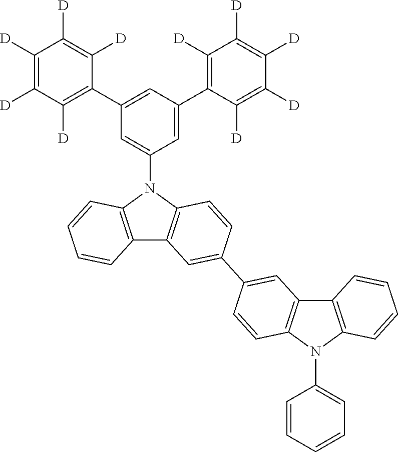 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H]]

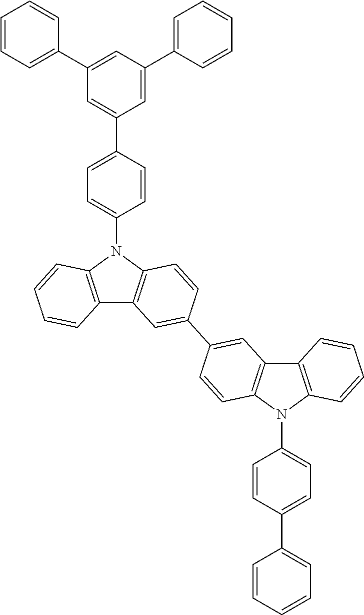 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

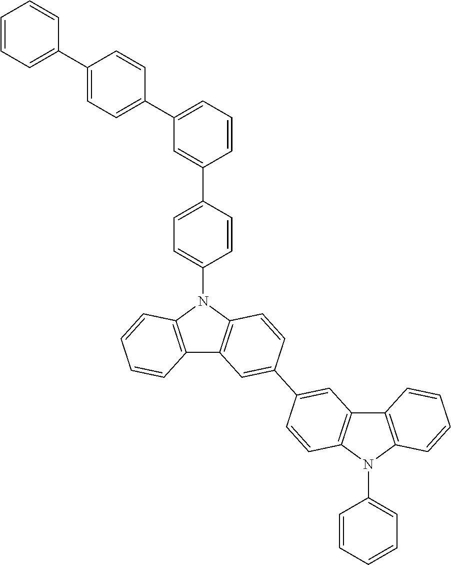 [SMILES: c1ccc(-c2ccc(-c3cccc(-c4ccc(-n5c6ccccc6c6cc(-c7ccc8c(c7)c7ccccc7n8-c7ccccc7)ccc65)cc4)c3)cc2)cc1]

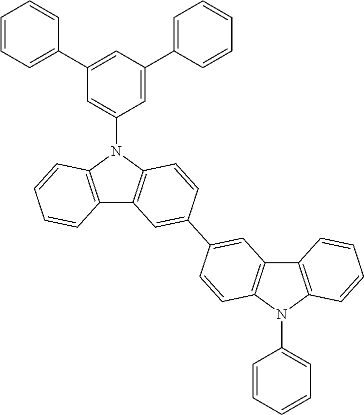 [SMILES: c1ccc(-c2cc(-c3ccccc3)cc(-n3c4ccccc4c4cc(-c5ccc6c(c5)c5ccccc5n6-c5ccccc5)ccc43)c2)cc1]

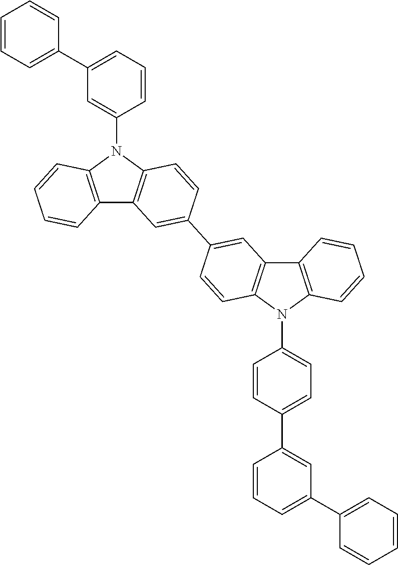 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

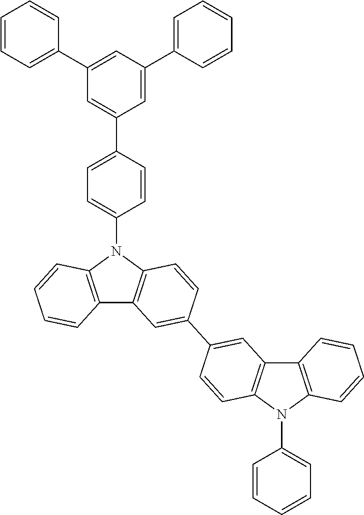 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

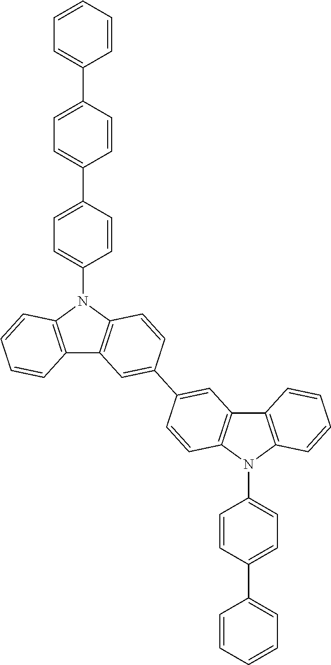 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

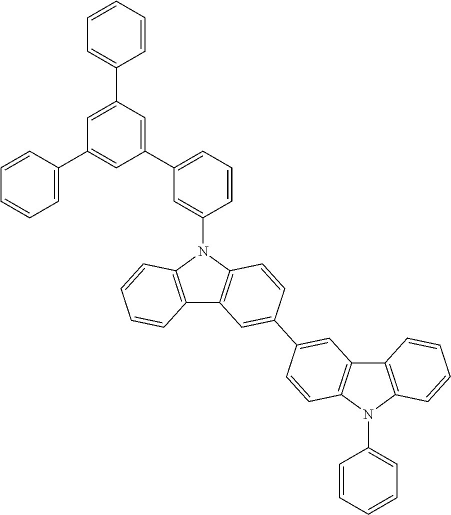 [SMILES: c1ccc(-c2cc(-c3ccccc3)cc(-c3cccc(-n4c5ccccc5c5cc(-c6ccc7c(c6)c6ccccc6n7-c6ccccc6)ccc54)c3)c2)cc1]

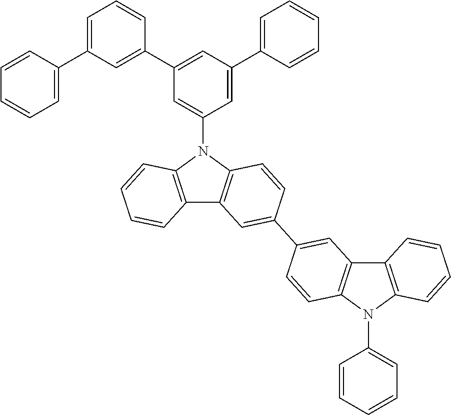 [SMILES: c1ccc(-c2cccc(-c3cc(-c4ccccc4)cc(-n4c5ccccc5c5cc(-c6ccc7c(c6)c6ccccc6n7-c6ccccc6)ccc54)c3)c2)cc1]

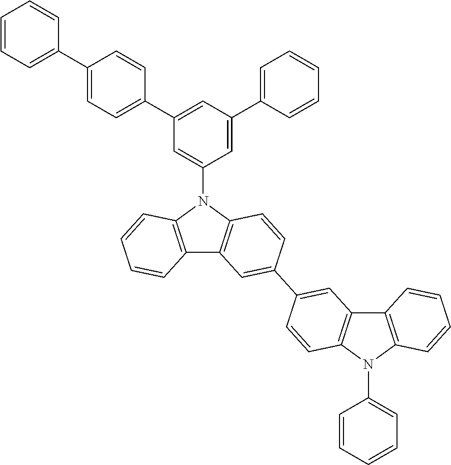 [SMILES: c1ccc(-c2ccc(-c3cc(-c4ccccc4)cc(-n4c5ccccc5c5cc(-c6ccc7c(c6)c6ccccc6n7-c6ccccc6)ccc54)c3)cc2)cc1]

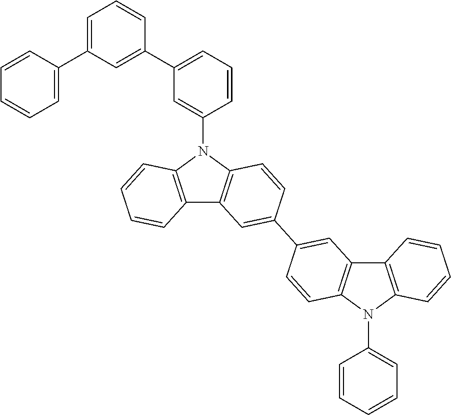 [SMILES: c1ccc(-c2cccc(-c3cccc(-n4c5ccccc5c5cc(-c6ccc7c(c6)c6ccccc6n7-c6ccccc6)ccc54)c3)c2)cc1]

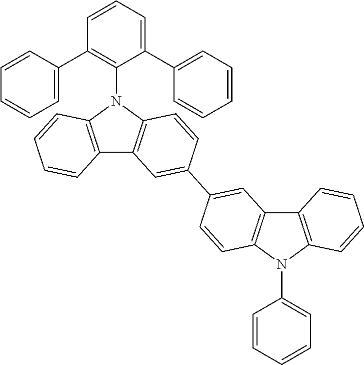 [SMILES: c1ccc(-c2cccc(-c3ccccc3)c2-n2c3ccccc3c3cc(-c4ccc5c(c4)c4ccccc4n5-c4ccccc4)ccc32)cc1]

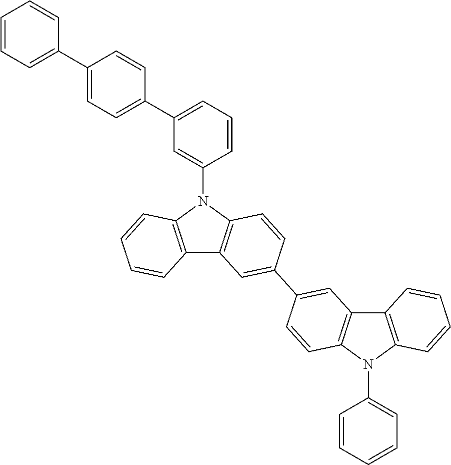 [SMILES: c1ccc(-c2ccc(-c3cccc(-n4c5ccccc5c5cc(-c6ccc7c(c6)c6ccccc6n7-c6ccccc6)ccc54)c3)cc2)cc1]

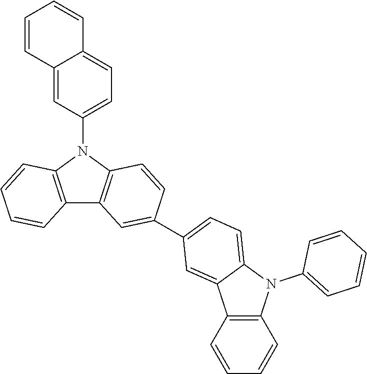 [SMILES: c1ccc(-n2c3ccccc3c3cc(-c4ccc5c(c4)c4ccccc4n5-c4ccc5ccccc5c4)ccc32)cc1]

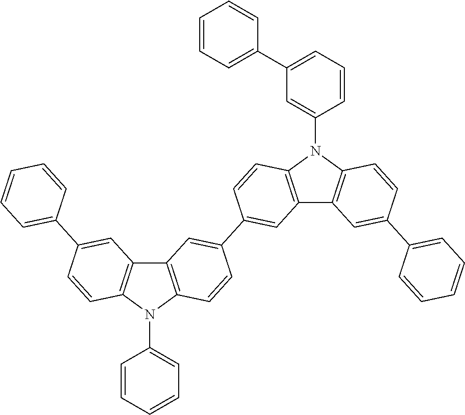 [SMILES: c1ccc(-c2cccc(-n3c4ccc(-c5ccccc5)cc4c4cc(-c5ccc6c(c5)c5cc(-c7ccccc7)ccc5n6-c5ccccc5)ccc43)c2)cc1]

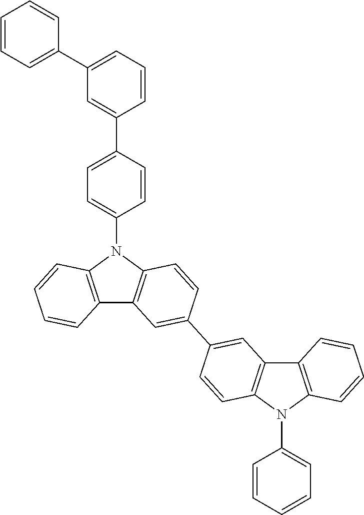 [SMILES: c1ccc(-c2cccc(-c3ccc(-n4c5ccccc5c5cc(-c6ccc7c(c6)c6ccccc6n7-c6ccccc6)ccc54)cc3)c2)cc1]

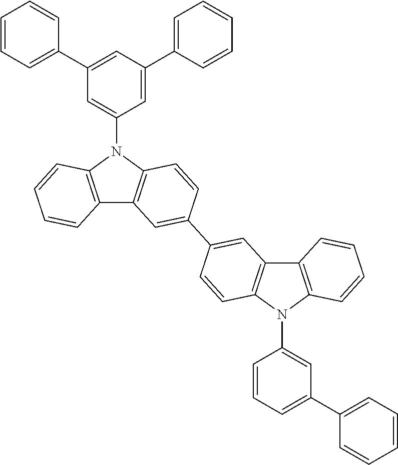 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

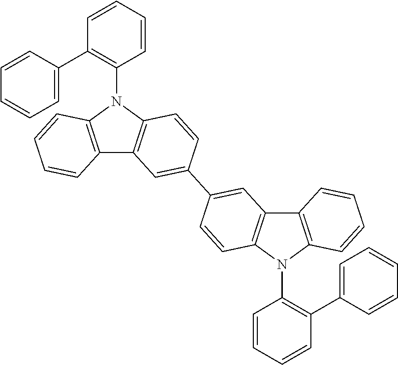 [SMILES: c1ccc(-c2ccccc2-n2c3ccccc3c3cc(-c4ccc5c(c4)c4ccccc4n5-c4ccccc4-c4ccccc4)ccc32)cc1]

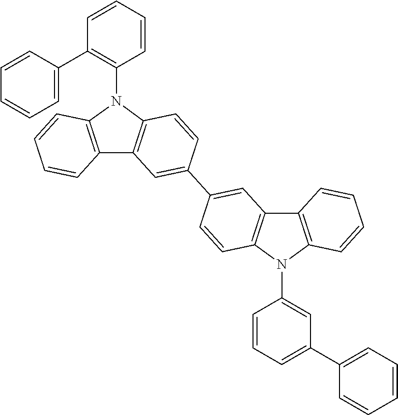 [SMILES: c1ccc(-c2cccc(-n3c4ccccc4c4cc(-c5ccc6c(c5)c5ccccc5n6-c5ccccc5-c5ccccc5)ccc43)c2)cc1]

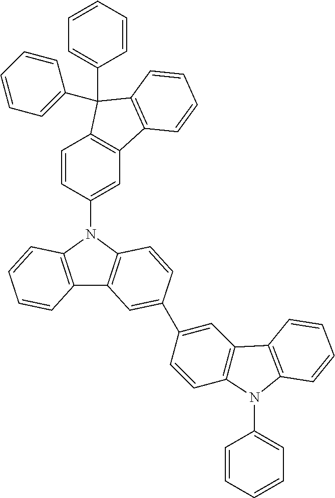 [SMILES: c1ccc(-n2c3ccccc3c3cc(-c4ccc5c(c4)c4ccccc4n5-c4ccc5c(c4)-c4ccccc4C5(c4ccccc4)c4ccccc4)ccc32)cc1]

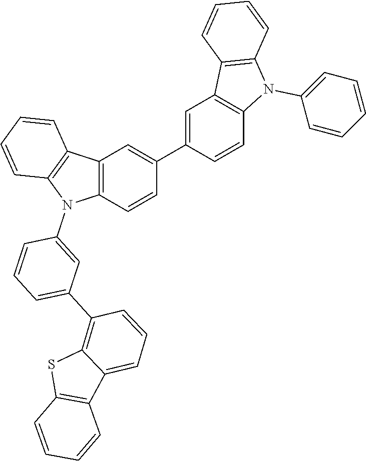 [SMILES: c1ccc(-n2c3ccccc3c3cc(-c4ccc5c(c4)c4ccccc4n5-c4cccc(-c5cccc6c5sc5ccccc56)c4)ccc32)cc1]

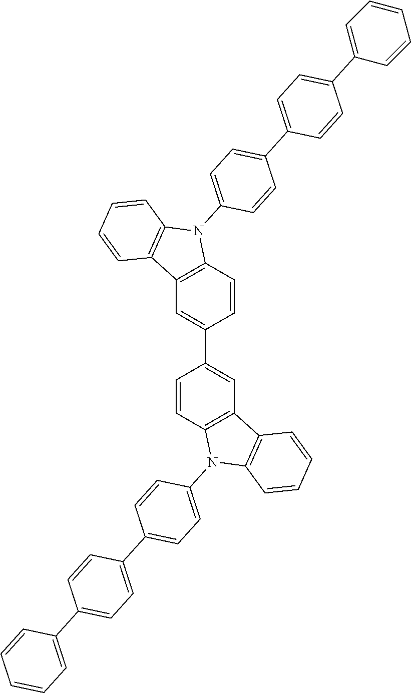 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

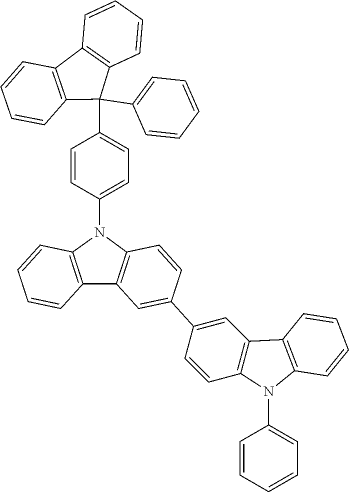 [SMILES: c1ccc(-n2c3ccccc3c3cc(-c4ccc5c(c4)c4ccccc4n5-c4ccc(C5(c6ccccc6)c6ccccc6-c6ccccc65)cc4)ccc32)cc1]

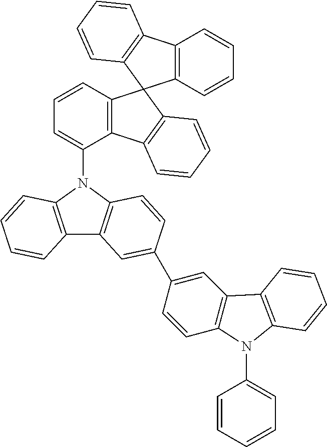 [SMILES: c1ccc(-n2c3ccccc3c3cc(-c4ccc5c(c4)c4ccccc4n5-c4cccc5c4-c4ccccc4C54c5ccccc5-c5ccccc54)ccc32)cc1]

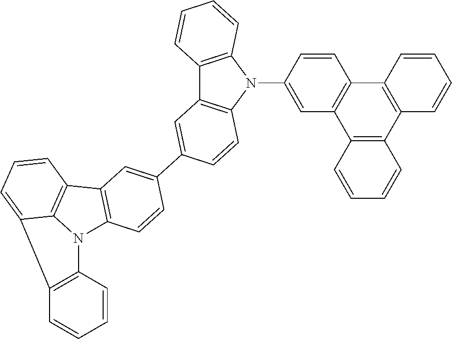 [SMILES: c1ccc2c(c1)c1ccccc1c1cc(-n3c4ccccc4c4cc(-c5ccc6c(c5)c5cccc7c8ccccc8n6c75)ccc43)ccc21]

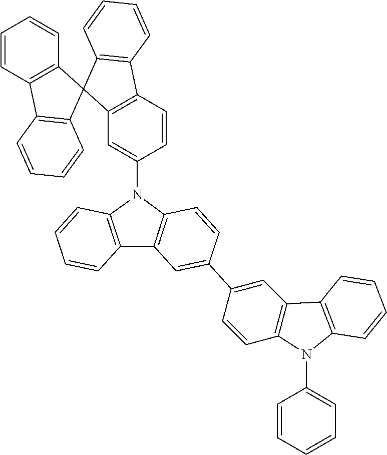 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

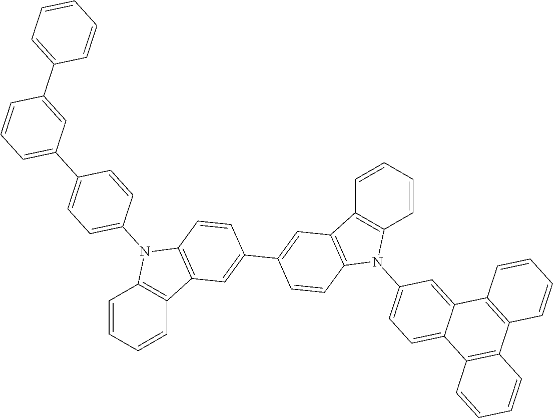 [SMILES: c1ccc(-c2cccc(-c3ccc(-n4c5ccccc5c5cc(-c6ccc7c(c6)c6ccccc6n7-c6ccc7c8ccccc8c8ccccc8c7c6)ccc54)cc3)c2)cc1]

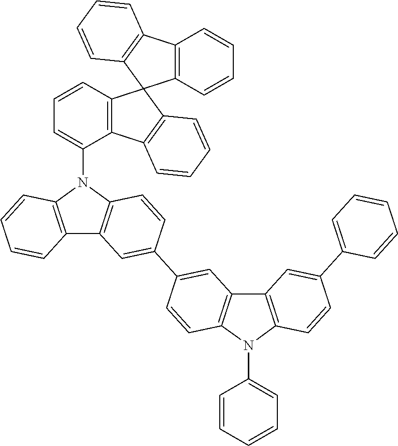 [SMILES: c1ccc(-c2ccc3c(c2)c2cc(-c4ccc5c(c4)c4ccccc4n5-c4cccc5c4-c4ccccc4C54c5ccccc5-c5ccccc54)ccc2n3-c2ccccc2)cc1]

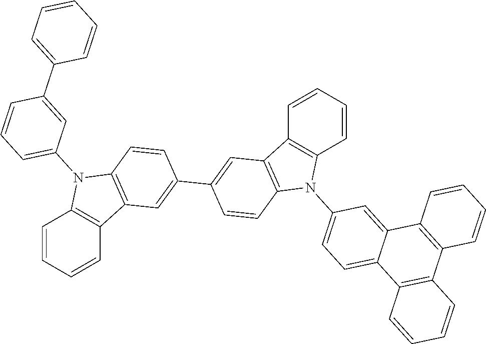 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1]

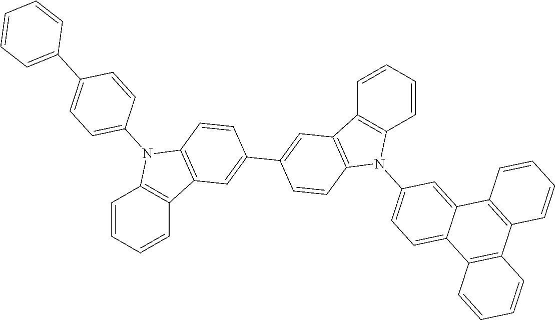 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1]

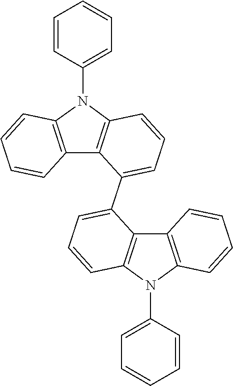 [SMILES: c1ccc(-n2c3ccccc3c3c(-c4cccc5c4c4ccccc4n5-c4ccccc4)cccc32)cc1]

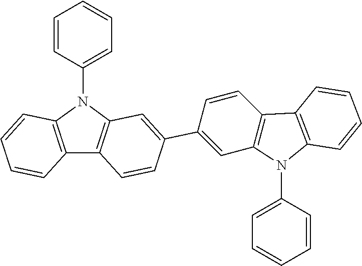 [SMILES: c1ccc(-n2c3ccccc3c3ccc(-c4ccc5c6ccccc6n(-c6ccccc6)c5c4)cc32)cc1]

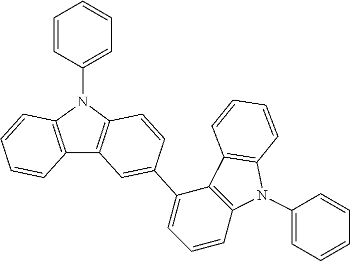 [SMILES: c1ccc(-n2c3ccccc3c3cc(-c4cccc5c4c4ccccc4n5-c4ccccc4)ccc32)cc1]

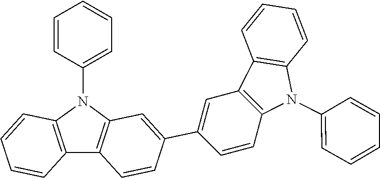 [SMILES: c1ccc(-n2c3ccccc3c3cc(-c4ccc5c6ccccc6n(-c6ccccc6)c5c4)ccc32)cc1]

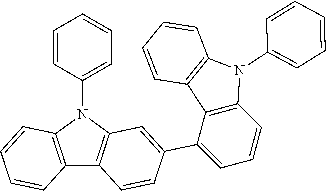 [SMILES: c1ccc(-n2c3ccccc3c3ccc(-c4cccc5c4c4ccccc4n5-c4ccccc4)cc32)cc1]

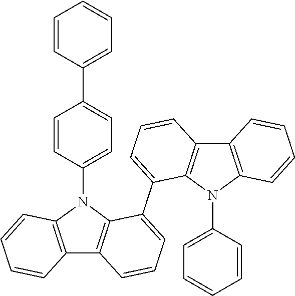 [SMILES: c1ccc(-c2ccc(-n3c4ccccc4c4cccc(-c5cccc6c7ccccc7n(-c7ccccc7)c56)c43)cc2)cc1]

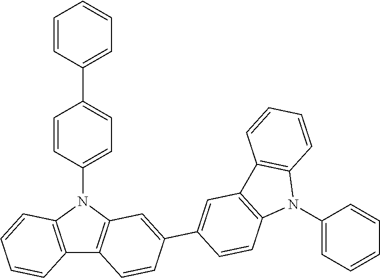 [SMILES: c1ccc(-c2ccc(-n3c4ccccc4c4ccc(-c5ccc6c(c5)c5ccccc5n6-c5ccccc5)cc43)cc2)cc1]

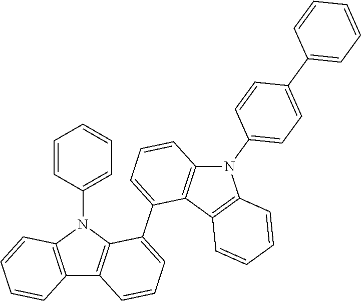 [SMILES: c1ccc(-c2ccc(-n3c4ccccc4c4c(-c5cccc6c7ccccc7n(-c7ccccc7)c56)cccc43)cc2)cc1]

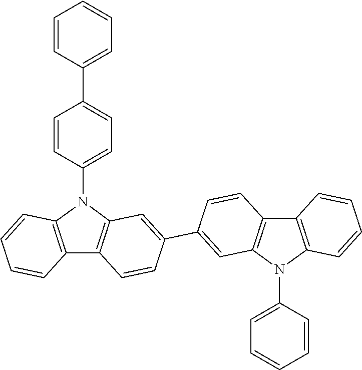 [SMILES: c1ccc(-c2ccc(-n3c4ccccc4c4ccc(-c5ccc6c7ccccc7n(-c7ccccc7)c6c5)cc43)cc2)cc1]

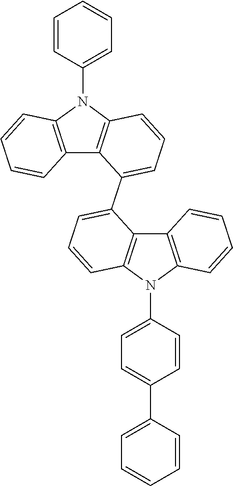 [SMILES: c1ccc(-c2ccc(-n3c4ccccc4c4c(-c5cccc6c5c5ccccc5n6-c5ccccc5)cccc43)cc2)cc1]

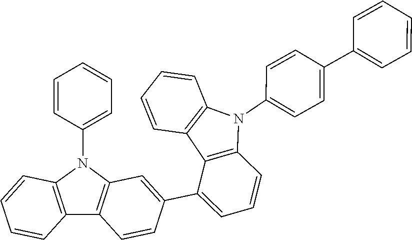 [SMILES: c1ccc(-c2ccc(-n3c4ccccc4c4c(-c5ccc6c7ccccc7n(-c7ccccc7)c6c5)cccc43)cc2)cc1]

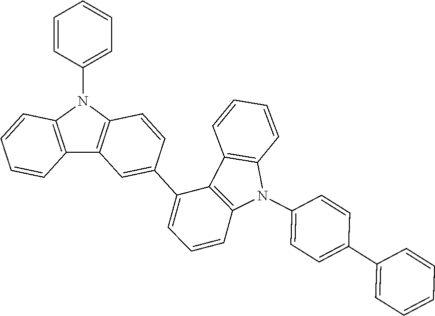 [SMILES: c1ccc(-c2ccc(-n3c4ccccc4c4c(-c5ccc6c(c5)c5ccccc5n6-c5ccccc5)cccc43)cc2)cc1]

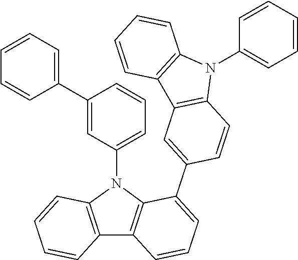 [SMILES: c1ccc(-c2cccc(-n3c4ccccc4c4cccc(-c5ccc6c(c5)c5ccccc5n6-c5ccccc5)c43)c2)cc1]

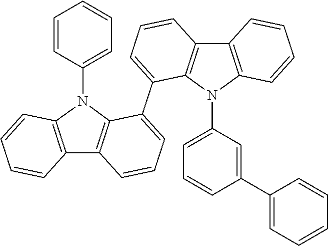 [SMILES: c1ccc(-c2cccc(-n3c4ccccc4c4cccc(-c5cccc6c7ccccc7n(-c7ccccc7)c56)c43)c2)cc1]

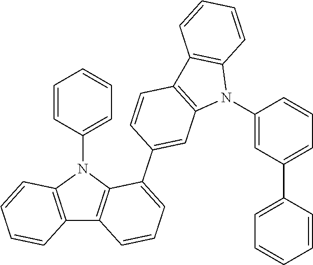 [SMILES: c1ccc(-c2cccc(-n3c4ccccc4c4ccc(-c5cccc6c7ccccc7n(-c7ccccc7)c56)cc43)c2)cc1]

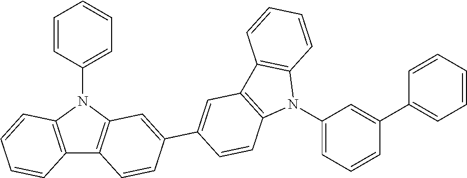 [SMILES: c1ccc(-c2cccc(-n3c4ccccc4c4cc(-c5ccc6c7ccccc7n(-c7ccccc7)c6c5)ccc43)c2)cc1]

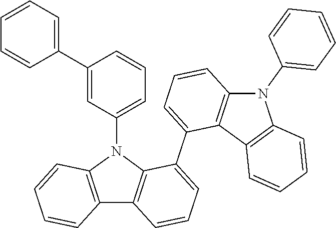 [SMILES: c1ccc(-c2cccc(-n3c4ccccc4c4cccc(-c5cccc6c5c5ccccc5n6-c5ccccc5)c43)c2)cc1]

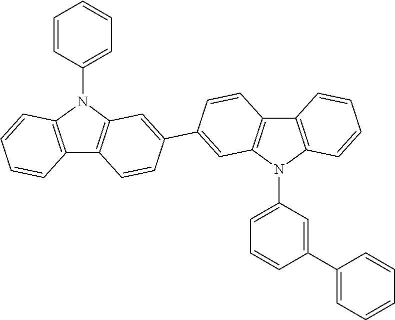 [SMILES: c1ccc(-c2cccc(-n3c4ccccc4c4ccc(-c5ccc6c7ccccc7n(-c7ccccc7)c6c5)cc43)c2)cc1]

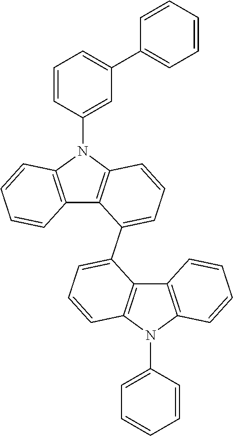 [SMILES: c1ccc(-c2cccc(-n3c4ccccc4c4c(-c5cccc6c5c5ccccc5n6-c5ccccc5)cccc43)c2)cc1]

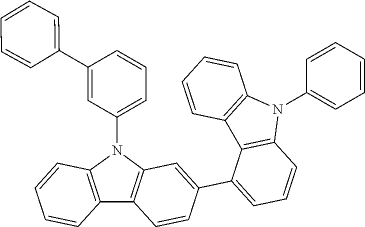 [SMILES: c1ccc(-c2cccc(-n3c4ccccc4c4ccc(-c5cccc6c5c5ccccc5n6-c5ccccc5)cc43)c2)cc1]

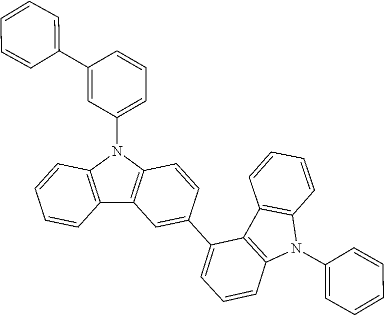 [SMILES: c1ccc(-c2cccc(-n3c4ccccc4c4cc(-c5cccc6c5c5ccccc5n6-c5ccccc5)ccc43)c2)cc1]

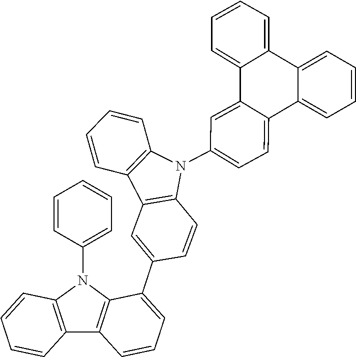 [SMILES: c1ccc(-n2c3ccccc3c3cccc(-c4ccc5c(c4)c4ccccc4n5-c4ccc5c6ccccc6c6ccccc6c5c4)c32)cc1]

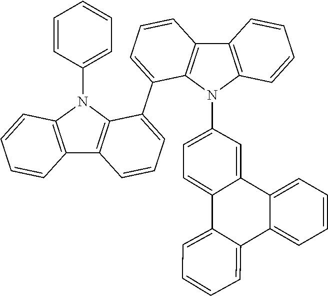 [SMILES: c1ccc(-n2c3ccccc3c3cccc(-c4cccc5c6ccccc6n(-c6ccc7c8ccccc8c8ccccc8c7c6)c45)c32)cc1]

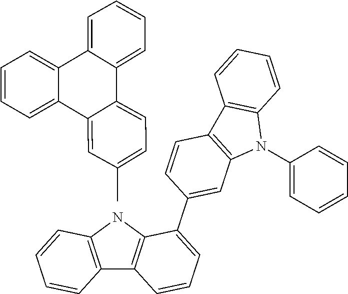

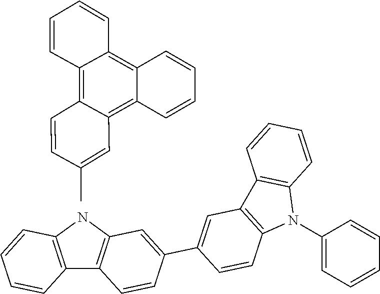

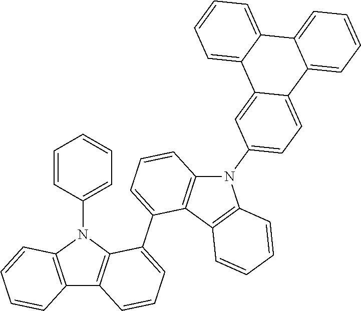 [SMILES: c1ccc(-n2c3ccccc3c3cccc(-c4cccc5c4c4ccccc4n5-c4ccc5c6ccccc6c6ccccc6c5c4)c32)cc1]

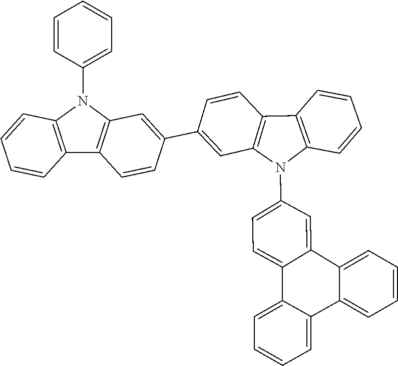 [SMILES: c1ccc(-n2c3ccccc3c3ccc(-c4ccc5c6ccccc6n(-c6ccc7c8ccccc8c8ccccc8c7c6)c5c4)cc32)cc1]

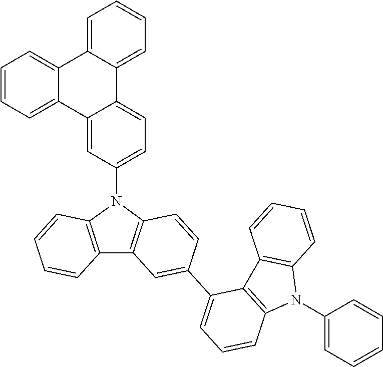 [SMILES: c1ccc(-n2c3ccccc3c3c(-c4ccc5c(c4)c4ccccc4n5-c4ccc5c6ccccc6c6ccccc6c5c4)cccc32)cc1]

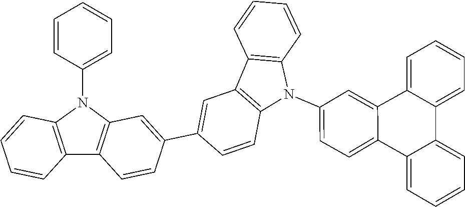 [SMILES: c1ccc(-n2c3ccccc3c3ccc(-c4ccc5c(c4)c4ccccc4n5-c4ccc5c6ccccc6c6ccccc6c5c4)cc32)cc1]

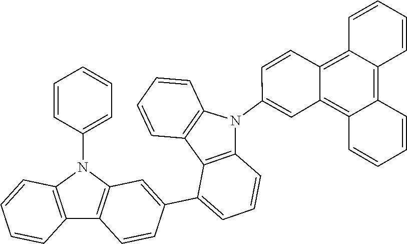 [SMILES: c1ccc(-n2c3ccccc3c3ccc(-c4cccc5c4c4ccccc4n5-c4ccc5c6ccccc6c6ccccc6c5c4)cc32)cc1]

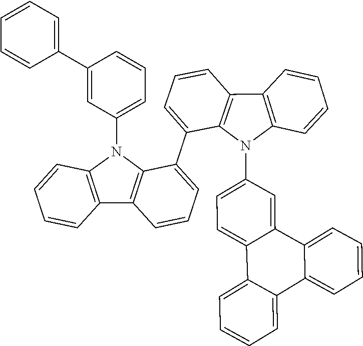 [SMILES: c1ccc(-c2cccc(-n3c4ccccc4c4cccc(-c5cccc6c7ccccc7n(-c7ccc8c9ccccc9c9ccccc9c8c7)c56)c43)c2)cc1]

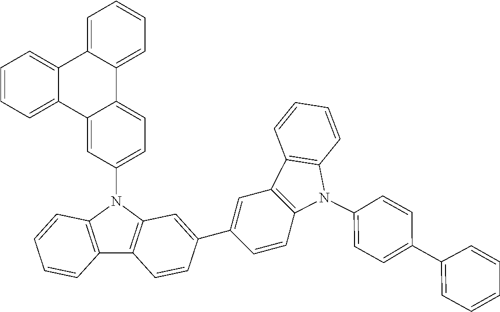 [SMILES: c1ccc(-c2ccc(-n3c4ccccc4c4cc(-c5ccc6c7ccccc7n(-c7ccc8c9ccccc9c9ccccc9c8c7)c6c5)ccc43)cc2)cc1]

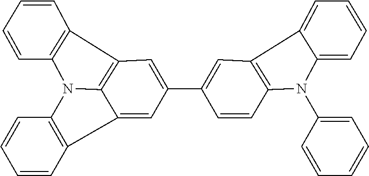 [SMILES: c1ccc(-n2c3ccccc3c3cc(-c4cc5c6ccccc6n6c7ccccc7c(c4)c56)ccc32)cc1]

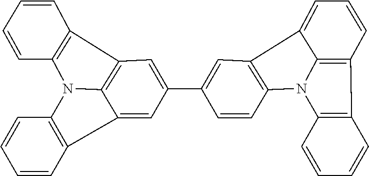 [SMILES: c1ccc2c(c1)c1cccc3c4cc(-c5cc6c7ccccc7n7c8ccccc8c(c5)c67)ccc4n2c13]

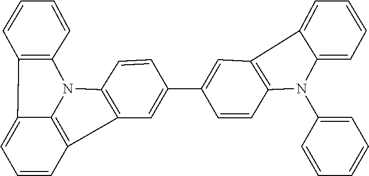 [SMILES: c1ccc(-n2c3ccccc3c3cc(-c4ccc5c(c4)c4cccc6c7ccccc7n5c64)ccc32)cc1]

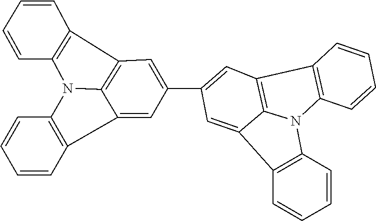 [SMILES: c1ccc2c(c1)c1cc(-c3cc4c5ccccc5n5c6ccccc6c(c3)c45)cc3c4ccccc4n2c13]

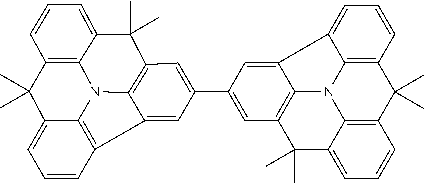

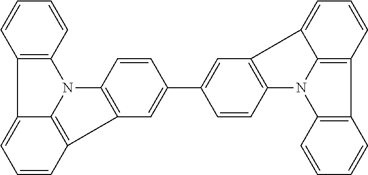 [SMILES: c1ccc2c(c1)c1cccc3c4cc(-c5ccc6c(c5)c5cccc7c8ccccc8n6c75)ccc4n2c13]

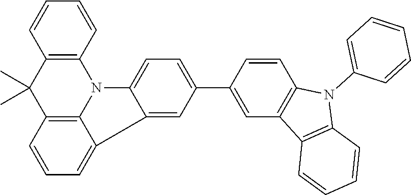 [SMILES: CC1(C)c2ccccc2-n2c3ccc(-c4ccc5c(c4)c4ccccc4n5-c4ccccc4)cc3c3cccc1c32]

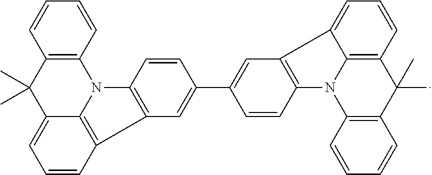

Particularly suitable compounds of the formula (2) that are preferably used in combination with at least one compound of the formula (1) in the electroluminescent device of the invention are the compounds H1 to H27:

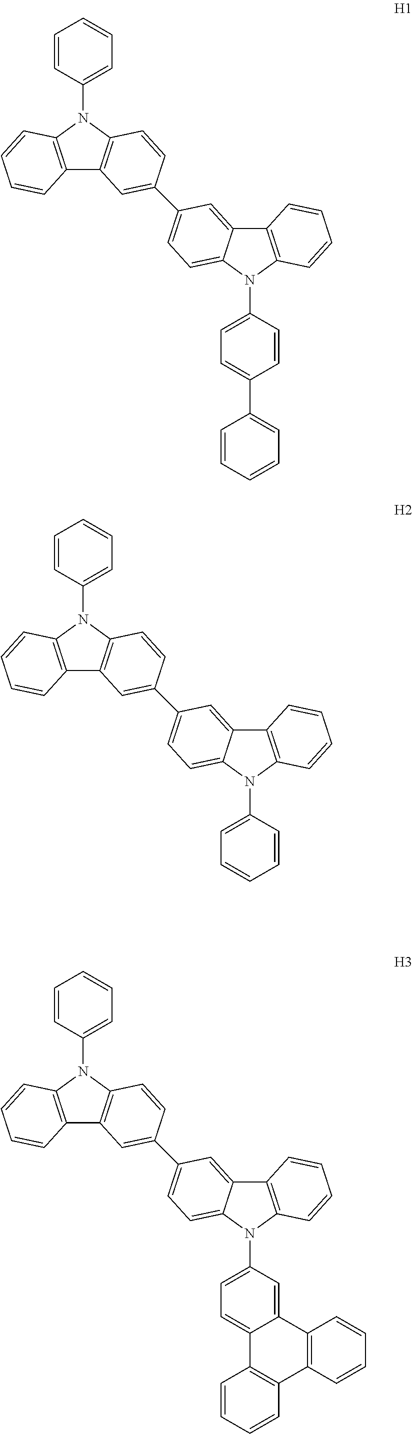 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

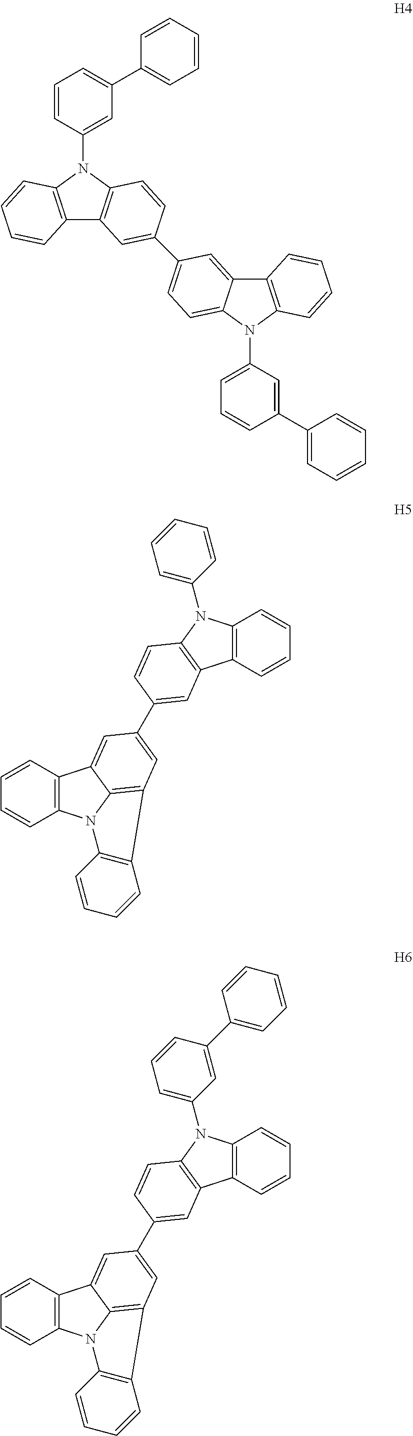 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4cc5c6ccccc6n6c7ccccc7c(c4)c56)ccc32)cc1 ; c1ccc(-c2cccc(-n3c4ccccc4c4cc(-c5cc6c7ccccc7n7c8ccccc8c(c5)c67)ccc43)c2)cc1]

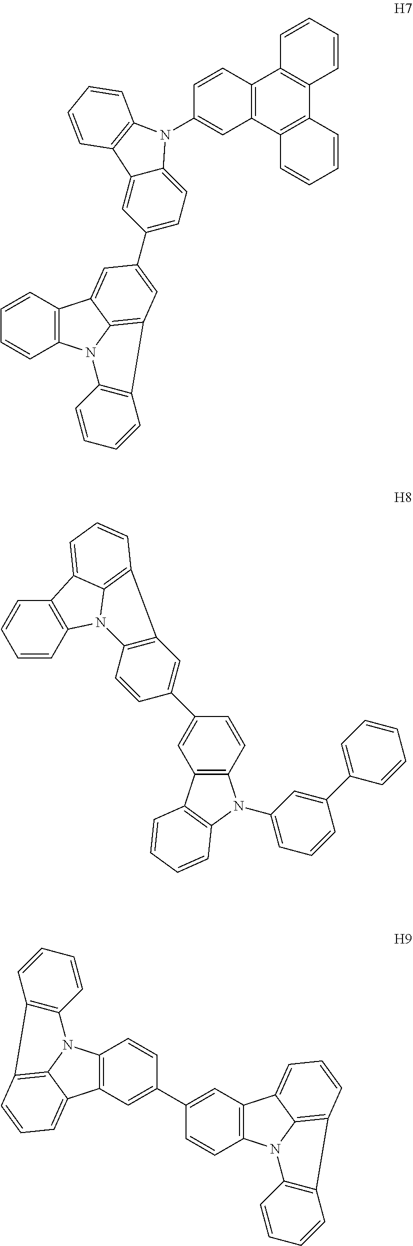 [SMILES: c1ccc2c(c1)c1ccccc1c1cc(-n3c4ccccc4c4cc(-c5cc6c7ccccc7n7c8ccccc8c(c5)c67)ccc43)ccc21 ; c1ccc(-c2cccc(-n3c4ccccc4c4cc(-c5ccc6c(c5)c5cccc7c8ccccc8n6c75)ccc43)c2)cc1 ; c1ccc2c(c1)c1cccc3c4cc(-c5ccc6c(c5)c5cccc7c8ccccc8n6c75)ccc4n2c13]

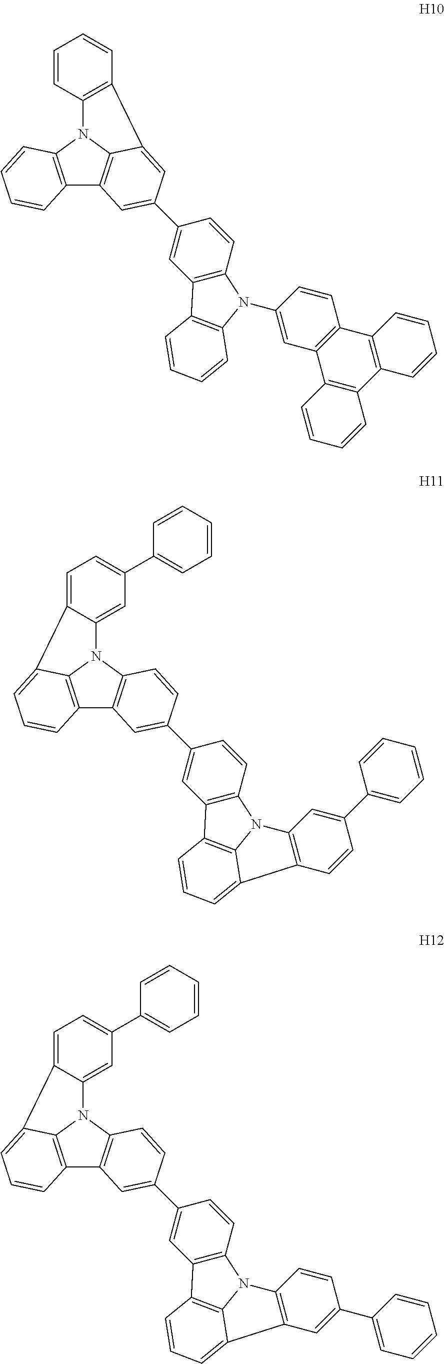 [SMILES: c1ccc2c(c1)c1ccccc1c1cc(-n3c4ccccc4c4cc(-c5cc6c7ccccc7n7c8ccccc8c(c5)c67)ccc43)ccc21 ; c1ccc(-c2ccc3c4cccc5c6cc(-c7ccc8c(c7)c7cccc9c%10ccc(-c%11ccccc%11)cc%10n8c79)ccc6n(c3c2)c54)cc1 ; c1ccc(-c2ccc3c(c2)c2cccc4c5cc(-c6ccc7c(c6)c6cccc8c9ccc(-c%10ccccc%10)cc9n7c68)ccc5n3c24)cc1]

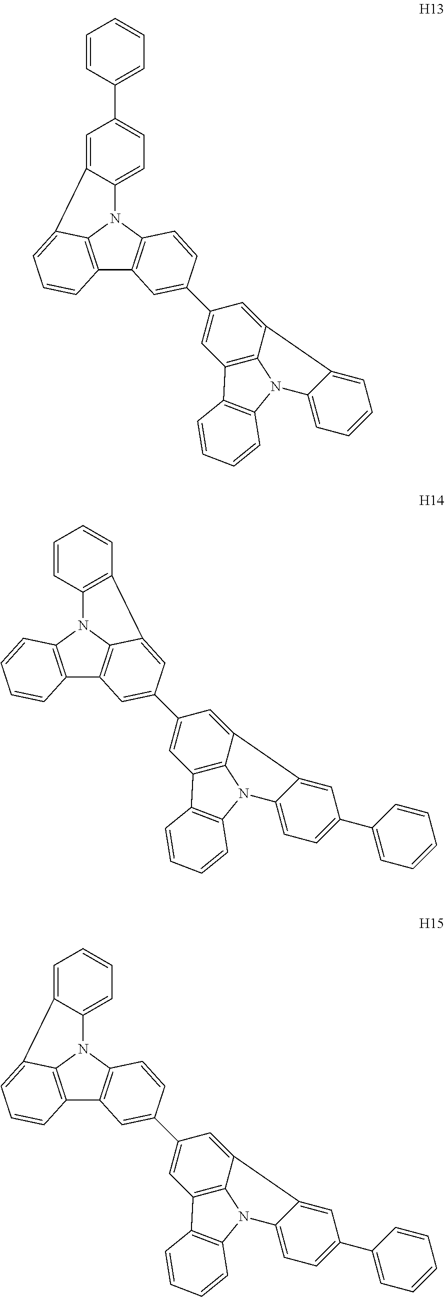 [SMILES: c1ccc(-c2ccc3c(c2)c2cccc4c5cc(-c6cc7c8ccccc8n8c9ccccc9c(c6)c78)ccc5n3c24)cc1 ; c1ccc(-c2ccc3c(c2)c2cc(-c4cc5c6ccccc6n6c7ccccc7c(c4)c56)cc4c5ccccc5n3c42)cc1 ; c1ccc(-c2ccc3c(c2)c2cc(-c4ccc5c(c4)c4cccc6c7ccccc7n5c64)cc4c5ccccc5n3c42)cc1]

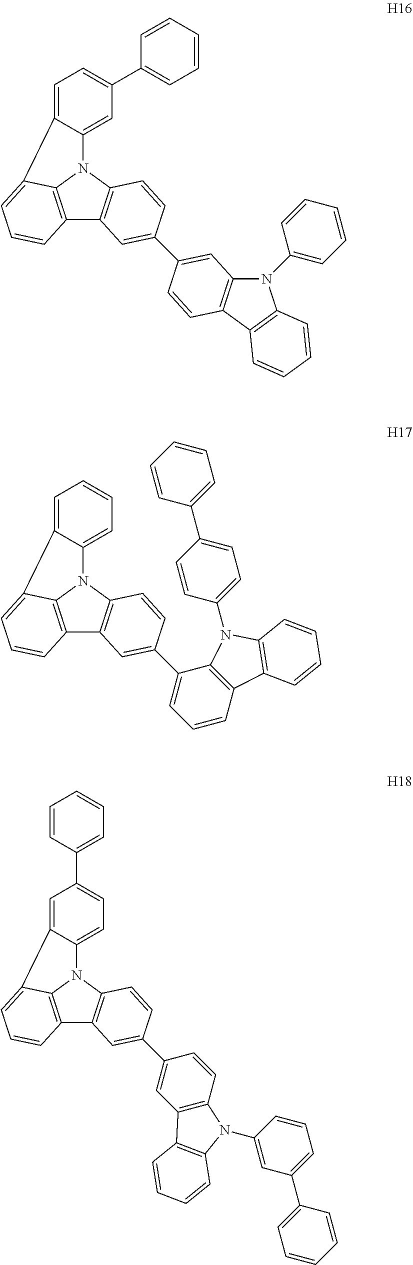 [SMILES: c1ccc(-c2ccc3c4cccc5c6cc(-c7ccc8c9ccccc9n(-c9ccccc9)c8c7)ccc6n(c3c2)c54)cc1 ; c1ccc(-c2ccc(-n3c4ccccc4c4cccc(-c5ccc6c(c5)c5cccc7c8ccccc8n6c75)c43)cc2)cc1 ; c1ccc(-c2cccc(-n3c4ccccc4c4cc(-c5ccc6c(c5)c5cccc7c8cc(-c9ccccc9)ccc8n6c75)ccc43)c2)cc1]

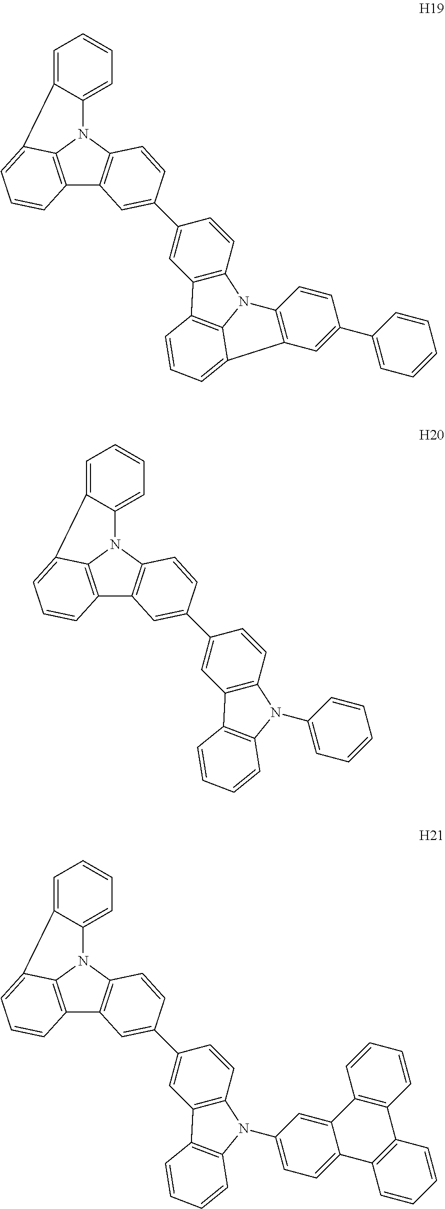 [SMILES: c1ccc(-c2ccc3c(c2)c2cccc4c5cc(-c6ccc7c(c6)c6cccc8c9ccccc9n7c86)ccc5n3c24)cc1 ; c1ccc(-n2c3ccccc3c3cc(-c4ccc5c(c4)c4cccc6c7ccccc7n5c64)ccc32)cc1 ; c1ccc2c(c1)c1ccccc1c1cc(-n3c4ccccc4c4cc(-c5ccc6c(c5)c5cccc7c8ccccc8n6c75)ccc43)ccc21]

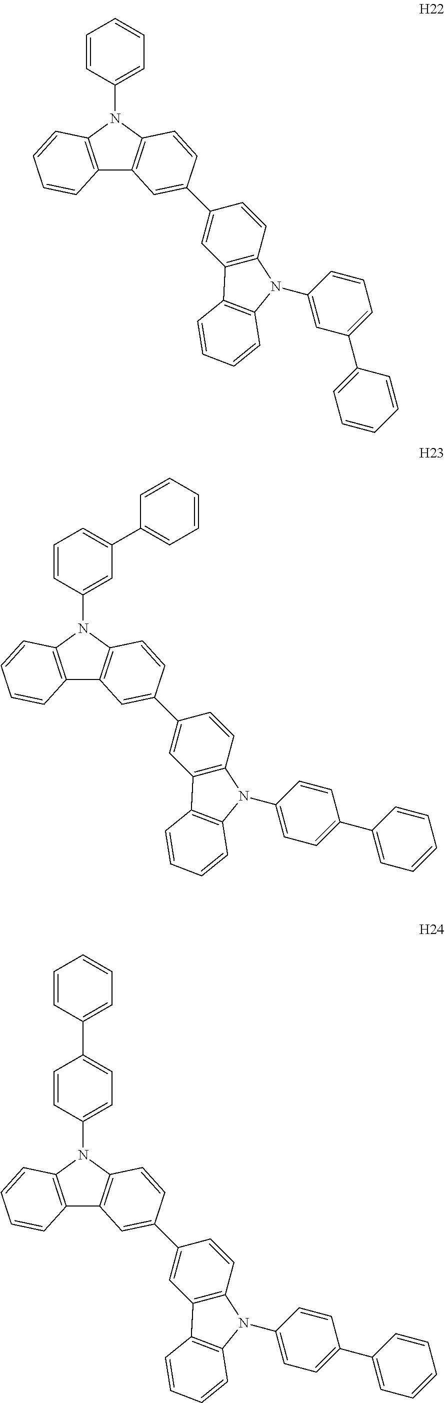 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

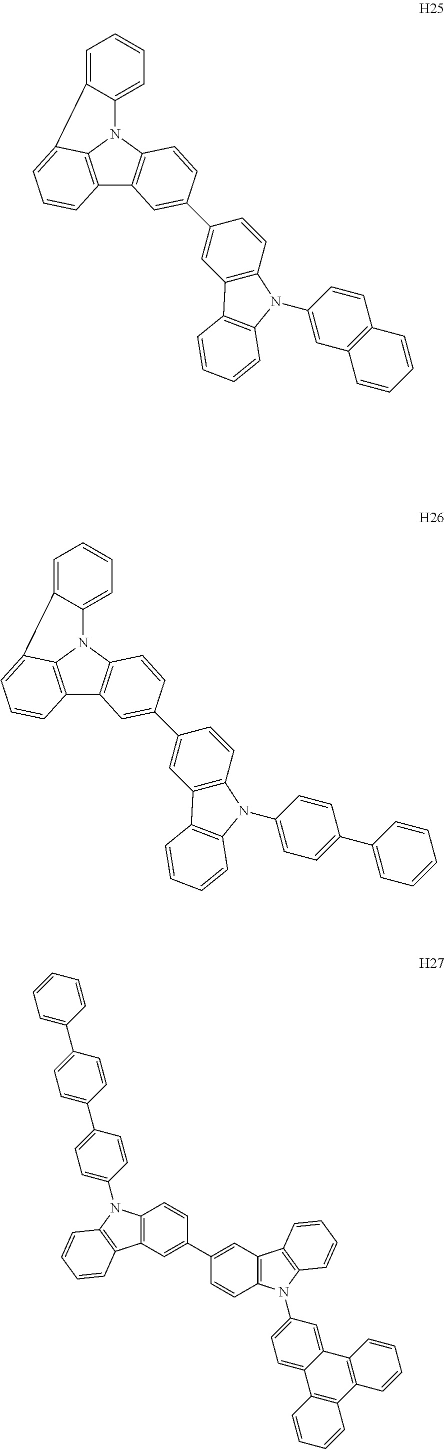 [SMILES: c1ccc2cc(-n3c4ccccc4c4cc(-c5ccc6c(c5)c5cccc7c8ccccc8n6c75)ccc43)ccc2c1 ; c1ccc(-c2ccc(-n3c4ccccc4c4cc(-c5ccc6c(c5)c5cccc7c8ccccc8n6c75)ccc43)cc2)cc1 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

The preparation of the compounds of the formula (2) or of the preferred compounds of the formulae (2), (2a), (2b), (2b-1), (2b-2), (2b-3), (2b-4), (2b-5) and (2c) and of the compounds from table 2 and H1 to H27 is known to the person skilled in the art. The compounds may be prepared by synthesis steps known to the person skilled in the art, for example halogenation, preferably bromination, and a subsequent organometallic coupling reaction, for example Suzuki coupling, Heck coupling or Hartwig-Buchwald coupling. Some of the compounds of the formula (2) are commercially available.

The aforementioned host materials of the formula (1) and the embodiments thereof that are described as preferred or the compounds from table 1 and the compounds E1 to E27 can be combined as desired in the device of the invention with the host materials of the formulae (2), (2a), (2b), (2), (2a), (2b), (2b-1), (2b-2), (2b-3), (2b-4), (2b-5), (2c), (2c-1), (2c-2), (2c-3), (2c-4), (2c-5), (2c-6), (2c-7) and (2c-8) mentioned and the embodiments thereof that are described as preferred or the compounds from table 2 or the compounds H1 to H27.

Aforementioned specific combinations of host materials of the formula (1) with host materials of the formula (2) are preferred as described above. Preferred combinations of host materials are likewise described hereinafter.

The invention likewise further provides mixtures comprising at least one compound of the formula (1) and at least one compound of the formula (2)

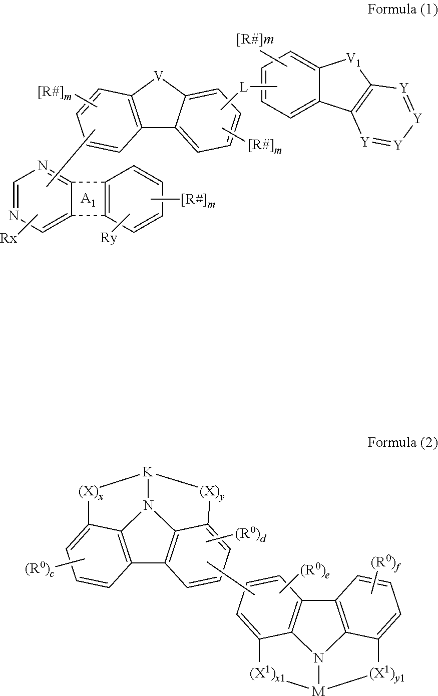

where the symbols and indices used are as follows:
ring A₁ in formula (1) conforms to the formula (1A)

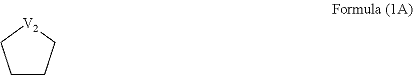

V is O or S;
V₁ is O, S, C(R¹)₂ or N-A;
V₂ is O or S;
Y is the same or different at each instance and is independently CH, CR or CA, or two adjacent Y groups together form a group of the formula (1B)

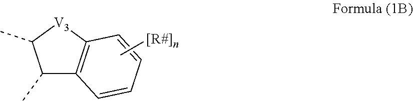

and the remaining Y groups are independently CH or CR, where V₃ is O, S, C(R¹)₂ or N-A and the dotted bonds show the linkage of this group to the remainder of the formula (1);
Rx is L₁-R*;
Ry is L₂-R*;
R* independently at each instance is an aromatic or heteroaromatic ring system which has 6 to 14 ring atoms and may be substituted by one or more R radicals;
L, L₁, L₂ are each independently a bond or a phenylene group which may be substituted by one or more R radicals;
A is the same or different at each instance and is an aromatic or heteroaromatic ring system which has 6 to 30 ring atoms and may be substituted by one or more R radicals;
n is 0, 1, 2, 3 or 4;
m is 0, 1, 2 or 3;
R # is D or an aryl group which has 6 to 12 carbon atoms and may be substituted by one or more R radicals;
R¹ is the same or different at each instance and is an alkyl group having 1 to 20 carbon atoms, phenyl which may be substituted by one or more R radicals, or the two R¹ radicals together with the carbon atom to which they bind form a spiro compound;
R is the same or different at each instance and is selected from D, F, CN, a straight-chain alkyl group having 1 to 20 carbon atoms or a branched or cyclic alkyl group having 3 to 20 carbon atoms, where one or more nonadjacent CH 2 groups may be replaced by R²C═CR², O or S and where one or more hydrogen atoms may be replaced by D, F, or CN;
R² is the same or different at each instance and is selected from the group consisting of H, D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
K, M are each independently an unsubstituted or partly or fully deuterated or R*-monosubstituted aromatic ring system having 6 to 40 ring atoms when x and y are 0 and when x1 and y1 are 0, or
K, M each independently together with X or X′ form a heteroaromatic ring system having 14 to 40 ring atoms when the value of x, x1, y and/or y1 is 1;
x, x1 at each instance are each independently 0 or 1;
y, y1 at each instance are each independently 0 or 1;
X and X′ at each instance are each independently a bond or C(R⁺)₂;
R⁰ at each instance is independently an unsubstituted or partly or fully deuterated aromatic ring system having 6 to 18 carbon atoms;
R⁺ at each instance is independently a straight-chain or branched alkyl group having 1 to 4 carbon atoms and
c, d, e and f are independently 0 or 1.

The remarks concerning the host materials of the formulae (1) and (2) and preferred embodiments thereof and the combination thereof are correspondingly also applicable to the mixture of the invention.

Particularly preferred mixtures of the host materials of the formula (1) with the host materials of the formula (2) for the device of the invention are obtained by combination of the compounds E1 to E27 with the compounds from table 2.

Very particularly preferred mixtures of the host materials of the formula (1) with the host materials of the formula (2) for the device of the invention are obtained by combination of the compounds E1 to E27 with the compounds H1 to H27 as shown hereinafter in table 3.

TABLE 3

| | | | | | | | | |
|---|---|---|---|---|---|---|---|---|
| M1 | E1 | H1 | M2 | E2 | H1 | M3 | E3 | H1 |
| M4 | E4 | H1 | M5 | E5 | H1 | M6 | E6 | H1 |
| M7 | E7 | H1 | M8 | E8 | H1 | M9 | E9 | H1 |
| M10 | E10 | H1 | M11 | E11 | H1 | M12 | E12 | H1 |
| M13 | E13 | H1 | M14 | E14 | H1 | M15 | E15 | H1 |
| M16 | E16 | H1 | M17 | E17 | H1 | M18 | E18 | H1 |
| M19 | E19 | H1 | M20 | E20 | H1 | M21 | E21 | H1 |
| M22 | E22 | H1 | M23 | E23 | H1 | M24 | E24 | H1 |
| M25 | E25 | H1 | M26 | E26 | H1 | M27 | E27 | H1 |
| M28 | E1 | H2 | M29 | E2 | H2 | M30 | E3 | H2 |
| M31 | E4 | H2 | M32 | E5 | H2 | M33 | E6 | H2 |
| M34 | E7 | H2 | M35 | E8 | H2 | M36 | E9 | H2 |
| M37 | E10 | H2 | M38 | E11 | H2 | M39 | E12 | H2 |
| M40 | E13 | H2 | M41 | E14 | H2 | M42 | E15 | H2 |
| M43 | E16 | H2 | M44 | E17 | H2 | M45 | E18 | H2 |
| M46 | E19 | H2 | M47 | E20 | H2 | M48 | E21 | H2 |
| M49 | E22 | H2 | M50 | E23 | H2 | M51 | E24 | H2 |
| M52 | E25 | H2 | M53 | E26 | H2 | M54 | E27 | H2 |
| M55 | E1 | H3 | M56 | E2 | H3 | M57 | E3 | H3 |
| M58 | E4 | H3 | M59 | E5 | H3 | M60 | E6 | H3 |
| M61 | E7 | H3 | M62 | E8 | H3 | M63 | E9 | H3 |
| M64 | E10 | H3 | M65 | E11 | H3 | M66 | E12 | H3 |
| M67 | E13 | H3 | M68 | E14 | H3 | M69 | E15 | H3 |
| M70 | E16 | H3 | M71 | E17 | H3 | M72 | E18 | H3 |
| M73 | E19 | H3 | M74 | E20 | H3 | M75 | E21 | H3 |
| M76 | E22 | H3 | M77 | E23 | H3 | M78 | E24 | H3 |
| M79 | E25 | H3 | M80 | E26 | H3 | M81 | E27 | H3 |
| M82 | E1 | H4 | M83 | E2 | H4 | M84 | E3 | H4 |
| M85 | E4 | H4 | M86 | E5 | H4 | M87 | E6 | H4 |
| M88 | E7 | H4 | M89 | E8 | H4 | M90 | E9 | H4 |
| M91 | E10 | H4 | M92 | E11 | H4 | M93 | E12 | H4 |
| M94 | E13 | H4 | M95 | E14 | H4 | M96 | E15 | H4 |
| M97 | E16 | H4 | M98 | E17 | H4 | M99 | E18 | H4 |
| M100 | E19 | H4 | M101 | E20 | H4 | M102 | E21 | H4 |
| M103 | E22 | H4 | M104 | E23 | H4 | M105 | E24 | H4 |
| M106 | E25 | H4 | M107 | E26 | H4 | M108 | E27 | H4 |
| M109 | E1 | H5 | M110 | E2 | H5 | M111 | E3 | H5 |
| M112 | E4 | H5 | M113 | E5 | H5 | M114 | E6 | H5 |
| M115 | E7 | H5 | M116 | E8 | H5 | M117 | E9 | H5 |
| M118 | E10 | H5 | M119 | E11 | H5 | M120 | E12 | H5 |
| M121 | E13 | H5 | M122 | E14 | H5 | M123 | E15 | H5 |
| M124 | E16 | H5 | M125 | E17 | H5 | M126 | E18 | H5 |
| M127 | E19 | H5 | M128 | E20 | H5 | M129 | E21 | H5 |
| M130 | E22 | H5 | M131 | E23 | H5 | M132 | E24 | H5 |
| M133 | E25 | H5 | M134 | E26 | H5 | M135 | E27 | H5 |
| M136 | E1 | H6 | M137 | E2 | H6 | M138 | E3 | H6 |
| M139 | E4 | H6 | M140 | E5 | H6 | M141 | E6 | H6 |
| M142 | E7 | H6 | M143 | E8 | H6 | M144 | E9 | H6 |
| M145 | E10 | H6 | M146 | E11 | H6 | M147 | E12 | H6 |
| M148 | E13 | H6 | M149 | E14 | H6 | M150 | E15 | H6 |
| M151 | E16 | H6 | M152 | E17 | H6 | M153 | E18 | H6 |
| M154 | E19 | H6 | M155 | E20 | H6 | M156 | E21 | H6 |
| M157 | E22 | H6 | M158 | E23 | H6 | M159 | E24 | H6 |
| M160 | E25 | H6 | M161 | E26 | H6 | M162 | E27 | H6 |
| M163 | E1 | H7 | M164 | E2 | H7 | M165 | E3 | H7 |
| M166 | E4 | H7 | M167 | E5 | H7 | M168 | E6 | H7 |
| M169 | E7 | H7 | M170 | E8 | H7 | M171 | E9 | H7 |
| M172 | E10 | H7 | M173 | E11 | H7 | M174 | E12 | H7 |
| M175 | E13 | H7 | M176 | E14 | H7 | M177 | E15 | H7 |
| M178 | E16 | H7 | M179 | E17 | H7 | M180 | E18 | H7 |
| M181 | E19 | H7 | M182 | E20 | H7 | M183 | E21 | H7 |
| M184 | E22 | H7 | M185 | E23 | H7 | M186 | E24 | H7 |
| M187 | E25 | H7 | M188 | E26 | H7 | M189 | E27 | H7 |
| M190 | E1 | H8 | M191 | E2 | H8 | M192 | E3 | H8 |
| M193 | E4 | H8 | M194 | E5 | H8 | M195 | E6 | H8 |
| M196 | E7 | H8 | M197 | E8 | H8 | M198 | E9 | H8 |
| M199 | E10 | H8 | M200 | E11 | H8 | M201 | E12 | H8 |
| M202 | E13 | H8 | M203 | E14 | H8 | M204 | E15 | H8 |
| M205 | E16 | H8 | M206 | E17 | H8 | M207 | E18 | H8 |
| M208 | E19 | H8 | M209 | E20 | H8 | M210 | E21 | H8 |
| M211 | E22 | H8 | M212 | E23 | H8 | M213 | E24 | H8 |
| M214 | E25 | H8 | M215 | E26 | H8 | M216 | E27 | H8 |
| M217 | E1 | H9 | M218 | E2 | H9 | M219 | E3 | H9 |
| M220 | E4 | H9 | M221 | E5 | H9 | M222 | E6 | H9 |
| M223 | E7 | H9 | M224 | E8 | H9 | M225 | E9 | H9 |
| M226 | E10 | H9 | M227 | E1 | H9 | M228 | E12 | H9 |
| M229 | E13 | H9 | M230 | E14 | H9 | M231 | E15 | H9 |
| M232 | E16 | H9 | M233 | E17 | H9 | M234 | E18 | H9 |
| M235 | E19 | H9 | M236 | E20 | H9 | M237 | E21 | H9 |
| M238 | E22 | H9 | M239 | E23 | H9 | M240 | E24 | H9 |
| M241 | E25 | H9 | M242 | E26 | H9 | M243 | E27 | H9 |
| M244 | E1 | H10 | M245 | E2 | H10 | M246 | E3 | H10 |
| M247 | E4 | H10 | M248 | E5 | H10 | M249 | E6 | H10 |
| M250 | E7 | H10 | M251 | E8 | H10 | M252 | E9 | H10 |
| M253 | E10 | H10 | M254 | E11 | H10 | M255 | E12 | H10 |
| M256 | E13 | H10 | M257 | E14 | H10 | M258 | E15 | H10 |
| M259 | E16 | H10 | M260 | E17 | H10 | M261 | E18 | H10 |
| M262 | E19 | H10 | M263 | E20 | H10 | M264 | E21 | H10 |
| M265 | E22 | H10 | M266 | E23 | H10 | M267 | E24 | H10 |
| M268 | E25 | H10 | M269 | E26 | H10 | M270 | E27 | H10 |
| M271 | E1 | H11 | M272 | E2 | H11 | M273 | E3 | H11 |
| M274 | E4 | H11 | M275 | E5 | H11 | M276 | E6 | H11 |
| M277 | E7 | H11 | M278 | E8 | H11 | M279 | E9 | H11 |
| M280 | E10 | H11 | M281 | E11 | H11 | M282 | E12 | H11 |
| M283 | E13 | H11 | M284 | E14 | H11 | M285 | E15 | H11 |
| M286 | E16 | H11 | M287 | E17 | H11 | M288 | E18 | H11 |
| M289 | E19 | H11 | M290 | E20 | H11 | M291 | E21 | H11 |
| M292 | E22 | H11 | M293 | E23 | H11 | M294 | E24 | H11 |
| M295 | E25 | H11 | M296 | E26 | H11 | M297 | E27 | H11 |
| M298 | E1 | H12 | M299 | E2 | H12 | M300 | E3 | H12 |
| M301 | E4 | H12 | M302 | E5 | H12 | M303 | E6 | H12 |
| M304 | E7 | H12 | M305 | E8 | H12 | M306 | E9 | H12 |
| M307 | E10 | H12 | M308 | E11 | H12 | M309 | E12 | H12 |
| M310 | E13 | H12 | M311 | E14 | H12 | M312 | E15 | H12 |
| M313 | E16 | H12 | M314 | E17 | H12 | M315 | E18 | H12 |
| M316 | E19 | H12 | M317 | E20 | H12 | M318 | E21 | H12 |
| M319 | E22 | H12 | M320 | E23 | H12 | M321 | E24 | H12 |
| M322 | E25 | H12 | M323 | E26 | H12 | M324 | E27 | H12 |
| M325 | E1 | H13 | M326 | E2 | H13 | M327 | E3 | H13 |
| M328 | E4 | H13 | M329 | E5 | H13 | M330 | E6 | H13 |
| M331 | E7 | H13 | M332 | E8 | H13 | M333 | E9 | H13 |
| M334 | E10 | H13 | M335 | E11 | H13 | M336 | E12 | H13 |
| M337 | E13 | H13 | M338 | E14 | H13 | M339 | E15 | H13 |
| M340 | E16 | H13 | M341 | E17 | H13 | M342 | E18 | H13 |
| M343 | E19 | H13 | M344 | E20 | H13 | M345 | E21 | H13 |
| M346 | E22 | H13 | M347 | E23 | H13 | M348 | E24 | H13 |
| M349 | E25 | H13 | M350 | E26 | H13 | M351 | E27 | H13 |
| M352 | E1 | H14 | M353 | E2 | H14 | M354 | E3 | H14 |
| M355 | E4 | H14 | M356 | E5 | H14 | M357 | E6 | H14 |
| M358 | E7 | H14 | M359 | E8 | H14 | M360 | E9 | H14 |
| M361 | E10 | H14 | M362 | E11 | H14 | M363 | E12 | H14 |
| M364 | E13 | H14 | M365 | E14 | H14 | M366 | E15 | H14 |
| M367 | E16 | H14 | M368 | E17 | H14 | M369 | E18 | H14 |
| M370 | E19 | H14 | M371 | E20 | H14 | M372 | E21 | H14 |
| M373 | E22 | H14 | M374 | E23 | H14 | M375 | E24 | H14 |
| M376 | E25 | H14 | M377 | E26 | H14 | M378 | E27 | H14 |
| M379 | E1 | H15 | M380 | E2 | H15 | M381 | E3 | H15 |
| M382 | E4 | H15 | M383 | E5 | H15 | M384 | E6 | H15 |
| M385 | E7 | H15 | M386 | E8 | H15 | M387 | E9 | H15 |
| M388 | E10 | H15 | M389 | E11 | H15 | M390 | E12 | H15 |
| M391 | E13 | H15 | M392 | E14 | H15 | M393 | E15 | H15 |
| M394 | E16 | H15 | M395 | E17 | H15 | M396 | E18 | H15 |
| M397 | E19 | H15 | M398 | E20 | H15 | M399 | E21 | H15 |
| M400 | E22 | H15 | M401 | E23 | H15 | M402 | E24 | H15 |
| M403 | E25 | H15 | M404 | E26 | H15 | M405 | E27 | H15 |
| M406 | E1 | H16 | M407 | E2 | H16 | M408 | E3 | H16 |
| M409 | E4 | H16 | M410 | E5 | H16 | M411 | E6 | H16 |
| M412 | E7 | H16 | M413 | E8 | H16 | M414 | E9 | H16 |
| M415 | E10 | H16 | M416 | E11 | H16 | M417 | E12 | H16 |
| M418 | E13 | H16 | M419 | E14 | H16 | M420 | E15 | H16 |
| M421 | E16 | H16 | M422 | E17 | H16 | M423 | E18 | H16 |
| M424 | E19 | H16 | M425 | E20 | H16 | M426 | E21 | H16 |
| M427 | E22 | H16 | M428 | E23 | H16 | M429 | E24 | H16 |
| M430 | E25 | H16 | M431 | E26 | H16 | M432 | E27 | H16 |
| M433 | E1 | H17 | M434 | E2 | H17 | M435 | E3 | H17 |
| M436 | E4 | H17 | M437 | E5 | H17 | M438 | E6 | H17 |
| M439 | E7 | H17 | M440 | E8 | H17 | M441 | E9 | H17 |
| M442 | E10 | H17 | M443 | E11 | H17 | M444 | E12 | H17 |
| M445 | E13 | H17 | M446 | E14 | H17 | M447 | E15 | H17 |
| M448 | E16 | H17 | M449 | E17 | H17 | M450 | E18 | H17 |
| M451 | E19 | H17 | M452 | E20 | H17 | M453 | E21 | H17 |
| M454 | E22 | H17 | M455 | E23 | H17 | M456 | E24 | H17 |
| M457 | E25 | H17 | M458 | E26 | H17 | M459 | E27 | H17 |
| M460 | E1 | H18 | M461 | E2 | H18 | M462 | E3 | H18 |
| M463 | E4 | H18 | M464 | E5 | H18 | M465 | E6 | H18 |
| M466 | E7 | H18 | M467 | E8 | H18 | M468 | E9 | H18 |
| M469 | E10 | H18 | M470 | E11 | H18 | M471 | E12 | H18 |
| M472 | E13 | H18 | M473 | E14 | H18 | M474 | E15 | H18 |
| M475 | E16 | H18 | M476 | E17 | H18 | M477 | E18 | H18 |
| M478 | E19 | H18 | M479 | E20 | H18 | M480 | E21 | H18 |
| M481 | E22 | H18 | M482 | E23 | H18 | M483 | E24 | H18 |
| M484 | E25 | H18 | M485 | E26 | H18 | M486 | E27 | H18 |
| M487 | E1 | H19 | M488 | E2 | H19 | M489 | E3 | H19 |
| M490 | E4 | H19 | M491 | E5 | H19 | M492 | E6 | H19 |
| M493 | E7 | H19 | M494 | E8 | H19 | M495 | E9 | H19 |
| M496 | E10 | H19 | M497 | E11 | H19 | M498 | E12 | H19 |
| M499 | E13 | H19 | M500 | E14 | H19 | M501 | E15 | H19 |
| M502 | E16 | H19 | M503 | E17 | H19 | M504 | E18 | H19 |
| M505 | E19 | H19 | M506 | E20 | H19 | M507 | E21 | H19 |
| M508 | E22 | H19 | M509 | E23 | H19 | M510 | E24 | H19 |
| M511 | E25 | H19 | M512 | E26 | H19 | M513 | E27 | H19 |
| M514 | E1 | H20 | M515 | E2 | H20 | M516 | E3 | H20 |
| M517 | E4 | H20 | M518 | E5 | H20 | M519 | E6 | H20 |
| M520 | E7 | H20 | M521 | E8 | H20 | M522 | E9 | H20 |
| M523 | E10 | H20 | M524 | E11 | H20 | M525 | E12 | H20 |
| M526 | E13 | H20 | M527 | E14 | H20 | M528 | E15 | H20 |
| M529 | E16 | H20 | M530 | E17 | H20 | M531 | E18 | H20 |
| M532 | E19 | H20 | M533 | E20 | H20 | M534 | E21 | H20 |
| M535 | E22 | H20 | M536 | E23 | H20 | M537 | E24 | H20 |
| M538 | E25 | H20 | M539 | E26 | H20 | M540 | E27 | H20 |
| M541 | E1 | H21 | M542 | E2 | H21 | M543 | E3 | H21 |
| M544 | E4 | H21 | M545 | E5 | H21 | M546 | E6 | H21 |
| M547 | E7 | H21 | M548 | E8 | H21 | M549 | E9 | H21 |
| M550 | E10 | H21 | M551 | E11 | H21 | M552 | E12 | H21 |
| M553 | E13 | H21 | M554 | E14 | H21 | M555 | E15 | H21 |
| M556 | E16 | H21 | M557 | E17 | H21 | M558 | E18 | H21 |
| M559 | E19 | H21 | M560 | E20 | H21 | M561 | E21 | H21 |
| M562 | E22 | H21 | M563 | E23 | H21 | M564 | E24 | H21 |
| M565 | E25 | H21 | M566 | E26 | H21 | M567 | E27 | H21 |
| M568 | E1 | H22 | M569 | E2 | H22 | M570 | E3 | H22 |
| M571 | E4 | H22 | M572 | E5 | H22 | M573 | E6 | H22 |
| M574 | E7 | H22 | M575 | E8 | H22 | M576 | E9 | H22 |
| M577 | E10 | H22 | M578 | E11 | H22 | M579 | E12 | H22 |
| M580 | E13 | H22 | M581 | E14 | H22 | M582 | E15 | H22 |
| M583 | E16 | H22 | M584 | E17 | H22 | M585 | E18 | H22 |
| M586 | E19 | H22 | M587 | E20 | H22 | M588 | E21 | H22 |
| M589 | E22 | H22 | M590 | E23 | H22 | M591 | E24 | H22 |
| M592 | E25 | H22 | M593 | E26 | H22 | M594 | E27 | H22 |
| M595 | E1 | H23 | M596 | E2 | H23 | M597 | E3 | H23 |
| M598 | E4 | H23 | M599 | E5 | H23 | M600 | E6 | H23 |
| M601 | E7 | H23 | M602 | E8 | H23 | M603 | E9 | H23 |
| M604 | E10 | H23 | M605 | E11 | H23 | M606 | E12 | H23 |
| M607 | E13 | H23 | M608 | E14 | H23 | M609 | E15 | H23 |
| M610 | E16 | H23 | M611 | E17 | H23 | M612 | E18 | H23 |
| M613 | E19 | H23 | M614 | E20 | H23 | M615 | E21 | H23 |
| M616 | E22 | H23 | M617 | E23 | H23 | M618 | E24 | H23 |
| M619 | E25 | H23 | M620 | E26 | H23 | M621 | E27 | H23 |
| M622 | E1 | H24 | M623 | E2 | H24 | M624 | E3 | H24 |
| M625 | E4 | H24 | M626 | E5 | H24 | M627 | E6 | H24 |
| M628 | E7 | H24 | M629 | E8 | H24 | M630 | E9 | H24 |
| M631 | E10 | H24 | M632 | E11 | H24 | M633 | E12 | H24 |
| M634 | E13 | H24 | M635 | E14 | H24 | M636 | E15 | H24 |
| M637 | E16 | H24 | M638 | E17 | H24 | M639 | E18 | H24 |
| M640 | E19 | H24 | M641 | E20 | H24 | M642 | E21 | H24 |
| M643 | E22 | H24 | M644 | E23 | H24 | M645 | E24 | H24 |
| M646 | E25 | H24 | M647 | E26 | H24 | M648 | E27 | H24 |
| M649 | E1 | H25 | M650 | E2 | H25 | M651 | E3 | H25 |
| M652 | E4 | H25 | M653 | E5 | H25 | M654 | E6 | H25 |
| M655 | E7 | H25 | M656 | E8 | H25 | M657 | E9 | H25 |
| M658 | E10 | H25 | M659 | E11 | H25 | M660 | E12 | H25 |
| M661 | E13 | H25 | M662 | E14 | H25 | M663 | E15 | H25 |
| M664 | E16 | H25 | M665 | E17 | H25 | M666 | E18 | H25 |
| M667 | E19 | H25 | M668 | E20 | H25 | M669 | E21 | H25 |
| M670 | E22 | H25 | M671 | E23 | H25 | M672 | E24 | H25 |
| M673 | E25 | H25 | M674 | E26 | H25 | M675 | E27 | H25 |
| M676 | E1 | H26 | M677 | E2 | H26 | M678 | E3 | H26 |
| M679 | E4 | H26 | M680 | E5 | H26 | M681 | E6 | H26 |
| M682 | E7 | H26 | M683 | E8 | H26 | M684 | E9 | H26 |
| M685 | E10 | H26 | M686 | E11 | H26 | M687 | E12 | H26 |
| M688 | E13 | H26 | M689 | E14 | H26 | M690 | E15 | H26 |
| M691 | E16 | H26 | M692 | E17 | H26 | M693 | E18 | H26 |
| M694 | E19 | H26 | M695 | E20 | H26 | M696 | E21 | H26 |
| M697 | E22 | H26 | M698 | E23 | H26 | M699 | E24 | H26 |
| M700 | E25 | H26 | M701 | E26 | H26 | M702 | E27 | H26 |
| M703 | E1 | H27 | M704 | E2 | H27 | M705 | E3 | H27 |
| M706 | E4 | H27 | M707 | E5 | H27 | M708 | E6 | H27 |
| M709 | E7 | H27 | M710 | E8 | H27 | M711 | E9 | H27 |
| M712 | E10 | H27 | M713 | E11 | H27 | M714 | E12 | H27 |
| M715 | E13 | H27 | M716 | E14 | H27 | M717 | E15 | H27 |
| M718 | E16 | H27 | M719 | E17 | H27 | M720 | E18 | H27 |
| M721 | E19 | H27 | M722 | E20 | H27 | M723 | E21 | H27 |
| M724 | E22 | H27 | M725 | E23 | H27 | M726 | E24 | H27 |
| M727 | E25 | H27 | M728 | E26 | H27 | M729 | E27 | H27 |

The concentration of the electron-transporting host material of the formula (1) as described above or described as preferred in the mixture of the invention or in the light-emitting layer of the device of the invention is in the range from 5% by weight to 90% by weight, preferably in the range from 10% by weight to 85% by weight, more preferably in the range from 20% by weight to 85% by weight, even more preferably in the range from 30% by weight to 80% by weight, very especially preferably in the range from 20% by weight to 60% by weight and most preferably in the range from 30% by weight to 50% by weight, based on the overall mixture or based on the overall composition of the light-emitting layer.

The concentration of the hole-transporting host material of the formula (2) as described above or described as preferred in the mixture of the invention or in the light-emitting layer of the device of the invention is in the range from 10% by weight to 95% by weight, preferably in the range from 15% by weight to 90% by weight, more preferably in the range from 15% by weight to 80% by weight, even more preferably in the range from 20% by weight to 70% by weight, very especially preferably in the range from 40% by weight to 80% by weight and most preferably in the range from 50% by weight to 70% by weight, based on the overall mixture or based on the overall composition of the light-emitting layer.

The present invention also relates to a mixture which, as well as the aforementioned host materials 1 and 2, as described above or described as preferred, especially mixtures M1 to M729, also contains at least one phosphorescent emitter.

The present invention also relates to an organic electroluminescent device as described above or described as preferred, wherein the light-emitting layer, as well as the aforementioned host materials 1 and 2, as described above or described as preferred, especially the material combinations M1 to M729, also comprises at least one phosphorescent emitter.

The term “phosphorescent emitters” typically encompasses compounds where the light is emitted through a spin-forbidden transition from an excited state having higher spin multiplicity, i.e. a spin state >1, for example through a transition from a triplet state or a state having an even higher spin quantum number, for example a quintet state. This is preferably understood to mean a transition from a triplet state.

Suitable phosphorescent emitters (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent emitters.

In general, all phosphorescent complexes as used for phosphorescent OLEDs according to the prior art and as known to those skilled in the art in the field of organic electroluminescent devices are suitable.

Preferred phosphorescent emitters according to the present invention conform to the formula (3),

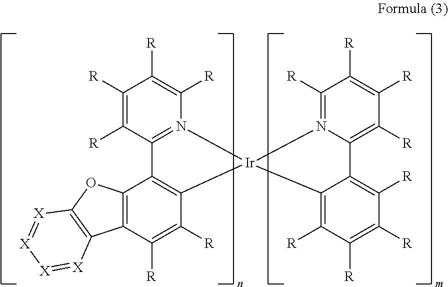

where the symbols and indices for this formula (3) are defined as follows:
n+m is 3, n is 1 or 2, m is 2 or 1,
X is N or CR,
R is H, D, or a branched or linear alkyl group having 1 to 10 carbon atoms or a partly or fully deuterated branched or linear alkyl group having 1 to 10 carbon atoms or a cycloalkyl group which has 4 to 7 carbon atoms and may be partly or fully substituted by deuterium.

The invention accordingly further provides an organic electroluminescent device as described above or described as preferred, characterized in that the light-emitting layer, as well as the host materials 1 and 2, comprises at least one phosphorescent emitter conforming to the formula (3) as described above.

In emitters of the formula (3), n is preferably 1 and m is preferably 2.

In emitters of the formula (3), preferably, one X is selected from N and the other X are CR.

In emitters of the formula (3) at least one R is preferably different from H. In emitters of the formula (3) preferably two R are different from H and have one of the other definitions given above for the emitters of the formula (3).

Preferred phosphorescent emitters according to the present invention conform to the formulae (I), (II), (Ill), (IV) or (V)

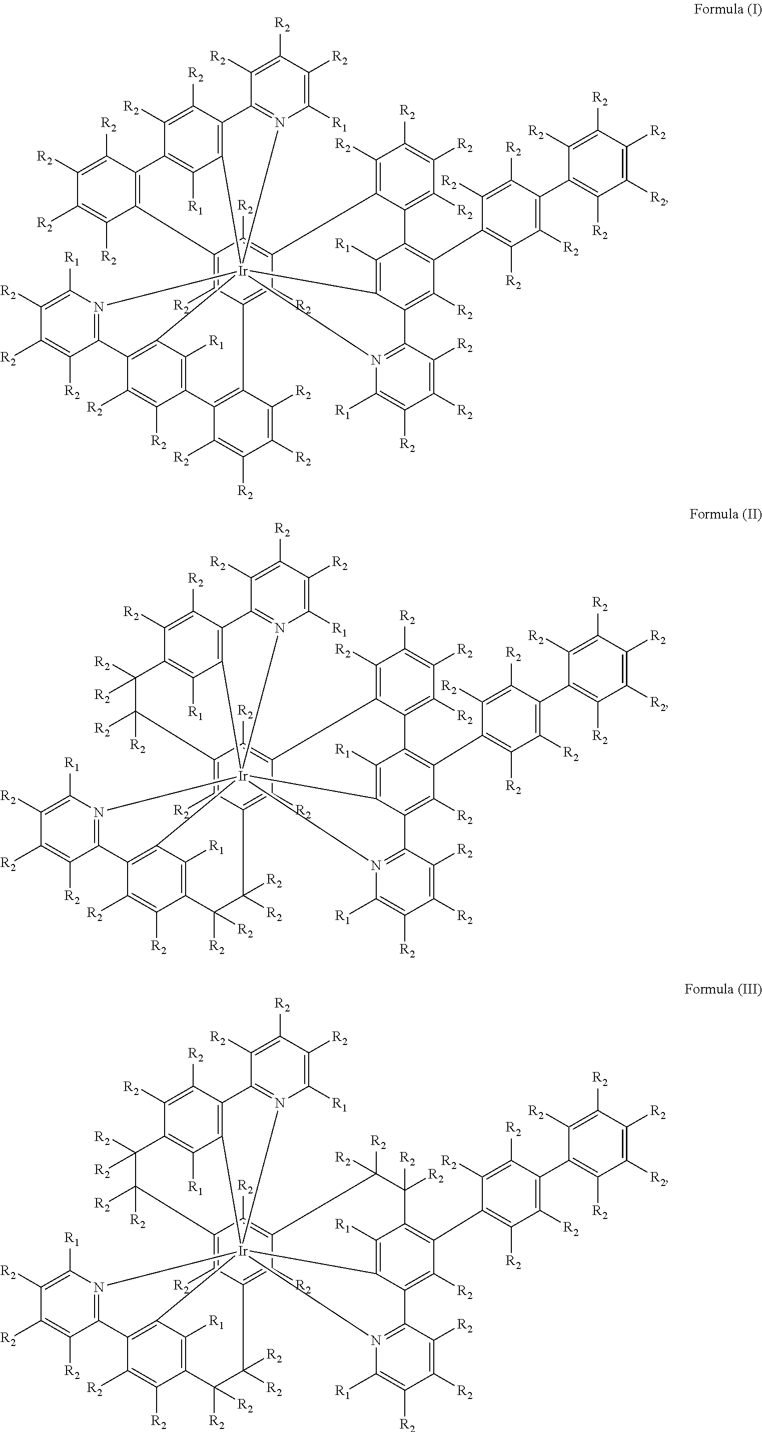

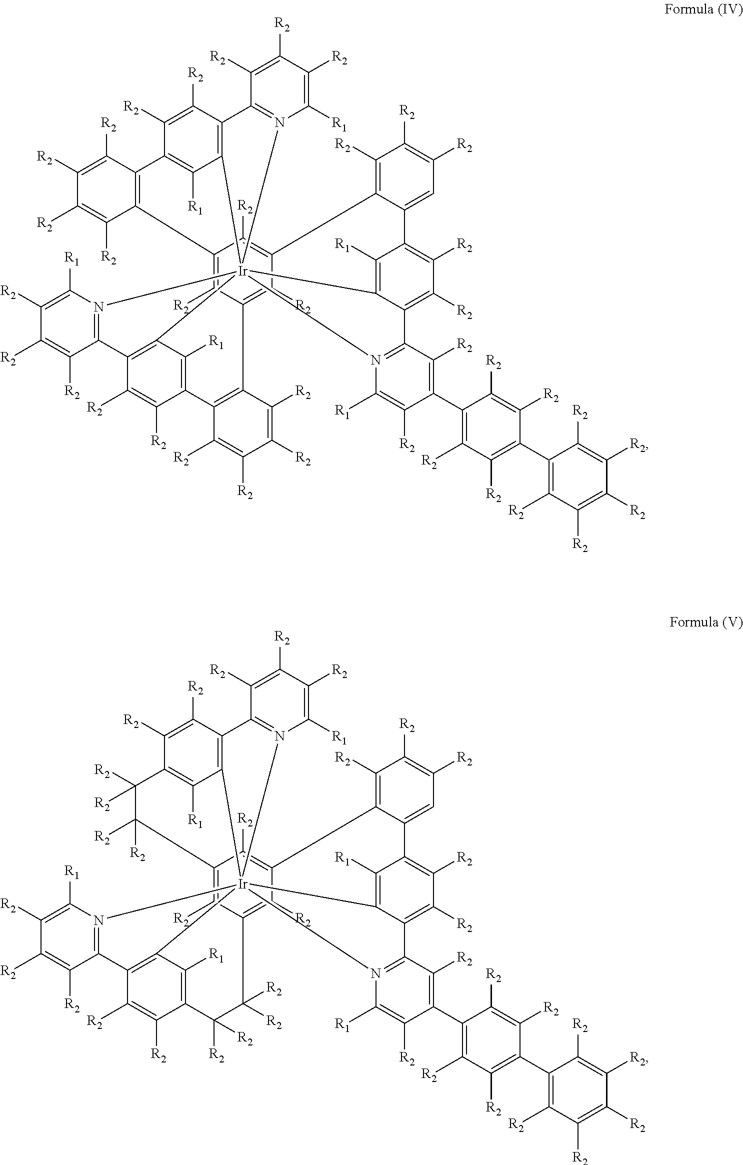

where the symbols and indices for these formulae (I), (II), (Ill), (IV) and (V) are defined as follows:
R¹ is H or D, R² is H, D, or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred phosphorescent emitters according to the present invention conform to the formulae (VI), (VII) or (VIII)

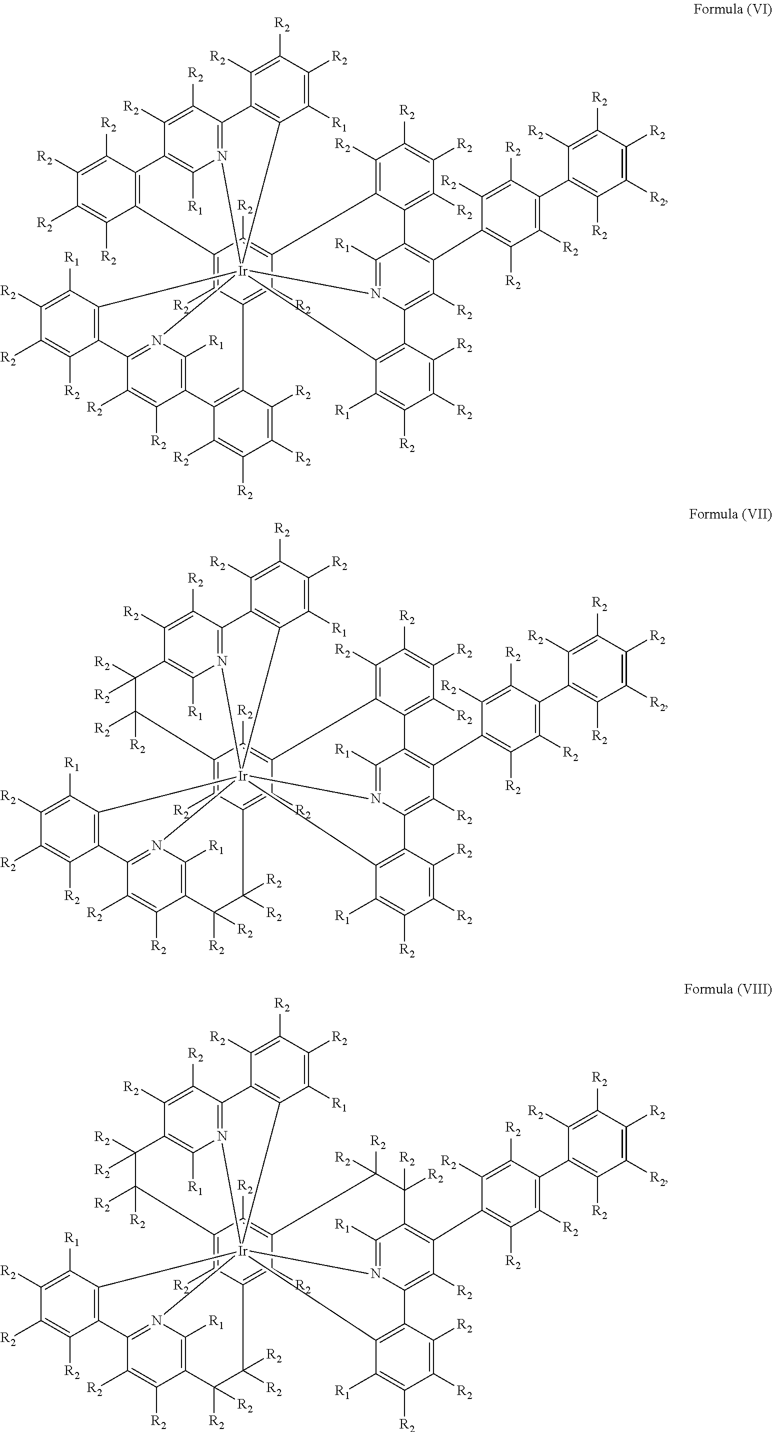

where the symbols and indices for these formulae (VI), (VII) and (VIII) are defined as follows:
R₁ is H or D, R₂ is H, D, F or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred examples of phosphorescent emitters are listed in table 4 below.

TABLE 4

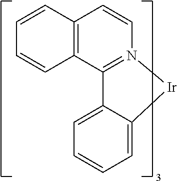

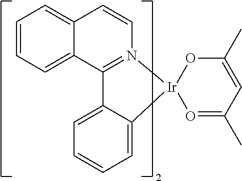

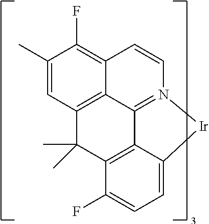

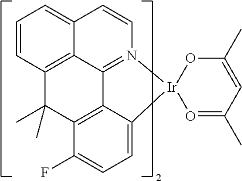

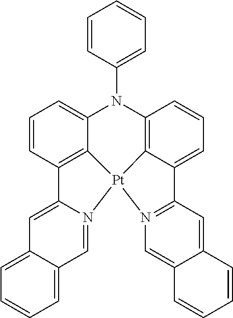

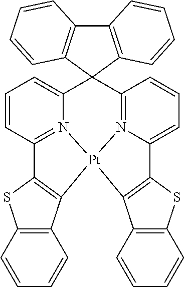

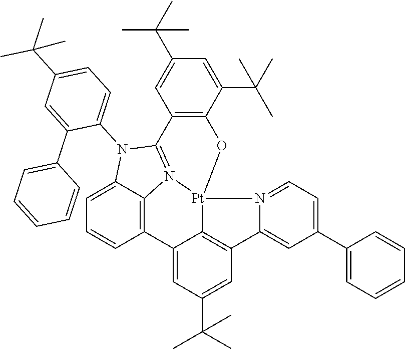

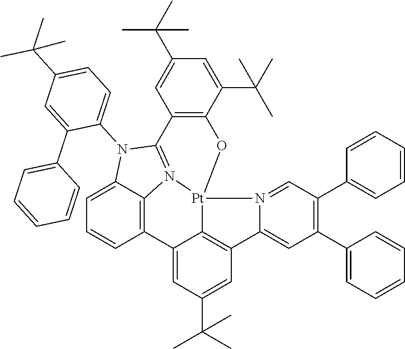

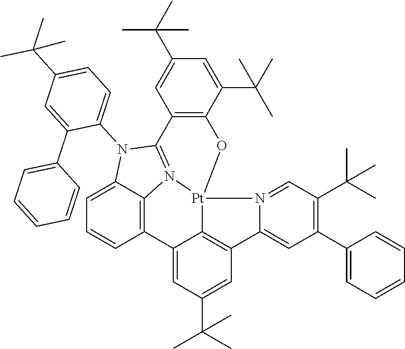

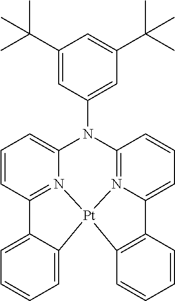

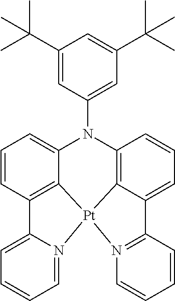

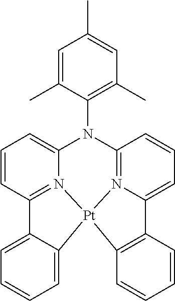

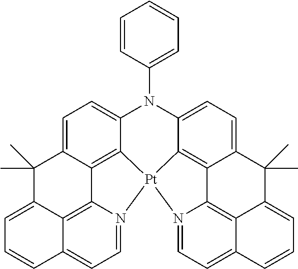

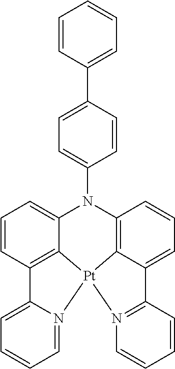

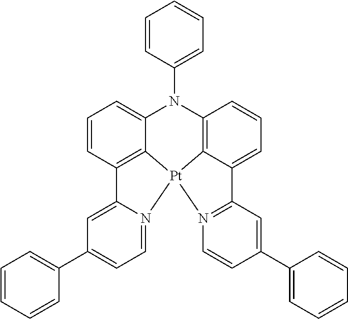

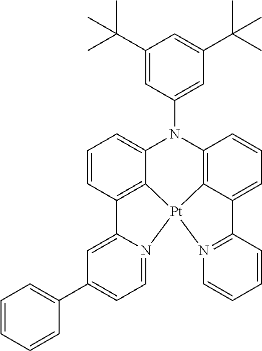

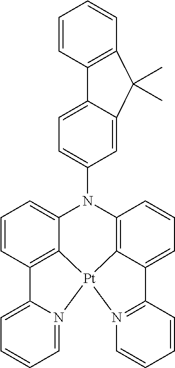

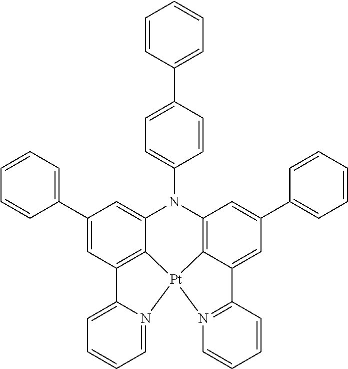

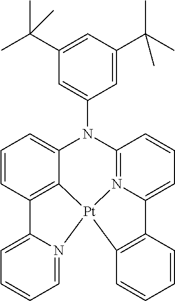

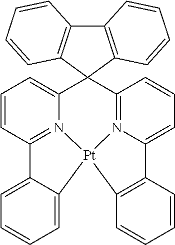

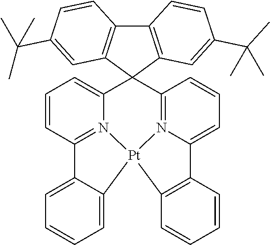

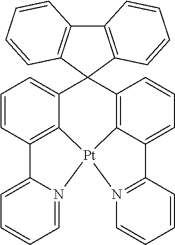

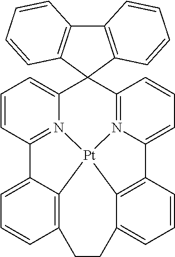

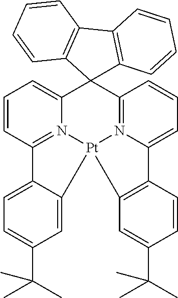

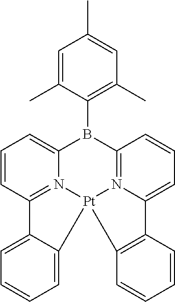

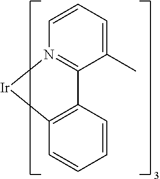

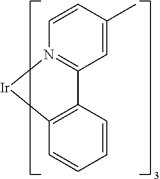

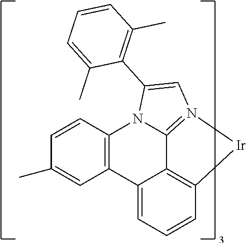

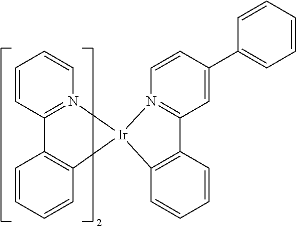

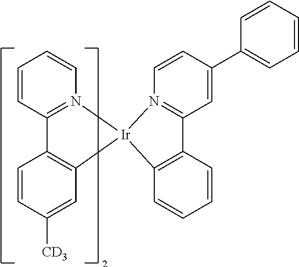

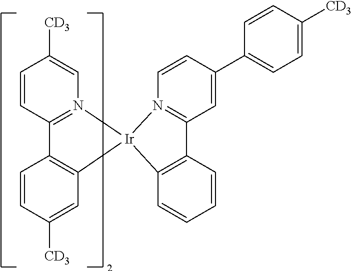

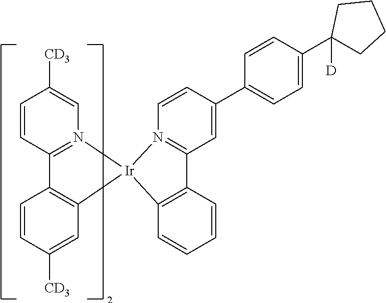

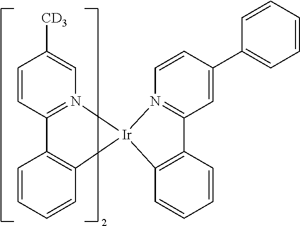

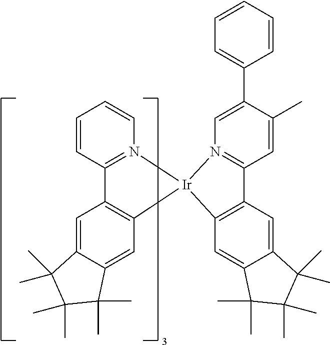

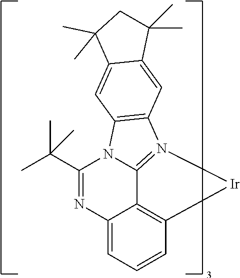

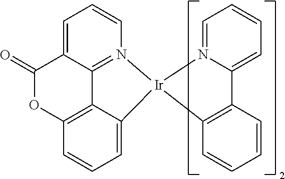

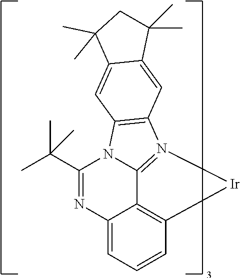

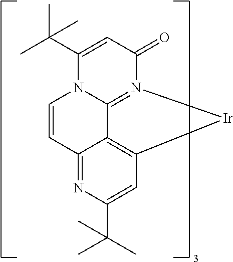

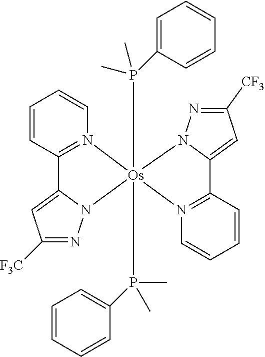

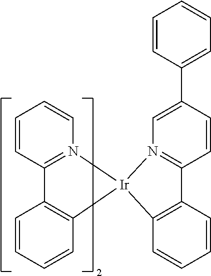

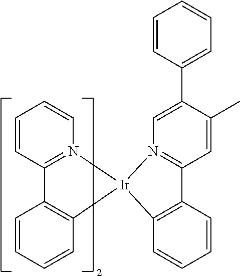

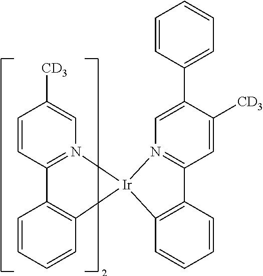

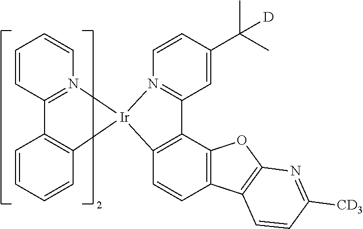

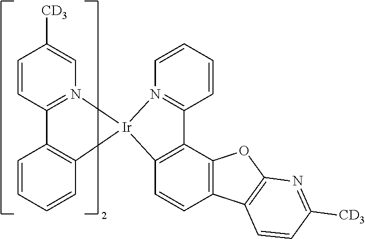

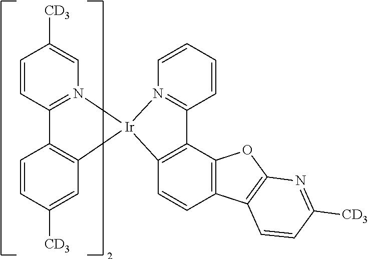

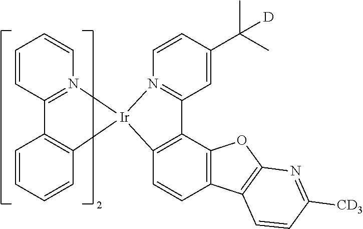

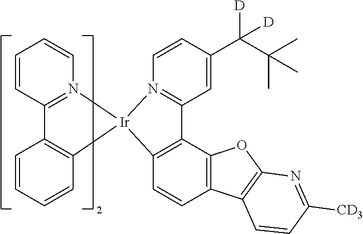

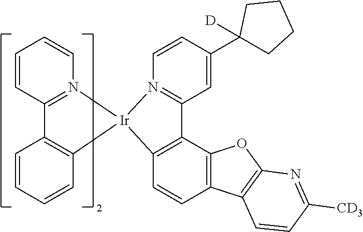

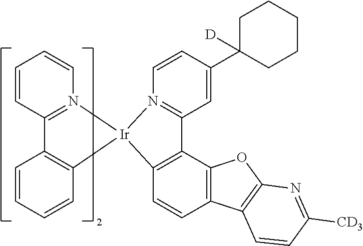

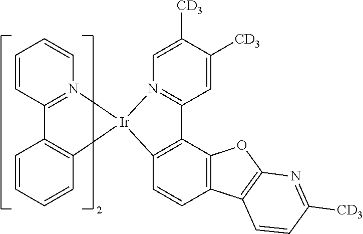

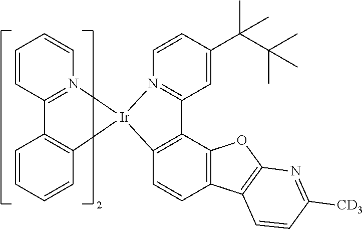

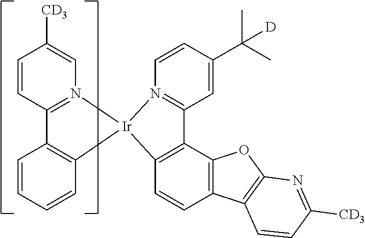

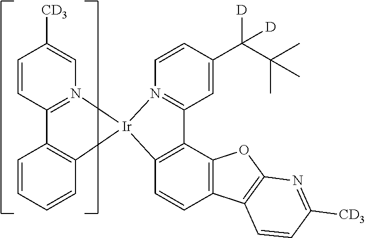

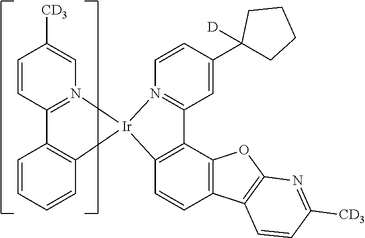

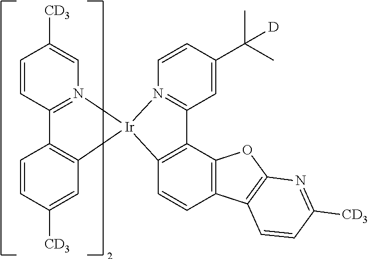

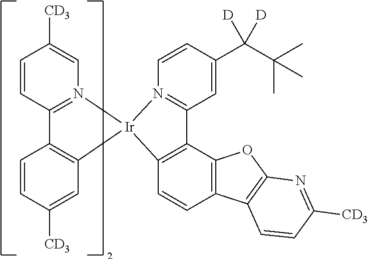

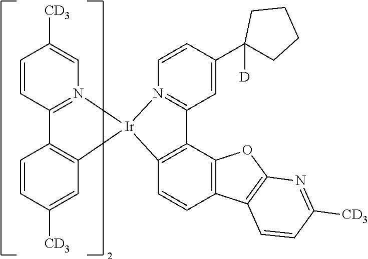

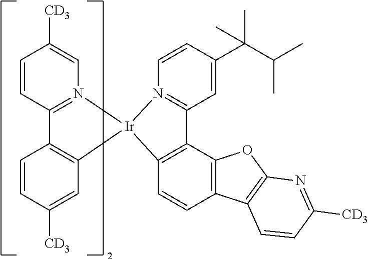

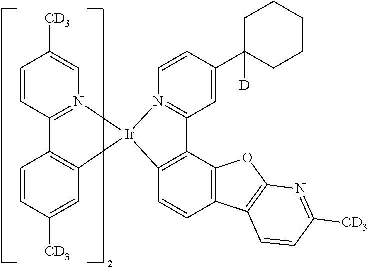

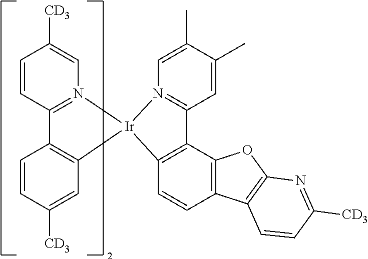

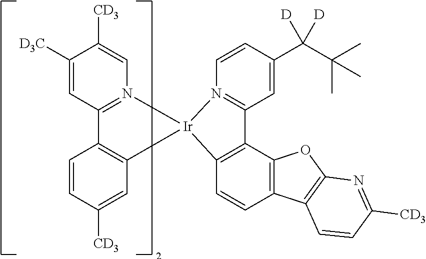

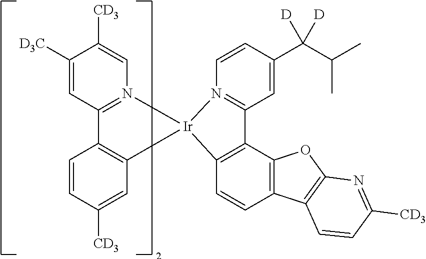

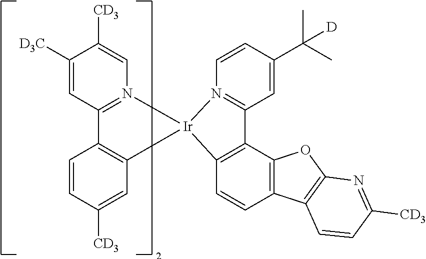

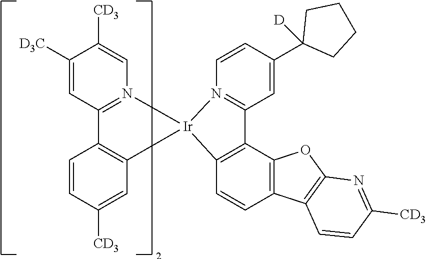

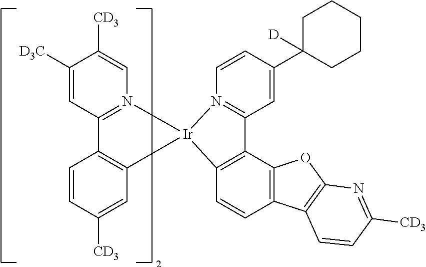

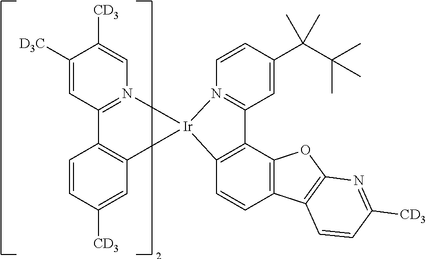

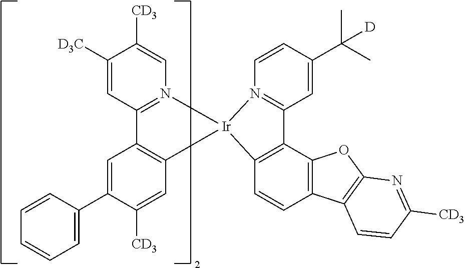

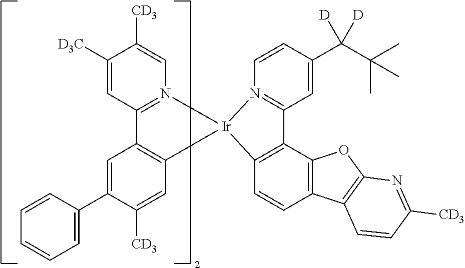

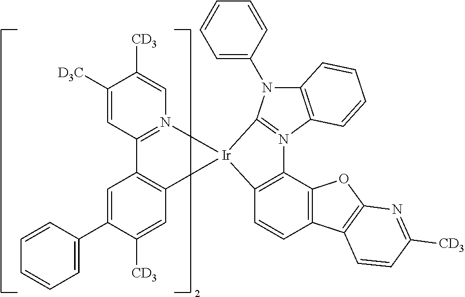

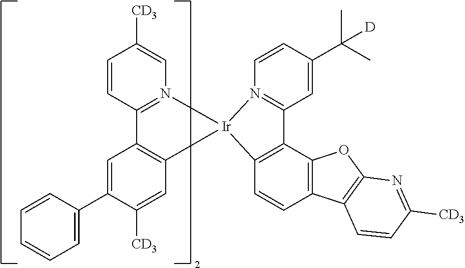

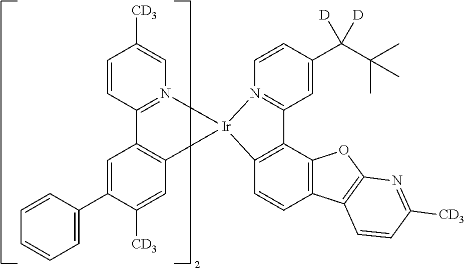

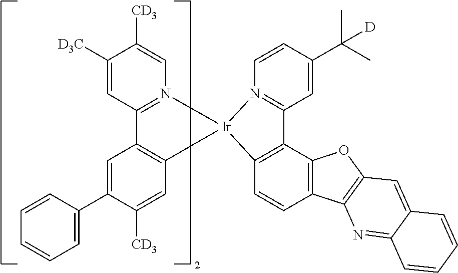

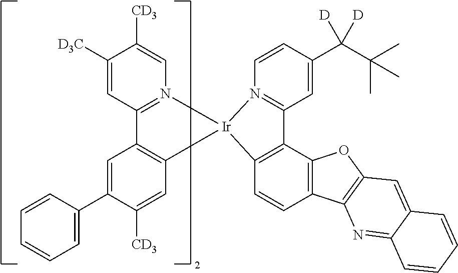

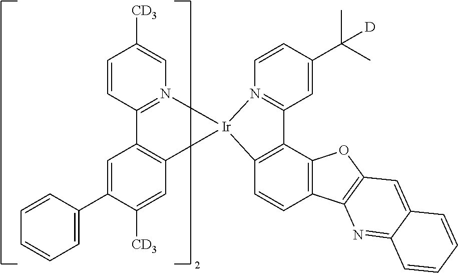

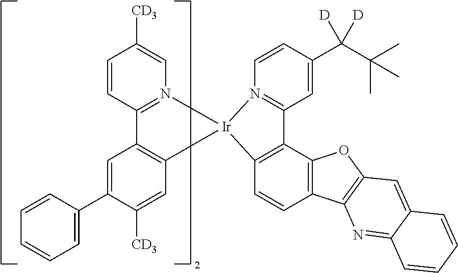

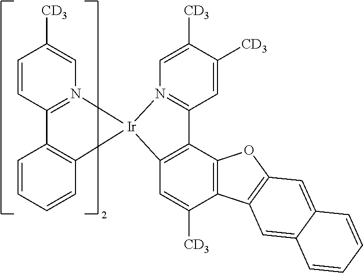

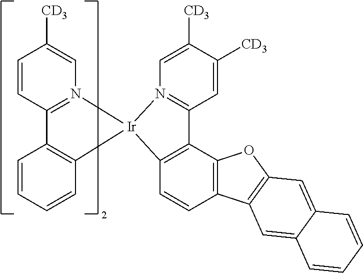

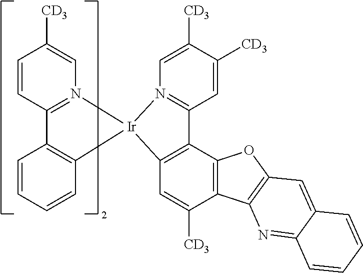

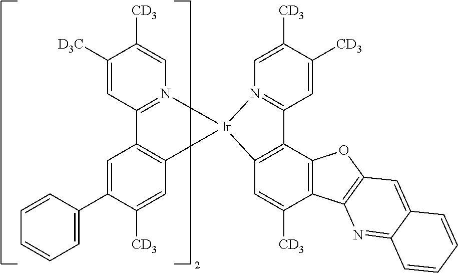

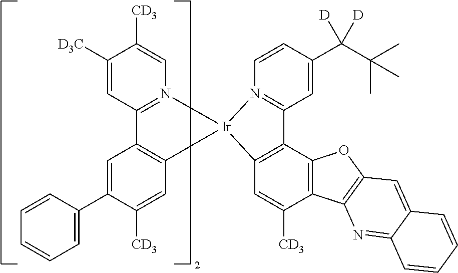

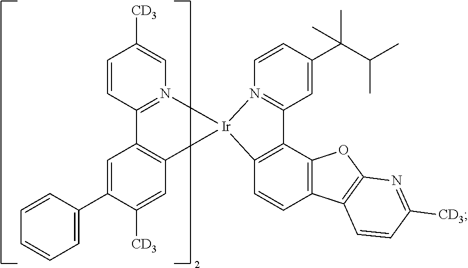

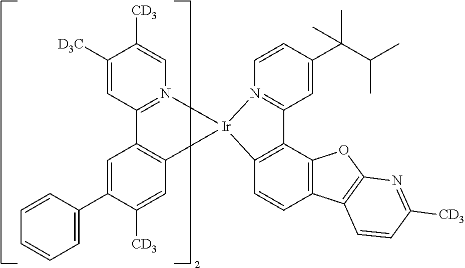

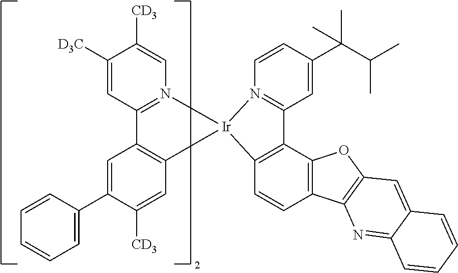

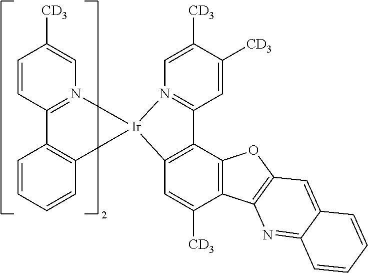

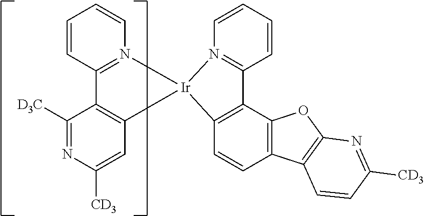

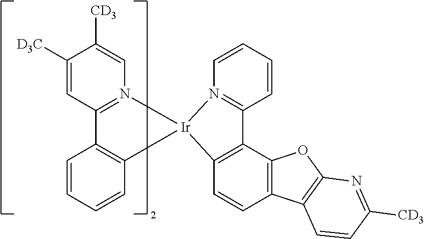

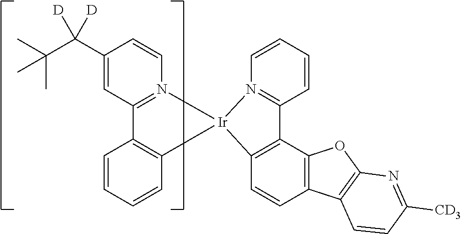

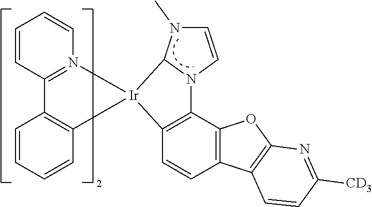

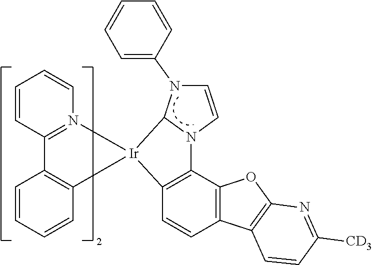

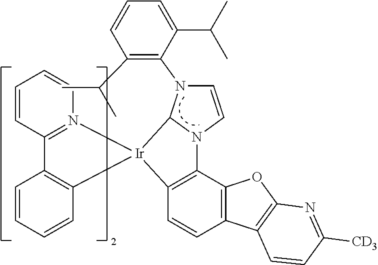

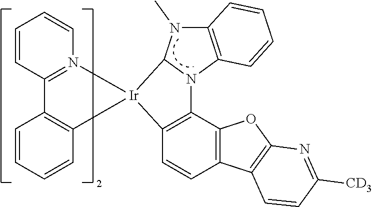

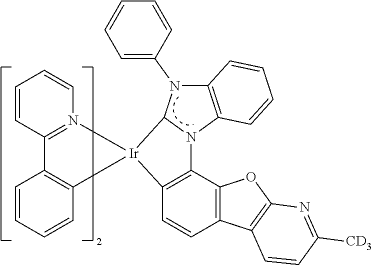

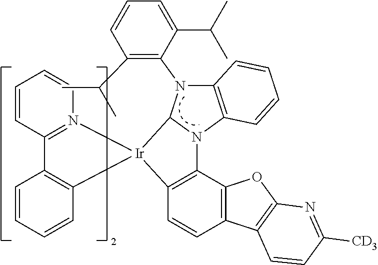

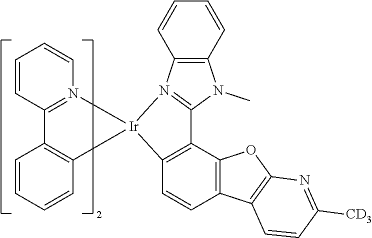

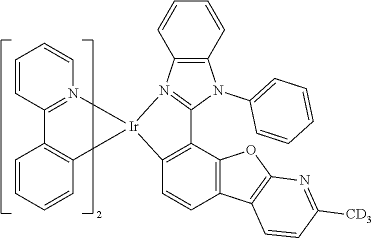

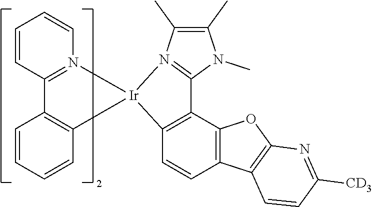

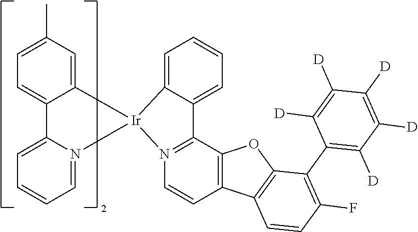

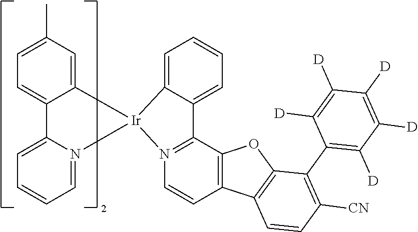

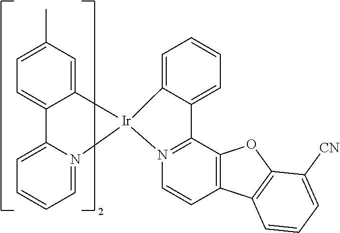

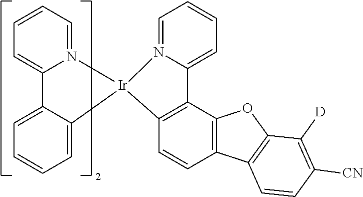

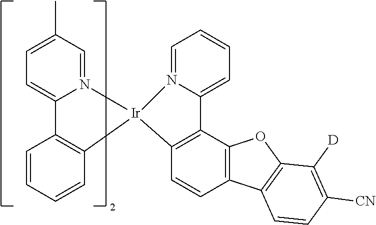

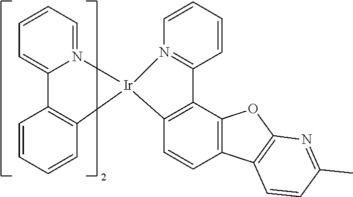

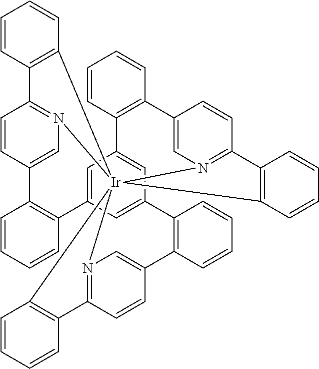

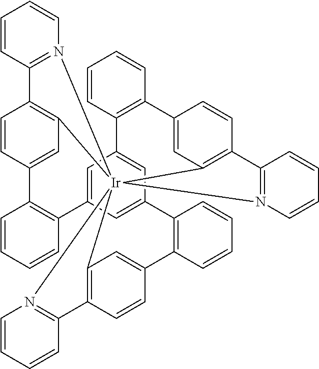

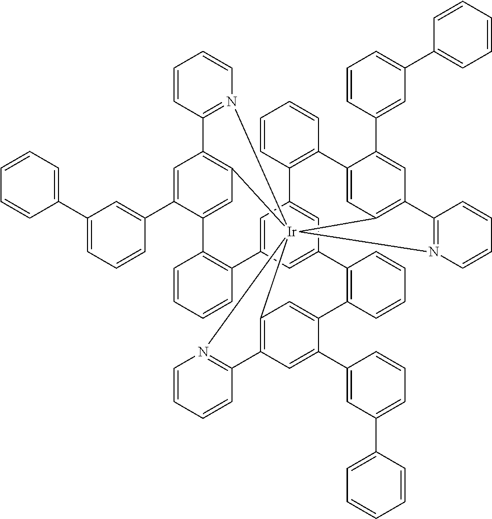

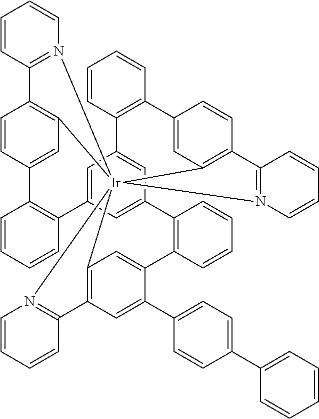

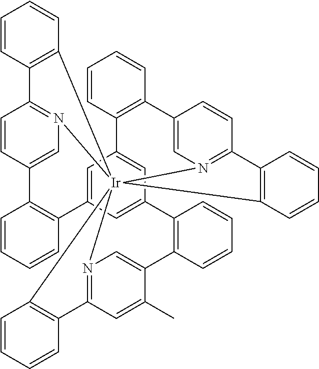

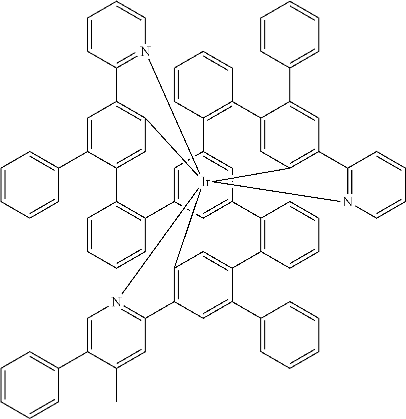

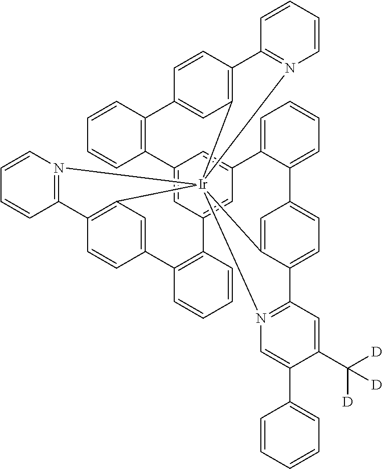

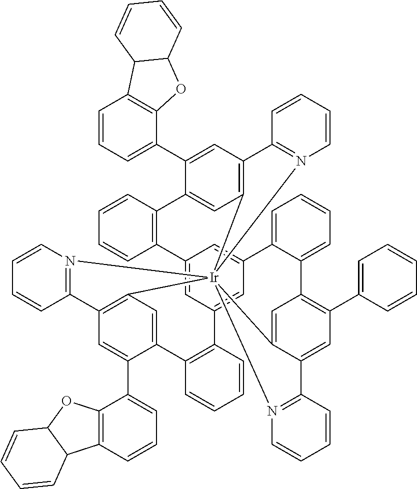

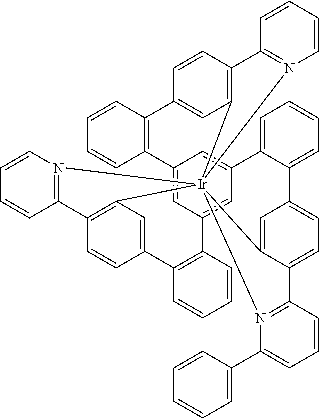

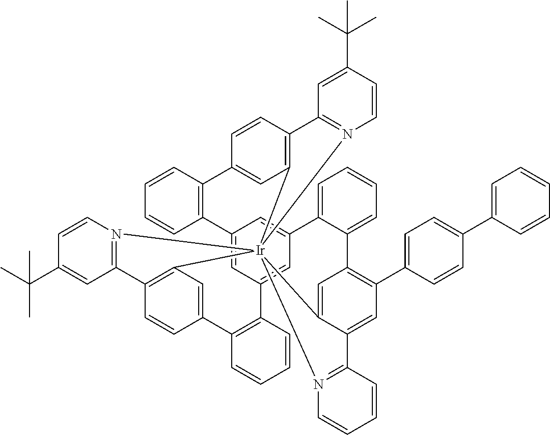

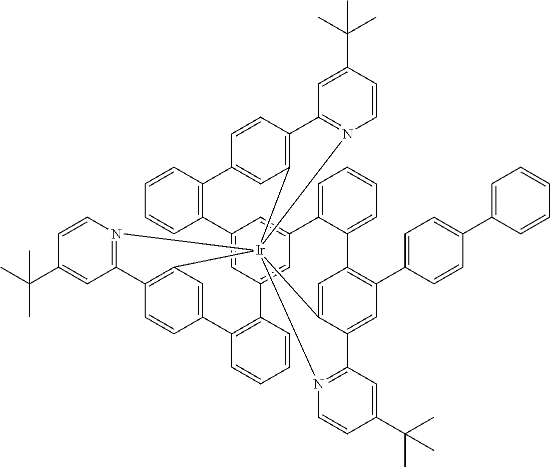

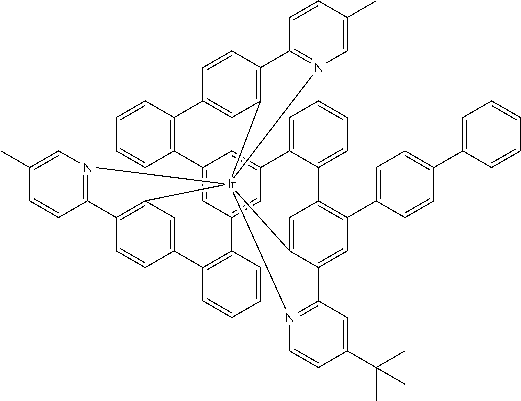

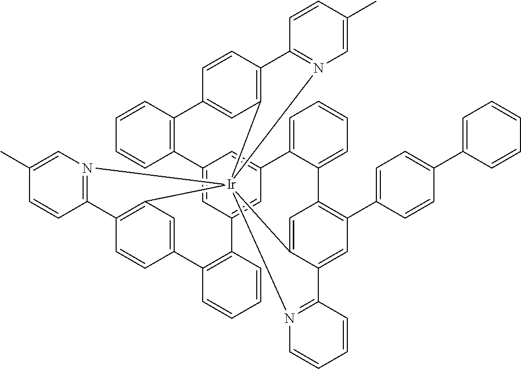

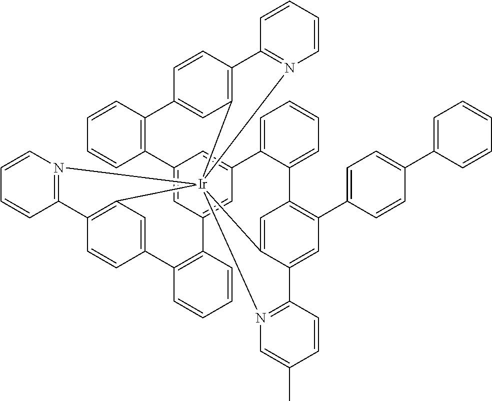

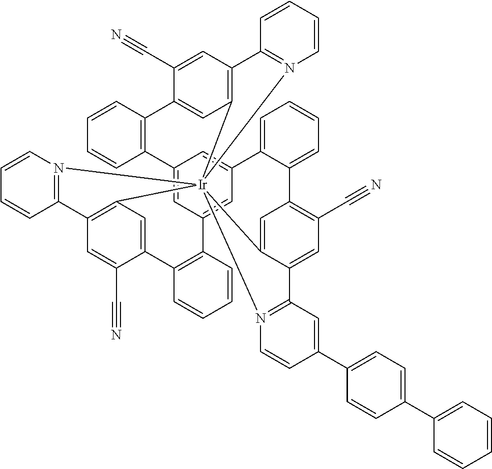

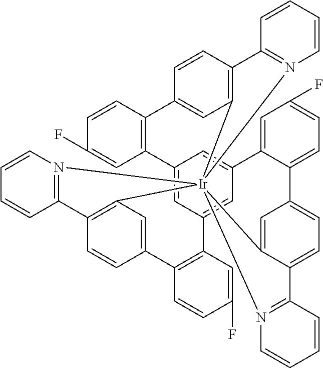

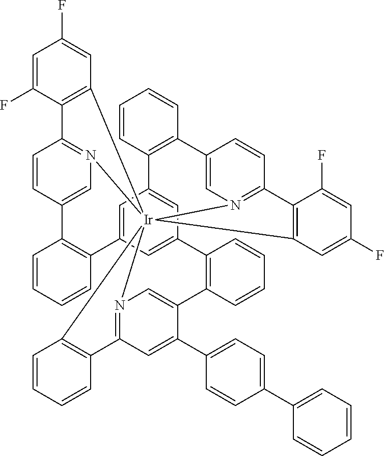

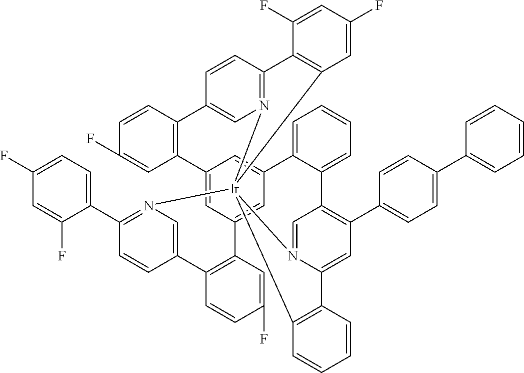

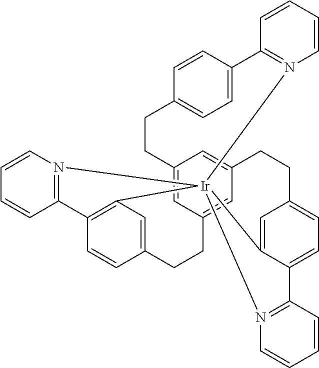

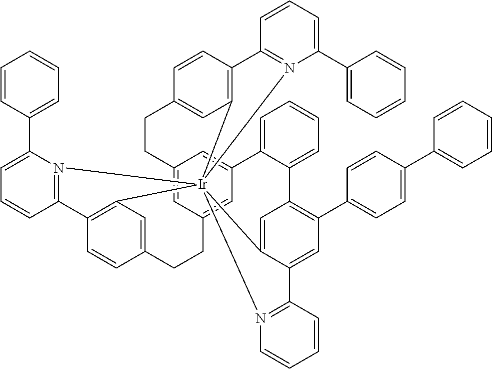

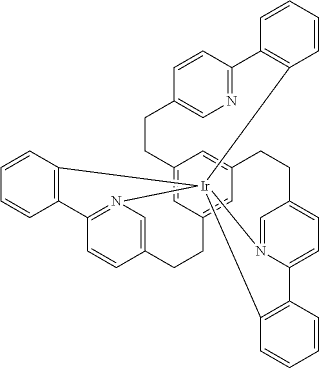

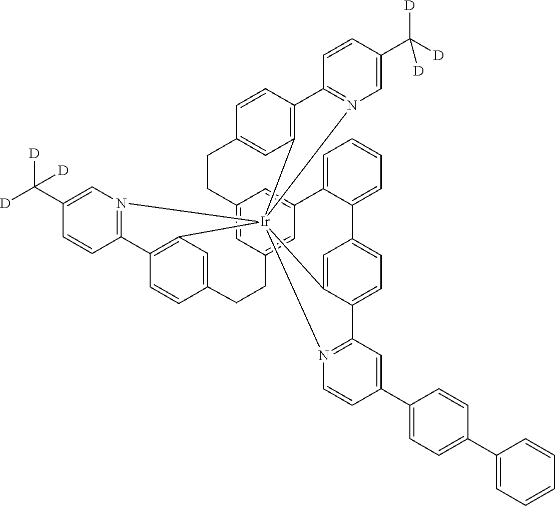

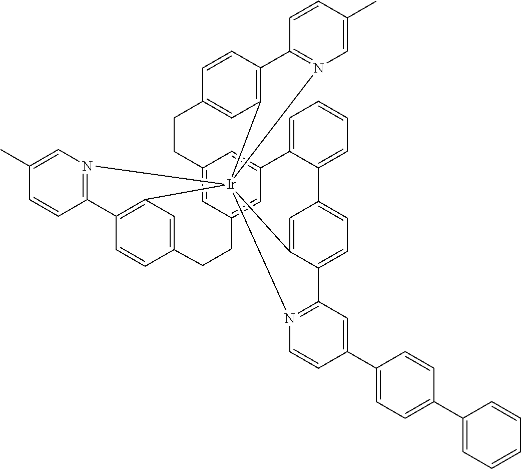

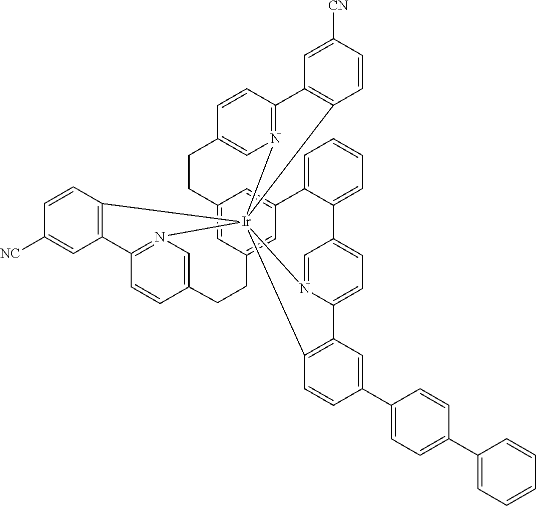

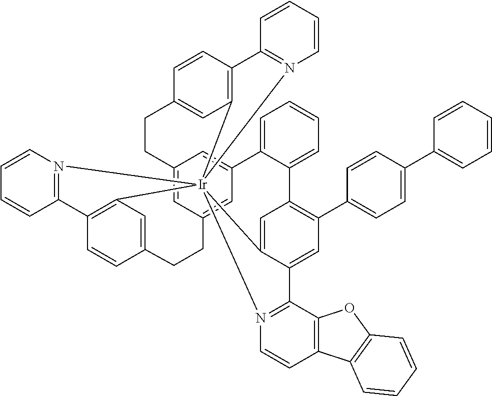

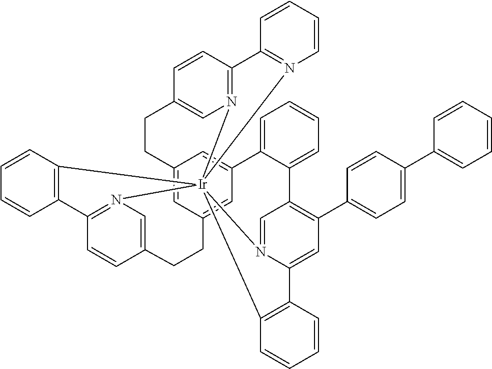

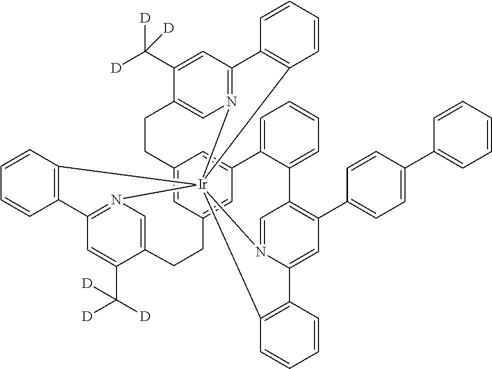

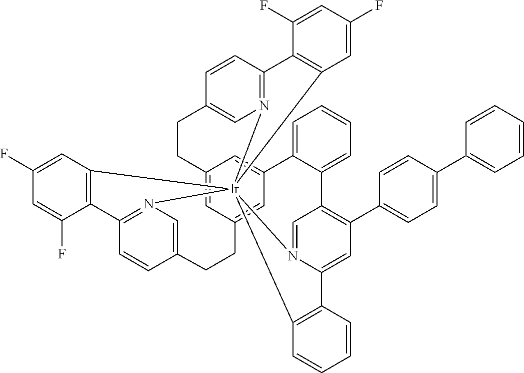

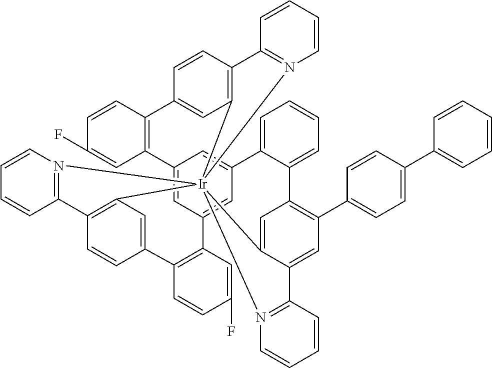

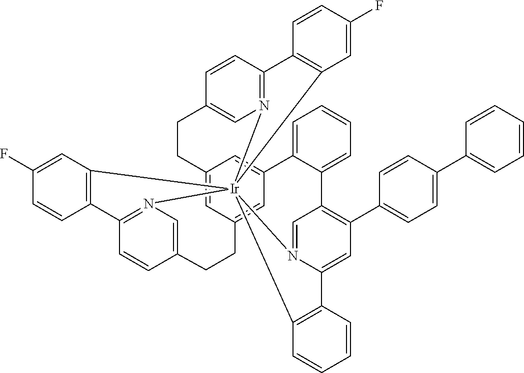

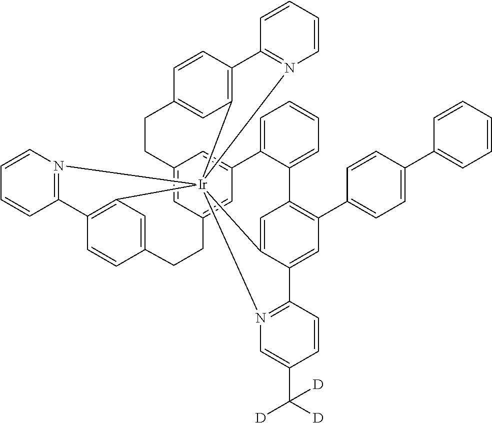

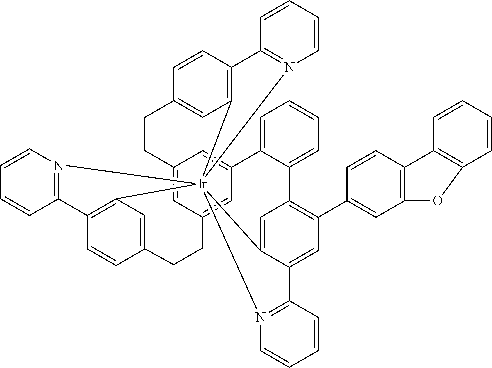

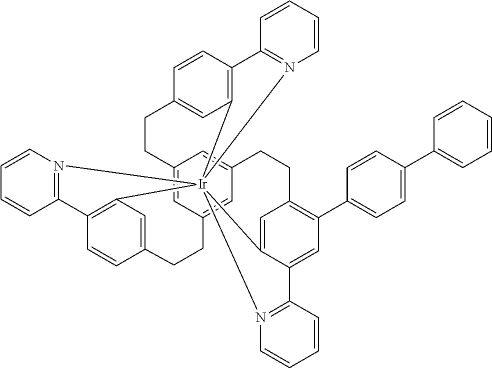

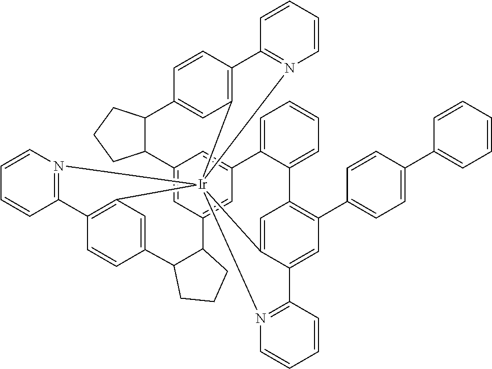

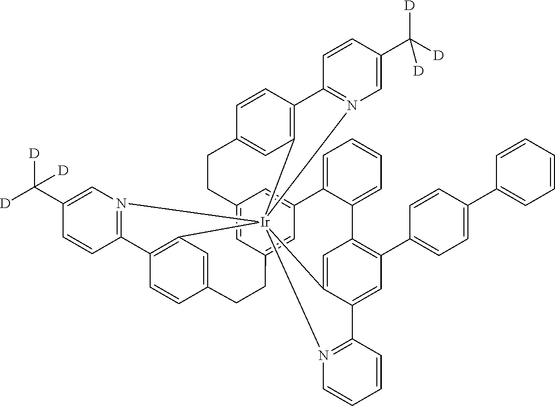

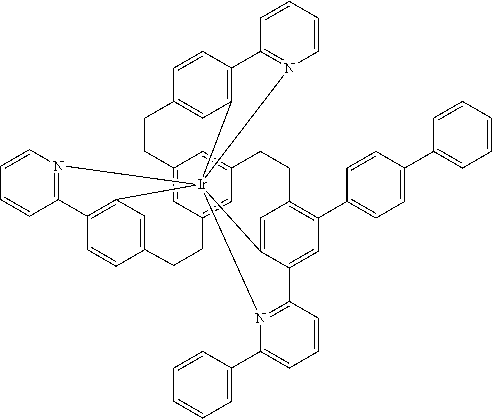

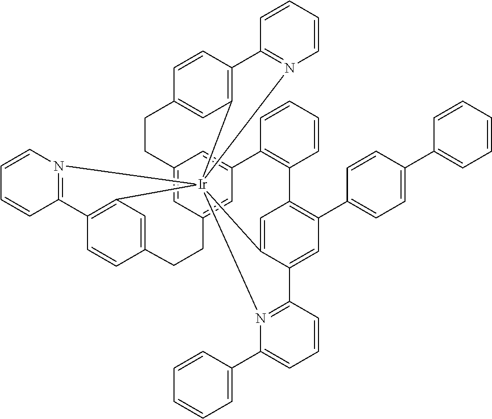

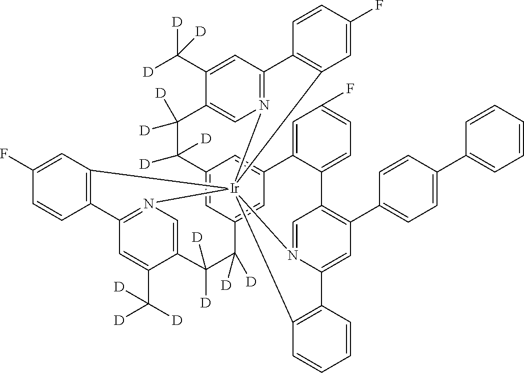

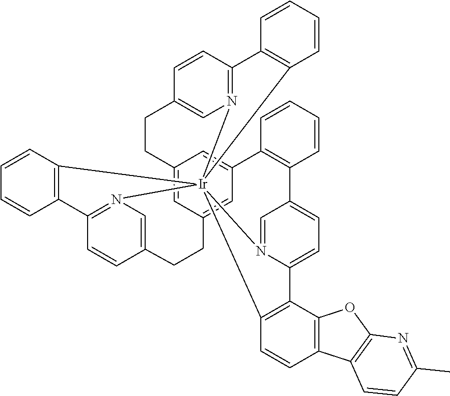

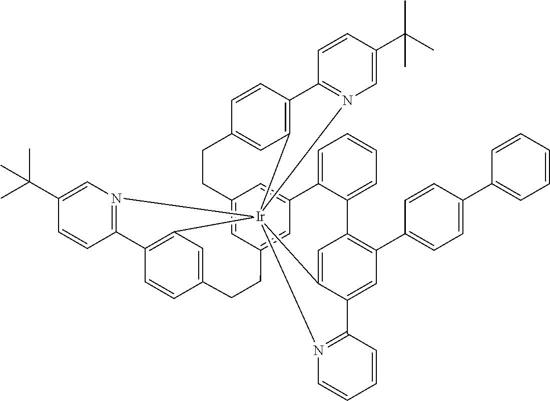

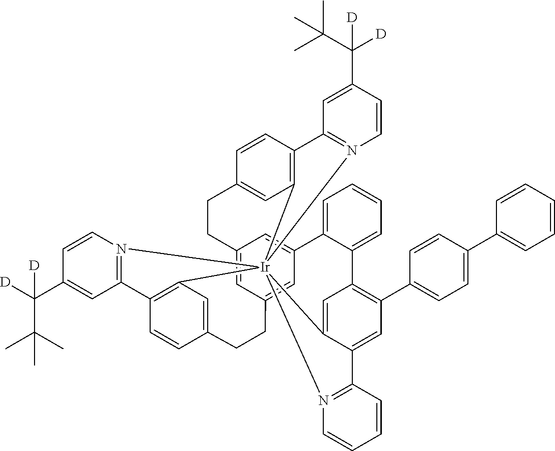

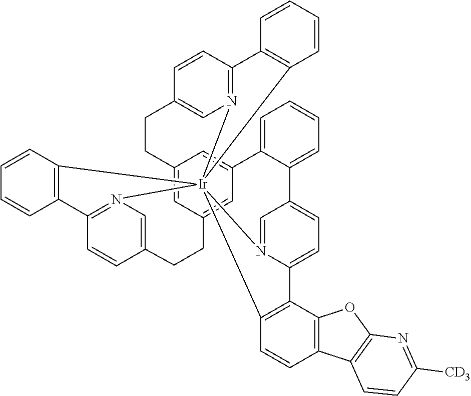

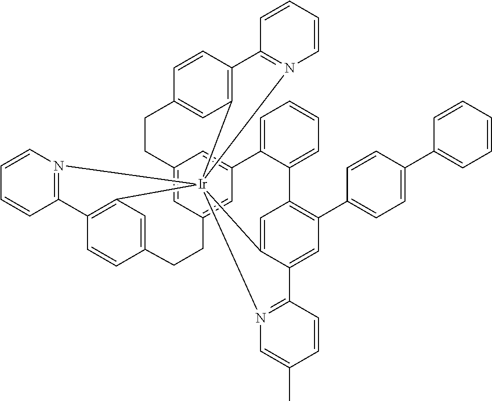

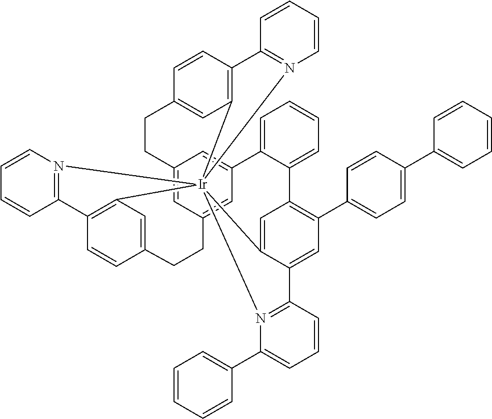

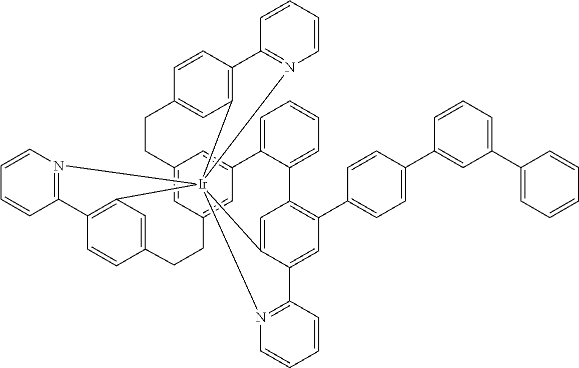

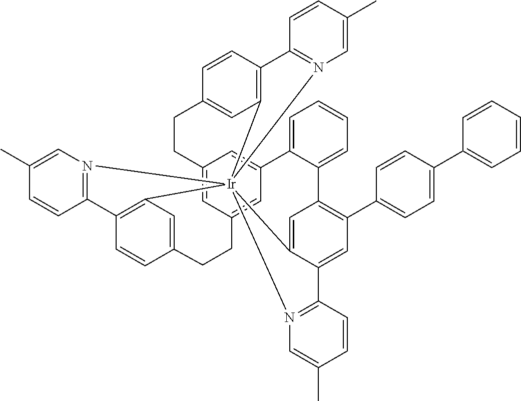

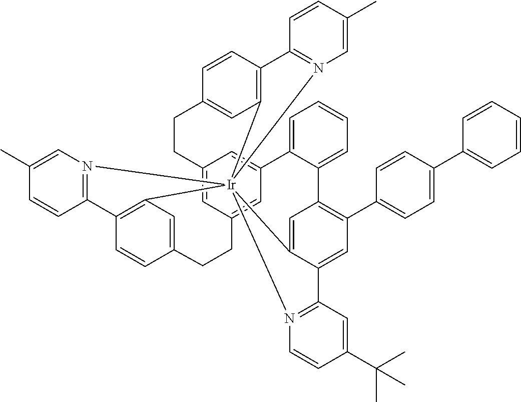

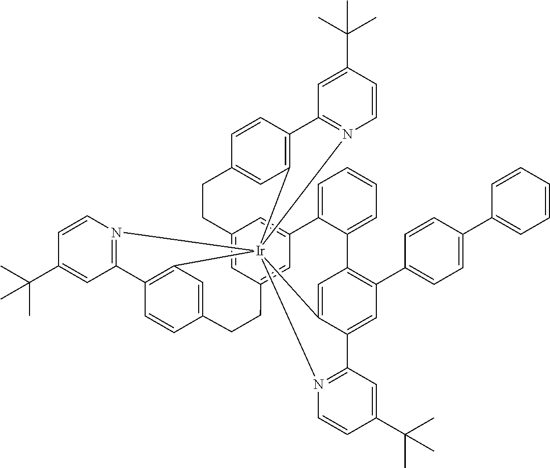

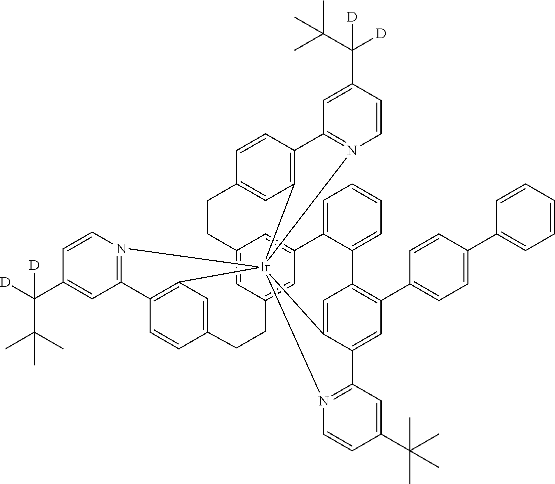

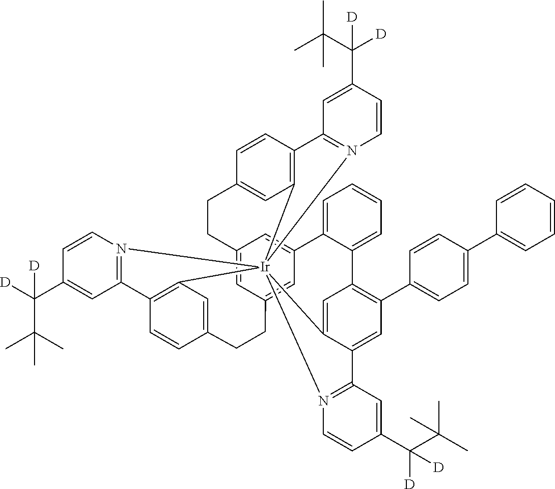

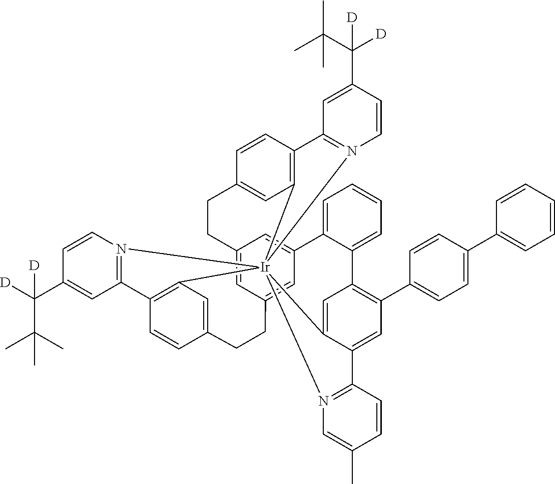

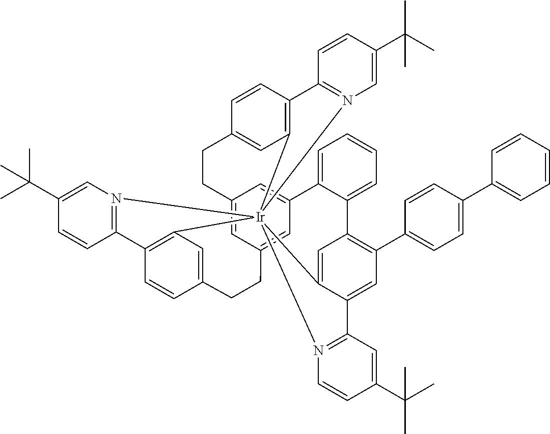

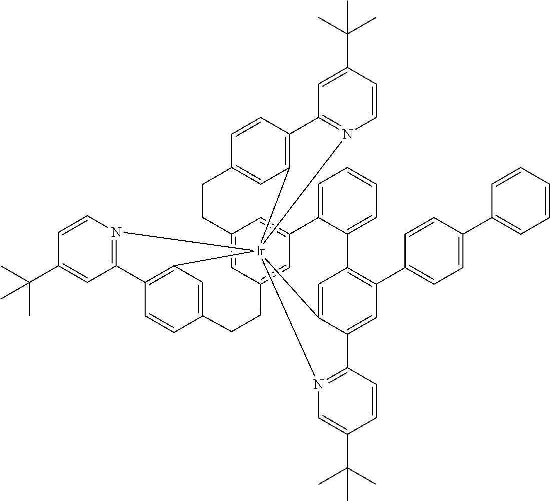

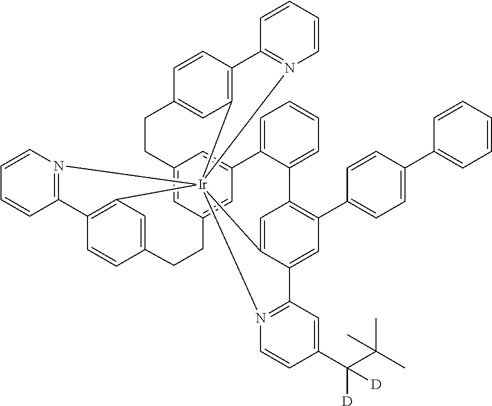

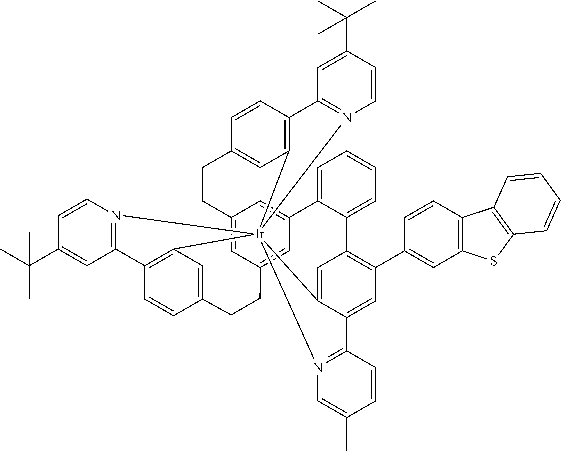

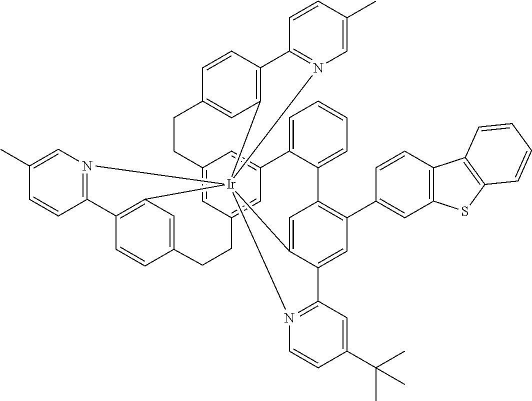

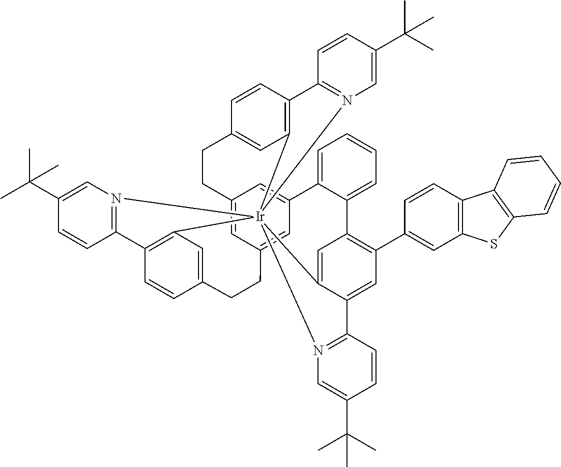

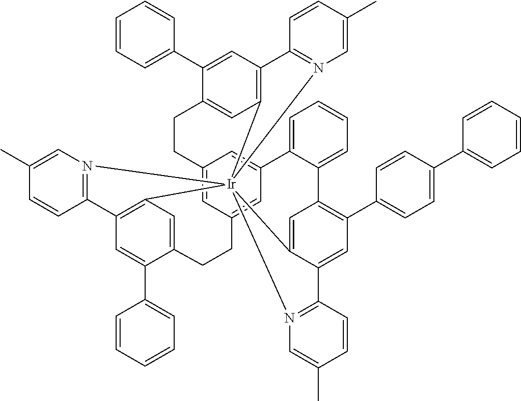

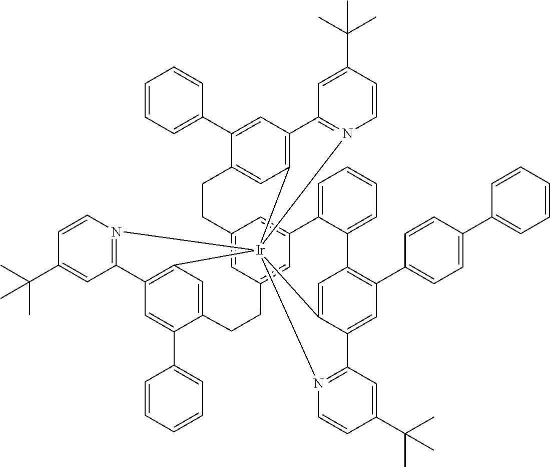

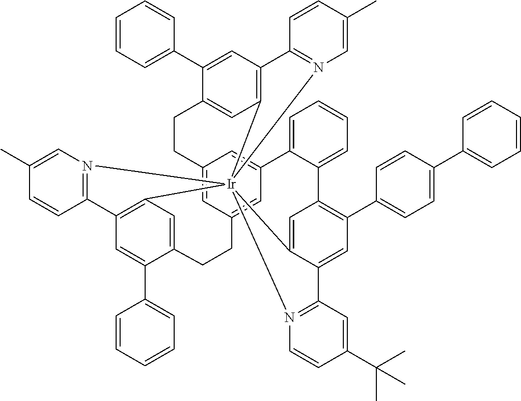

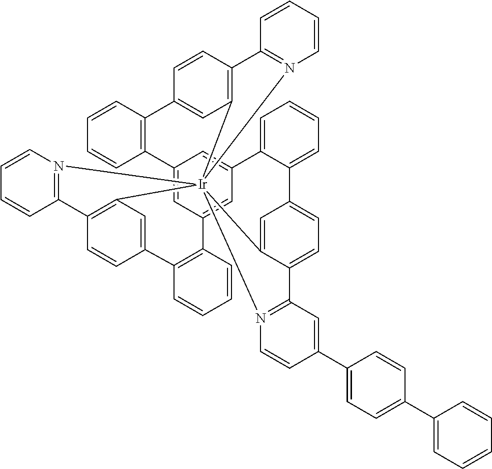

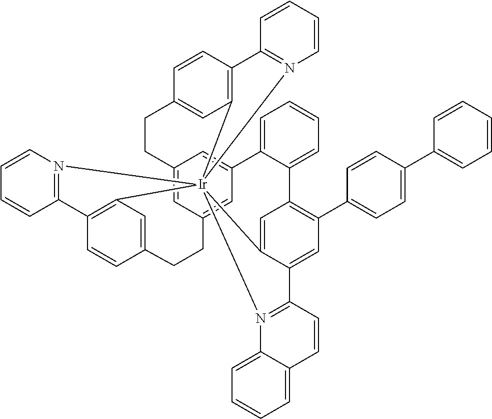

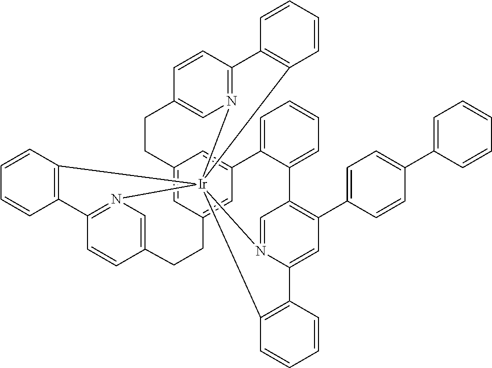

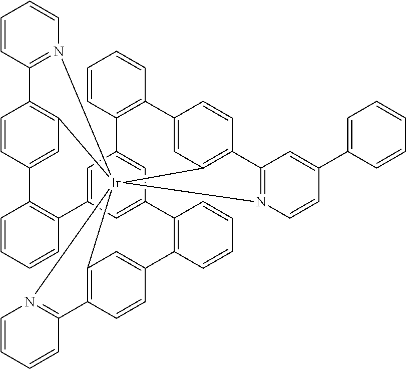

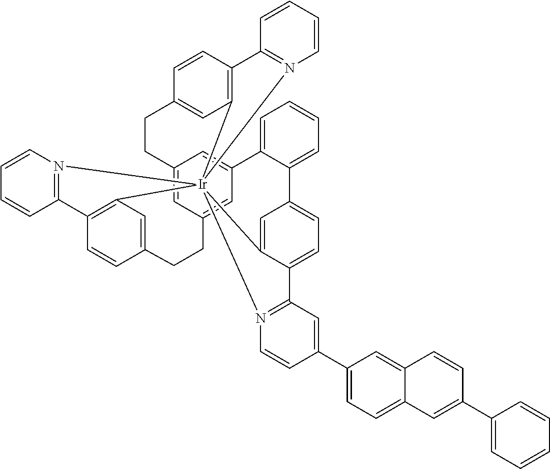

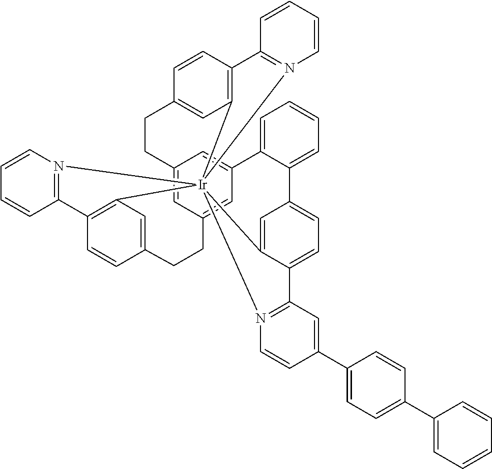

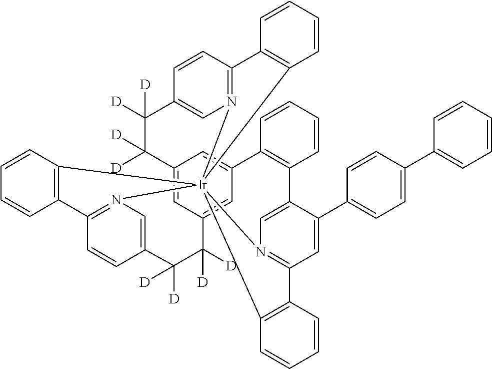

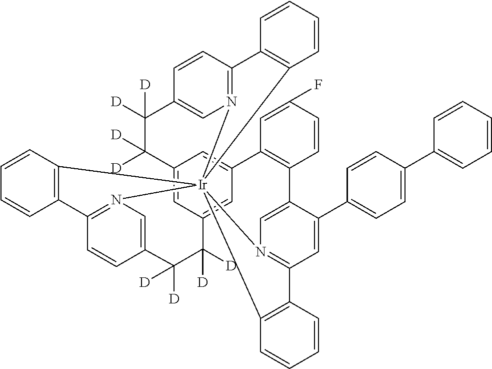

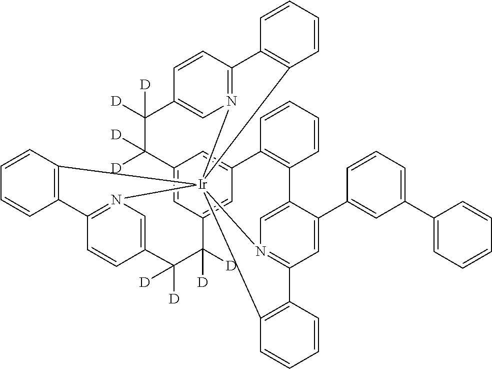

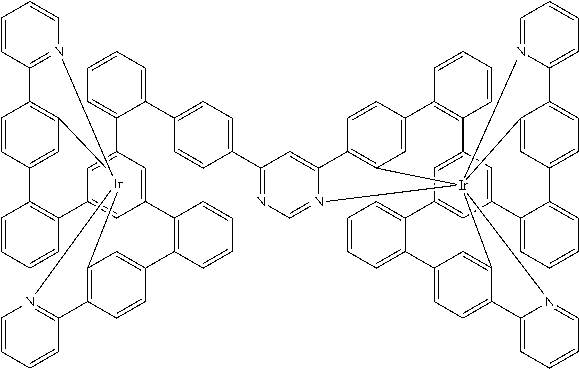

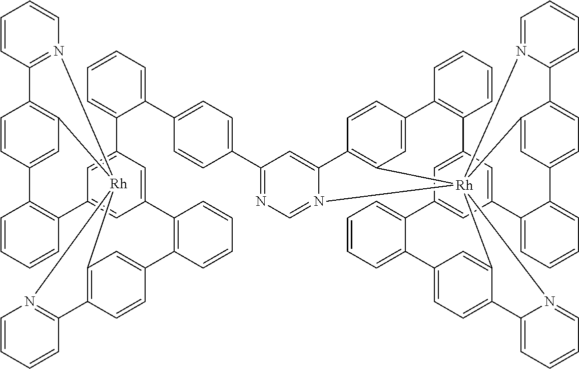

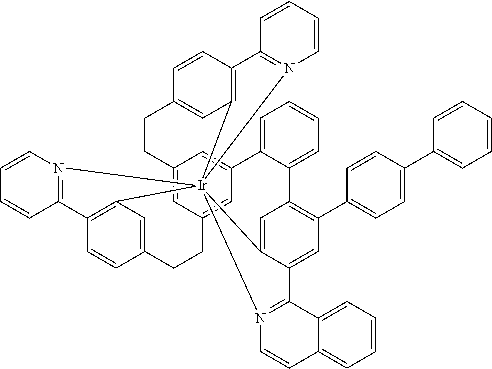

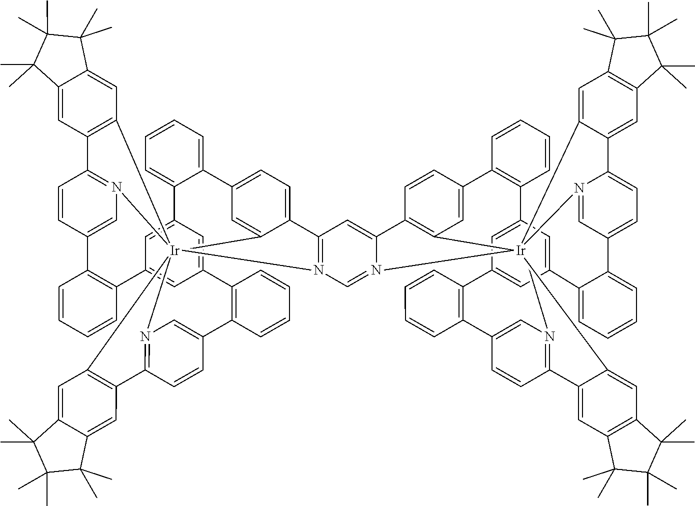

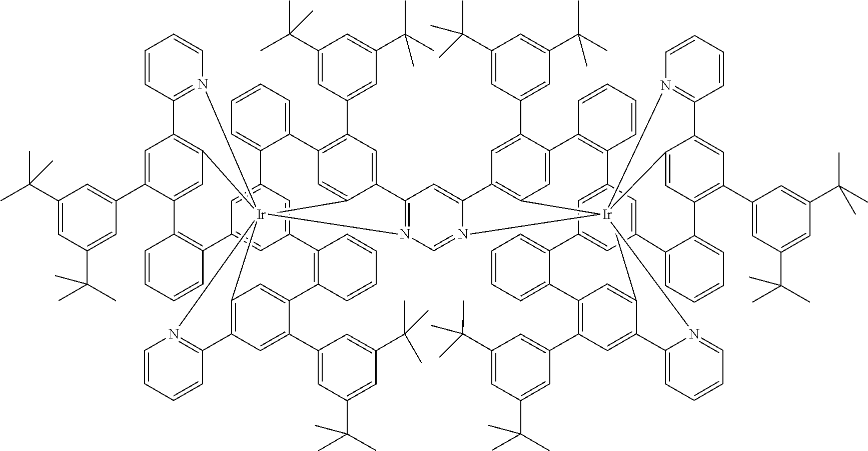

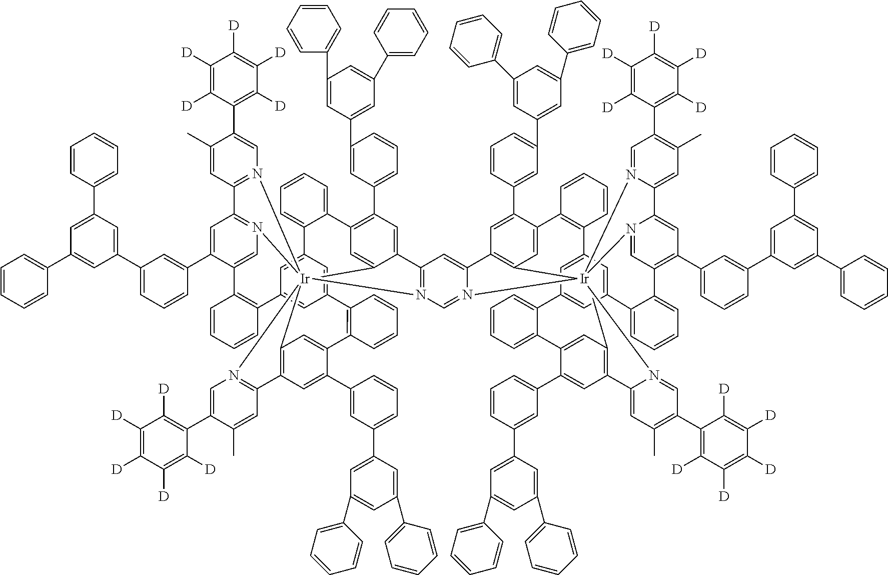

In the mixtures of the invention or in the light-emitting layer of the device of the invention, any mixture selected from the sum of the mixtures M1 to M729 is preferably combined with a compound of the formula (3) or a compound of the formulae (I) to (VIII) or a compound from table 4.

The light-emitting layer in the organic electroluminescent device of the invention, comprising at least one phosphorescent emitter, is preferably an infrared-emitting or yellow-, orange-, red-, green-, blue- or ultraviolet-emitting layer, more preferably a yellow- or green-emitting layer and most preferably a green-emitting layer.

A yellow-emitting layer is understood here to mean a layer having a photoluminescence maximum within the range from 540 to 570 nm. An orange-emitting layer is understood to mean a layer having a photoluminescence maximum within the range from 570 to 600 nm. A red-emitting layer is understood to mean a layer having a photoluminescence maximum within the range from 600 to 750 nm. A green-emitting layer is understood to mean a layer having a photoluminescence maximum within the range from 490 to 540 nm. A blue-emitting layer is understood to mean a layer having a photoluminescence maximum within the range from 440 to 490 nm. The photoluminescence maximum of the layer is determined here by measuring the photoluminescence spectrum of the layer having a layer thickness of 50 nm at room temperature, said layer having the inventive combination of the host materials of the formulae (1) and (2) and the appropriate emitter.

The photoluminescence spectrum of the layer is recorded, for example, with a commercial photoluminescence spectrometer.

The photoluminescence spectrum of the emitter chosen is generally measured in oxygen-free solution, 10-5 molar, at room temperature, a suitable solvent being any in which the chosen emitter dissolves in the concentration mentioned. Particularly suitable solvents are typically toluene or 2-methyl-THF, but also dichloromethane. Measurement is effected with a commercial photoluminescence spectrometer. The triplet energy T1 in eV is determined from the photoluminescence spectra of the emitters. First the peak maximum Plmax. (in nm) of the photoluminescence spectrum is determined. The peak maximum Plmax. (in nm) is then converted to eV by: E(T1 in eV)=1240/E(T1 in nm)=1240/PLmax. (in nm).

Preferred phosphorescent emitters are accordingly yellow emitters, preferably of the formula (3), of the formulae (I) to (VIII) or from table 4, the triplet energy T₁ of which is preferably ˜2.3 eV to ˜2.1 eV.

Preferred phosphorescent emitters are accordingly green emitters, preferably of the formula (3), of the formulae (I) to (VIII) or from table 4, the triplet energy T₁ of which is preferably ˜2.5 eV to ˜2.3 eV.

Particularly preferred phosphorescent emitters are accordingly green emitters, preferably of the formula (3), of the formulae (I) to (VIII) or from table 4 as described above, the triplet energy T₁ of which is preferably ˜2.5 eV to ˜2.3 eV.

Most preferably, green emitters, preferably of the formula (3), of the formulae (I) to (VIII) or from table 4, as described above, are selected for the composition of the invention or emitting layer of the invention.

It is also possible for fluorescent emitters to be present in the light-emitting layer of the device of the invention.

Preferred fluorescent emitters are selected from the class of the arylamines. An arylamine or an aromatic amine in the context of this invention is understood to mean a compound containing three substituted or unsubstituted aromatic or heteroaromatic ring systems bonded directly to the nitrogen.

In a further preferred embodiment of the invention, the at least one light-emitting layer of the organic electroluminescent device, as well as the host materials 1 and 2, as described above or described as preferred, may comprise further host materials or matrix materials, called mixed matrix systems. The mixed matrix systems preferably comprise three or four different matrix materials, more preferably three different matrix materials (in other words, one further matrix component in addition to the host materials 1 and 2, as described above). Particularly suitable matrix materials which can be used in combination as matrix component in a mixed matrix system are selected from wide-band gap materials, bipolar host materials, electron transport materials (ETM) and hole transport materials (HTM).

A wide-band gap material is understood herein to mean a material within the scope of the disclosure of U.S. Pat. No. 7,294,849 which is characterized by a band gap of at least 3.5 eV, the band gap being understood to mean the gap between the HOMO and LUMO energy of a material.

One source of more detailed information about mixed matrix systems is the application WO 2010/108579. Particularly suitable matrix materials which can be used in combination with the host materials 1 and 2 as described above or described as preferred, as matrix components of a mixed matrix system in phosphorescent or fluorescent organic electroluminescent devices, are selected from the preferred matrix materials specified below for phosphorescent emitters or the preferred matrix materials for fluorescent emitters, according to what type of emitter is used. Preferably, the mixed matrix system is optimized for an emitter of the formula (3), the formulae (I) to (VIII), or from table 4.

In one embodiment of the present invention, the mixture does not comprise any further constituents, i.e. functional materials, aside from the constituents of electron-transporting host material of the formula (1) and hole-transporting host material of the formula (2). These are material mixtures that are used as such for production of the light-emitting layer. These mixtures are also referred to as premix systems that are used as the sole material source in the vapour deposition of the host materials for the light-emitting layer and have a constant mixing ratio in the vapour deposition. In this way, it is possible in a simple and rapid manner to achieve the vapour deposition of a layer with homogeneous distribution of the components without the need for precise actuation of a multitude of material sources.

In an alternative embodiment of the present invention, the mixture also comprises the phosphorescent emitter, as described above, in addition to the constituents of electron-transporting host material of the formula (1) and hole-transporting host material of the formula (2). In the case of a suitable mixing ratio in the vapour deposition, this mixture may also be used as the sole material source, as described above.

The components or constituents of the light-emitting layer of the device of the invention may thus be processed by vapour deposition or from solution. The material combination of host materials 1 and 2, as described above or described as preferred, optionally with the phosphorescent emitter, as described above or described as preferred, are provided for that purpose in a formulation containing at least one solvent. These formulations may, for example, be solutions, dispersions or emulsions. For this purpose, it may be preferable to use mixtures of two or more solvents.

The present invention therefore further provides a formulation comprising an inventive mixture of host materials 1 and 2, as described above, optionally in combination with a phosphorescent emitter, as described above or described as preferred, and at least one solvent.

Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The formulation here may also comprise at least one further organic or inorganic compound which is likewise used in the light-emitting layer of the device of the invention, especially a further emitting compound and/or a further matrix material. Suitable emitting compounds and further matrix materials have already been detailed above.

The light-emitting layer in the device of the invention, according to the preferred embodiments and the emitting compound, contains preferably between 99.9% and 1% by volume, further preferably between 99% and 10% by volume, especially preferably between 98% and 60% by volume, very especially preferably between 97% and 80% by volume, of matrix material composed of at least one compound of the formula (1) and at least one compound of the formula (2) according to the preferred embodiments, based on the overall composition of emitter and matrix material. Correspondingly, the light-emitting layer in the device of the invention preferably contains between 0.1% and 99% by volume, further preferably between 1% and 90% by volume, more preferably between 2% and 40% by volume, most preferably between 3% and 20% by volume, of the emitter based on the overall composition of the light-emitting layer composed of emitter and matrix material. If the compounds are processed from solution, preference is given to using the corresponding amounts in % by weight rather than the above-specified amounts in % by volume.

The light-emitting layer in the device of the invention, according to the preferred embodiments and the emitting compound, preferably contains the matrix material of the formula (1) and the matrix material of the formula (2) in a percentage by volume ratio between 3:1 and 1:3, preferably between 1:2.5 and 1:1, more preferably between 1:2 and 1:1. If the compounds are processed from solution, preference is given to using the corresponding ratio in % by weight rather than the above-specified ratio in % by volume.

The present invention also relates to an organic electroluminescent device as described above or described as preferred, wherein the organic layer comprises a hole injection layer (HIL) and/or a hole transport layer (HTL), the hole-injecting material and hole-transporting material of which is a monoamine that does not contain a carbazole unit. The hole-injecting material and hole-transporting material preferably comprises a monoamine containing a fluorenyl or bispirofluorenyl group, but no carbazole unit.

Preferred monoamines which are used in accordance with the invention in the organic layer of the device of the invention may be described by the formula (4)

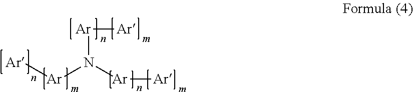

where the symbols and indices for this formula (4) are defined as follows:
Ar and Ar′ are independently at each instance an aromatic ring system having 6 to 40 ring atoms or a heteroaromatic ring system having 7 to 40 ring atoms, where carbazole units in the heteroaromatic ring system are excluded;
n at each instance is independently 0 or 1;
m at each instance is independently 0 or 1.

Preferably at least one Ar′ in formula (4) is a group of the following formulae (4a) or (4b)

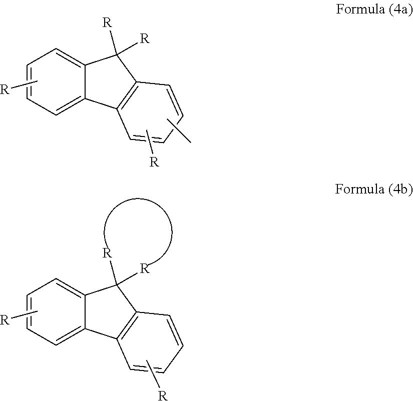

where R in formulae (4a) and (4b) is the same or different at each instance and is selected from H, D, F, CN, a straight-chain alkyl group having 1 to 20 carbon atoms or a branched or cyclic alkyl group having 3 to 20 carbon atoms, where one or more nonadjacent CH₂ groups may be replaced by R²C═CR², O or S and where one or more hydrogen atoms may be replaced by D, F, or CN and where two R may form a cyclic or polycyclic ring and * denotes the attachment to the remainder of the formula (4).

Preferred monoamines which are used in accordance with the invention in the organic layer of the device of the invention are described in table 5.

TABLE 5

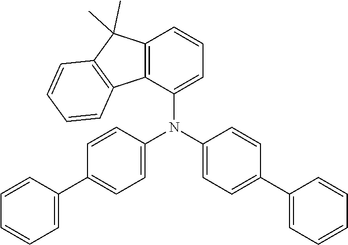 [SMILES: CC1(C)c2ccccc2-c2c(N(c3ccc(-c4ccccc4)cc3)c3ccc(-c4ccccc4)cc3)cccc21]

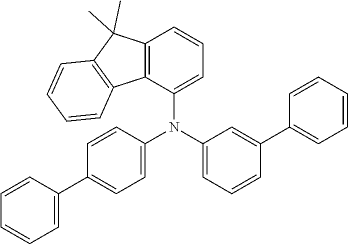 [SMILES: CC1(C)c2ccccc2-c2c(N(c3ccc(-c4ccccc4)cc3)c3cccc(-c4ccccc4)c3)cccc21]

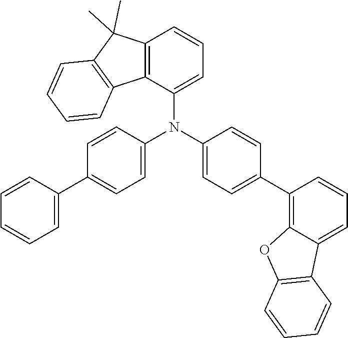 [SMILES: CC1(C)c2ccccc2-c2c(N(c3ccc(-c4ccccc4)cc3)c3ccc(-c4cccc5c4oc4ccccc45)cc3)cccc21]

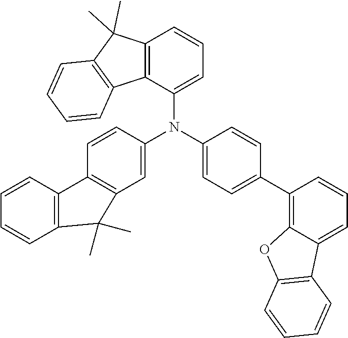 [SMILES: CC1(C)c2ccccc2-c2ccc(N(c3ccc(-c4cccc5c4oc4ccccc45)cc3)c3cccc4c3-c3ccccc3C4(C)C)cc21]

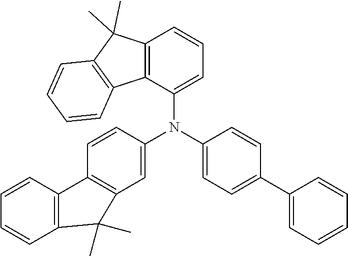 [SMILES: CC1(C)c2ccccc2-c2ccc(N(c3ccc(-c4ccccc4)cc3)c3cccc4c3-c3ccccc3C4(C)C)cc21]

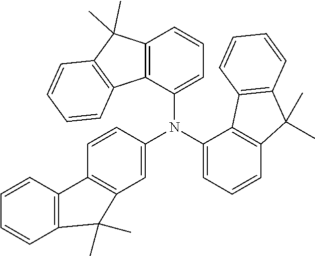 [SMILES: CC1(C)c2ccccc2-c2ccc(N(c3cccc4c3-c3ccccc3C4(C)C)c3cccc4c3-c3ccccc3C4(C)C)cc21]

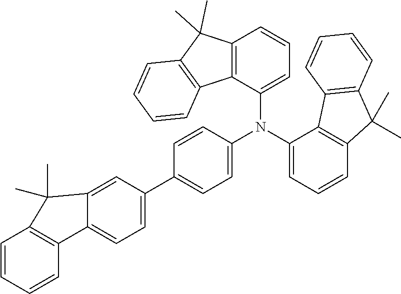 [SMILES: CC1(C)c2ccccc2-c2ccc(-c3ccc(N(c4cccc5c4-c4ccccc4C5(C)C)c4cccc5c4-c4ccccc4C5(C)C)cc3)cc21]

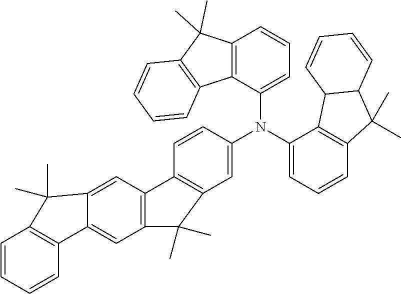 [SMILES: CC1(C)c2ccccc2-c2cc3c(cc21)-c1ccc(N(c2cccc4c2-c2ccccc2C4(C)C)c2cccc4c2C2C=CC=CC2C4(C)C)cc1C3(C)C]

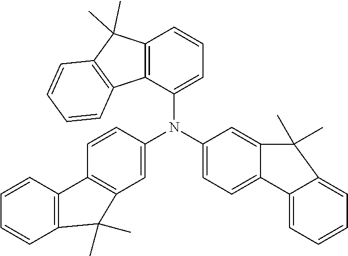 [SMILES: CC1(C)c2ccccc2-c2ccc(N(c3ccc4c(c3)C(C)(C)c3ccccc3-4)c3cccc4c3-c3ccccc3C4(C)C)cc21]

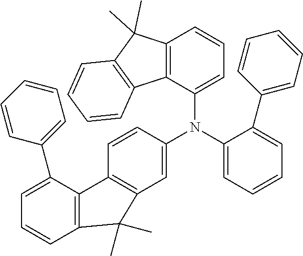 [SMILES: CC1(C)c2cc(N(c3ccccc3-c3ccccc3)c3cccc4c3-c3ccccc3C4(C)C)ccc2-c2c(-c3ccccc3)cccc21]

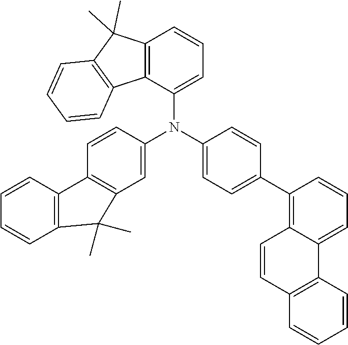 [SMILES: CC1(C)c2ccccc2-c2ccc(N(c3ccc(-c4cccc5c4ccc4ccccc45)cc3)c3cccc4c3-c3ccccc3C4(C)C)cc21]

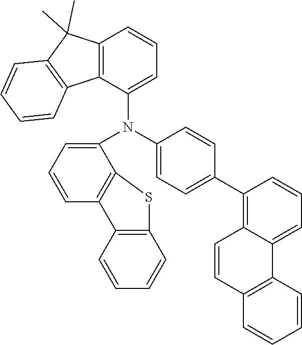 [SMILES: CC1(C)c2ccccc2-c2c(N(c3ccc(-c4cccc5c4ccc4ccccc45)cc3)c3cccc4c3sc3ccccc34)cccc21]

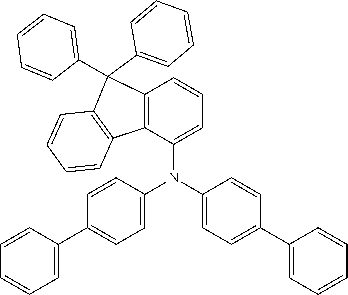 [SMILES: c1ccc(-c2ccc(N(c3ccc(-c4ccccc4)cc3)c3cccc4c3-c3ccccc3C4(c3ccccc3)c3ccccc3)cc2)cc1]

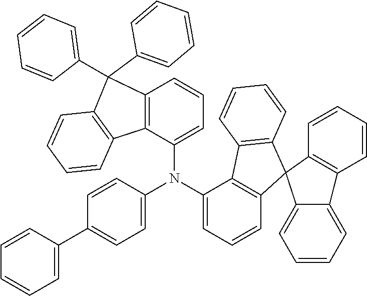 [SMILES: c1ccc(-c2ccc(N(c3cccc4c3-c3ccccc3C4(c3ccccc3)c3ccccc3)c3cccc4c3-c3ccccc3C43c4ccccc4-c4ccccc43)cc2)cc1]

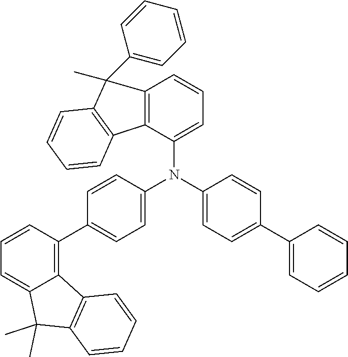 [SMILES: CC1(C)c2ccccc2-c2c(-c3ccc(N(c4ccc(-c5ccccc5)cc4)c4cccc5c4-c4ccccc4C5(C)c4ccccc4)cc3)cccc21]

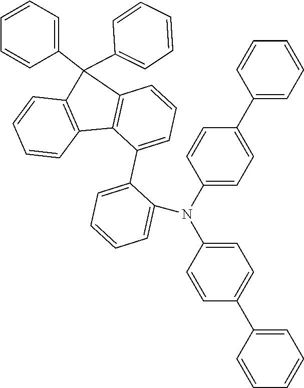 [SMILES: c1ccc(-c2ccc(N(c3ccc(-c4ccccc4)cc3)c3ccccc3-c3cccc4c3-c3ccccc3C4(c3ccccc3)c3ccccc3)cc2)cc1]

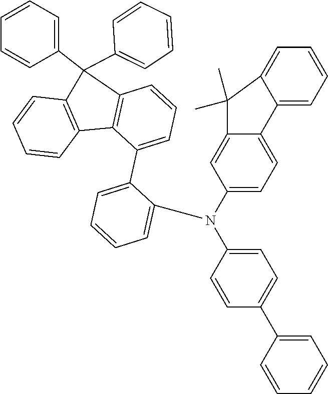 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21]

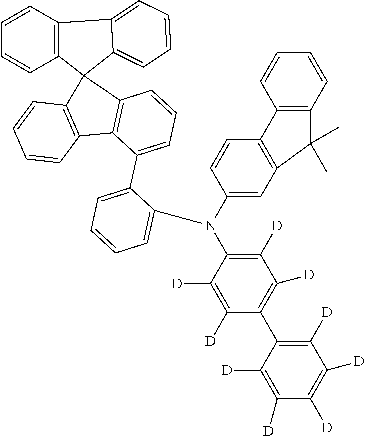 [SMILES: [2H]c1c([2H])c([2H])c(-c2c([2H])c([2H])c(N(c3ccc4c(c3)C(C)(C)c3ccccc3-4)c3ccccc3-c3cccc4c3-c3ccccc3C43c4ccccc4-c4ccccc43)c([2H])c2[2H])c([2H])c1[2H]]

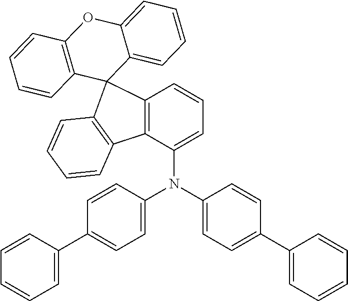 [SMILES: c1ccc(-c2ccc(N(c3ccc(-c4ccccc4)cc3)c3cccc4c3-c3ccccc3C43c4ccccc4Oc4ccccc43)cc2)cc1]

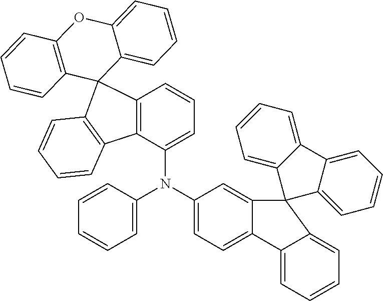 [SMILES: c1ccc(N(c2ccc3c(c2)C2(c4ccccc4-c4ccccc42)c2ccccc2-3)c2cccc3c2-c2ccccc2C32c3ccccc3Oc3ccccc32)cc1]

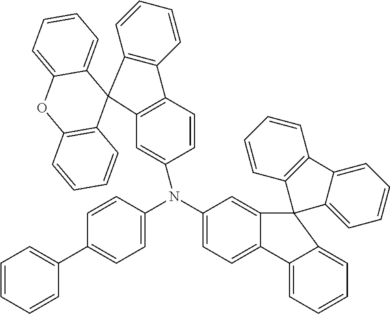 [SMILES: c1ccc(-c2ccc(N(c3ccc4c(c3)C3(c5ccccc5Oc5ccccc53)c3ccccc3-4)c3ccc4c(c3)C3(c5ccccc5-c5ccccc53)c3ccccc3-4)cc2)cc1]

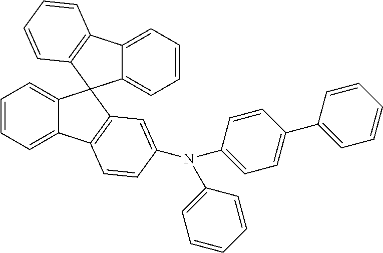 [SMILES: c1ccc(-c2ccc(N(c3ccccc3)c3ccc4c(c3)C3(c5ccccc5-c5ccccc53)c3ccccc3-4)cc2)cc1]

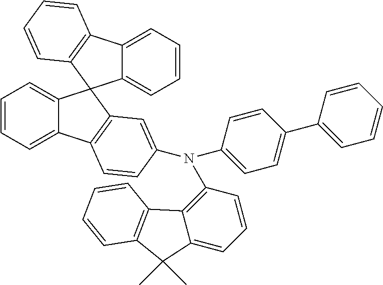 [SMILES: CC1(C)c2ccccc2-c2c(N(c3ccc(-c4ccccc4)cc3)c3ccc4c(c3)C3(c5ccccc5-c5ccccc53)c3ccccc3-4)cccc21]

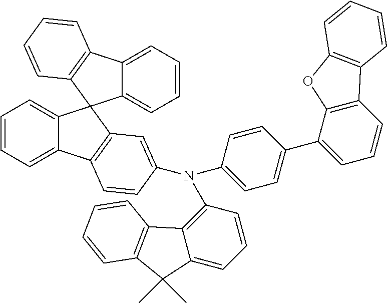 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

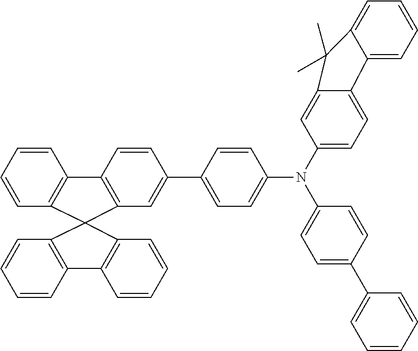 [SMILES: CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21]

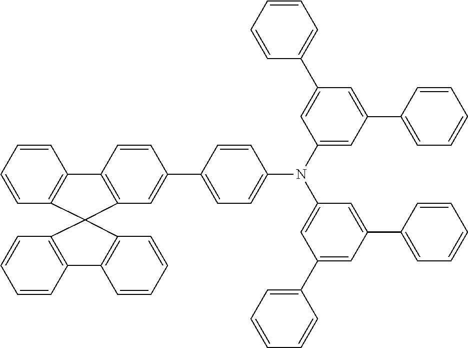 [SMILES: c1ccc(-c2cc(-c3ccccc3)cc(N(c3ccc(-c4ccc5c(c4)C4(c6ccccc6-c6ccccc64)c4ccccc4-5)cc3)c3cc(-c4ccccc4)cc(-c4ccccc4)c3)c2)cc1]

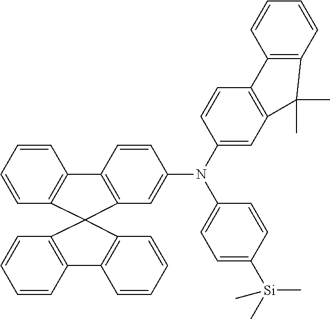 [SMILES: CC1(C)c2ccccc2-c2ccc(N(c3ccc([Si](C)(C)C)cc3)c3ccc4c(c3)C3(c5ccccc5-c5ccccc53)c3ccccc3-4)cc21]

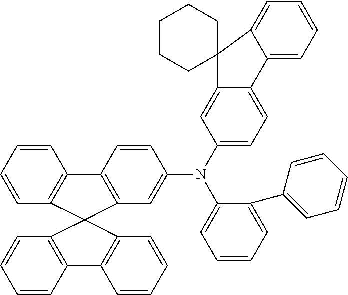 [SMILES: c1ccc(-c2ccccc2N(c2ccc3c(c2)C2(CCCCC2)c2ccccc2-3)c2ccc3c(c2)C2(c4ccccc4-c4ccccc42)c2ccccc2-3)cc1]

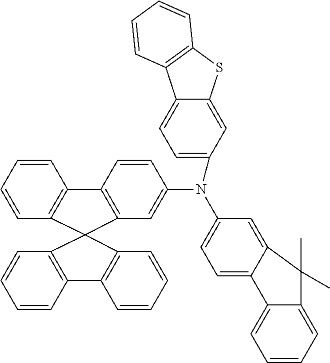 [SMILES: CC1(C)c2ccccc2-c2ccc(N(c3ccc4c(c3)C3(c5ccccc5-c5ccccc53)c3ccccc3-4)c3ccc4c(c3)sc3ccccc34)cc21]

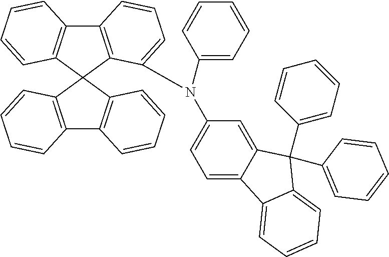 [SMILES: c1ccc(N(c2ccc3c(c2)C(c2ccccc2)(c2ccccc2)c2ccccc2-3)c2cccc3c2C2(c4ccccc4-c4ccccc42)c2ccccc2-3)cc1]

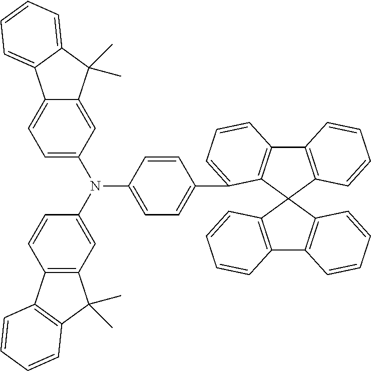 [SMILES: CC1(C)c2ccccc2-c2ccc(N(c3ccc(-c4cccc5c4C4(c6ccccc6-c6ccccc64)c4ccccc4-5)cc3)c3ccc4c(c3)C(C)(C)c3ccccc3-4)cc21]

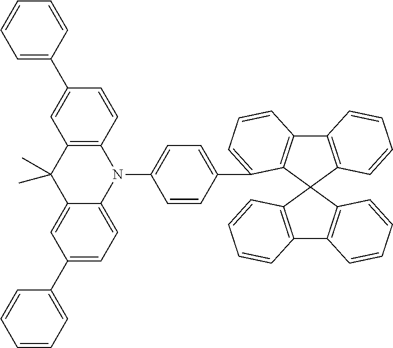 [SMILES: CC1(C)c2cc(-c3ccccc3)ccc2N(c2ccc(-c3cccc4c3C3(c5ccccc5-c5ccccc53)c3ccccc3-4)cc2)c2ccc(-c3ccccc3)cc21]

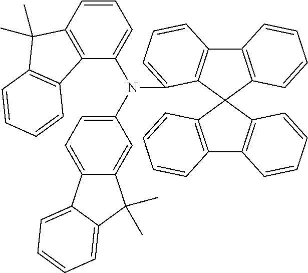 [SMILES: CC1(C)c2ccccc2-c2ccc(N(c3cccc4c3-c3ccccc3C4(C)C)c3cccc4c3C3(c5ccccc5-c5ccccc53)c3ccccc3-4)cc21]

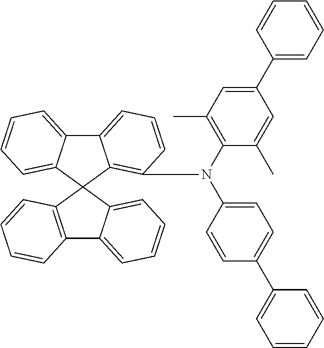 [SMILES: Cc1cc(-c2ccccc2)cc(C)c1N(c1ccc(-c2ccccc2)cc1)c1cccc2c1C1(c3ccccc3-c3ccccc31)c1ccccc1-2]

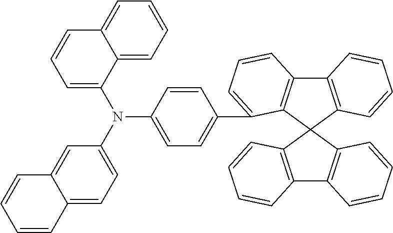 [SMILES: c1ccc2c(c1)-c1ccccc1C21c2ccccc2-c2cccc(-c3ccc(N(c4ccc5ccccc5c4)c4cccc5ccccc45)cc3)c21]

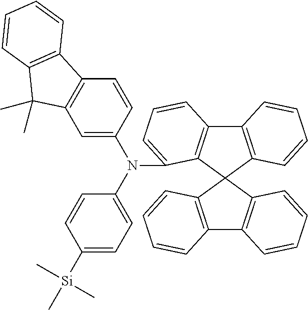 [SMILES: CC1(C)c2ccccc2-c2ccc(N(c3ccc([Si](C)(C)C)cc3)c3cccc4c3C3(c5ccccc5-c5ccccc53)c3ccccc3-4)cc21]

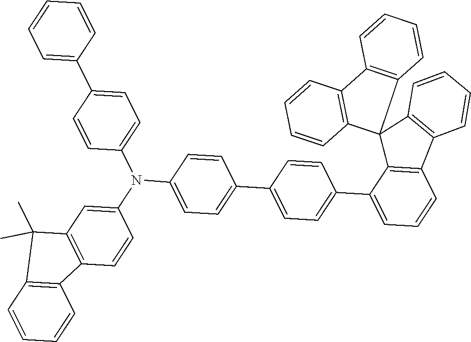 [SMILES: CC1(C)c2ccccc2-c2ccc(N(c3ccc(-c4ccccc4)cc3)c3ccc(-c4ccc(-c5cccc6c5C5(c7ccccc7-c7ccccc75)c5ccccc5-6)cc4)cc3)cc21]

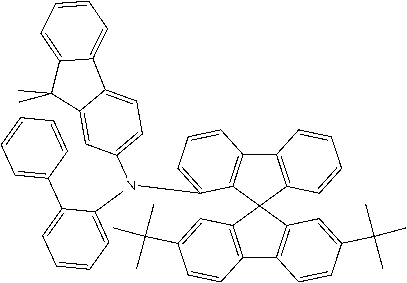 [SMILES: CC(C)(C)c1ccc2c(c1)C1(c3cc(C(C)(C)C)ccc3-2)c2ccccc2-c2cccc(N(c3ccc4c(c3)C(C)(C)c3ccccc3-4)c3ccccc3-c3ccccc3)c21]

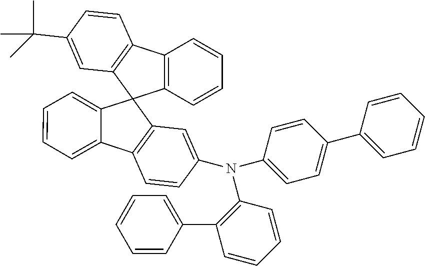 [SMILES: CC(C)(C)c1ccc2c(c1)C1(c3ccccc3-c3ccc(N(c4ccc(-c5ccccc5)cc4)c4ccccc4-c4ccccc4)cc31)c1ccccc1-2]

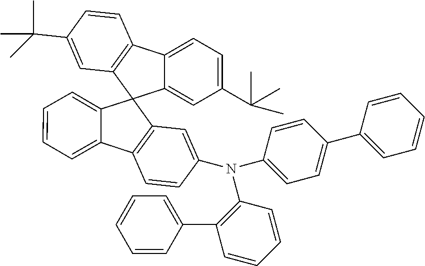 [SMILES: CC(C)(C)c1ccc2c(c1)C1(c3ccccc3-c3ccc(N(c4ccc(-c5ccccc5)cc4)c4ccccc4-c4ccccc4)cc31)c1cc(C(C)(C)C)ccc1-2]

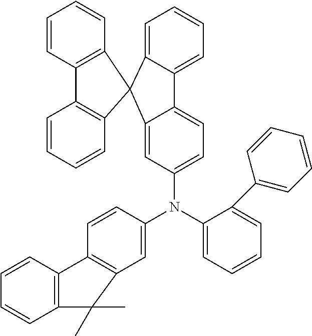 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

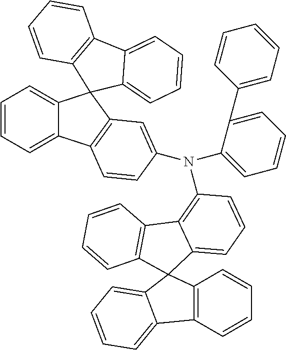 [SMILES: c1ccc(-c2ccccc2N(c2ccc3c(c2)C2(c4ccccc4-c4ccccc42)c2ccccc2-3)c2cccc3c2-c2ccccc2C32c3ccccc3-c3ccccc32)cc1]

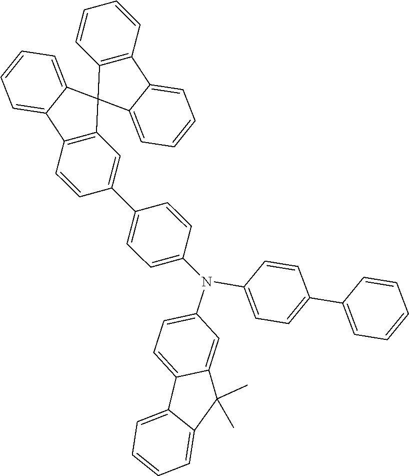 [SMILES: CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21]

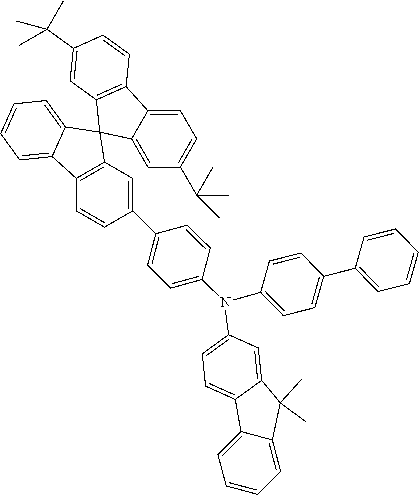 [SMILES: CC(C)(C)c1ccc2c(c1)C1(c3ccccc3-c3ccc(-c4ccc(N(c5ccc(-c6ccccc6)cc5)c5ccc6c(c5)C(C)(C)c5ccccc5-6)cc4)cc31)c1cc(C(C)(C)C)ccc1-2]

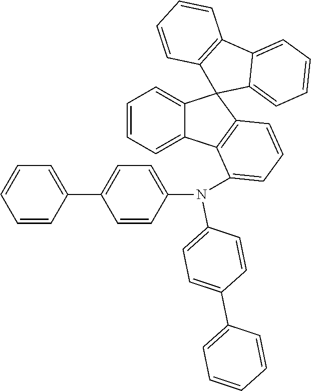 [SMILES: c1ccc(-c2ccc(N(c3ccc(-c4ccccc4)cc3)c3cccc4c3-c3ccccc3C43c4ccccc4-c4ccccc43)cc2)cc1]

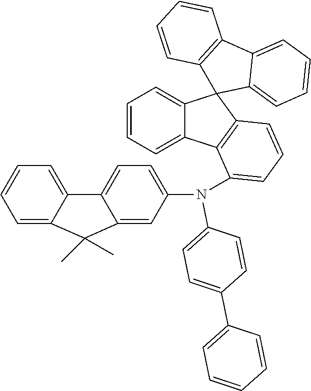 [SMILES: CC1(C)c2ccccc2-c2ccc(N(c3ccc(-c4ccccc4)cc3)c3cccc4c3-c3ccccc3C43c4ccccc4-c4ccccc43)cc21]

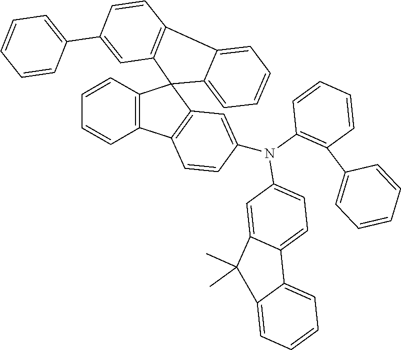 [SMILES: CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21]

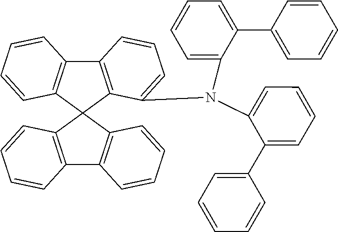 [SMILES: c1ccc(-c2ccccc2N(c2ccccc2-c2ccccc2)c2cccc3c2C2(c4ccccc4-c4ccccc42)c2ccccc2-3)cc1]

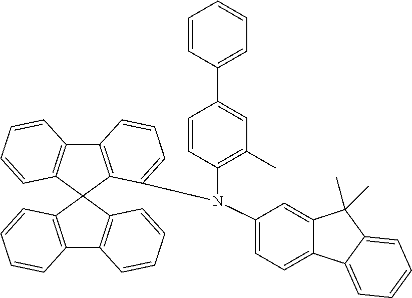 [SMILES: Cc1cc(-c2ccccc2)ccc1N(c1ccc2c(c1)C(C)(C)c1ccccc1-2)c1cccc2c1C1(c3ccccc3-c3ccccc31)c1ccccc1-2]

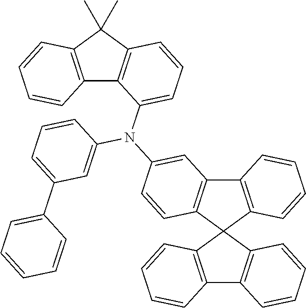 [SMILES: CC1(C)c2ccccc2-c2c(N(c3cccc(-c4ccccc4)c3)c3ccc4c(c3)-c3ccccc3C43c4ccccc4-c4ccccc43)cccc21]

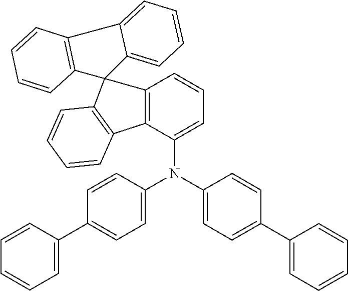 [SMILES: c1ccc(-c2ccc(N(c3ccc(-c4ccccc4)cc3)c3cccc4c3-c3ccccc3C43c4ccccc4-c4ccccc43)cc2)cc1]

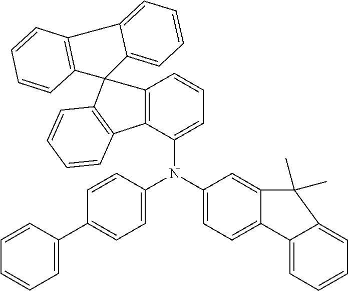 [SMILES: CC1(C)c2ccccc2-c2ccc(N(c3ccc(-c4ccccc4)cc3)c3cccc4c3-c3ccccc3C43c4ccccc4-c4ccccc43)cc21]

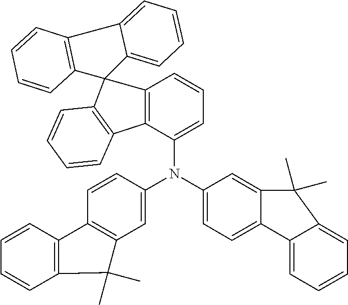 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21]

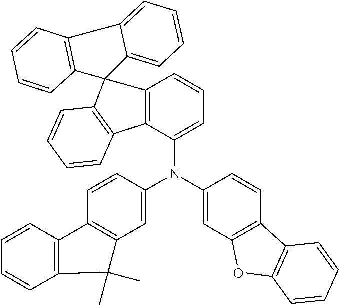 [SMILES: CC1(C)c2ccccc2-c2ccc(N(c3ccc4c(c3)oc3ccccc34)c3cccc4c3-c3ccccc3C43c4ccccc4-c4ccccc43)cc21]

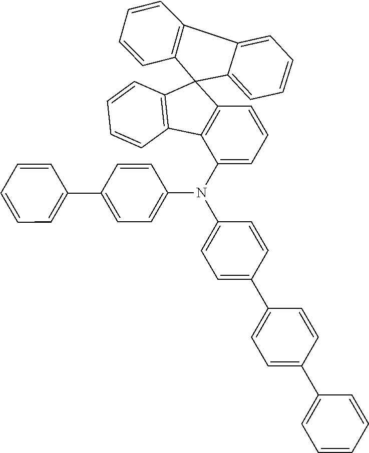 [SMILES: c1ccc(-c2ccc(-c3ccc(N(c4ccc(-c5ccccc5)cc4)c4cccc5c4-c4ccccc4C54c5ccccc5-c5ccccc54)cc3)cc2)cc1]

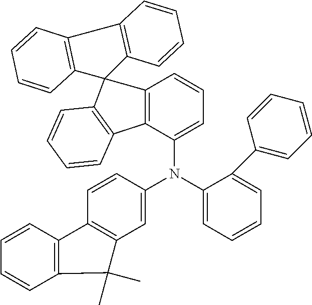 [SMILES: CC1(C)c2ccccc2-c2ccc(N(c3ccccc3-c3ccccc3)c3cccc4c3-c3ccccc3C43c4ccccc4-c4ccccc43)cc21]

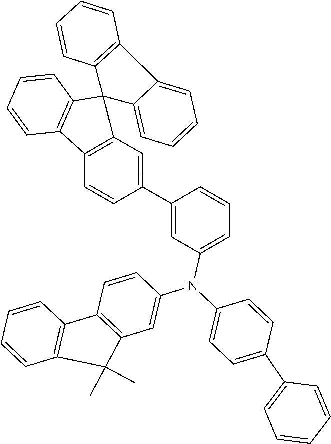 [SMILES: CC1(C)c2ccccc2-c2ccc(N(c3ccc(-c4ccccc4)cc3)c3cccc(-c4ccc5c(c4)C4(c6ccccc6-c6ccccc64)c4ccccc4-5)c3)cc21]

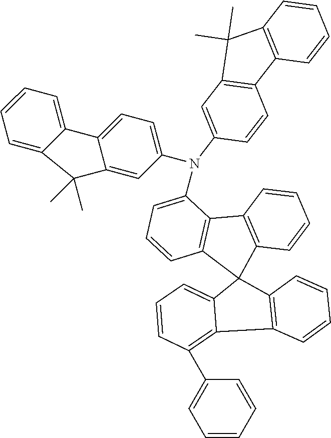 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccccc5)cccc43)cc21]

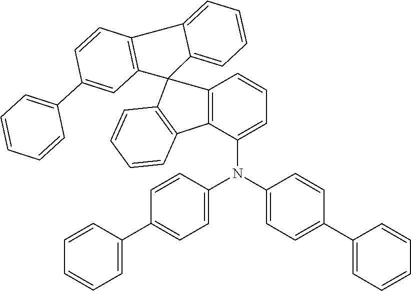 [SMILES: c1ccc(-c2ccc(N(c3ccc(-c4ccccc4)cc3)c3cccc4c3-c3ccccc3C43c4ccccc4-c4ccc(-c5ccccc5)cc43)cc2)cc1]

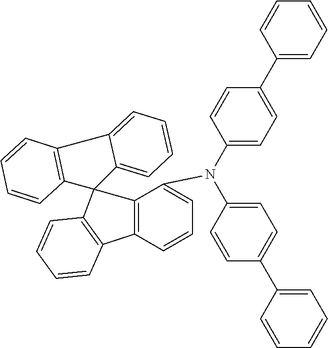 [SMILES: c1ccc(-c2ccc(N(c3ccc(-c4ccccc4)cc3)c3cccc4c3C3(c5ccccc5-c5ccccc53)c3ccccc3-4)cc2)cc1]

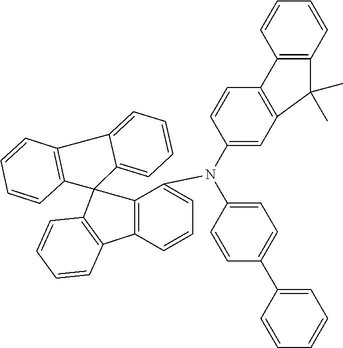 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

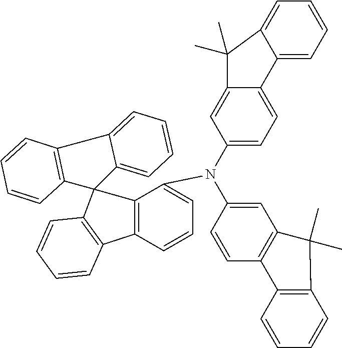 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

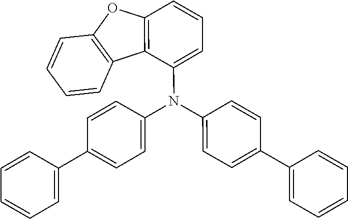 [SMILES: c1ccc(-c2ccc(N(c3ccc(-c4ccccc4)cc3)c3cccc4oc5ccccc5c34)cc2)cc1]

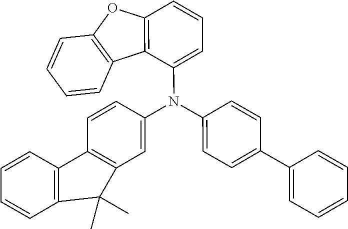 [SMILES: CC1(C)c2ccccc2-c2ccc(N(c3ccc(-c4ccccc4)cc3)c3cccc4oc5ccccc5c34)cc21]

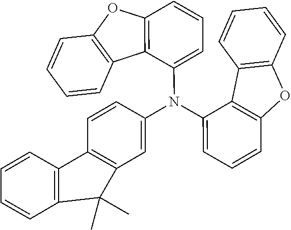 [SMILES: CC1(C)c2ccccc2-c2ccc(N(c3cccc4oc5ccccc5c34)c3cccc4oc5ccccc5c34)cc21]

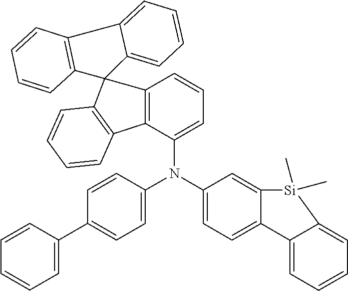 [SMILES: C[Si]1(C)c2ccccc2-c2ccc(N(c3ccc(-c4ccccc4)cc3)c3cccc4c3-c3ccccc3C43c4ccccc4-c4ccccc43)cc21]

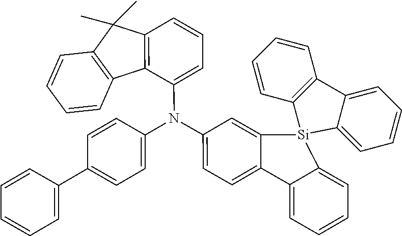 [SMILES: CC1(C)c2ccccc2-c2c(N(c3ccc(-c4ccccc4)cc3)c3ccc4c(c3)[Si]3(c5ccccc5-c5ccccc53)c3ccccc3-4)cccc21]

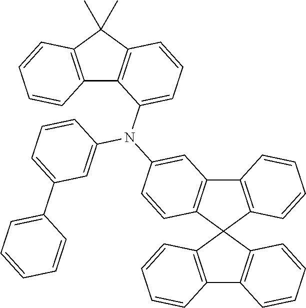 [SMILES: CC1(C)c2ccccc2-c2c(N(c3cccc(-c4ccccc4)c3)c3ccc4c(c3)-c3ccccc3C43c4ccccc4-c4ccccc43)cccc21]

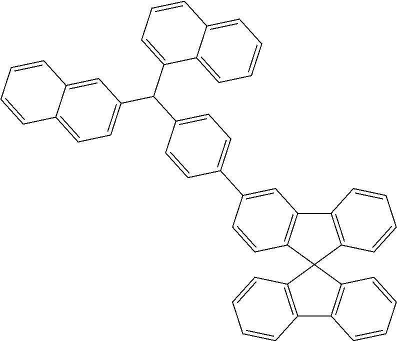 [SMILES: c1ccc2c(c1)-c1ccccc1C21c2ccccc2-c2cc(-c3ccc(C(c4ccc5ccccc5c4)c4cccc5ccccc45)cc3)ccc21]

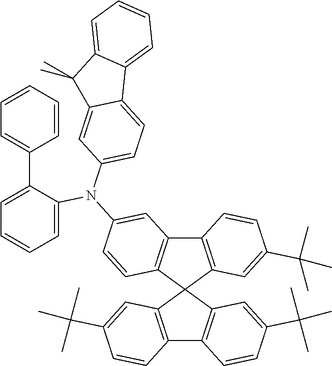 [SMILES: CC(C)(C)c1ccc2c(c1)C1(c3ccc(N(c4ccc5c(c4)C(C)(C)c4ccccc4-5)c4ccccc4-c4ccccc4)cc3-2)c2cc(C(C)(C)C)ccc2-c2ccc(C(C)(C)C)cc21]

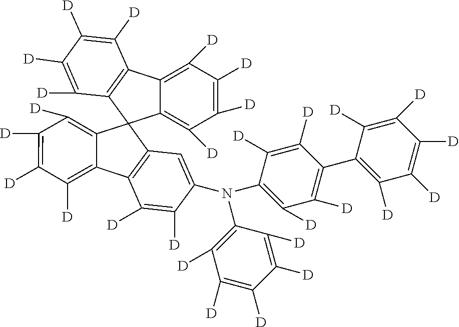 [SMILES: [2H]c1c(N(c2c([2H])c([2H])c([2H])c([2H])c2[2H])c2c([2H])c([2H])c(-c3c([2H])c([2H])c([2H])c([2H])c3[2H])c([2H])c2[2H])cc2c(c1[2H])-c1c([2H])c([2H])c([2H])c([2H])c1C21c2c([2H])c([2H])c([2H])c([2H])c2-c2c([2H])c([2H])c([2H])c([2H])c21]

Preferred hole transport materials further include in combination with the compounds of table 5 or as alternatives for compounds of the table 5 materials that may be used in a hole transport, hole injection or electron blocker layer, such as indenofluorenamine derivatives, hexaazatriphenylene derivatives, monobenzoindenofluorenamines, dibenzoindenofluorenamines, dihydroacridine derivatives.

The sequence of layers in the organic electroluminescent device of the invention is preferably as follows:
anode/hole injection layer/hole transport layer/emitting layer/electron transport layer/electron injection layer/cathode.

This sequence of the layers is a preferred sequence.

At the same time, it should be pointed out again that not all the layers mentioned need be present and/or that further layers may additionally be present.

The organic electroluminescent device of the invention may contain two or more emitting layers. At least one of the emitting layers is the light-emitting layer of the invention containing at least one compound of the formula (1) as host material 1 and at least one compound of the formula (2) as host material 2 as described above. It is particularly preferable when these emission layers in this case altogether exhibit a plurality of emission maxima between 380 nm and 750 nm, so that altogether white emission results. It should be noted that, for the production of white light, rather than a plurality of colour-emitting emitter compounds, an emitter compound used individually which emits over a broad wavelength range may also be suitable.

Materials used for the electron transport layer may be any materials as used according to the prior art as electron transport materials in the electron transport layer. Especially suitable are aluminium complexes, for example Alq₃, zirconium complexes, for example Zrq₄, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Suitable cathodes of the device of the invention are metals having a low work function, metal alloys or multilayer structures composed of various metals, for example alkaline earth metals, alkali metals, main group metals or lanthanoids (e.g. Ca, Ba, Mg, Al, In, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The organic electroluminescent device of the invention, in the course of production, is appropriately (according to the application) structured, contact-connected and finally sealed, since the lifetime of the devices of the invention is shortened in the presence of water and/or air.

The production of the device of the invention is not restricted here. It is possible that one or more organic layers, including the light-emitting layer, are coated by a sublimation method. In this case, the materials are applied by vapour deposition in vacuum sublimation systems at an initial pressure of less than 10-5 mbar, preferably less than 10-6 mbar. In this case, however, it is also possible that the initial pressure is even lower, for example less than 10-7 mbar.

The organic electroluminescent device of the invention is preferably characterized in that one or more layers are coated by the OVPD (organic vapour phase deposition) method or with the aid of a carrier gas sublimation. In this case, the materials are applied at a pressure between 10-5 mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

The organic electroluminescent device of the invention is further preferably characterized in that one or more organic layers comprising the composition of the invention are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble host materials 1 and 2 and phosphorescent emitters are needed. Processing from solution has the advantage that, for example, the light-emitting layer can be applied in a very simple and inexpensive manner. This technique is especially suitable for the mass production of organic electroluminescent devices.

In addition, hybrid methods are possible, in which, for example, one or more layers are applied from solution and one or more further layers are applied by vapour deposition.

These methods are known in general terms to those skilled in the art and can be applied to organic electroluminescent devices.

The invention therefore further provides a process for producing the organic electroluminescent device of the invention as described above or described as preferred, characterized in that the organic layer, preferably the light-emitting layer, the hole injection layer and/or hole transport layer, is applied by gas phase deposition, especially by a sublimation method and/or by an OVPD (organic vapour phase deposition) method and/or with the aid of a carrier gas sublimation, or from solution, especially by spin-coating or by a printing method.

In the case of production by means of gas phase deposition, there are in principle two ways in which the organic layer, preferably the light-emitting layer, of the invention can be applied or vapour-deposited onto any substrate or the prior layer. Firstly, the materials used can each be initially charged in a material source and ultimately evaporated from the different material sources (“co-evaporation”). Secondly, the various materials can be premixed (premix systems) and the mixture can be initially charged in a single material source from which it is ultimately evaporated (“premix evaporation”). In this way, it is possible in a simple and rapid manner to achieve the vapour deposition of the light-emitting layer with homogeneous distribution of the components without the need for precise actuation of a multitude of material sources.

The invention accordingly further provides a process for producing the device of the invention, characterized in that the at least one compound of the formula (1) as described above or described as preferred and the at least one compound of the formula (2) as described above or described as preferred are deposited from the gas phase successively or simultaneously from at least two material sources, optionally with the at least one phosphorescent emitter as described above or described as preferred, and form the light-emitting layer.

In a preferred embodiment of the present invention, the light-emitting layer is applied by means of gas phase deposition, wherein the constituents of the composition are premixed and evaporated from a single material source.

The invention accordingly further provides a process for producing the device of the invention, characterized in that the at least one compound of the formula (1) and the at least one compound of the formula (2) are deposited from the gas phase as a mixture, successively or simultaneously with the at least one phosphorescent emitter, and form the light-emitting layer.

The invention further provides a process for producing the device of the invention, as described above or described as preferred, characterized in that the at least one compound of the formula (1) and the at least one compound of the formula (2), as described above or described as preferred, are applied from solution together with the at least one phosphorescent emitter in order to form the light-emitting layer.

The devices of the invention feature the following surprising advantages over the prior art:

The use of the described material combination of host materials 1 and 2, as described above, especially leads to an increase in the lifetime of the devices.

As apparent in the example given hereinafter, it is possible to determine by comparison of the data for OLEDs with combinations from the prior art that the inventive combinations of matrix materials in the EML lead to devices having a significantly increased lifetime, irrespective of the emitter concentration.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Any feature disclosed in the present invention, unless stated otherwise, should therefore be considered as an example from a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

The technical teaching disclosed with the present invention may be abstracted and combined with other examples.

The invention is illustrated in more detail by the examples which follow, without any intention of restricting it thereby.

### EXAMPLES

General Methods:

In all quantum-chemical calculations, the Gaussian16 (Rev. B.01) software package is used. The neutral singlet ground state is optimized at the B3LYP/6-31G(d) level. HOMO and LUMO values are determined at the B3LYP/6-31G(d) level for the B3LYP/6-31G(d)-optimized ground state energy. Then TD-DFT singlet and triplet excitations (vertical excitations) are calculated by the same method (B3LYP/6-31G(d)) and with the optimized ground state geometry. The standard settings for SCF and gradient convergence are used.

From the energy calculation, the HOMO is obtained as the last orbital occupied by two electrons (alpha occ. eigenvalues) and LUMO as the first unoccupied orbital (alpha virt. eigenvalues) in Hartree units, where HEh and LEh represent the HOMO energy in Hartree units and the LUMO energy in Hartree units respectively. This is used to determine the HOMO and LUMO value in electron volts, calibrated by cyclic voltammetry measurements, as follows:

HOMOcorr=0.90603*HOMO−0.84836

LUMOcorr=0.99687*LUMO−0.72445

The triplet level T1 of a material is defined as the relative excitation energy (in eV) of the triplet state having the lowest energy which is found by the quantum-chemical energy calculation.

The singlet level S1 of a material is defined as the relative excitation energy (in eV) of the singlet state having the second-lowest energy which is found by the quantum-chemical energy calculation.

The energetically lowest singlet state is referred to as S0.

The method described herein is independent of the software package used and always gives the same results. Examples of frequently utilized programs for this purpose are “Gaussian09” (Gaussian Inc.) and Q-Chem 4.1 (Q-Chem, Inc.). In the present case, the energies are calculated using the software package “Gaussian16 (Rev. B.01)”.

### Example 1: Production of the OLEDs

Pretreatment for production of the OLEDs: Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm.

All materials are applied by thermal vapour deposition in a vacuum chamber. In this case, the emission layer always consists of at least one matrix material (host material), for the purposes of the invention at least two matrix materials and an emitting dopant (emitter) which is added to the matrix material(s) in a particular proportion by volume by co-evaporation. Analogously, the electron transport layer may also consist of a mixture of two materials.

The OLEDs are characterized in a standard manner. For this purpose, the electroluminescence spectra and current-voltage-luminance characteristics (IUL characteristics) are measured. EQE and current efficiency SE (in cd/A) are calculated therefrom. SE is calculated assuming Lambertian emission characteristics.

The lifetime LT is defined as the time after which the luminance drops from a starting luminance L0 (in cd/m²) to a certain proportion L1 (in cd/m²) in the course of operation with constant current density j₀ in mA/cm². A figure of L1/L0=80% in table 7 means that the lifetime reported in the LT column corresponds to the time (in h) after which the luminance falls to 80% of its starting value (L0).

Use of Mixtures of the Invention in OLEDs

Examples V1 to V15, V15a, V16 to V18 and V18a and B1 to B30 below (see tables 6 and 7) present the use of the inventive material combinations in OLEDs compared to material combinations from the prior art. The material combinations of the invention can be used in the emission layer in phosphorescent green OLEDs.

The construction of the OLEDs is apparent from table 6. The materials required for production of the OLEDs are shown in table 8 if not already described above. The device data of the OLEDs are listed in table 7.

Details given in such a form as VG1:H2:TEG1 (33%:60%:7%) 30 nm indicate the presence of comparative material 1 (VG1) in a proportion by volume of 33% as host material 1, compound H2 as host material 2 in a proportion of 60%, and TEG1 in a proportion of 7% in a layer of thickness 30 nm.

Examples V1 to V15, V17 and V18 are comparative examples with an electron-transporting host according to the prior art or, in V15a and V16, with an electron-transporting host according to the prior art in combination with the host H0 and, in V18a, with the host HA. Examples B1 to B30 use inventive material combinations in the EML.

On comparison of the inventive examples with the corresponding comparative examples, it is clearly apparent that the inventive examples each show a distinct advantage in device lifetime.

TABLE 6

| | HIL | HTL | EBL | EML | HBL | ETL | EIL |
|---|---|---|---|---|---|---|---|
| Ex. | Thickness | Thickness | Thickness | Thickness | Thickness | Thickness | Thickness |
| V1 | SpMA1:P | SpMA1 | SpMA2 | VG1:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B1 | SpMA1:P | SpMA1 | SpMA2 | E1:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V2 | SpMA1:P | SpMA1 | SpMA2 | VG2:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B2 | SpMA1:P | SpMA1 | SpMA2 | E2:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V3 | SpMA1:P | SpMA1 | SpMA2 | VG3:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B3 | SpMA1:P | SpMA1 | SpMA2 | E4:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V4 | SpMA1:P | SpMA1 | SpMA2 | VG4:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B4 | SpMA1:P | SpMA1 | SpMA2 | E5:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V5 | SpMA1:P | SpMA1 | SpMA2 | VG5:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B5 | SpMA1:P | SpMA1 | SpMA2 | E11:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V6 | SpMA1:P | SpMA1 | SpMA2 | VG6:H1:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B6 | SpMA1:P | SpMA1 | SpMA2 | E19:H1:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V7 | SpMA1:P | SpMA1 | SpMA2 | VG7:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B7 | SpMA1:P | SpMA1 | SpMA2 | E14:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V8 | SpMA1:P | SpMA1 | SpMA2 | VG8:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B8 | SpMA1:P | SpMA1 | SpMA2 | E1:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V9 | SpMA1:P | SpMA1 | SpMA2 | VG9:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| B9 | SpMA1:P | SpMA1 | SpMA2 | E12:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V10 | SpMA1:P | SpMA1 | SpMA2 | VG10:H2:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B10 | SpMA1:P | SpMA1 | SpMA2 | E14:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V11 | SpMA1:P | SpMA1 | SpMA2 | VG11:H1:TEG | ST2 | ST2:LiQ | LiQ |
| | D1 | 215 nm | 20 nm | 1 | 10 nm | (50%:50%) | 1 nm |
| | (95%:5%) | | | (46%:47%:7%) | | 30 nm | |
| | 20 nm | | | 30 nm | | | |
| B11 | SpMA1:P | SpMA1 | SpMA2 | E1:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V12 | SpMA1:P | SpMA1 | SpMA2 | VG12:H23:TE | ST2 | ST2:LiQ | LiQ |
| | D1 | 215 nm | 20 nm | G1 | 10 nm | (50%:50%) | 1 nm |
| | (95%:5%) | | | (33%:60%:7%) | | 30 nm | |
| | 20 nm | | | 30 nm | | | |
| B12 | SpMA1:P | SpMA1 | SpMA2 | E1:H23:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V13 | SpMA1:P | SpMA1 | SpMA2 | VG13:H1:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B13 | SpMA1:P | SpMA1 | SpMA2 | E15:H1:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V14 | SpMA1:P | SpMA1 | SpMA2 | VG14:H2:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B14 | SpMA1:P | SpMA1 | SpMA2 | E17:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V15 | SpMA1:P | SpMA1 | SpMA2 | VG15:H2:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| V15a | SpMA1:P | SpMA1 | SpMA2 | VG15:HO:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B15 | SpMA1:P | SpMA1 | SpMA2 | E1:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V16 | SpMA1:P | SpMA1 | SpMA2 | VG16:HO:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B16 | SpMA1:P | SpMA1 | SpMA2 | E20:H2:TEG1 | ST2 | ST2:LiQ | LiQ |
| | D1 | 215 nm | 20 nm 2 | (33%:60%:7%) | 10 nm | (50%:50%) | 1 nm |
| | (95%:5%) | | | 30 nm | | 30 nm | |
| | 20 nm | | | | | | |
| V17 | SpMA1:P | SpMA1 | SpMA2 | VG17:H2:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| B17 | SpMA1:P | SpMA1 | SpMA2 | E13:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |
| V18 | SpMA1:P | SpMA1 | SpMA2 | VG18:H2:TEG | ST2 | ST2:LiQ | LiQ |
| | D1 | 215 nm | 20 nm | 1 | 10 nm | (50%:50%) | 1 nm |
| | (95%:5%) | | | (33%:60%:7%) | | 30 nm | |
| | 20 nm | | | 30 nm | | | |
| V18a | SpMA1:P | SpMA1 | SpMA2 | VG18:HA:TEG | ST2 | ST2:LiQ | LiQ |
| | D1 | | | 1 | 10 nm | (50%:50%) | |
| | (95%:5%) | 215 nm | 20 nm | (33%:60%:7%) | | 30 nm | 1 nm |
| | 20 nm | | | 30 nm | | | |
| B18 | SpMA1:P | SpMA1 | SpMA2 | E18:H2:TEG1 | ST2 | ST2:LiQ | LiQ |
| | D1 | 215 nm | 20 nm | (33%:60%:7%) | 10 nm | (50%:50%) | 1 nm |
| | (95%:5%) | | | 30 nm | | 30 nm | |
| | 20 nm | | | | | | |

TABLE 7
Data of the OLEDs

| | | | | CIE x/y | j₀ | | |
|---|---|---|---|---|---|---|---|
| | U1000 | SE1000 | EQE1000 | at 1000 | (mA/ | L1 | LT |
| Ex. | (V) | (cd/A) | (%) | cd/m2 | cm²) | (%) | (h) |
| V1 | 3.5 | 67 | 18 | 0.33/0.63 | 20 | 80 | 360 |
| B1 | 3.3 | 76 | 19 | 0.35/0.62 | 20 | 80 | 620 |
| V2 | 3.6 | 71 | 16 | 0.34/0.61 | 20 | 80 | 340 |
| B2 | 3.2 | 72 | 19 | 0.32/0.62 | 20 | 80 | 630 |
| V3 | 3.8 | 69 | 17 | 0.35/0.62 | 20 | 80 | 345 |
| B3 | 3.4 | 72 | 18.5 | 0.33/0.63 | 20 | 80 | 600 |
| V4 | 3.5 | 68 | 18 | 0.32/0.62 | 20 | 80 | 380 |
| B4 | 3.2 | 70 | 18 | 0.34/0.61 | 20 | 80 | 595 |
| V5 | 3.7 | 71 | 17 | 0.34/0.61 | 20 | 80 | 375 |
| B5 | 3.5 | 75 | 18 | 0.33/0.63 | 20 | 80 | 590 |
| V6 | 3.5 | 70 | 17 | 0.35/0.62 | 20 | 80 | 380 |
| B6 | 3.3 | 75 | 18 | 0.33/0.63 | 20 | 80 | 585 |
| V7 | 3.5 | 68 | 18 | 0.33/0.63 | 20 | 80 | 375 |
| B7 | 3.2 | 75 | 18.5 | 0.33/0.63 | 20 | 80 | 615 |
| V8 | 3.4 | 70 | 17 | 0.34/0.61 | 20 | 80 | 325 |
| B8 | 3.3 | 76 | 19 | 0.35/0.62 | 20 | 80 | 620 |
| V9 | 3.7 | 69 | 16.5 | 0.35/0.62 | 20 | 80 | 310 |
| B9 | 3.3 | 75 | 17.5 | 0.33/0.63 | 20 | 80 | 515 |
| V10 | 3.4 | 67 | 18.5 | 0.32/0.62 | 20 | 80 | 395 |
| B10 | 3.2 | 75 | 18.5 | 0.33/0.63 | 20 | 80 | 615 |
| V11 | 3.2 | 74 | 20.1 | 0.32/0.63 | 20 | 80 | 650 |
| B11 | 3.3 | 76 | 19 | 0.35/0.62 | 20 | 80 | 620 |
| V12 | 3.9 | 67 | 16 | 0.35/0.62 | 20 | 80 | 290 |
| B12 | 3.3 | 76 | 19 | 0.35/0.62 | 20 | 80 | 620 |
| V13 | 3.8 | 70 | 17 | 0.34/0.61 | 20 | 80 | 280 |
| B13 | 3.5 | 76 | 18 | 0.35/0.62 | 20 | 80 | 610 |
| V14 | 4.1 | 69 | 16 | 0.35/0.62 | 20 | 80 | 170 |
| B14 | 3.4 | 76 | 18.5 | 0.34/0.61 | 20 | 80 | 570 |
| V15 | 3.6 | 70 | 16 | 0.34/0.61 | 20 | 80 | 310 |
| V15a | 3.5 | 68 | 18.5 | 0.32/0.62 | 20 | 80 | 390 |
| B15 | 3.3 | 76 | 19 | 0.33/0.63 | 20 | 80 | 620 |
| V16 | 3.4 | 70 | 18 | 0.34/0.61 | 20 | 80 | 394 |
| V16a | 3.6 | 70 | 17 | 0.33/0.63 | 20 | 80 | 315 |
| B16 | 3.3 | 70 | 17.5 | 0.34/0.61 | 20 | 80 | 575 |
| V17 | 3.6 | 73 | 16.5 | 0.35/0.61 | 20 | 80 | 320 |
| 17 | 3.4 | 76 | 18.5 | 0.35/0.62 | 20 | 80 | 575 |
| V18 | 3.5 | 73 | 18.5 | 0.35/0.61 | 20 | 80 | 365 |
| V18a | 3.4 | 68 | 18 | 0.32/0.62 | 20 | 80 | 310 |
| B18 | 3.4 | 76 | 18.5 | 0.33/0.63 | 20 | 80 | 595 |
| B19 | 3.4 | 76 | 18 | 0.33/0.63 | 20 | 80 | 595 |
| B20 | 3.5 | 67 | 18.5 | 0.34/0.61 | 20 | 80 | 610 |
| B21 | 3.5 | 71 | 17 | 0.35/0.63 | 20 | 80 | 555 |
| B22 | 3.4 | 72 | 17.5 | 0.35/0.61 | 20 | 80 | 545 |
| B23 | 3.5 | 69 | 18 | 0.34/0.62 | 20 | 80 | 615 |
| B24 | 3.4 | 67 | 17 | 0.33/0.63 | 20 | 80 | 545 |
| B25 | 3.6 | 76 | 17.5 | 0.35/0.62 | 20 | 80 | 565 |
| B26 | 3.3 | 72 | 18 | 0.35/0.63 | 20 | 80 | 625 |
| B27 | 3.3 | 71 | 18.5 | 0.34/0.62 | 20 | 80 | 630 |
| B28 | 3.1 | 69 | 19 | 0.34/0.62 | 20 | 80 | 635 |
| B29 | 3.2 | 67 | 19 | 0.34/0.61 | 20 | 80 | 630 |
| B30 | 3.1 | 68 | 18 | 0.34/0.61 | 20 | 80 | 630 |

TABLE 8
Materials used, if not already described:

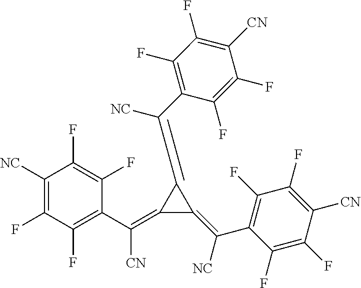 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

PD1 (1224447-88-4)

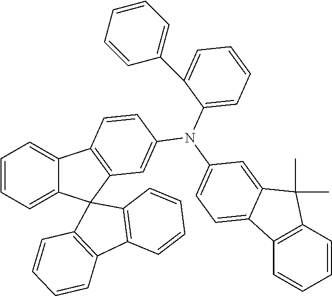 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

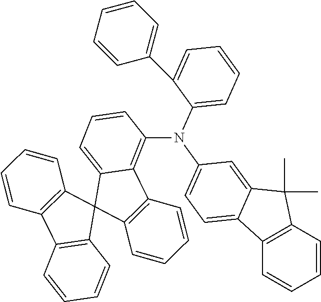 [SMILES: CC1(C)c2ccccc2-c2ccc(N(c3ccccc3-c3ccccc3)c3cccc4c3-c3ccccc3C43c4ccccc4-c4ccccc43)cc21]

SpMA2

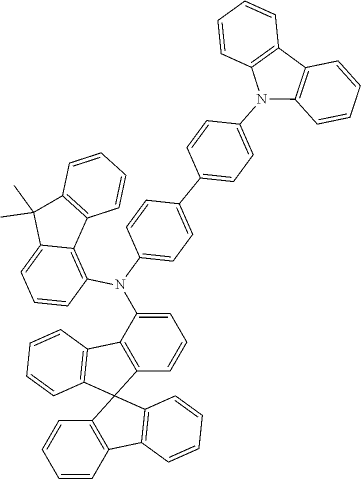 [SMILES: CC1(C)c2ccccc2-c2c(N(c3ccc(-c4ccc(-n5c6ccccc6c6ccccc65)cc4)cc3)c3cccc4c3-c3ccccc3C43c4ccccc4-c4ccccc43)cccc21]

SpMA5

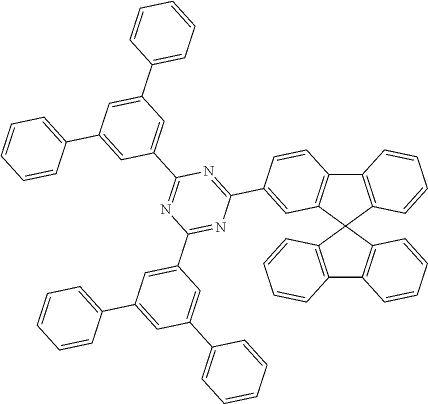 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

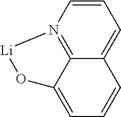

LiQ

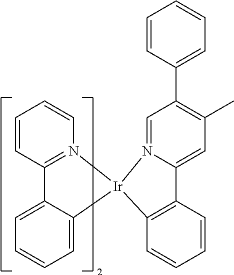

TEG1

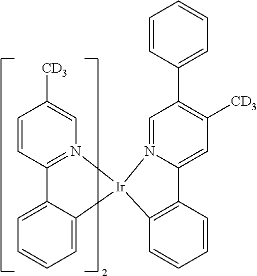

TEG2

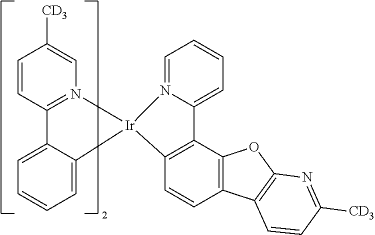

TEG3

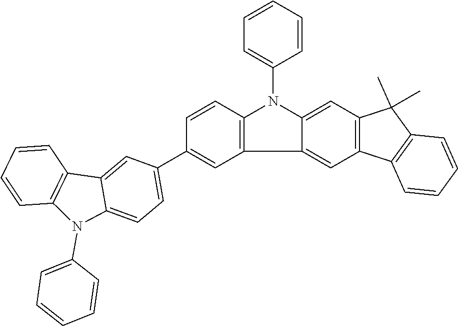 [SMILES: CC1(C)c2ccccc2-c2cc3c4cc(-c5ccc6c(c5)c5ccccc5n6-c5ccccc5)ccc4n(-c4ccccc4)c3cc21]

H0

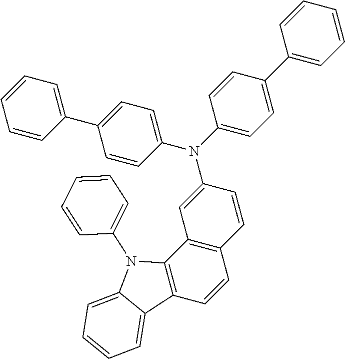 [SMILES: c1ccc(-c2ccc(N(c3ccc(-c4ccccc4)cc3)c3ccc4ccc5c6ccccc6n(-c6ccccc6)c5c4c3)cc2)cc1]

HA [WO20067657]

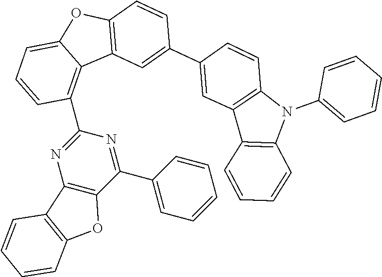 [SMILES: c1ccc(-c2nc(-c3cccc4oc5ccc(-c6ccc7c(c6)c6ccccc6n7-c6ccccc6)cc5c34)nc3c2oc2ccccc23)cc1]

VG1 [KR2018010165]

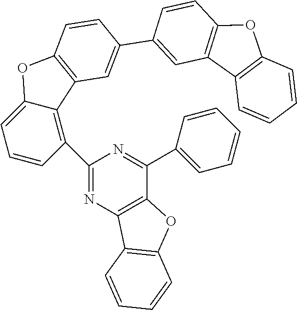 [SMILES: c1ccc(-c2nc(-c3cccc4oc5ccc(-c6ccc7oc8ccccc8c7c6)cc5c34)nc3c2oc2ccccc23)cc1]

VG2 [KR2018010165]

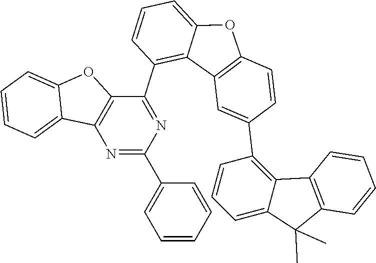 [SMILES: CC1(C)c2ccccc2-c2c(-c3ccc4oc5cccc(-c6nc(-c7ccccc7)nc7c6oc6ccccc67)c5c4c3)cccc21]

VG3 [KR2018010165]

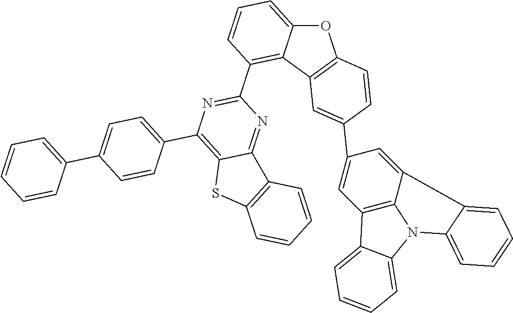 [SMILES: c1ccc(-c2ccc(-c3nc(-c4cccc5oc6ccc(-c7cc8c9ccccc9n9c%10ccccc%10c(c7)c89)cc6c45)nc4c3sc3ccccc34)cc2)cc1]

VG4 [KR20170113320]

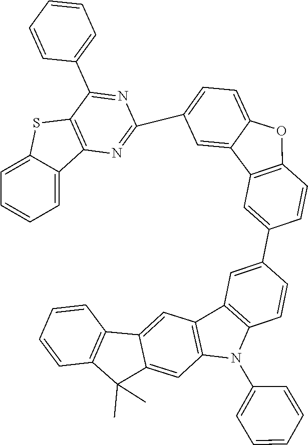 [SMILES: CC1(C)c2ccccc2-c2cc3c4cc(-c5ccc6oc7ccc(-c8nc(-c9ccccc9)c9sc%10ccccc%10c9n8)cc7c6c5)ccc4n(-c4ccccc4)c3cc21]

VG5 [KR2018010165]

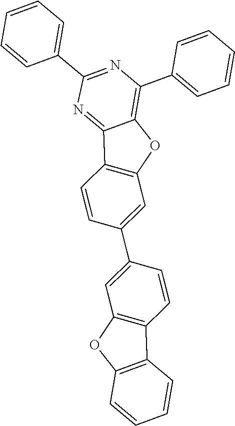 [SMILES: c1ccc(-c2nc(-c3ccccc3)c3oc4cc(-c5ccc6c(c5)oc5ccccc56)ccc4c3n2)cc1]

VG6 [WO19059577]

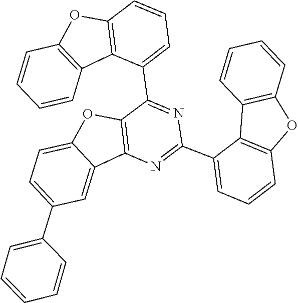 [SMILES: c1ccc(-c2ccc3oc4c(-c5cccc6oc7ccccc7c56)nc(-c5cccc6oc7ccccc7c56)nc4c3c2)cc1]

VG7 [WO19229584]

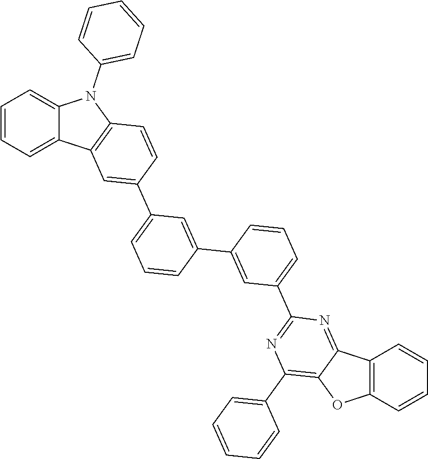 [SMILES: c1ccc(-c2nc(-c3cccc(-c4cccc(-c5ccc6c(c5)c5ccccc5n6-c5ccccc5)c4)c3)nc3c2oc2ccccc23)cc1]

VG8 [WO15105315]

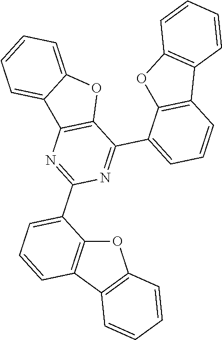 [SMILES: c1ccc2c(c1)oc1c(-c3nc(-c4cccc5c4oc4ccccc45)c4oc5ccccc5c4n3)cccc12]

VG9 [WO15105315]

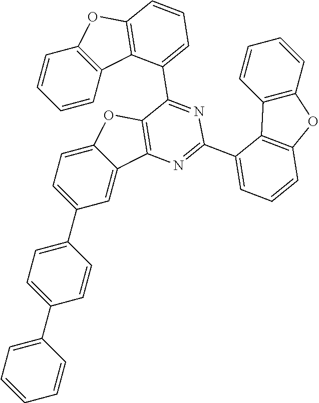 [SMILES: c1ccc(-c2ccc(-c3ccc4oc5c(-c6cccc7oc8ccccc8c67)nc(-c6cccc7oc8ccccc8c67)nc5c4c3)cc2)cc1]

VG10 [WO19229583]

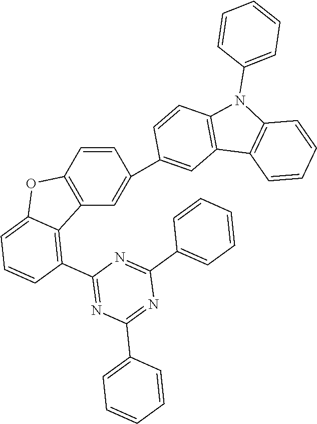 [SMILES: c1ccc(-c2nc(-c3ccccc3)nc(-c3cccc4oc5ccc(-c6ccc7c(c6)c6ccccc6n7-c6ccccc6)cc5c34)n2)cc1]

VG11 [WO2015169412]

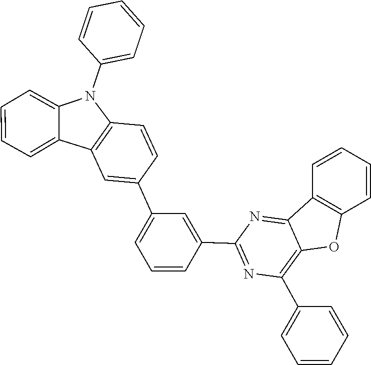 [SMILES: c1ccc(-c2nc(-c3cccc(-c4ccc5c(c4)c4ccccc4n5-c4ccccc4)c3)nc3c2oc2ccccc23)cc1]

VG12 [WO15105315]

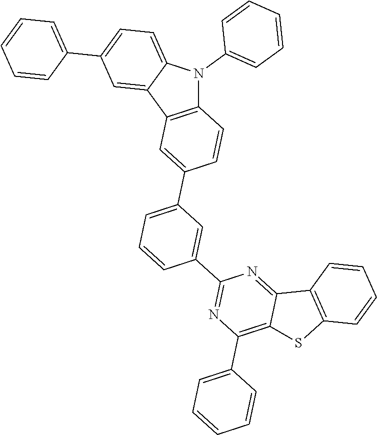 [SMILES: c1ccc(-c2ccc3c(c2)c2cc(-c4cccc(-c5nc(-c6ccccc6)c6sc7ccccc7c6n5)c4)ccc2n3-c2ccccc2)cc1]

VG13 [US2016072078]

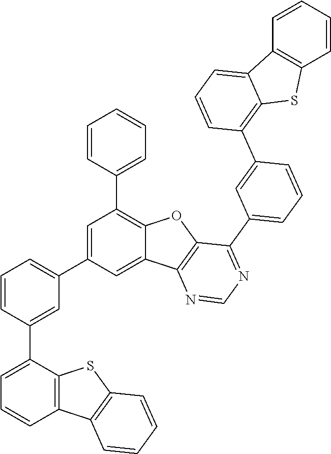 [SMILES: c1ccc(-c2cc(-c3cccc(-c4cccc5c4sc4ccccc45)c3)cc3c2oc2c(-c4cccc(-c5cccc6c5sc5ccccc56)c4)ncnc23)cc1]

VG14 [WO17109637]

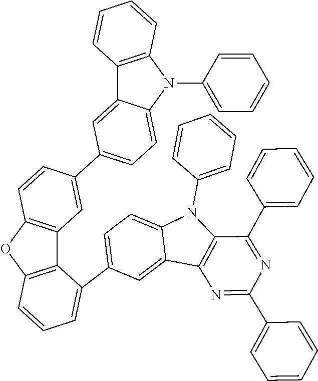 [SMILES: c1ccc(-c2nc(-c3ccccc3)c3c(n2)c2cc(-c4cccc5oc6ccc(-c7ccc8c(c7)c7ccccc7n8-c7ccccc7)cc6c45)ccc2n3-c2ccccc2)cc1]

VG15 [WO17186760]

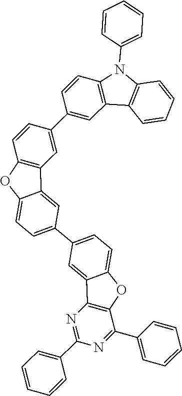 [SMILES: c1ccc(-c2nc(-c3ccccc3)c3oc4ccc(-c5ccc6oc7ccc(-c8ccc9c(c8)c8ccccc8n9-c8ccccc8)cc7c6c5)cc4c3n2)cc1]

VG16 [WO18060307]

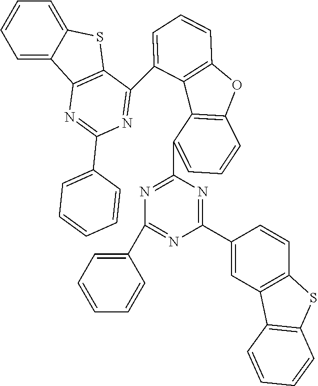 [SMILES: c1ccc(-c2nc(-c3ccc4sc5ccccc5c4c3)nc(-c3cccc4oc5cccc(-c6nc(-c7ccccc7)nc7c6sc6ccccc67)c5c34)n2)cc1]

VG17 [US20200010476]

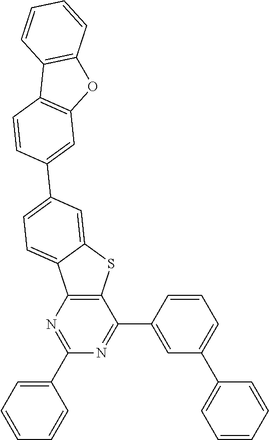 [SMILES: c1ccc(-c2cccc(-c3nc(-c4ccccc4)nc4c3sc3cc(-c5ccc6c(c5)oc5ccccc56)ccc34)c2)cc1]

VG18 [WO20067657]

### Example 2: Synthesis of Host Materials and Precursors Thereof

The syntheses which follow, unless stated otherwise, are conducted under a protective gas atmosphere in dried solvents. The compounds of the invention can be prepared by means of synthesis methods known to those skilled in the art.

### a) 2-Chloro-4,8-diphenylbenzofuro[3,2-d]pyrimidine

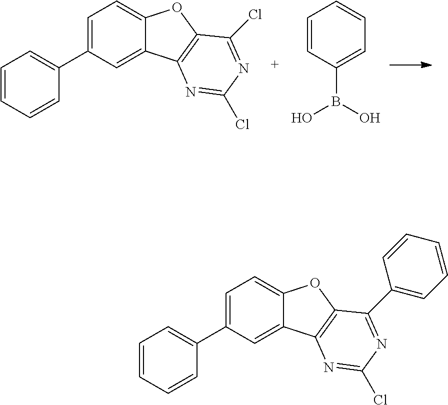 [SMILES: Clc1nc(Cl)c2oc3ccc(-c4ccccc4)cc3c2n1 ; OB(O)c1ccccc1 ; Clc1nc(-c2ccccc2)c2oc3ccc(-c4ccccc4)cc3c2n1]

31.4 g (100 mmol) of 2,4-dichloro-8-phenylbenzofuro[3,2-d]pyrimidine, 12.2 g (100 mmol) of phenylboronic acid and 11.8 g (111 mmol) of sodium carbonate are dissolved in 800 ml of 1,4-dioxane, 800 ml water and 250 ml of toluene, and stirred under an argon atmosphere. 1.2 g (1 mmol) of tetrakis(triphenylphosphine)palladium is added to the flask. The reaction mixture is stirred under reflux overnight. After cooling, the mixture is quenched. The organic phase is separated, washed three times with 300 ml of water, dried over MgSO₄ and filtered, and the solvent is removed under reduced pressure. The residue is purified by column chromatography using silica gel (eluent: DCM/heptane (1:10)). The yield is 28 g (80 mmol), corresponding to 80% of theory.

The following compounds are prepared in an analogous manner:

| | Reactant 1 | Reactant 1 | Product | Yield |
|---|---|---|---|---|
| 1a | 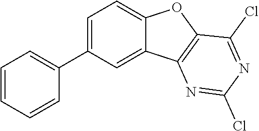 [SMILES: Clc1nc(Cl)c2oc3ccc(-c4ccccc4)cc3c2n1] [1835207-37-8] | 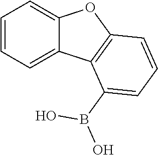 [SMILES: OB(O)c1cccc2oc3ccccc3c12] [162607-19-4] | 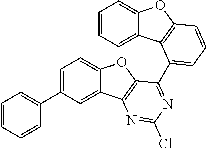 [SMILES: Clc1nc(-c2cccc3oc4ccccc4c23)c2oc3ccc(-c4ccccc4)cc3c2n1] | 75% |
| 2a | 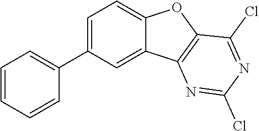 [SMILES: Clc1nc(Cl)c2oc3ccc(-c4ccccc4)cc3c2n1] [1835207-37-8] | 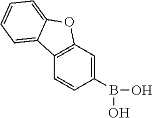 [SMILES: OB(O)c1ccc2c(c1)oc1ccccc12] [395087-89-5] | 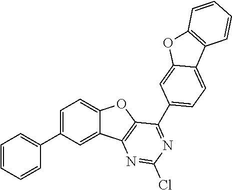 [SMILES: Clc1nc(-c2ccc3c(c2)oc2ccccc23)c2oc3ccc(-c4ccccc4)cc3c2n1] | 73% |
| 3a | 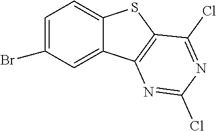 [SMILES: Clc1nc(Cl)c2sc3ccc(Br)cc3c2n1] [2201128-35-8] | 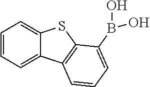 [SMILES: OB(O)c1cccc2c1sc1ccccc12] [108847-20-7] | 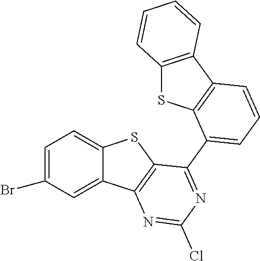 [SMILES: Clc1nc(-c2cccc3c2sc2ccccc23)c2sc3ccc(Br)cc3c2n1] | 67% |
| 4a | 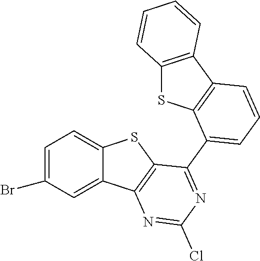 [SMILES: Clc1nc(-c2cccc3c2sc2ccccc23)c2sc3ccc(Br)cc3c2n1] | 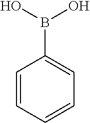 | 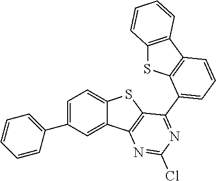 [SMILES: Clc1nc(-c2cccc3c2sc2ccccc23)c2sc3ccc(-c4ccccc4)cc3c2n1] | 80% |
| 5a | 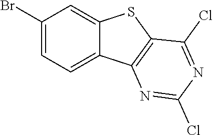 [SMILES: Clc1nc(Cl)c2sc3cc(Br)ccc3c2n1] [2201128-36-9] | 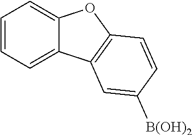 [SMILES: OB(O)c1ccc2oc3ccccc3c2c1] [402936-15-6] | 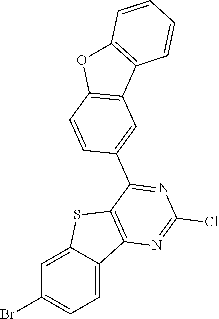 [SMILES: Clc1nc(-c2ccc3oc4ccccc4c3c2)c2sc3cc(Br)ccc3c2n1] | 73% |
| 6a | 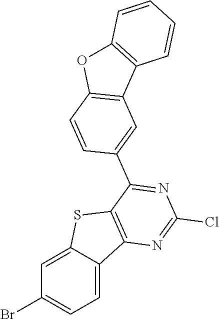 [SMILES: Clc1nc(-c2ccc3oc4ccccc4c3c2)c2sc3cc(Br)ccc3c2n1] | 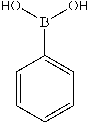 | 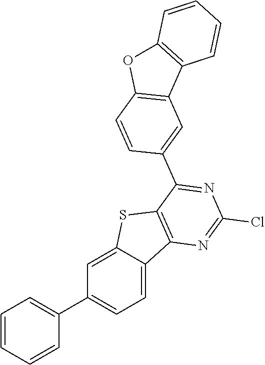 [SMILES: Clc1nc(-c2ccc3oc4ccccc4c3c2)c2sc3cc(-c4ccccc4)ccc3c2n1] | 86% |
| 7a | 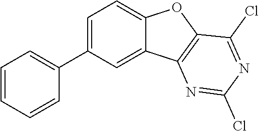 [SMILES: Clc1nc(Cl)c2oc3ccc(-c4ccccc4)cc3c2n1] [1835207-37-8] | 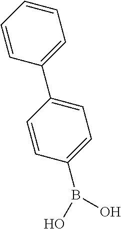 [SMILES: OB(O)c1ccc(-c2ccccc2)cc1] [5122-94-1] | 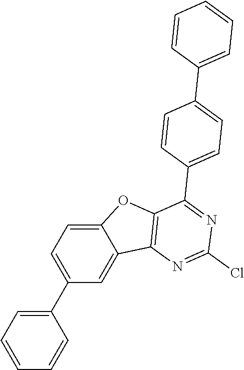 [SMILES: Clc1nc(-c2ccc(-c3ccccc3)cc2)c2oc3ccc(-c4ccccc4)cc3c2n1] | 84% |
| 8a | 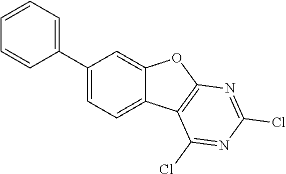 [SMILES: Clc1nc(Cl)c2c(n1)oc1cc(-c3ccccc3)ccc12] [2201128-28-9] | 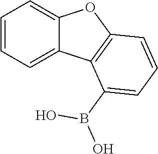 [SMILES: OB(O)c1cccc2oc3ccccc3c12] [162607-19-4] | 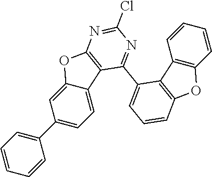 [SMILES: Clc1nc(-c2cccc3oc4ccccc4c23)c2c(n1)oc1cc(-c3ccccc3)ccc12] | 76% |
| 9a | 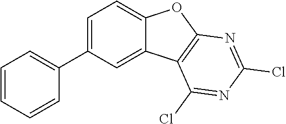 [SMILES: Clc1nc(Cl)c2c(n1)oc1ccc(-c3ccccc3)cc12] [2201128-33-6] | 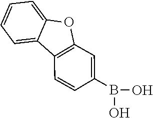 [SMILES: OB(O)c1ccc2c(c1)oc1ccccc12] [395087-89-5] | 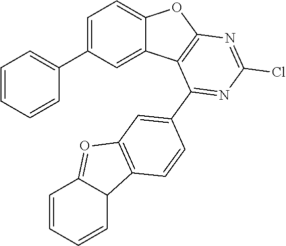 | 73% |
| 10a | 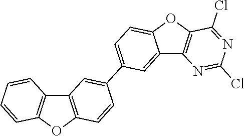 [SMILES: Clc1nc(Cl)c2oc3ccc(-c4ccc5oc6ccccc6c5c4)cc3c2n1] [2219361-31-4] | 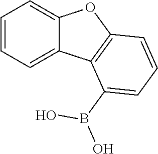 [SMILES: OB(O)c1cccc2oc3ccccc3c12] [162607-19-4] | 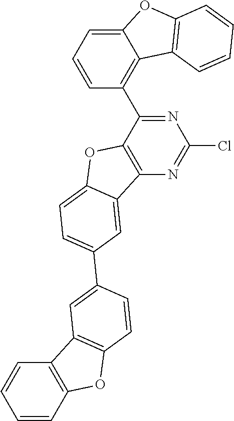 [SMILES: Clc1nc(-c2cccc3oc4ccccc4c23)c2oc3ccc(-c4ccc5oc6ccccc6c5c4)cc3c2n1] | 79% |
| 11a | 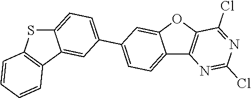 [SMILES: Clc1nc(Cl)c2oc3cc(-c4ccc5sc6ccccc6c5c4)ccc3c2n1] [2201128-2-2] | 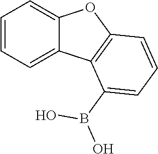 [SMILES: OB(O)c1cccc2oc3ccccc3c12] [162607-19-4] | 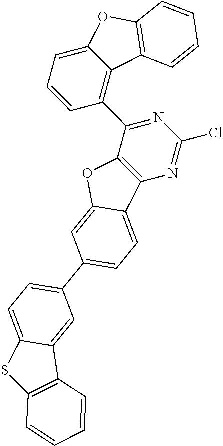 [SMILES: Clc1nc(-c2cccc3oc4ccccc4c23)c2oc3cc(-c4ccc5sc6ccccc6c5c4)ccc3c2n1] | 81% |
| 12a | 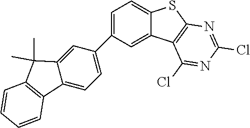 [SMILES: CC1(C)c2ccccc2-c2ccc(-c3ccc4sc5nc(Cl)nc(Cl)c5c4c3)cc21] [2219361-27-8] | 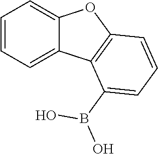 [SMILES: OB(O)c1cccc2oc3ccccc3c12] [162607-19-4] | 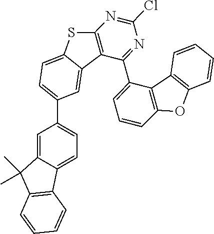 [SMILES: CC1(C)c2ccccc2-c2ccc(-c3ccc4sc5nc(Cl)nc(-c6cccc7oc8ccccc8c67)c5c4c3)cc21] | 76% |
| 13a | 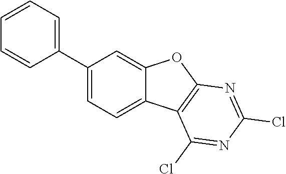 [SMILES: Clc1nc(Cl)c2c(n1)oc1cc(-c3ccccc3)ccc12] [2201128-28-9] | 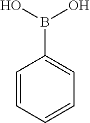 | 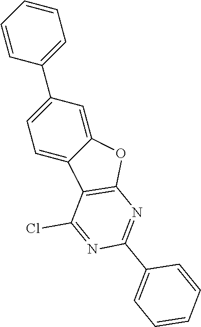 [SMILES: Clc1nc(-c2ccccc2)nc2oc3cc(-c4ccccc4)ccc3c12] | 75% |
| | | | [2201128-28-9] | |
| 14a | 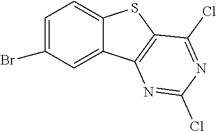 [SMILES: Clc1nc(Cl)c2sc3ccc(Br)cc3c2n1] [2201128-35-8] | 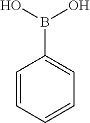 | 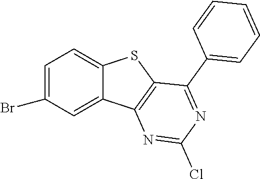 [SMILES: Clc1nc(-c2ccccc2)c2sc3ccc(Br)cc3c2n1] | 77% |
| 15a | 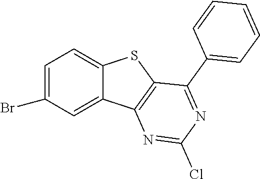 [SMILES: Clc1nc(-c2ccccc2)c2sc3ccc(Br)cc3c2n1] | 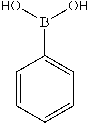 | 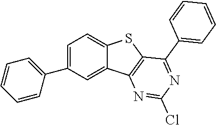 [SMILES: Clc1nc(-c2ccccc2)c2sc3ccc(-c4ccccc4)cc3c2n1] | 81% |
| 16a | 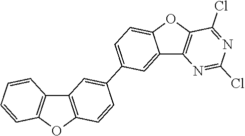 [SMILES: Clc1nc(Cl)c2oc3ccc(-c4ccc5oc6ccccc6c5c4)cc3c2n1] [2219361-31-4] | 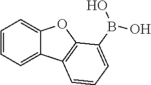 [SMILES: OB(O)c1cccc2c1oc1ccccc12] [100124-06-9] | 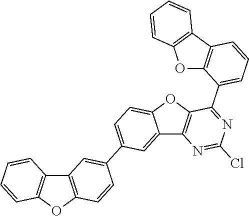 [SMILES: Clc1nc(-c2cccc3c2oc2ccccc23)c2oc3ccc(-c4ccc5oc6ccccc6c5c4)cc3c2n1] | 63% |
| 17a | 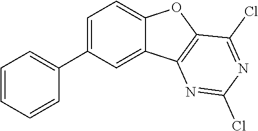 [SMILES: Clc1nc(Cl)c2oc3ccc(-c4ccccc4)cc3c2n1] [1835207-37-8] | 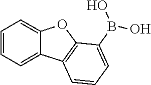 [SMILES: OB(O)c1cccc2c1oc1ccccc12] [100124-06-9] | 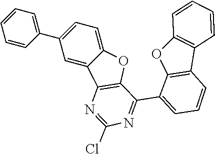 [SMILES: Clc1nc(-c2cccc3c2oc2ccccc23)c2oc3ccc(-c4ccccc4)cc3c2n1] | 82% |
| 18a | 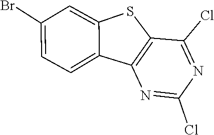 [SMILES: Clc1nc(Cl)c2sc3cc(Br)ccc3c2n1] [2201128-36-9] | 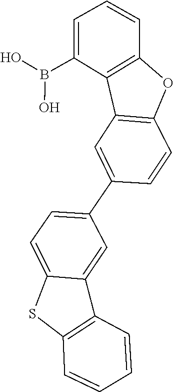 [SMILES: OB(O)c1cccc2oc3ccc(-c4ccc5sc6ccccc6c5c4)cc3c12] [2412400-98-5] | 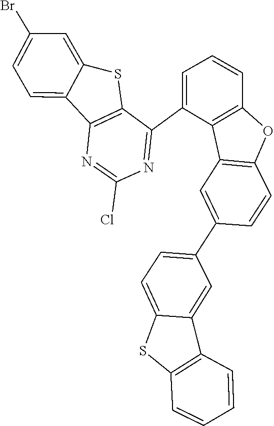 [SMILES: Clc1nc(-c2cccc3oc4ccc(-c5ccc6sc7ccccc7c6c5)cc4c23)c2sc3cc(Br)ccc3c2n1] | 64% |
| 19a | 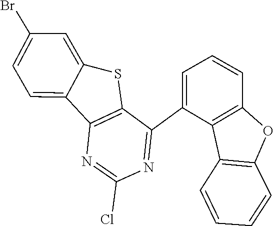 [SMILES: Clc1nc(-c2cccc3oc4ccccc4c23)c2sc3cc(Br)ccc3c2n1] | 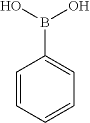 | 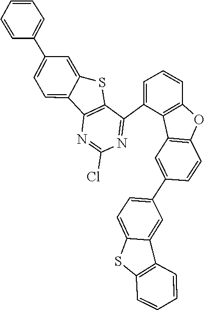 [SMILES: Clc1nc(-c2cccc3oc4ccc(-c5ccc6sc7ccccc7c6c5)cc4c23)c2sc3cc(-c4ccccc4)ccc3c2n1] | 84% |
| 20a | 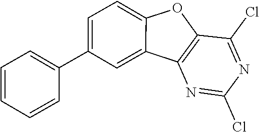 [SMILES: Clc1nc(Cl)c2oc3ccc(-c4ccccc4)cc3c2n1] [1835207-37-8] | 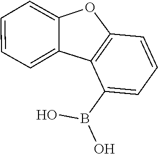 [SMILES: OB(O)c1cccc2oc3ccccc3c12] [162607-19-4] | 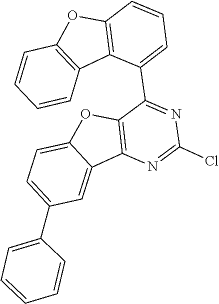 [SMILES: Clc1nc(-c2cccc3oc4ccccc4c23)c2oc3ccc(-c4ccccc4)cc3c2n1] | 74% |
| 21a | 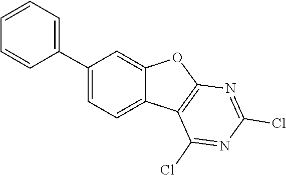 [SMILES: Clc1nc(Cl)c2c(n1)oc1cc(-c3ccccc3)ccc12] [2201128-28-9] | 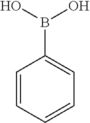 | 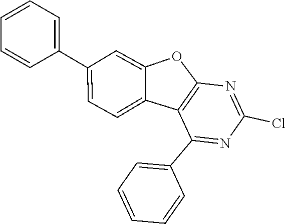 [SMILES: Clc1nc(-c2ccccc2)c2c(n1)oc1cc(-c3ccccc3)ccc12] | 62% |
| 22a | 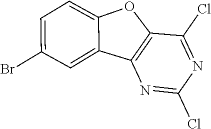 [SMILES: Clc1nc(Cl)c2oc3ccc(Br)cc3c2n1] [1169560-03-5] | 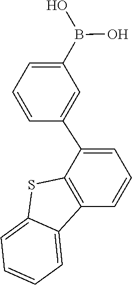 [SMILES: OB(O)c1cccc(-c2cccc3c2sc2ccccc23)c1] [1307859-67-1] | 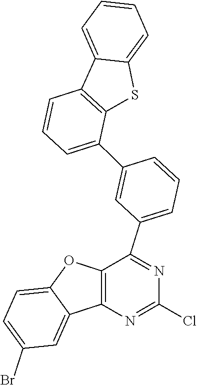 [SMILES: Clc1nc(-c2cccc(-c3cccc4c3sc3ccccc34)c2)c2oc3ccc(Br)cc3c2n1] | 65% |
| 23a | 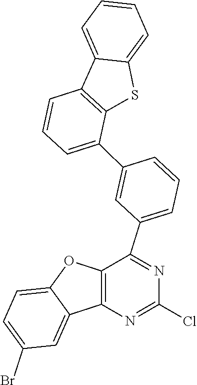 [SMILES: Clc1nc(-c2cccc(-c3cccc4c3sc3ccccc34)c2)c2oc3ccc(Br)cc3c2n1] | 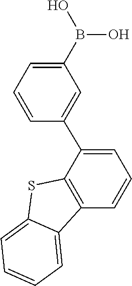 [SMILES: OB(O)c1cccc(-c2cccc3c2sc2ccccc23)c1] [1307859-67-1] | 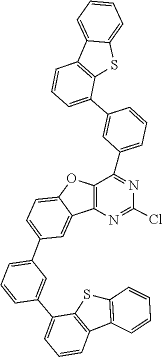 [SMILES: Clc1nc(-c2cccc(-c3cccc4c3sc3ccccc34)c2)c2oc3ccc(-c4cccc(-c5cccc6c5sc5ccccc56)c4)cc3c2n1] | 72% |
| 24a | 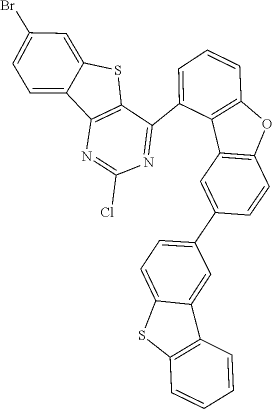 [SMILES: Clc1nc(-c2cccc3oc4ccc(-c5ccc6sc7ccccc7c6c5)cc4c23)c2sc3cc(Br)ccc3c2n1] | 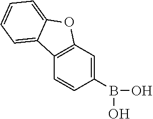 [SMILES: OB(O)c1ccc2c(c1)oc1ccccc12] [395087-89-5] | 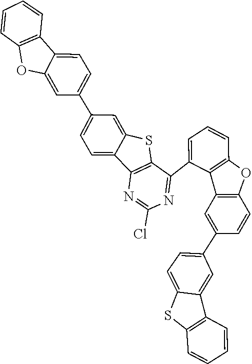 [SMILES: Clc1nc(-c2cccc3oc4ccc(-c5ccc6sc7ccccc7c6c5)cc4c23)c2sc3cc(-c4ccc5c(c4)oc4ccccc45)ccc3c2n1] | 65% |
| 25a | 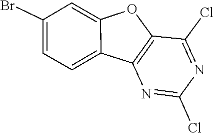 [SMILES: Clc1nc(Cl)c2oc3cc(Br)ccc3c2n1] [2201128-38-1] | 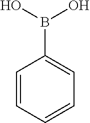 | 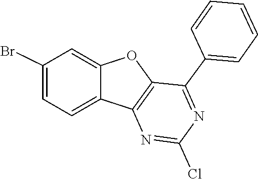 [SMILES: Clc1nc(-c2ccccc2)c2oc3cc(Br)ccc3c2n1] | 56% |
| 26a | 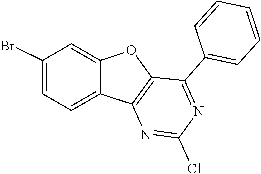 [SMILES: Clc1nc(-c2ccccc2)c2oc3cc(Br)ccc3c2n1] | 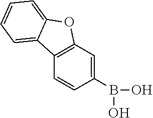 [SMILES: OB(O)c1ccc2c(c1)oc1ccccc12] [395087-89-5] | 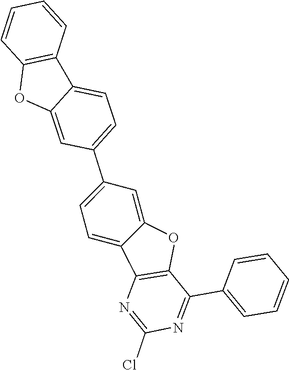 [SMILES: Clc1nc(-c2ccccc2)c2oc3cc(-c4ccc5c(c4)oc4ccccc45)ccc3c2n1] | 77% |
| 27a | 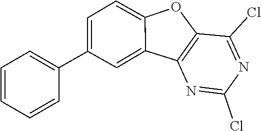 [SMILES: Clc1nc(Cl)c2oc3ccc(-c4ccccc4)cc3c2n1] [1835207-37-8] | 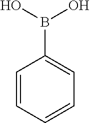 | 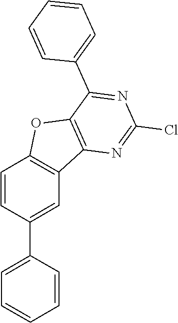 [SMILES: Clc1nc(-c2ccccc2)c2oc3ccc(-c4ccccc4)cc3c2n1] | 65% |

### b) 4,8-Diphenyl-2-[8-(9-phenylcarbazol-3-yl)dibenzofuran-1-yl]benzofuro[3,2-d]pyrimidine

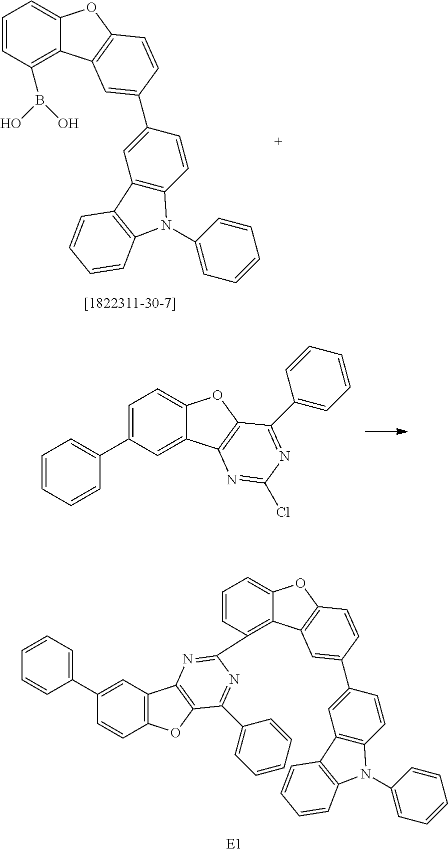 [SMILES: OB(O)c1cccc2oc3ccc(-c4ccc5c(c4)c4ccccc4n5-c4ccccc4)cc3c12 ; Clc1nc(-c2ccccc2)c2oc3ccc(-c4ccccc4)cc3c2n1 ; c1ccc(-c2ccc3oc4c(-c5ccccc5)nc(-c5cccc6oc7ccc(-c8ccc9c(c8)c8ccccc8n9-c8ccccc8)cc7c56)nc4c3c2)cc1]

35.6 g (100 mmol) of 2-chloro-4,8-diphenylbenzofuro[3,2-d]pyrimidine, 54.2 g (100 mmol) of [8-(9-phenylcarbazol-3-yl)dibenzofuran-1-yl]boronic acid and 11.8 g (111 mmol) of sodium carbonate are dissolved in 800 ml of 1,4-dioxane, 800 ml water and 250 ml of toluene, and stirred under an argon atmosphere. 1.2 g (1 mmol) of tetrakis(triphenylphosphine)palladium is added to the flask. The reaction mixture is stirred under reflux overnight. After cooling, the mixture is quenched. The organic phase is separated, washed three times with 300 ml of water, dried over MgSO₄ and filtered, and the solvent is removed under reduced pressure. The residue is purified by column chromatography using silica gel (eluent: DCM/heptane (1:10)). The yield is 51 g (71 mmol), corresponding to 71% of theory.

The following compounds are prepared in an analogous manner:

| | Reactant 1 | Reactant 2 | Product | Yield |
|---|---|---|---|---|
| 1b | 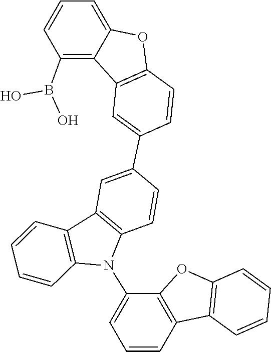 [SMILES: OB(O)c1cccc2oc3ccc(-c4ccc5c(c4)c4ccccc4n5-c4cccc5c4oc4ccccc45)cc3c12] [1822311-33-0] | 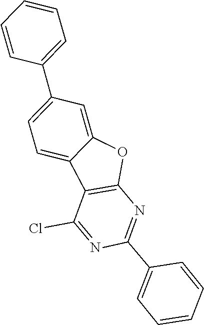 [SMILES: Clc1nc(-c2ccccc2)nc2oc3cc(-c4ccccc4)ccc3c12] [2201128-28-9] | 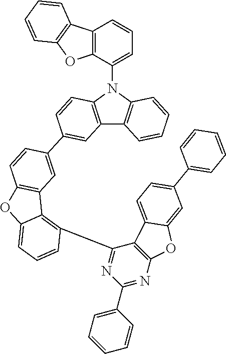 [SMILES: c1ccc(-c2ccc3c(c2)oc2nc(-c4ccccc4)nc(-c4cccc5oc6ccc(-c7ccc8c(c7)c7ccccc7n8-c7cccc8c7oc7ccccc78)cc6c45)c23)cc1] | 72% |
| 2b | 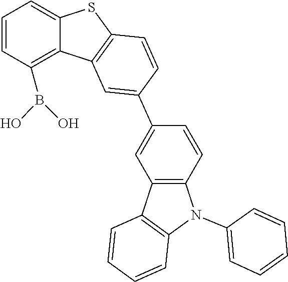 [SMILES: OB(O)c1cccc2sc3ccc(-c4ccc5c(c4)c4ccccc4n5-c4ccccc4)cc3c12] [1822311-32-9] | 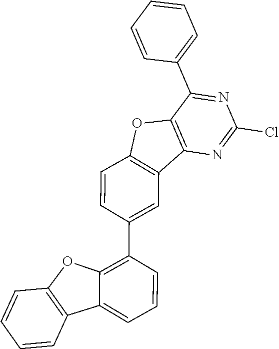 [SMILES: Clc1nc(-c2ccccc2)c2oc3ccc(-c4cccc5c4oc4ccccc45)cc3c2n1] [2268732-49-4] | 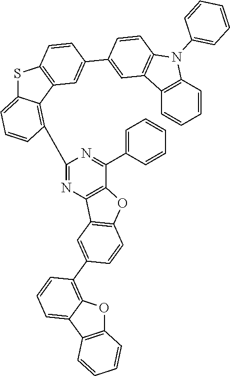 [SMILES: c1ccc(-c2nc(-c3cccc4sc5ccc(-c6ccc7c(c6)c6ccccc6n7-c6ccccc6)cc5c34)nc3c2oc2ccc(-c4cccc5c4oc4ccccc45)cc23)cc1] | 75% |
| 3b | 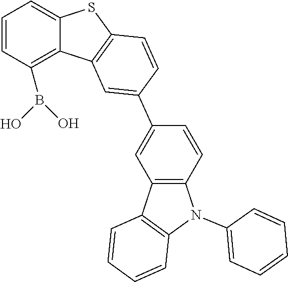 [SMILES: OB(O)c1cccc2sc3ccc(-c4ccc5c(c4)c4ccccc4n5-c4ccccc4)cc3c12] [1822311-32-9] | 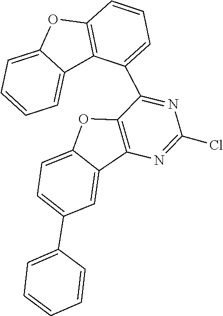 [SMILES: Clc1nc(-c2cccc3oc4ccccc4c23)c2oc3ccc(-c4ccccc4)cc3c2n1] | 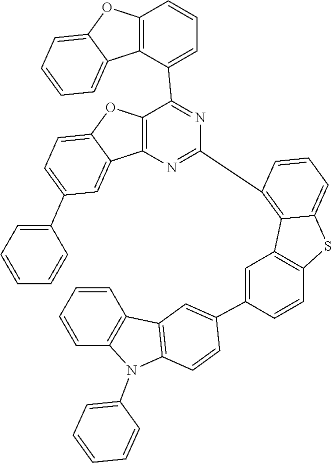 [SMILES: c1ccc(-c2ccc3oc4c(-c5cccc6oc7ccccc7c56)nc(-c5cccc6sc7ccc(-c8ccc9c(c8)c8ccccc8n9-c8ccccc8)cc7c56)nc4c3c2)cc1] | 70% |
| | | | E25 | |
| 4b | 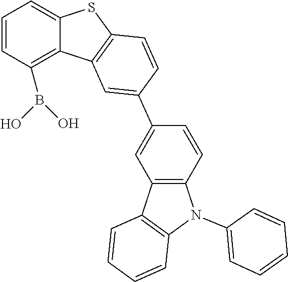 [SMILES: OB(O)c1cccc2sc3ccc(-c4ccc5c(c4)c4ccccc4n5-c4ccccc4)cc3c12] [1822311-32-9] | 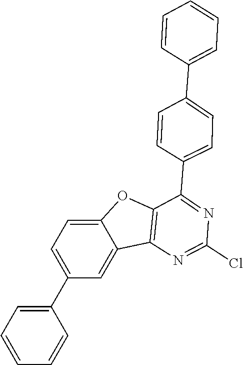 [SMILES: Clc1nc(-c2ccc(-c3ccccc3)cc2)c2oc3ccc(-c4ccccc4)cc3c2n1] | 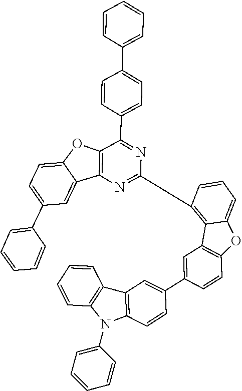 [SMILES: c1ccc(-c2ccc(-c3nc(-c4cccc5oc6ccc(-c7ccc8c(c7)c7ccccc7n8-c7ccccc7)cc6c45)nc4c3oc3ccc(-c5ccccc5)cc34)cc2)cc1] | 68% |
| | | | E5 | |
| 5b | 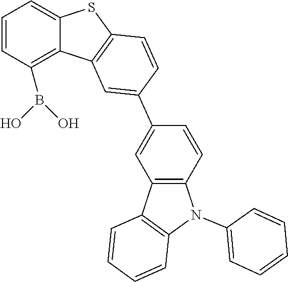 [SMILES: OB(O)c1cccc2sc3ccc(-c4ccc5c(c4)c4ccccc4n5-c4ccccc4)cc3c12] [1822311-32-9] | 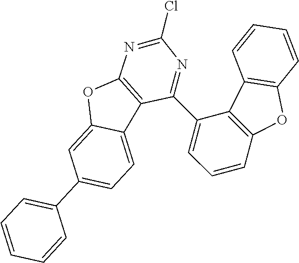 [SMILES: Clc1nc(-c2cccc3oc4ccccc4c23)c2c(n1)oc1cc(-c3ccccc3)ccc12] | 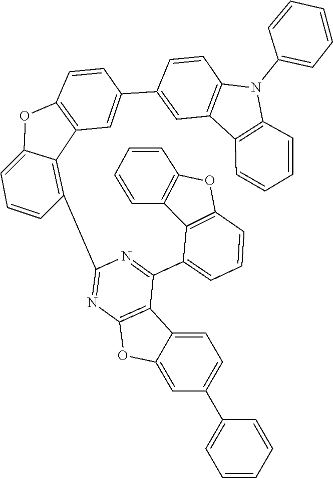 [SMILES: c1ccc(-c2ccc3c(c2)oc2nc(-c4cccc5oc6ccc(-c7ccc8c(c7)c7ccccc7n8-c7ccccc7)cc6c45)nc(-c4cccc5oc6ccccc6c45)c23)cc1] | 67% |
| 6b | 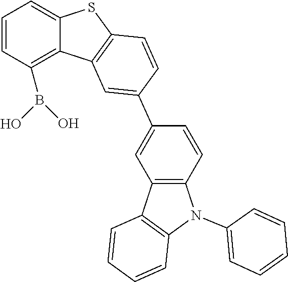 [SMILES: OB(O)c1cccc2sc3ccc(-c4ccc5c(c4)c4ccccc4n5-c4ccccc4)cc3c12] [1822311-32-9] | 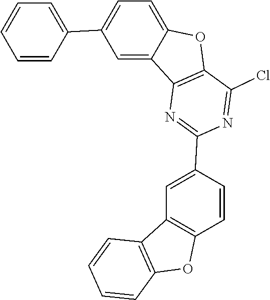 [SMILES: Clc1nc(-c2ccc3oc4ccccc4c3c2)nc2c1oc1ccc(-c3ccccc3)cc12] [2268733-22-6] | 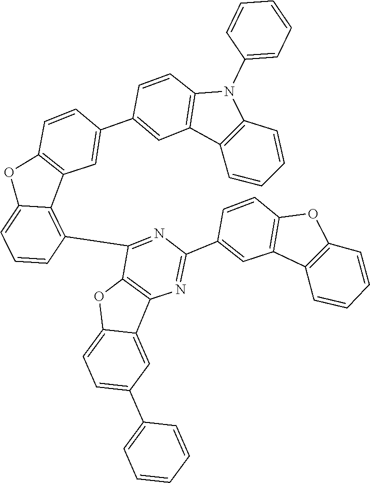 [SMILES: c1ccc(-c2ccc3oc4c(-c5cccc6oc7ccc(-c8ccc9c(c8)c8ccccc8n9-c8ccccc8)cc7c56)nc(-c5ccc6oc7ccccc7c6c5)nc4c3c2)cc1] | 82% |
| | | | E27 | |
| 7b | 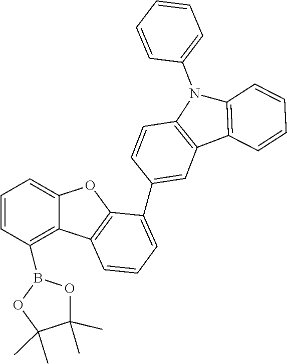 [SMILES: CC1(C)OB(c2cccc3oc4c(-c5ccc6c(c5)c5ccccc5n6-c5ccccc5)cccc4c23)OC1(C)C] | 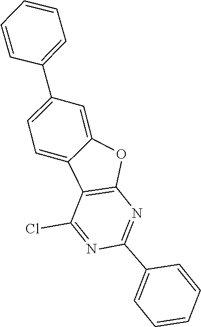 [SMILES: Clc1nc(-c2ccccc2)nc2oc3cc(-c4ccccc4)ccc3c12] [2201128-28-9] | 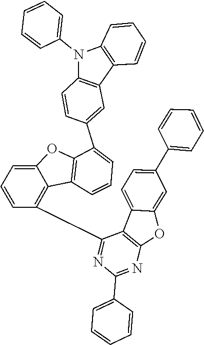 [SMILES: c1ccc(-c2ccc3c(c2)oc2nc(-c4ccccc4)nc(-c4cccc5oc6c(-c7ccc8c(c7)c7ccccc7n8-c7ccccc7)cccc6c45)c23)cc1] | 73% |
| | [2096512-72-8] | | | |
| 8b | 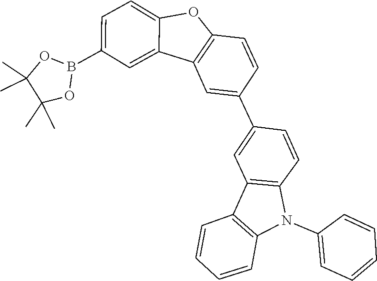 [SMILES: CC1(C)OB(c2ccc3oc4ccc(-c5ccc6c(c5)c5ccccc5n6-c5ccccc5)cc4c3c2)OC1(C)C] [1582802-05-8] | 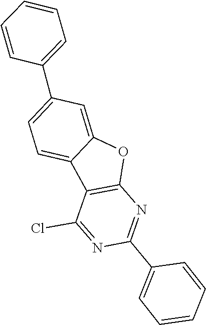 [SMILES: Clc1nc(-c2ccccc2)nc2oc3cc(-c4ccccc4)ccc3c12] [2201128-28-9] | 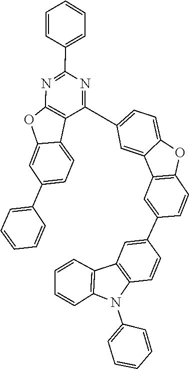 [SMILES: c1ccc(-c2ccc3c(c2)oc2nc(-c4ccccc4)nc(-c4ccc5oc6ccc(-c7ccc8c(c7)c7ccccc7n8-c7ccccc7)cc6c5c4)c23)cc1] | 79% |
| 9b | 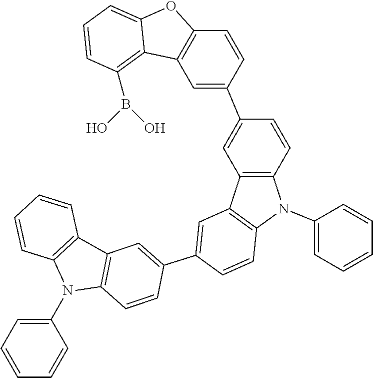 [SMILES: OB(O)c1cccc2oc3ccc(-c4ccc5c(c4)c4cc(-c6ccc7c(c6)c6ccccc6n7-c6ccccc6)ccc4n5-c4ccccc4)cc3c12] | 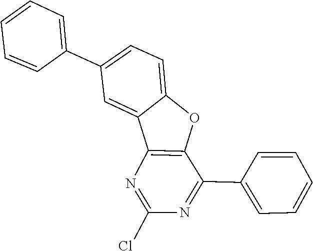 [SMILES: Clc1nc(-c2ccccc2)c2oc3ccc(-c4ccccc4)cc3c2n1] | 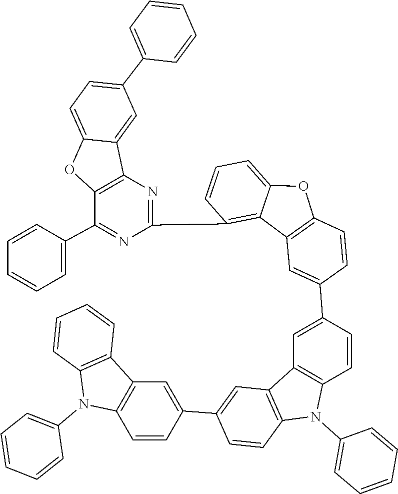 [SMILES: c1ccc(-c2ccc3oc4c(-c5ccccc5)nc(-c5cccc6oc7ccc(-c8ccc9c(c8)c8cc(-c%10ccc%11c(c%10)c%10ccccc%10n%11-c%10ccccc%10)ccc8n9-c8ccccc8)cc7c56)nc4c3c2)cc1] | 69% |
| 11b | 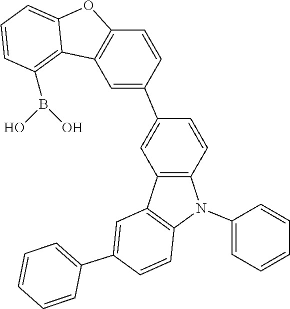 [SMILES: OB(O)c1cccc2oc3ccc(-c4ccc5c(c4)c4cc(-c6ccccc6)ccc4n5-c4ccccc4)cc3c12] | 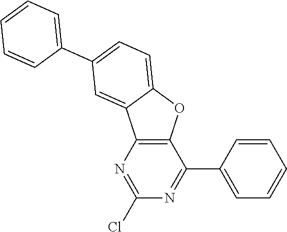 [SMILES: Clc1nc(-c2ccccc2)c2oc3ccc(-c4ccccc4)cc3c2n1] | 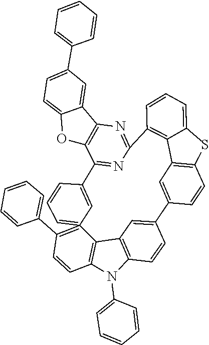 | 76% |
| 12b | 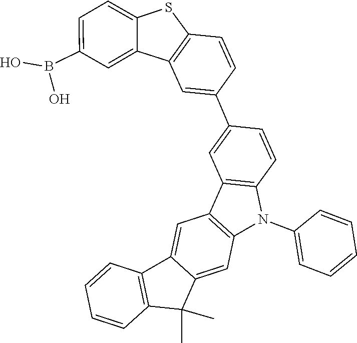 [SMILES: CC1(C)c2ccccc2-c2cc3c4cc(-c5ccc6sc7ccc(B(O)O)cc7c6c5)ccc4n(-c4ccccc4)c3cc21] [1307793-77-6] | 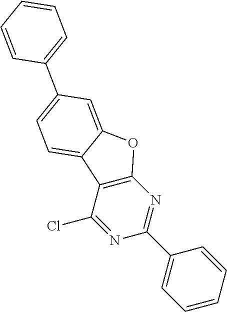 [SMILES: Clc1nc(-c2ccccc2)nc2oc3cc(-c4ccccc4)ccc3c12] [2201128-28-9] | 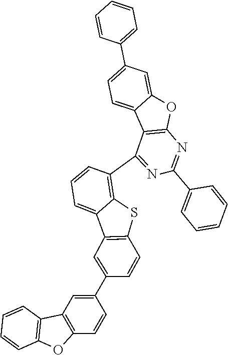 [SMILES: c1ccc(-c2ccc3c(c2)oc2nc(-c4ccccc4)nc(-c4cccc5c4sc4ccc(-c6ccc7oc8ccccc8c7c6)cc45)c23)cc1] | 74% |
| 13b | 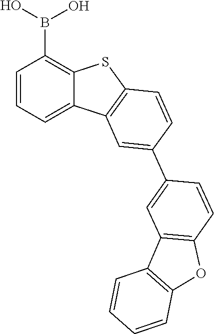 [SMILES: OB(O)c1cccc2c1sc1ccc(-c3ccc4oc5ccccc5c4c3)cc12] [2411332-76-6] | 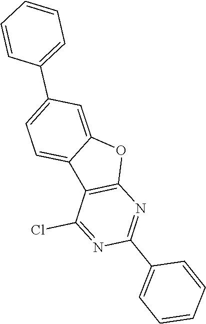 [SMILES: Clc1nc(-c2ccccc2)nc2oc3cc(-c4ccccc4)ccc3c12] [2201128-28-9] | 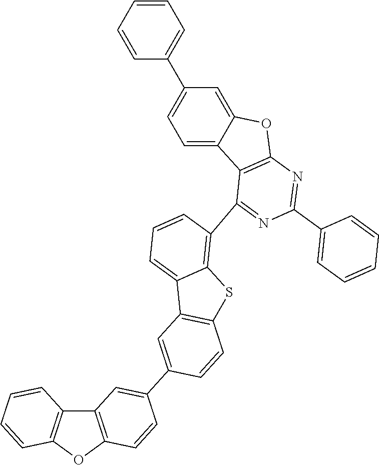 [SMILES: c1ccc(-c2ccc3c(c2)oc2nc(-c4ccccc4)nc(-c4cccc5c4sc4ccc(-c6ccc7oc8ccccc8c7c6)cc45)c23)cc1] | 77% |
| 14b | 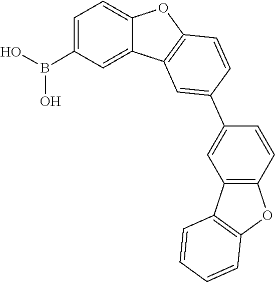 [SMILES: OB(O)c1ccc2oc3ccc(-c4ccc5oc6ccccc6c5c4)cc3c2c1] [1427560-52-8] | 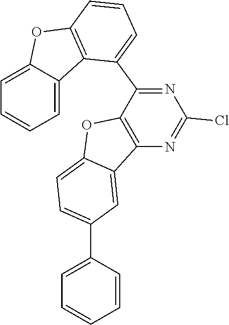 [SMILES: Clc1nc(-c2cccc3oc4ccccc4c23)c2oc3ccc(-c4ccccc4)cc3c2n1] | 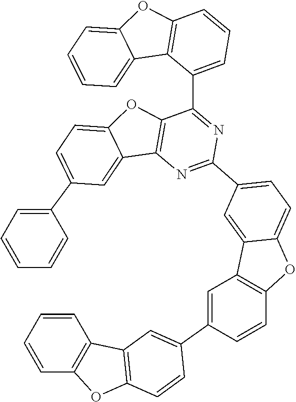 [SMILES: c1ccc(-c2ccc3oc4c(-c5cccc6oc7ccccc7c56)nc(-c5ccc6oc7ccc(-c8ccc9oc%10ccccc%10c9c8)cc7c6c5)nc4c3c2)cc1] | 81% |
| | | | E26 | |
| 15b | 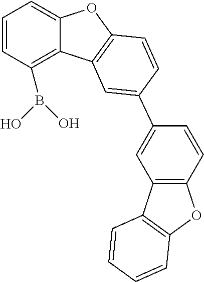 [SMILES: OB(O)c1cccc2oc3ccc(-c4ccc5oc6ccccc6c5c4)cc3c12] [1822320-55-7] | 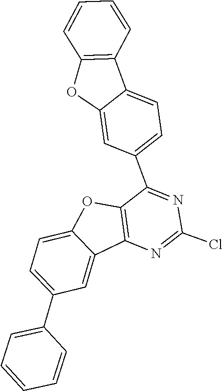 [SMILES: Clc1nc(-c2ccc3c(c2)oc2ccccc23)c2oc3ccc(-c4ccccc4)cc3c2n1] | 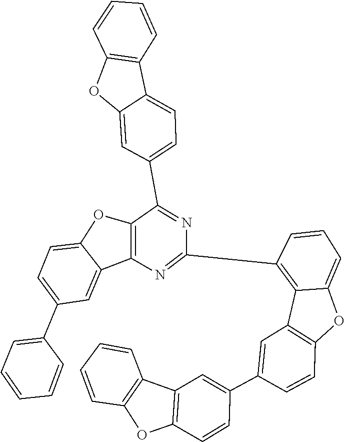 [SMILES: c1ccc(-c2ccc3oc4c(-c5ccc6c(c5)oc5ccccc56)nc(-c5cccc6oc7ccc(-c8ccc9oc%10ccccc%10c9c8)cc7c56)nc4c3c2)cc1] | 80% |
| | | | E6 | |
| 16b | 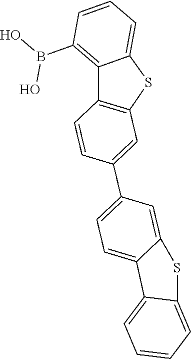 [SMILES: OB(O)c1cccc2sc3cc(-c4ccc5c(c4)sc4ccccc45)ccc3c12] | 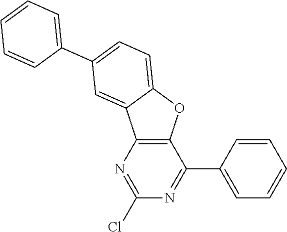 [SMILES: Clc1nc(-c2ccccc2)c2oc3ccc(-c4ccccc4)cc3c2n1] | 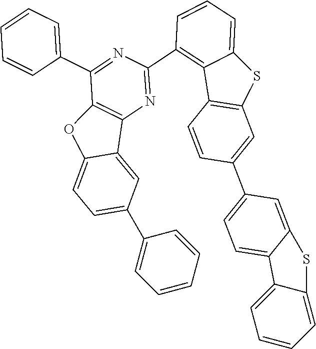 [SMILES: c1ccc(-c2ccc3oc4c(-c5ccccc5)nc(-c5cccc6sc7cc(-c8ccc9c(c8)sc8ccccc89)ccc7c56)nc4c3c2)cc1] | 83% |
| | [2412401-01-3] | | | |
| 17b | 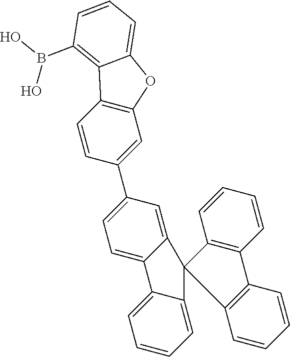 [SMILES: OB(O)c1cccc2oc3cc(-c4ccc5c(c4)C4(c6ccccc6-c6ccccc64)c4ccccc4-5)ccc3c12] 1822311-38-5] | 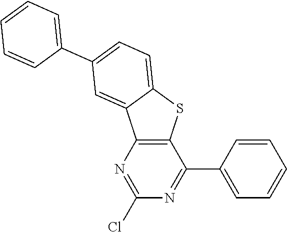 [SMILES: Clc1nc(-c2ccccc2)c2sc3ccc(-c4ccccc4)cc3c2n1] | 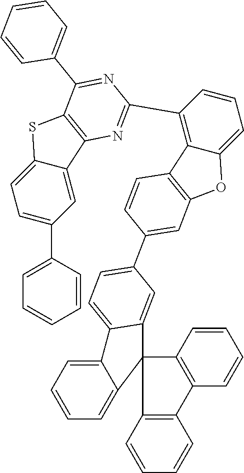 [SMILES: c1ccc(-c2ccc3sc4c(-c5ccccc5)nc(-c5cccc6oc7cc(-c8ccc9c(c8)C8(c%10ccccc%10-c%10ccccc%108)c8ccccc8-9)ccc7c56)nc4c3c2)cc1] | 78% |
| | | | E24 | |
| 18b | 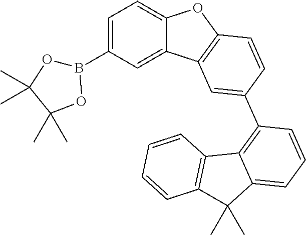 [SMILES: CC1(C)c2ccccc2-c2c(-c3ccc4oc5ccc(B6OC(C)(C)C(C)(C)O6)cc5c4c3)cccc21] [2392930-01-5] | 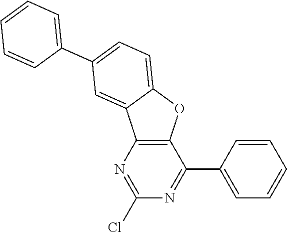 [SMILES: Clc1nc(-c2ccccc2)c2oc3ccc(-c4ccccc4)cc3c2n1] | 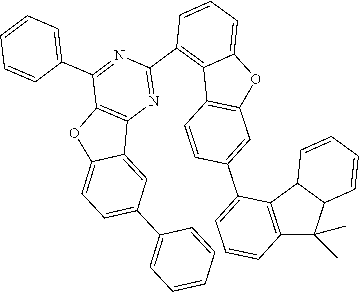 [SMILES: CC1(C)c2cccc(-c3ccc4c(c3)oc3cccc(-c5nc(-c6ccccc6)c6oc7ccc(-c8ccccc8)cc7c6n5)c34)c2C2C=CC=CC21] | 81% |
| | | | E4 | |
| 19b | 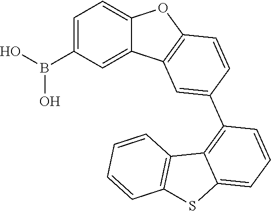 [SMILES: OB(O)c1ccc2oc3ccc(-c4cccc5sc6ccccc6c45)cc3c2c1] [1779497-51-6] | 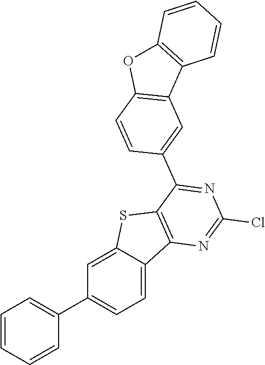 [SMILES: Clc1nc(-c2ccc3oc4ccccc4c3c2)c2sc3cc(-c4ccccc4)ccc3c2n1] | 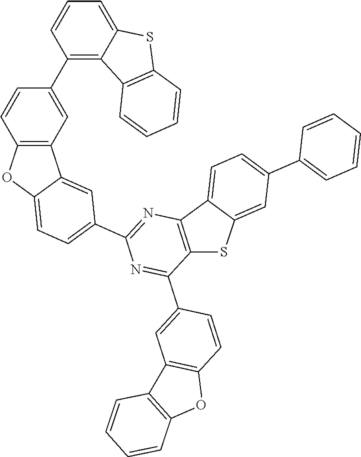 [SMILES: c1ccc(-c2ccc3c(c2)sc2c(-c4ccc5oc6ccccc6c5c4)nc(-c4ccc5oc6ccc(-c7cccc8sc9ccccc9c78)cc6c5c4)nc23)cc1] | 80% |
| 20b | 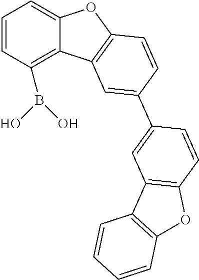 [SMILES: OB(O)c1cccc2oc3ccc(-c4ccc5oc6ccccc6c5c4)cc3c12] [1822320-55-7] | 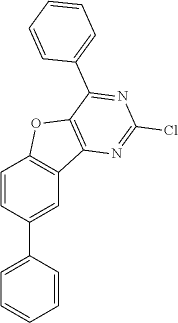 [SMILES: Clc1nc(-c2ccccc2)c2oc3ccc(-c4ccccc4)cc3c2n1] | 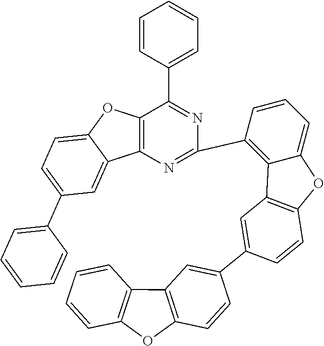 [SMILES: c1ccc(-c2ccc3oc4c(-c5ccccc5)nc(-c5cccc6oc7ccc(-c8ccc9oc%10ccccc%10c9c8)cc7c56)nc4c3c2)cc1] | 79% |
| | | | E2 | |
| 21b | 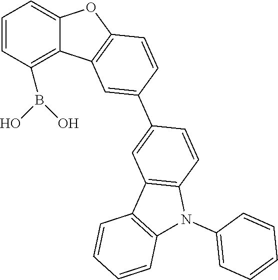 [SMILES: OB(O)c1cccc2oc3ccc(-c4ccc5c(c4)c4ccccc4n5-c4ccccc4)cc3c12] [1822311-30-7] | 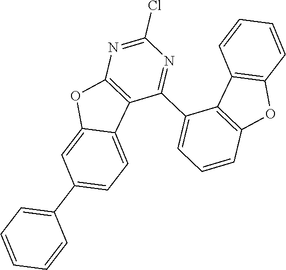 [SMILES: Clc1nc(-c2cccc3oc4ccccc4c23)c2c(n1)oc1cc(-c3ccccc3)ccc12] | 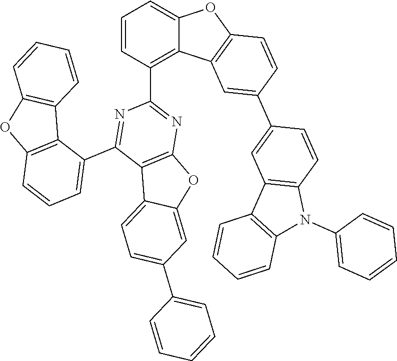 [SMILES: c1ccc(-c2ccc3c(c2)oc2nc(-c4cccc5oc6ccc(-c7ccc8c(c7)c7ccccc7n8-c7ccccc7)cc6c45)nc(-c4cccc5oc6ccccc6c45)c23)cc1] | 76% |
| | | | E23 | |
| 22b | 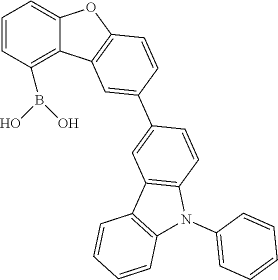 [SMILES: OB(O)c1cccc2oc3ccc(-c4ccc5c(c4)c4ccccc4n5-c4ccccc4)cc3c12] [1822311-30-7] | 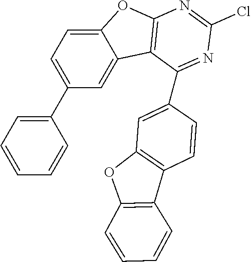 [SMILES: Clc1nc(-c2ccc3c(c2)oc2ccccc23)c2c(n1)oc1ccc(-c3ccccc3)cc12] | 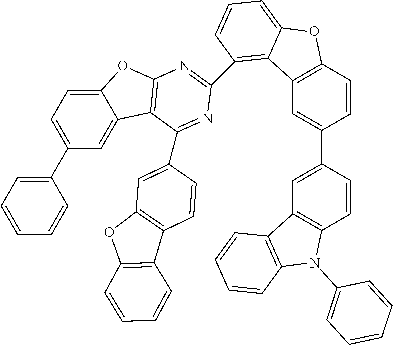 [SMILES: c1ccc(-c2ccc3oc4nc(-c5cccc6oc7ccc(-c8ccc9c(c8)c8ccccc8n9-c8ccccc8)cc7c56)nc(-c5ccc6c(c5)oc5ccccc56)c4c3c2)cc1] | 65% |
| | | | E7 | |
| 23b | 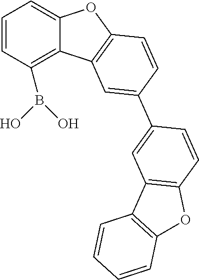 [SMILES: OB(O)c1cccc2oc3ccc(-c4ccc5oc6ccccc6c5c4)cc3c12] [1822320-55-7] | 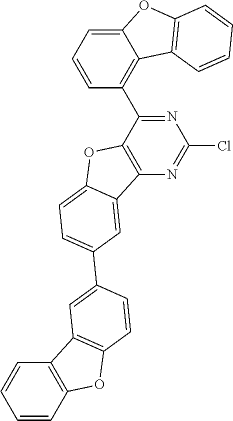 [SMILES: Clc1nc(-c2cccc3oc4ccccc4c23)c2oc3ccc(-c4ccc5oc6ccccc6c5c4)cc3c2n1] | 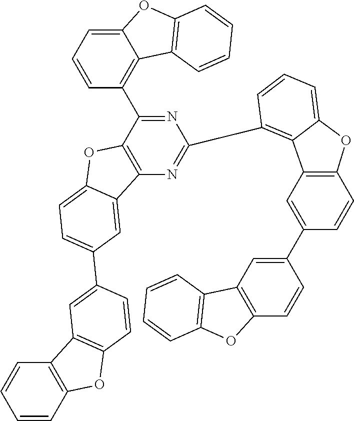 [SMILES: c1ccc2c(c1)oc1ccc(-c3ccc4oc5c(-c6cccc7oc8ccccc8c67)nc(-c6cccc7oc8ccc(-c9ccc%10oc%11ccccc%11c%10c9)cc8c67)nc5c4c3)cc12] | 78% |
| | | | E22 | |
| 24b | 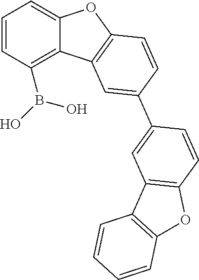 [SMILES: OB(O)c1cccc2oc3ccc(-c4ccc5oc6ccccc6c5c4)cc3c12] [1822320-55-7] | 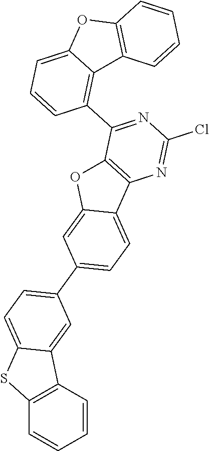 [SMILES: Clc1nc(-c2cccc3oc4ccccc4c23)c2oc3cc(-c4ccc5sc6ccccc6c5c4)ccc3c2n1] | 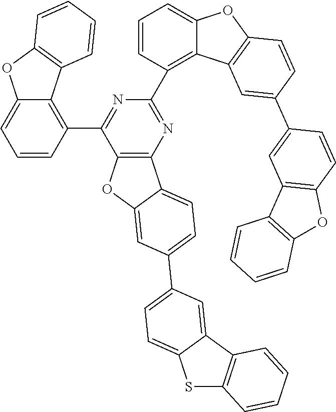 [SMILES: c1ccc2c(c1)oc1ccc(-c3ccc4oc5cccc(-c6nc(-c7cccc8oc9ccccc9c78)c7oc8cc(-c9ccc%10sc%11ccccc%11c%10c9)ccc8c7n6)c5c4c3)cc12] E9 | 68% |
| 25b | 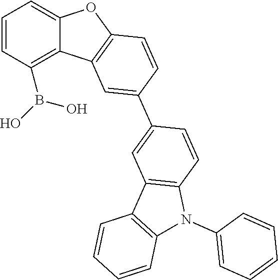 [SMILES: OB(O)c1cccc2oc3ccc(-c4ccc5c(c4)c4ccccc4n5-c4ccccc4)cc3c12] [1822311-30-7] | 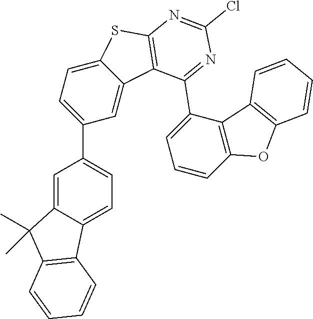 [SMILES: CC1(C)c2ccccc2-c2ccc(-c3ccc4sc5nc(Cl)nc(-c6cccc7oc8ccccc8c67)c5c4c3)cc21] | 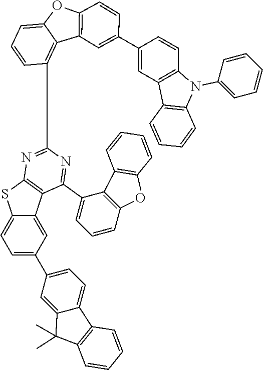 [SMILES: CC1(C)c2ccccc2-c2ccc(-c3ccc4sc5nc(-c6cccc7oc8ccc(-c9ccc%10c(c9)c9ccccc9n%10-c9ccccc9)cc8c67)nc(-c6cccc7oc8ccccc8c67)c5c4c3)cc21] | 67% |
| 26b | 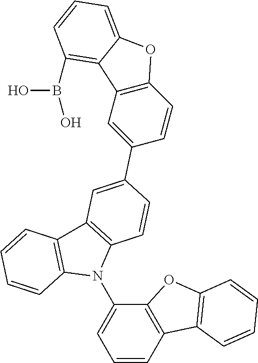 [SMILES: OB(O)c1cccc2oc3ccc(-c4ccc5c(c4)c4ccccc4n5-c4cccc5c4oc4ccccc45)cc3c12] [1822311-33-0] | 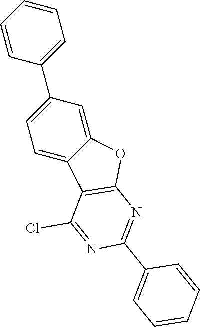 [SMILES: Clc1nc(-c2ccccc2)nc2oc3cc(-c4ccccc4)ccc3c12] [2201128-28-9] | 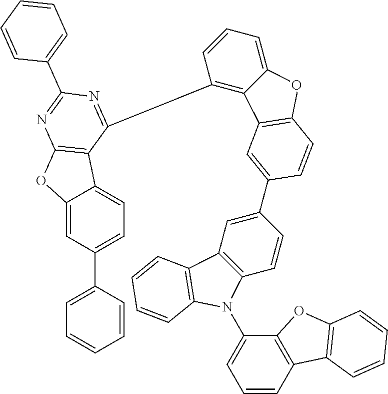 [SMILES: c1ccc(-c2ccc3c(c2)oc2nc(-c4ccccc4)nc(-c4cccc5oc6ccc(-c7ccc8c(c7)c7ccccc7n8-c7cccc8c7oc7ccccc78)cc6c45)c23)cc1] | 81% |
| 27b | 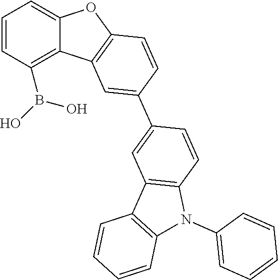 [SMILES: OB(O)c1cccc2oc3ccc(-c4ccc5c(c4)c4ccccc4n5-c4ccccc4)cc3c12] [1822311-30-7] | 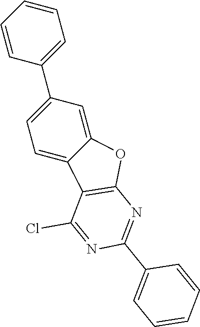 [SMILES: Clc1nc(-c2ccccc2)nc2oc3cc(-c4ccccc4)ccc3c12] [2201128-28-9] | 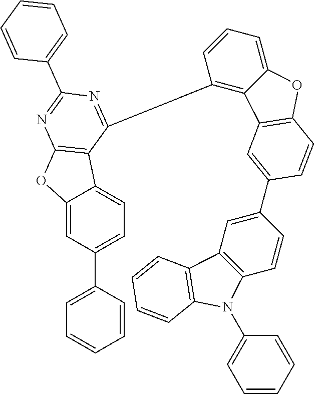 [SMILES: c1ccc(-c2ccc3c(c2)oc2nc(-c4ccccc4)nc(-c4cccc5oc6ccc(-c7ccc8c(c7)c7ccccc7n8-c7ccccc7)cc6c45)c23)cc1] | 76% |
| | | | E10 | |
| 28b | 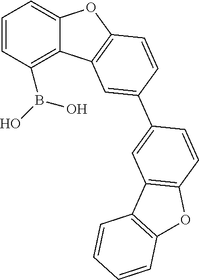 [SMILES: OB(O)c1cccc2oc3ccc(-c4ccc5oc6ccccc6c5c4)cc3c12] [1822320-55-7] | 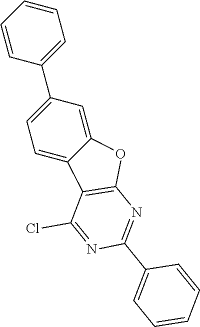 [SMILES: Clc1nc(-c2ccccc2)nc2oc3cc(-c4ccccc4)ccc3c12] [2201128-28-9] | 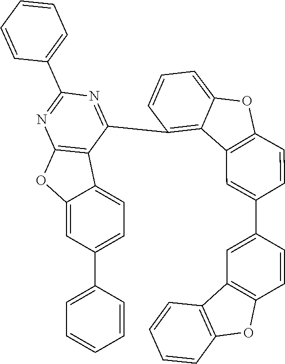 [SMILES: c1ccc(-c2ccc3c(c2)oc2nc(-c4ccccc4)nc(-c4cccc5oc6ccc(-c7ccc8oc9ccccc9c8c7)cc6c45)c23)cc1] | 79% |
| | | | E8 | |
| 29b | 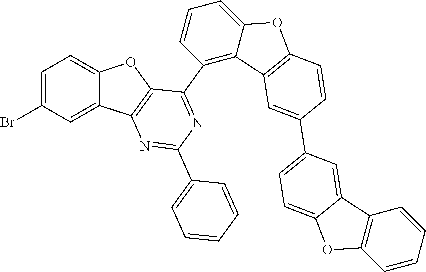 [SMILES: Brc1ccc2oc3c(-c4cccc5oc6ccc(-c7ccc8oc9ccccc9c8c7)cc6c45)nc(-c4ccccc4)nc3c2c1] | 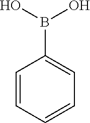 | 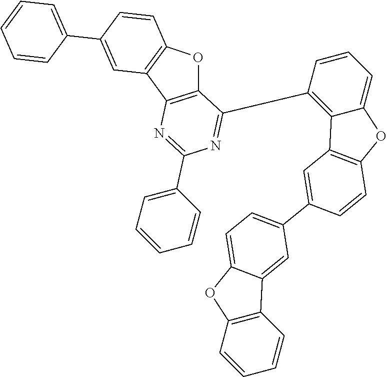 [SMILES: c1ccc(-c2ccc3oc4c(-c5cccc6oc7ccc(-c8ccc9oc%10ccccc%10c9c8)cc7c56)nc(-c5ccccc5)nc4c3c2)cc1] | 81% |
| | | | E3 | |
| 30b | 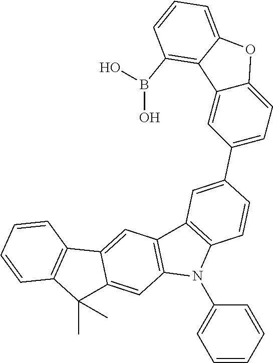 [SMILES: CC1(C)c2ccccc2-c2cc3c4cc(-c5ccc6oc7cccc(B(O)O)c7c6c5)ccc4n(-c4ccccc4)c3cc21] [1822311-35-2] | 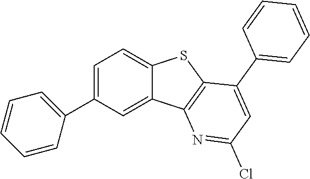 [SMILES: Clc1cc(-c2ccccc2)c2sc3ccc(-c4ccccc4)cc3c2n1] | 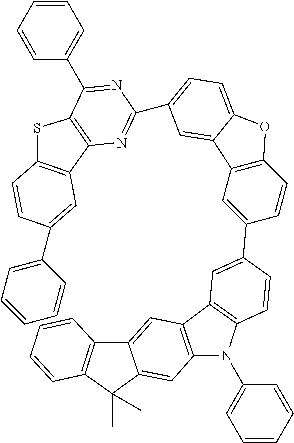 [SMILES: CC1(C)c2ccccc2-c2cc3c4cc(-c5ccc6oc7ccc(-c8nc(-c9ccccc9)c9sc%10ccc(-c%11ccccc%11)cc%10c9n8)cc7c6c5)ccc4n(-c4ccccc4)c3cc21] | 70% |
| | | | E11 | |
| 31b | 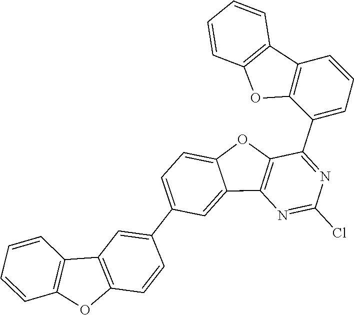 [SMILES: Clc1nc(-c2cccc3c2oc2ccccc23)c2oc3ccc(-c4ccc5oc6ccccc6c5c4)cc3c2n1] | 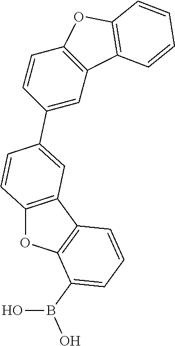 [SMILES: OB(O)c1cccc2c1oc1ccc(-c3ccc4oc5ccccc5c4c3)cc12] [2411326-31-1] | 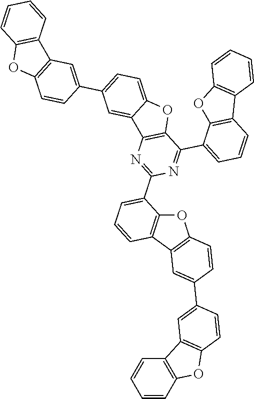 [SMILES: c1ccc2c(c1)oc1ccc(-c3ccc4oc5c(-c6nc(-c7cccc8c7oc7ccccc78)c7oc8ccc(-c9ccc%10oc%11ccccc%11c%10c9)cc8c7n6)cccc5c4c3)cc12] | 75% |
| 32b | 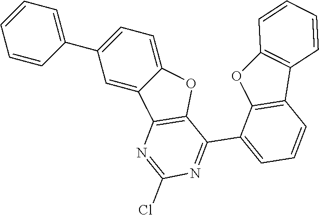 [SMILES: Clc1nc(-c2cccc3c2oc2ccccc23)c2oc3ccc(-c4ccccc4)cc3c2n1] | 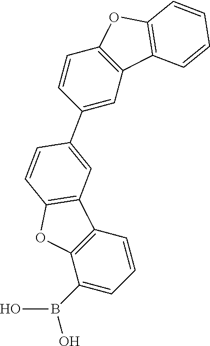 [2411326-31-1] | 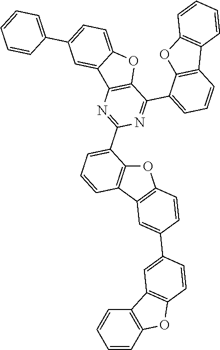 [SMILES: c1ccc(-c2ccc3oc4c(-c5cccc6c5oc5ccccc56)nc(-c5cccc6c5oc5ccc(-c7ccc8oc9ccccc9c8c7)cc56)nc4c3c2)cc1] E12 | 66% |
| 33b | 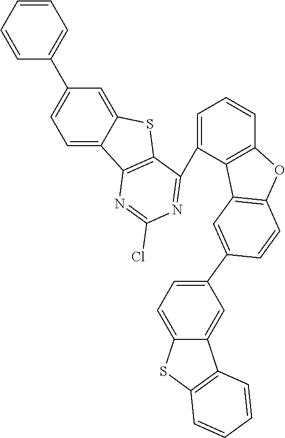 [SMILES: Clc1nc(-c2cccc3oc4ccc(-c5ccc6sc7ccccc7c6c5)cc4c23)c2sc3cc(-c4ccccc4)ccc3c2n1] | 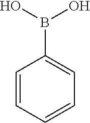 | 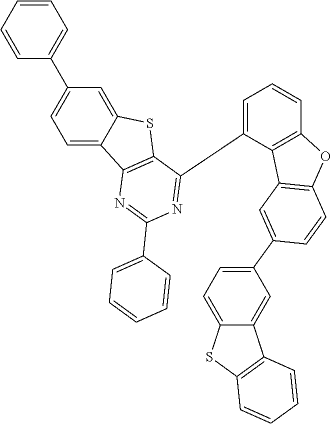 [SMILES: c1ccc(-c2ccc3c(c2)sc2c(-c4cccc5oc6ccc(-c7ccc8sc9ccccc9c8c7)cc6c45)nc(-c4ccccc4)nc23)cc1] E13 | 69% |
| 34b | 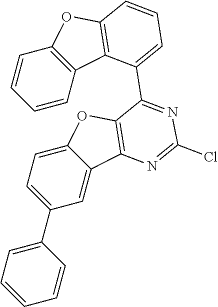 [SMILES: Clc1nc(-c2cccc3oc4ccccc4c23)c2oc3ccc(-c4ccccc4)cc3c2n1] | 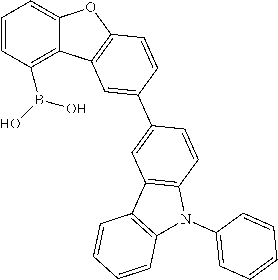 [SMILES: OB(O)c1cccc2oc3ccc(-c4ccc5c(c4)c4ccccc4n5-c4ccccc4)cc3c12] [1822311-30-7] | 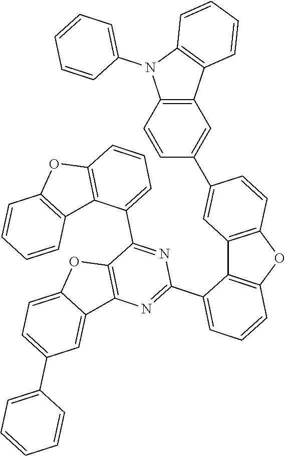 [SMILES: c1ccc(-c2ccc3oc4c(-c5cccc6oc7ccccc7c56)nc(-c5cccc6oc7ccc(-c8ccc9c(c8)c8ccccc8n9-c8ccccc8)cc7c56)nc4c3c2)cc1] | 68% |
| | | | E14 | |
| 35b | 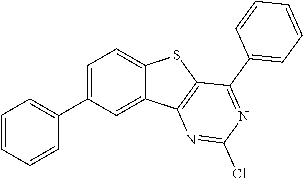 [SMILES: Clc1nc(-c2ccccc2)c2sc3ccc(-c4ccccc4)cc3c2n1] | 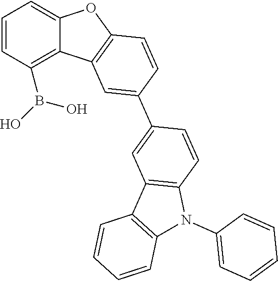 [SMILES: OB(O)c1cccc2oc3ccc(-c4ccc5c(c4)c4ccccc4n5-c4ccccc4)cc3c12] [1822311-30-7] | 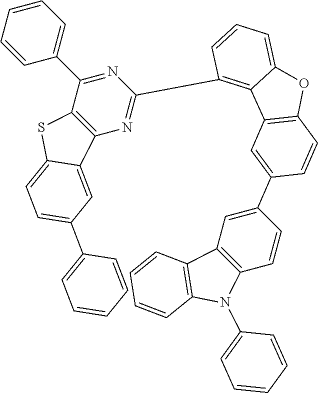 [SMILES: c1ccc(-c2ccc3sc4c(-c5ccccc5)nc(-c5cccc6oc7ccc(-c8ccc9c(c8)c8ccccc8n9-c8ccccc8)cc7c56)nc4c3c2)cc1] | 64% |
| | | | E15 | |
| 36b | 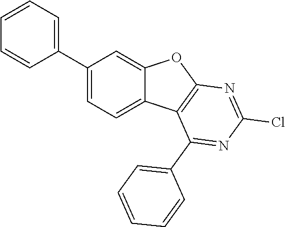 [SMILES: Clc1nc(-c2ccccc2)c2c(n1)oc1cc(-c3ccccc3)ccc12] | 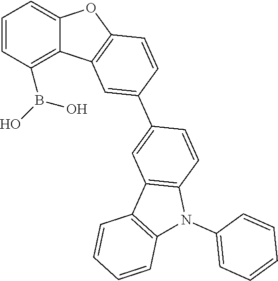 [SMILES: OB(O)c1cccc2oc3ccc(-c4ccc5c(c4)c4ccccc4n5-c4ccccc4)cc3c12] [1822311-30-7] | 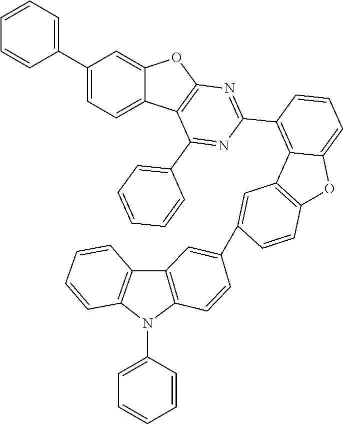 [SMILES: c1ccc(-c2ccc3c(c2)oc2nc(-c4cccc5oc6ccc(-c7ccc8c(c7)c7ccccc7n8-c7ccccc7)cc6c45)nc(-c4ccccc4)c23)cc1] | 63% |
| | | | E16 | |
| 37b | 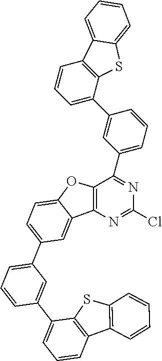 [SMILES: Clc1nc(-c2cccc(-c3cccc4c3sc3ccccc34)c2)c2oc3ccc(-c4cccc(-c5cccc6c5sc5ccccc56)c4)cc3c2n1] | 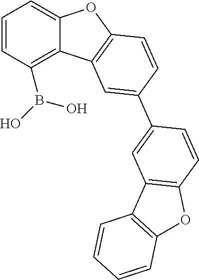 [SMILES: OB(O)c1cccc2oc3ccc(-c4ccc5oc6ccccc6c5c4)cc3c12] [1822320-55-7] | 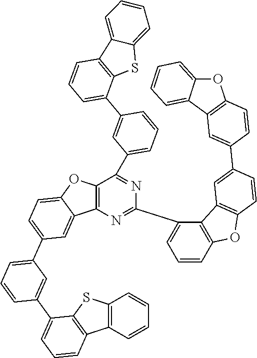 [SMILES: c1cc(-c2ccc3oc4c(-c5cccc(-c6cccc7c6sc6ccccc67)c5)nc(-c5cccc6oc7ccc(-c8ccc9oc%10ccccc%10c9c8)cc7c56)nc4c3c2)cc(-c2cccc3c2sc2ccccc23)c1] | 72% |
| | | | E17 | |
| 38b | 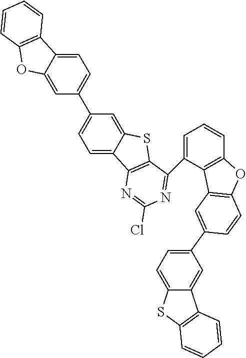 [SMILES: Clc1nc(-c2cccc3oc4ccc(-c5ccc6sc7ccccc7c6c5)cc4c23)c2sc3cc(-c4ccc5c(c4)oc4ccccc45)ccc3c2n1] | 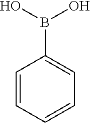 | 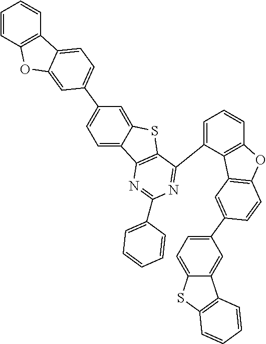 [SMILES: c1ccc(-c2nc(-c3cccc4oc5ccc(-c6ccc7sc8ccccc8c7c6)cc5c34)c3sc4cc(-c5ccc6c(c5)oc5ccccc56)ccc4c3n2)cc1] E18 | 70% |
| 39b | 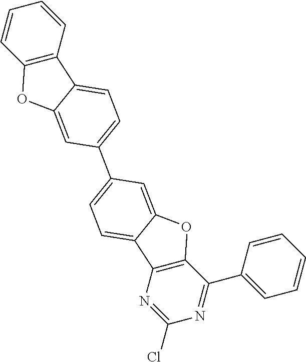 [SMILES: Clc1nc(-c2ccccc2)c2oc3cc(-c4ccc5c(c4)oc4ccccc45)ccc3c2n1] | 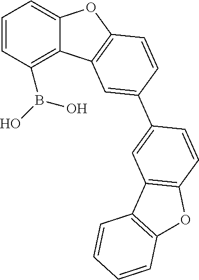 [SMILES: OB(O)c1cccc2oc3ccc(-c4ccc5oc6ccccc6c5c4)cc3c12] [1822320-55-7] | 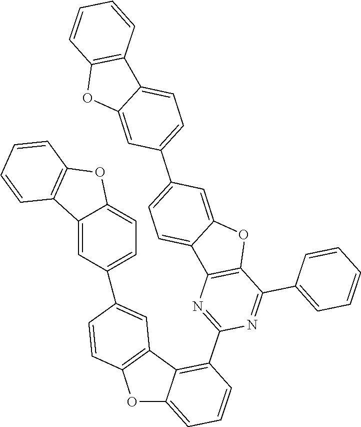 [SMILES: c1ccc(-c2nc(-c3cccc4oc5ccc(-c6ccc7oc8ccccc8c7c6)cc5c34)nc3c2oc2cc(-c4ccc5c(c4)oc4ccccc45)ccc23)cc1] | 73% |
| | | | E19 | |
| 40b | 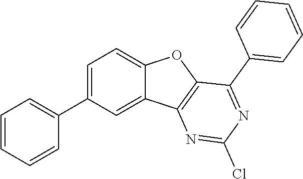 [SMILES: Clc1nc(-c2ccccc2)c2oc3ccc(-c4ccccc4)cc3c2n1] | 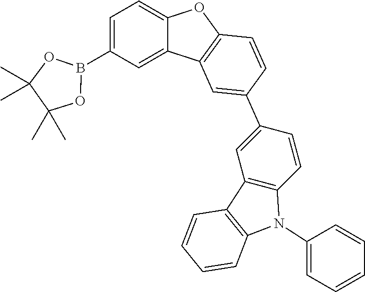 [SMILES: CC1(C)OB(c2ccc3oc4ccc(-c5ccc6c(c5)c5ccccc5n6-c5ccccc5)cc4c3c2)OC1(C)C] [1582802-05-8 ] | 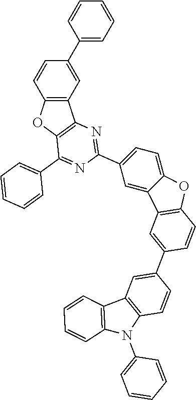 [SMILES: c1ccc(-c2ccc3oc4c(-c5ccccc5)nc(-c5ccc6oc7ccc(-c8ccc9c(c8)c8ccccc8n9-c8ccccc8)cc7c6c5)nc4c3c2)cc1] | 76% |
| | | | E20 | |

1.-15. (canceled)

16. An organic electroluminescent device comprising an anode, a cathode and at least one organic layer containing at least one light-emitting layer, wherein the at least one light-emitting layer contains at least one compound of the formula (1) as host material 1 and at least one compound of the formula (2) as host material 2, 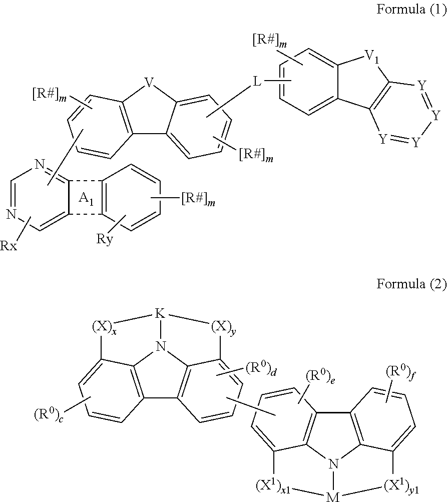
where the symbols and indices used are as follows:
ring A₁ in formula (1) conforms to the formula (1A) 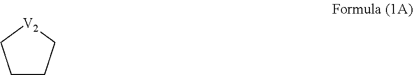
V is O or S;
V₁ is O, S, C(R¹)₂ or N-A;
V₂ is O or S;
Y is the same or different at each instance and is independently CH, CR or CA, or two adjacent Y groups together form a group of the formula (1B) 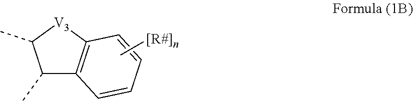
and the remaining Y groups are independently CH or CR, where V₃ is O, S, C(R¹)₂ or N-A and the dotted bonds show the linkage of this group to the remainder of the formula (1);
Rx is L₁-R*;
Ry is L₂-R*;
R* independently at each instance is an aromatic or heteroaromatic ring system which has 6 to 14 ring atoms and may be substituted by one or more R radicals;
L, L₁, L₂ are each independently a bond or a phenylene group which may each be substituted by one or more R radicals;
A is the same or different at each instance and is an aromatic or heteroaromatic ring system which has 6 to 30 ring atoms and may be substituted by one or more R radicals;
n is 0, 1, 2, 3 or 4;
m is 0, 1, 2 or 3;
R # is D or an aryl group which has 6 to 12 carbon atoms and may be substituted by one or more R radicals;
R¹ is the same or different at each instance and is an alkyl group having 1 to 20 carbon atoms, phenyl which may be substituted by one or more R radicals, or the two R¹ radicals together with the carbon atom to which they bind form a spiro compound;
R is the same or different at each instance and is selected from D, F, CN, a straight-chain alkyl group having 1 to 20 carbon atoms or a branched or cyclic alkyl group having 3 to carbon atoms, where one or more nonadjacent CH₂ groups may be replaced by R²C═CR², O or S and where one or more hydrogen atoms may be replaced by D, F, or CN;
R² is the same or different at each instance and is selected from the group consisting of H, D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
K, M are each independently an unsubstituted or partly or fully deuterated or R*-monosubstituted aromatic ring system having 6 to 40 ring atoms when x and y are 0 and when x1 and y1 are 0, or
K, M each independently together with X or X¹ form a heteroaromatic ring system having 14 to 40 ring atoms when the value of x, x1, y and/or y1 is 1;
x, x1 at each instance are each independently 0 or 1;
y, y1 at each instance are each independently 0 or 1;
X and X¹ at each instance are each independently a bond or C(R⁺)₂;
R⁰ at each instance is independently an unsubstituted or partly or fully deuterated aromatic ring system having 6 to 18 carbon atoms;
R⁺ at each instance is independently a straight-chain or branched alkyl group having 1 to 4 carbon atoms and
c, d, e and f are independently 0 or 1.

17. The organic electroluminescent device according to claim 16, characterized in that the host material 1 conforms to the formula (1a) 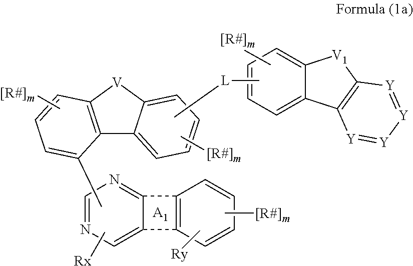
where the symbols Y, V, V₁, R #, m, Rx, Ry, L and ring A₁ used are as defined in claim 16.

18. The organic electroluminescent device according to claim 16, characterized in that V₁ is O or NA.

19. The organic electroluminescent device according to claim 16, characterized in that the host material 2 conforms to one of the formulae (2a), (2b) and (2c) 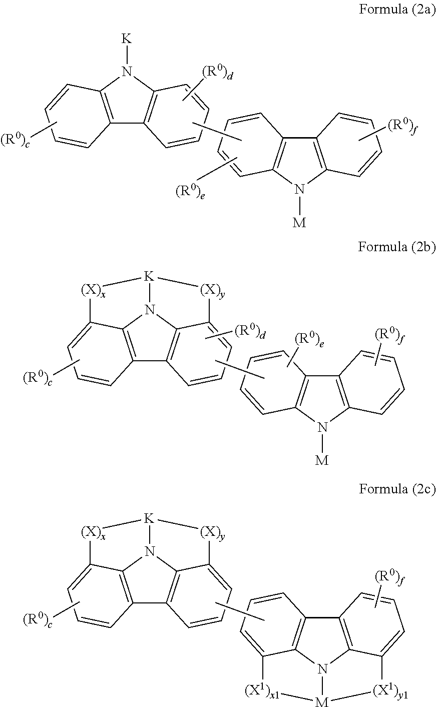
where the symbols and indices X, X¹, R⁰, c, d, e and f used are as defined in claim 16 and
K and M in compounds of the formula (2a) are each independently an unsubstituted or partly or fully deuterated or R*-monosubstituted aromatic ring system having 6 to 40 ring atoms;
M in compounds of the formula (2b) is an unsubstituted or partly or fully deuterated or R*-monosubstituted aromatic ring system having 6 to 40 ring atoms;
K in compounds of the formula (2b) together with X forms a heteroaromatic ring system having 14 to 40 ring atoms and x and y in compounds of the formula (2b) each independently represent 0 or 1 and the sum of x and y is at least 1; and
K and M in compounds of the formula (2c) each independently together with X or X¹ form a heteroaromatic ring system having 14 to 40 ring atoms; and
x, x1, y and y1 in compounds of the formula (2c) each independently represent 0 or 1 and the sum of x and y is at least 1 and the sum of x1 and y1 is at least 1.

20. The organic electroluminescent device according to claim 16, wherein the organic electroluminescent device is selected from the group consisting of organic light-emitting transistors (OLETs), organic field quench devices (OFQDs), organic light-emitting electrochemical cells (OLECs, LECs, LEECs), organic laser diodes (O-lasers) and organic light-emitting diodes (OLEDs).

21. The organic electroluminescent device according to claim 16, characterized in that this organic layer comprises, in addition to the light-emitting layer (EML), a hole injection layer (HIL), a hole transport layer (HTL), an electron transport layer (ETL), an electron injection layer (EIL) and/or a hole blocker layer (HBL).

22. The organic electroluminescent device according to claim 16, characterized in that the light-emitting layer, as well as the at least one host material 1 and the at least one host material 2, contains at least one phosphorescent emitter.

23. The organic electroluminescent device according to claim 16, characterized in that the organic layer comprises a hole injection layer (HIL) and/or a hole transport layer (HTL), the hole-injecting material and hole-transporting material of which is a monoamine that does not contain a carbazole unit.

24. A process for producing a device according to claim 16, which comprises apply the organic layer by gas phase deposition or from solution.

25. The process according to claim 24, characterized in that to produce the light-emitting layer the at least one compound of the formula (1) and the at least one compound of the formula (2) are deposited from the gas phase successively or simultaneously from at least two material sources, optionally with the at least one phosphorescent emitter.

26. The process according to claim 24, characterized in that to produce the light-emitting layer the at least one compound of the formula (1) and the at least one compound of the formula (2) are deposited from the gas phase as a mixture, successively or simultaneously with the at least one phosphorescent emitter.

27. The process according to claim 24, characterized in that to produce the light-emitting layer the at least one compound of the formula (1) and the at least one compound of the formula (2) are applied from a solution together with the at least one phosphorescent emitter.

28. A mixture comprising at least one compound of the formula (1) and at least one compound of the formula (2), 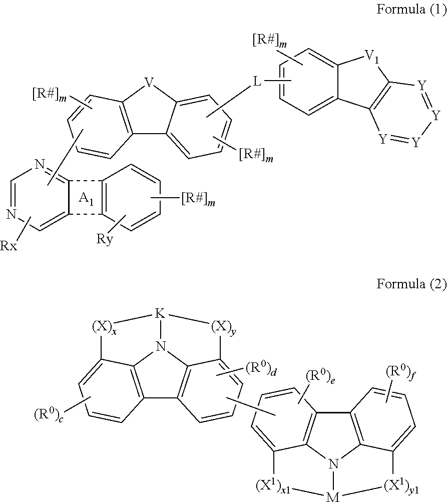
where the symbols and indices used are as follows:
ring A₁ in formula (1) conforms to the formula (1A) 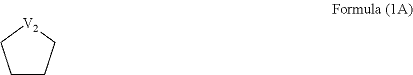
V is O or S;
V₁ is O, S, C(R¹)₂ or N-A;
V₂ is O or S;
Y is the same or different at each instance and is independently CH, CR or CA, or two adjacent Y groups together form a group of the formula (1B) 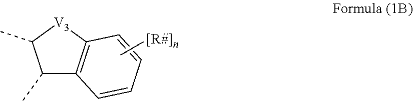
and the remaining Y groups are independently CH or CR, where V₃ is O, S, C(R¹)₂ or N-A and the dotted bonds show the linkage of this group to the remainder of the formula (1);
Rx is L₁-R*;
Ry is L₂-R*;
R* independently at each instance is an aromatic or heteroaromatic ring system which has 6 to 14 ring atoms and may be substituted by one or more R radicals;
L, L₁, L₂ are each independently a bond or a phenylene group which may each be substituted by one or more R radicals;
A is the same or different at each instance and is an aromatic or heteroaromatic ring system which has 6 to 30 ring atoms and may be substituted by one or more R radicals;
n is 0, 1, 2, 3 or 4;
m is 0, 1, 2 or 3;
R # is D or an aryl group which has 6 to 12 carbon atoms and may be substituted by one or more R radicals;
R¹ is the same or different at each instance and is an alkyl group having 1 to 20 carbon atoms, phenyl which may be substituted by one or more R radicals, or the two R¹ radicals together with the carbon atom to which they bind form a spiro compound;
R is the same or different at each instance and is selected from D, F, CN, a straight-chain alkyl group having 1 to 20 carbon atoms or a branched or cyclic alkyl group having 3 to 20 carbon atoms, where one or more nonadjacent CH₂ groups may be replaced by R²C═CR², O or S and where one or more hydrogen atoms may be replaced by D, F, or CN;
R² is the same or different at each instance and is selected from the group consisting of H, D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
K, M are each independently an unsubstituted or partly or fully deuterated or R*-monosubstituted aromatic ring system having 6 to 40 ring atoms when x and y are 0 and when x1 and y1 are 0, or
K, M each independently together with X or X¹ form a heteroaromatic ring system having 14 to 40 ring atoms when the value of x, x1, y and/or y1 is 1;
x, x1 at each instance are each independently 0 or 1;
y, y1 at each instance are each independently 0 or 1;
X and X¹ at each instance are each independently a bond or C(R⁺)₂;
R⁰ at each instance is independently an unsubstituted or partly or fully deuterated aromatic ring system having 6 to 18 carbon atoms;
R⁺ at each instance is independently a straight-chain or branched alkyl group having 1 to 4 carbon atoms and
c, d, e and f are independently 0 or 1.

29. The mixture according to claim 28, characterized in that the mixture consists of at least one compound of the formula (1), at least one compound of the formula (2) and a phosphorescent emitter.

30. formulation comprising the mixture according to claim 28 and at least one solvent.
